(19) United States
(12) Patent Application Publication
(10) Pub. No.: US 2022/0380834 A1
(43) Pub. Date: Dec. 1, 2022

(54) METHODS AND COMPOSITIONS FOR HIGH SENSITIVITY DETECTION OF DRUG RESISTANCE MARKERS

(71) Applicant: T2 Biosystems, Inc., Lexington, MA (US)

(72) Inventors: Jessica Lee SNYDER, Woburn, MA (US); Brendan John MANNING, Arlington, MA (US); Daniel GAMERO, Medford, MA (US); Heidi Susanne GIESE, Newburyport, MA (US); Thomas J. LOWERY, Jr., Belmont, MA (US)

(21) Appl. No.: 17/282,305

(22) PCT Filed: Oct. 4, 2019
(86) PCT No.: PCT/US2019/054623

§ 371 Date: Apr. 1, 2021

Related U.S. Application Data
(60) Provisional application No. 62/863,582, filed on Jun. 19, 2019.
(60) Provisional application No. 62/741,369, filed on Oct. 4, 2018.

(51) Int. Cl. C12Q 1/689; G01N 27/74

(52) U.S. Cl. CPC C12Q 1/689; C12Q 2600/16; G01N 27/745

(57) ABSTRACT

Provided herein are methods of amplifying and detecting drug resistance markers (e.g., antibiotic resistance genes) from pathogens in complex samples (e.g., blood), as well as related panels and compositions (e.g., systems, cartridges, and kits).

### SEQUENCE LISTING

The instant application contains a Sequence Listing which has been submitted electronically in ASCII format and is hereby incorporated by reference in its entirety. Said ASCII copy, created on Oct. 3, 2019, is named 50713-127WO3_Sequence_Listing_10.03.19_ST25 and is 18,749 bytes in size.

### FIELD OF THE INVENTION

The invention features methods and compositions for amplifying and detecting drug resistance markers (e.g., antibiotic resistance genes) in complex samples, for example, blood (e.g., whole blood). The methods and compositions can be used, e.g., to inform treatment decisions.

### BACKGROUND OF THE INVENTION

Drug resistance, such as antibiotic resistance in bacteria, is one of the biggest threats to global health, food security, and development. Antibiotic-resistant bacteria lead to increased mortality, longer hospital stays, and higher medical costs, and can affect patients of all ages and demographics. In particular, bacterial resistance to a class of important antibiotic agents known as carbapenems is a growing concern. Carbapenemase-producing Enterobacteriaceae (CPEs) are members of a large family of Gram-negative bacteria (including Escherichia, Klebsiella, and Enterobacter) that have developed resistance to carbapenems. In 2015, in the European Union countries of Italy, Greece, Romania, Malta, and Cyprus, between 5% and 62% of invasive Klebsiella pneumoniae isolates were resistant to carbapenems. The proportion of drug-resistant organisms is expected to increase under the use of current diagnostic tools and empirical therapy. This is problematic because most CPEs are also resistant to all first-line anti-Gram-negative antibiotics, e.g., cephalosporins, fluoroquinolones, and β-lactam-β-lactamase inhibitors, and are thus difficult to treat.

Appropriate therapy for infection by CPEs started within five days of infection is associated with lower mortality. However, current diagnostic methods typically require 1-5 days of blood culture before diagnostic tests (e.g., antimicrobial susceptibility testing or genomic testing) can be performed. This delay in detection of CPEs can negatively impact patient outcomes, and inappropriate disease treatment preceding detection may also contribute to the spread of multidrug-resistant organisms.

Thus, there is an unmet need in the art for methods for the rapid detection of antibiotic resistance genes (e.g., Gram-negative carbapenem resistance genes, extended spectrum beta lactamase (ESBL) resistance genes, and Gram-positive resistance genes) as well as pathogens directly from complex samples (e.g., whole blood).

### SUMMARY OF THE INVENTION

The invention features, inter alia, methods of amplifying and detecting drug resistance markers (e.g., antibiotic resistance genes) from pathogens (e.g., drug-resistant pathogens, e.g., carbapenem-resistant Gram-negative bacteria) in complex samples (e.g., blood), as well as related panels and compositions (e.g., systems, cartridges, and kits).

In one aspect, the invention features a method for detecting the presence of an antibiotic resistance gene in a biological sample, the method comprising: (a) amplifying in a biological sample or a fraction thereof one or more antibiotic resistance target nucleic acids in a multiplexed amplification reaction, wherein the multiplexed amplification reaction is configured to amplify target nucleic acids characteristic of two or more antibiotic resistance genes selected from the group consisting of NDM, KPC, IMP, VIM, OXA-48-like, DHA, CMY, FOX, mecA, mecC, vanA, vanB, CTX-M 14, CTX-M 15, mefA, mefE, ermA, ermB, SHV, and TEM, wherein the two or more antibiotic resistance genes comprises at least one of IMP, OXA-48-like, DHA, CMY, FOX, CTX-M 14, CTX-M 15, mecC, mefA, mefE, ermA, ermB, or TEM; and (b) detecting the one or more amplified antibiotic resistance target nucleic acids to determine whether one or more of the antibiotic resistance genes is present in the biological sample, wherein the method individually detects an antibiotic resistance gene of a pathogen present at a concentration of 10 cells/mL of biological sample or less.

In some embodiments: (i) the method comprises amplifying and/or detecting at least three, at least four, at least five, at least six, or all seven antibiotic resistance genes selected from the group consisting of NDM, KPC, IMP, VIM, OXA-48-like, DHA, and CMY; and/or (ii) the multiplexed amplification reaction is configured to amplify at least three, at least four, at least five, at least six, or all seven antibiotic resistance genes selected from the group consisting of NDM, KPC, IMP, VIM, OXA-48-like, DHA, and CMY. In some embodiments: (i) the method comprises amplifying and/or detecting NDM, KPC, IMP, VIM, OXA-48-like, DHA, and CMY; and/or (ii) the multiplexed amplification reaction is configured to amplify NDM, KPC, IMP, VIM, OXA-48-like, DHA, and CMY. In some embodiments: (i) NDM is amplified in the presence of a forward primer comprising the nucleotide sequence CCAGCTCGCACCGAATGTCT (SEQ ID NO: 1) and a reverse primer comprising the nucleotide sequence CATCTTGTCCTGATGCGCGTGAGTCA (SEQ ID NO: 2); (ii) KPC is amplified in the presence of a forward primer comprising the nucleotide sequence TTTCTGCCACCGCGCTGAC (SEQ ID NO: 3) and a reverse primer comprising the nucleotide sequence GCAGCAAGAAAGCCCTTGAATG (SEQ ID NO: 4); (iii) IMP is amplified in the presence of a forward primer comprising the nucleotide sequence ACATTTCCATAGCGACAGCACGGGCGGAAT (SEQ ID NO: 5) and a reverse primer comprising the nucleotide sequence GGACTTTGGCCAAGCTTCTAAATTTGCGTC (SEQ ID NO: 6); (iv) VIM is amplified in the presence of a forward primer comprising the nucleotide sequence CGTGCAGTCTCCACGCACT (SEQ ID NO: 7) and a reverse primer comprising the nucleotide sequence TCGAATGCGCAGCACCGGGATAG (SEQ ID NO: 8); (v) OXA-48-like is amplified in the presence of a forward primer comprising the nucleotide sequence GGCTGTGTTTTGGTGGCATCGATTATC (SEQ ID NO: 9) and a reverse primer comprising the nucleotide sequence TCCCACTTAAAGACTTGGTGTTCATCC (SEQ ID NO: 10); (vi) DHA is amplified in the presence of a forward primer comprising the nucleotide sequence ACGGGCCGGTAATGCGGATCTGGA (SEQ ID NO: 11) and a reverse primer comprising the nucleotide sequence TATTCGCCAGAATCACAATCGCCACCTGT (SEQ ID NO: 12); and/or (vii) CMY is amplified in the presence of a forward primer comprising the nucleotide sequence CCGCGGCGAAATTAAGCTCAGCGA (SEQ ID NO: 13) and a reverse primer comprising the nucleotide sequence CCAAACAGACCAATGCTGGAGTTAG (SEQ ID NO: 14).

In some embodiments: (i) the method comprises amplifying and/or detecting at least three, at least four, at least five, at least six, at least seven, at least eight, at least nine, at least ten, at least eleven, at least twelve, or all thirteen antibiotic resistance genes selected from the group consisting of KPC, CTX-M 14, CTX-M 15, NDM, VIM, IMP, vanA, vanB, mecA, mecC, OXA-48-like, CMY, and DHA; and/or (ii) the multiplexed amplification reaction is configured to amplify at least three, at least four, at least five, at least six, at least seven, at least eight, at least nine, at least ten, at least eleven, at least twelve, or all thirteen antibiotic resistance genes selected from the group consisting of KPC, CTX-M 14, CTX-M 15, NDM, VIM, IMP, vanA, vanB, mecA, mecC, OXA-48-like, CMY, and DHA. In some embodiments: (i) the method comprises amplifying and/or detecting KPC, CTX-M 14, CTX-M 15, NDM, VIM, IMP, vanA, vanB, mecA, mecC, OXA-48-like, CMY, and DHA; and/or (ii) the multiplexed amplification reaction is configured to amplify KPC, CTX-M 14, CTX-M 15, NDM, VIM, IMP, vanA, vanB, mecA, mecC, OXA-48-like, CMY, and DHA. In some embodiments: (i) KPC is amplified in the presence of a forward primer comprising the nucleotide sequence TTTCTGCCACCGCGCTGAC (SEQ ID NO: 3) and a reverse primer comprising the nucleotide sequence GCAGCAAGAAAGCCCTTGAATG (SEQ ID NO: 4); (ii) CTX-M 14 is amplified in the presence of a forward primer comprising the nucleotide sequence ACGCTTTCCAATGTGCAGTACCAGTA (SEQ ID NO: 33) and a reverse primer comprising the nucleotide sequence TGCGATCCAGACGAAACGTCTCATCG (SEQ ID NO: 34); (iii) CTX-M 15 is amplified in the presence of a forward primer comprising the nucleotide sequence CCTCGGGCAATGGCGCAAAC (SEQ ID NO: 81) and a reverse primer comprising the nucleotide sequence ATCGCGACGGCTTTCTGCCTTA (SEQ ID NO: 82); (iv) NDM is amplified in the presence of a forward primer comprising the nucleotide sequence CCAGCTCGCACCGAATGTCT (SEQ ID NO: 1) and a reverse primer comprising the nucleotide sequence CATCTTGTCCTGATGCGCGTGAGTCA (SEQ ID NO: 2); (v) VIM is amplified in the presence of a forward primer comprising the nucleotide sequence CGTGCAGTCTCCACGCACT (SEQ ID NO: 7) and a reverse primer comprising the nucleotide sequence TCGAATGCGCAGCACCGGGATAG (SEQ ID NO: 8); (vi) IMP is amplified in the presence of a forward primer comprising the nucleotide sequence ACATTTCCATAGCGACAGCACGGGCGGAAT (SEQ ID NO: 5) and a reverse primer comprising the nucleotide sequence GGACTTTGGCCAAGCTTCTAAATTTGCGTC (SEQ ID NO: 6); (vii) vanA is amplified in the presence of a forward primer comprising the nucleotide sequence TATTCATCAGGAAGTCGAGCCGGA (SEQ ID NO: 85) and a reverse primer comprising the nucleotide sequence CAGTTCGGGAAGTGCAATACCTGCA (SEQ ID NO: 50); (viii) vanB is amplified in the presence of a forward primer comprising the nucleotide sequence AATTGAGCAAGCGATTTCGGGCTGT (SEQ ID NO: 51) and a reverse primer comprising the nucleotide sequence AAGATCAACACGGGCAAGCCCTCT (SEQ ID NO: 88); (ix) mecA is amplified in the presence of a forward primer comprising the nucleotide sequence ATGTTGGTCCCATTAACTCTGAAGAA (SEQ ID NO: 41) and a reverse primer comprising the nucleotide sequence CACCTGTTTGAGGGTGGATAGCAGTA (SEQ ID NO: 42); (x) mecC is amplified in the presence of a forward primer comprising the nucleotide sequence ATGTGGGTCCAATTAATTCTGACGAG (SEQ ID NO: 91) and a reverse primer comprising the nucleotide sequence CTCCAGTTTTGGTTGTAATGCTGTA (SEQ ID NO: 92); (xi) OXA-48-like is amplified in the presence of a forward primer comprising the nucleotide sequence GGCTGTGTTTITGGTGGCATCGATTATC (SEQ ID NO: 9) and a reverse primer comprising the nucleotide sequence TCCCACTTAAAGACTTGGTGTTCATCC (SEQ ID NO: 10); (xii) CMY is amplified in the presence of a forward primer comprising the nucleotide sequence CCGCGGCGAAATTAAGCTCAGCGA (SEQ ID NO: 13) and a reverse primer comprising the nucleotide sequence CCAAACAGACCAATGCTGGAGTTAG (SEQ ID NO: 14); and/or (xiii) DHA is amplified in the presence of a forward primer comprising the nucleotide sequence ACGGGCCGGTAATGCGGATCTGGA (SEQ ID NO: 11) and a reverse primer comprising the nucleotide sequence TATTCGCCAGAATCACAATCGCCACCTGT (SEQ ID NO: 12).

In some embodiments: (i) the method comprises amplifying and/or detecting at least three, at least four, at least five, at least six, at least seven, at least eight, at least nine, or all ten antibiotic resistance genes selected from the group consisting of CTX-M 14, CTX-M 15, ermA, ermB, mecA, mefA, SHV, TEM, vanA, and vanB; and/or (ii) the multiplexed amplification reaction is configured to amplify at least three, at least four, at least five, at least six, at least seven, at least eight, at least nine, or all ten antibiotic resistance genes selected from the group consisting of CTX-M 14, CTX-M 15, ermA, ermB, mecA, mefA, SHV, TEM, vanA, and vanB. In some embodiments: (i) the method comprises amplifying and/or detecting CTX-M 14, CTX-M 15, ermA, ermB, mecA, mefA, SHV, TEM, vanA, and vanB; and/or (ii) the multiplexed amplification reaction is configured to amplify CTX-M 14, CTX-M 15, ermA, ermB, mecA, mefA, SHV, TEM, vanA, and vanB. In some embodiments: (i) CTX-M 14 is amplified in the presence of a forward primer comprising the nucleotide sequence ACGCTTTCCAATGTGCAGTACCAGTA (SEQ ID NO: 33) and a reverse primer comprising the nucleotide sequence TGCGATCCAGACGAAACGTCTCATCG (SEQ ID NO: 34); (ii) CTX-M 15 is amplified in the presence of a forward primer comprising the nucleotide sequence GTGATACCACTTCACCTCGGGCAA (SEQ ID NO: 35) and a reverse primer comprising the nucleotide sequence AATACATCGCGACGGCTTTCTGCC (SEQ ID NO: 36); (iii) ermA is amplified in the presence of a forward primer comprising the nucleotide sequence AGAATTACCTTTGAAAGTCAGGC (SEQ ID NO: 37) and a reverse primer comprising the nucleotide sequence GCTTCAAAGCCTGTCGGAATTGGTTT (SEQ ID NO: 38); (iv) ermB is amplified in the presence of a forward primer comprising the nucleotide sequence GGGCATTTAACGACGAAACTGGCTA (SEQ ID NO: 39) and a reverse primer comprising the nucleotide sequence GTGTTCGGTGAATATCCAAGGTACGC (SEQ ID NO: 40); (v) mecA is amplified in the presence of a forward primer comprising the nucleotide sequence ATGTTGGTCCCATTAACTCTGAAGAA (SEQ ID NO: 41) and a reverse primer comprising the nucleotide sequence CACCTGTTTGAGGGTGGATAGCAGTA (SEQ ID NO: 42); (vi) mefA is amplified in the presence of a forward primer comprising the nucleotide sequence GCAGGGCAAGCAGTATCATTAATCAC (SEQ ID NO: 43) and a reverse primer comprising the nucleotide sequence AATTAAATCAGCACCAATCATTATCTTCTTCC (SEQ ID NO: 44); (vii) SHV is amplified in the presence of a forward primer comprising the nucleotide sequence AAGCTGCTGACCAGCCAGCGTCTGA (SEQ ID NO: 45) and a reverse primer comprising the nucleotide sequence CGGCGATTTGCTGATTTCGCTCG (SEQ ID NO: 46); (viii) TEM is amplified in the presence of a forward primer comprising the nucleotide sequence TGCAGTGCTGCCATAACCATGAGTGA (SEQ ID NO: 47) and a reverse primer comprising the nucleotide sequence AGCGCAGAAGTGGTCCTGCAACTTT (SEQ ID NO: 48); (ix) vanA is amplified in the presence of a forward primer comprising the nucleotide sequence CAGTACGGAATCTTTCGTATTCATCAGGA (SEQ ID NO: 49) and a reverse primer comprising the nucleotide sequence CAGTTCGGGAAGTGCAATACCTGCA (SEQ ID NO: 50); and/or (x) vanB is amplified in the presence of a forward primer comprising the nucleotide sequence AATTGAGCAAGCGATTTCGGGCTGT (SEQ ID NO: 51) and a reverse primer comprising the nucleotide sequence CGTTTAGAACGATGCCGCCATCCT (SEQ ID NO: 52).

In some embodiments: (i) amplifying step (a) further comprises amplifying a target nucleic acid characteristic of a bacterial pathogen, and detecting step (b) further comprises detecting the amplified target nucleic acid characteristic of a bacterial pathogen to determine whether the bacterial pathogen is present; and/or (ii) the multiplexed amplification reaction is configured to amplify a target nucleic acid characteristic of a bacterial pathogen. In some embodiments, the target nucleic acid characteristic of a bacterial pathogen is characteristic of an Enterobacter spp., a Klebsiella spp., or Streptococcus pneumoniae. In some embodiments, the target nucleic acid characteristic of a bacterial pathogen is characteristic of Enterobacter spp. and Klebsiella spp. In some embodiments, the target nucleic acid characteristic of Enterobacter spp. and Klebsiella spp. is amplified in the presence of a forward primer comprising the nucleotide sequence ATTCGTTGCACTATCGTTAACTGAATACA (SEQ ID NO: 15) and a reverse primer comprising the nucleotide sequence CTGTACCGTCGGACTTTCCAGAC (SEQ ID NO: 16). In some embodiments, the target nucleic acid characteristic of a bacterial pathogen is characteristic of Streptococcus pneumoniae. In some embodiments, the target nucleic acid characteristic of Streptococcus pneumoniae is amplified in the presence of a forward primer comprising the nucleotide sequence CCTTGGACGGAAATGTAGCTGGCA (SEQ ID NO: 53) and a reverse primer comprising the nucleotide sequence AATCACATGGTTGACACCTGCTGTG (SEQ ID NO: 54).

In some embodiments: (i) amplifying step (a) further comprises amplifying an internal amplification control (IC) target nucleic acid and detecting step (b) further comprises detecting the amplified IC target nucleic acid; and/or (ii) the multiplexed amplification reaction is configured to amplify an amplified IC target nucleic acid.

In some embodiments, the method detects an antibiotic resistance gene of a pathogen present at a concentration of 2 cells/mL of biological sample or less. In some embodiments, the method detects an antibiotic resistance gene of a pathogen present at a concentration of 1 cells/mL of biological sample.

In some embodiments, the detecting of step (b) comprises magnetic, sequencing, optical, fluorescent, mass, density, chromatographic, and/or electrochemical detection.

In some embodiments, the detecting of step (b) comprises T2 magnetic resonance (T2MR).

In some embodiments, the detecting of step (b) comprises sequencing.

In another aspect, featured herein is a method for detecting the presence of an antibiotic resistance gene in a biological sample, the method comprising: (a) providing a biological sample; (b) lysing pathogen cells in the biological sample; (c) amplifying in the product of step (b) one or more antibiotic resistance target nucleic acids in a multiplexed amplification reaction to form an amplified biological sample, wherein the multiplexed amplification reaction is configured to amplify target nucleic acids characteristic of two or more antibiotic resistance genes selected from the group consisting of NDM, KPC, IMP, VIM, OXA-48-like, DHA, CMY, FOX, mecA, vanA, vanB, CTX-M 14, CTX-M 15, mefA, mecC, mefE, ermA, ermB, SHV, and TEM, wherein the two or more antibiotic resistance genes comprises at least one of IMP, OXA-48-like, DHA, CMY, FOX, CTX-M 14, CTX-M 15, mecC, mefA, mefE, ermA, ermB, or TEM; (d) preparing a first assay sample by contacting a portion of the amplified biological sample with a first population of magnetic particles, wherein the magnetic particles of the first population have binding moieties characteristic of a first antibiotic resistance gene on their surface, the binding moieties operative to alter aggregation of the magnetic particles in the presence of a first amplified antibiotic resistance target nucleic acid; (e) preparing a second assay sample by contacting a portion of the amplified biological sample with a second population of magnetic particles, wherein the magnetic particles of the second population have binding moieties characteristic of a second antibiotic resistance gene on their surface, the binding moieties operative to alter aggregation of the magnetic particles in the presence of a second amplified antibiotic resistance target nucleic acid; (f) providing each assay sample in a detection tube within a device, the device comprising a support defining a well for holding the detection tube comprising the assay sample, and having an RF coil configured to detect a signal produced by exposing the mixture to a bias magnetic field created using one or more magnets and an RF pulse sequence; (g) exposing each assay sample to a bias magnetic field and an RF pulse sequence; (h) following step (g), measuring the signal produced by each assay sample; and (i) on the basis of the result of step (h), detecting whether one or more of the antibiotic resistance genes is present in the biological sample.

In some embodiments: the method comprises amplifying and/or detecting at least three, at least four, at least five, at least six, or all seven antibiotic resistance genes selected from the group consisting of NDM, KPC, IMP, VIM, OXA-48-like, DHA, and CMY; and/or the multiplexed amplification reaction is configured to amplify at least three, at least four, at least five, at least six, or all seven antibiotic resistance genes selected from the group consisting of NDM, KPC, IMP, VIM, OXA-48-like, DHA, and CMY. In some embodiments: the method comprises amplifying and/or detecting NDM, KPC, IMP, VIM, OXA-48-like, DHA, and CMY; and/or the multiplexed amplification reaction is configured to amplify NDM, KPC, IMP, VIM, OXA-48-like, DHA, and CMY. In some embodiments: (i) NDM is amplified in the presence of a forward primer comprising the nucleotide sequence CCAGCTCGCACCGAATGTCT (SEQ ID NO: 1) and a reverse primer comprising the nucleotide sequence CATCTTGTCCTGATGCGCGTGAGTCA (SEQ ID NO: 2); (ii) KPC is amplified in the presence of a forward primer comprising the nucleotide sequence TTTCTGCCACCGCGCTGAC (SEQ ID NO: 3) and a reverse primer comprising the nucleotide sequence GCAGCAAGAAAGCCCTTGAATG (SEQ ID NO: 4); (iii) IMP is amplified in the presence of a forward primer comprising the nucleotide sequence ACATTTCCATAGCGACAGCACGGGCGGAAT (SEQ ID NO: 5) and a reverse primer comprising the nucleotide sequence GGACTTTGGCCAAGCTTCTAAATTTGCGTC (SEQ ID NO: 6); (iv) VIM is amplified in the presence of a forward primer comprising the nucleotide sequence CGTGCAGTCTCCACGCACT (SEQ ID NO: 7) and a reverse primer comprising the nucleotide sequence TCGAATGCGCAGCACCGGGATAG (SEQ ID NO: 8); (v) OXA-48-like is amplified in the presence of a forward primer comprising the nucleotide sequence GGCTGTGTTTITGGTGGCATCGATTATC (SEQ ID NO: 9) and a reverse primer comprising the nucleotide sequence TCCCACTTAAAGACTTGGTGTTCATCC (SEQ ID NO: 10); (vi) DHA is amplified in the presence of a forward primer comprising the nucleotide sequence ACGGGCCGGTAATGCGGATCTGGA (SEQ ID NO: 11) and a reverse primer comprising the nucleotide sequence TATTCGCCAGAATCACAATCGCCACCTGT (SEQ ID NO: 12); and/or (vii) CMY is amplified in the presence of a forward primer comprising the nucleotide sequence CCGCGGCGAAATTAAGCTCAGCGA (SEQ ID NO: 13) and a reverse primer comprising the nucleotide sequence CCAAACAGACCAATGCTGGAGTTAG (SEQ ID NO: 14).

In some embodiments: (i) the method comprises amplifying and/or detecting at least three, at least four, at least five, at least six, at least seven, at least eight, at least nine, at least ten, at least eleven, at least twelve, or all thirteen antibiotic resistance genes selected from the group consisting of KPC, CTX-M 14, CTX-M 15, NDM, VIM, IMP, vanA, vanB, mecA, mecC, OXA-48-like, CMY, and DHA; and/or (ii) the multiplexed amplification reaction is configured to amplify at least three, at least four, at least five, at least six, at least seven, at least eight, at least nine, at least ten, at least eleven, at least twelve, or all thirteen antibiotic resistance genes selected from the group consisting of KPC, CTX-M 14, CTX-M 15, NDM, VIM, IMP, vanA, vanB, mecA, mecC, OXA-48-like, CMY, and DHA. In some embodiments: (i) the method comprises amplifying and/or detecting KPC, CTX-M 14, CTX-M 15, NDM, VIM, IMP, vanA, vanB, mecA, mecC, OXA-48-like, CMY, and DHA; and/or (ii) the multiplexed amplification reaction is configured to amplify KPC, CTX-M 14, CTX-M 15, NDM, VIM, IMP, vanA, vanB, mecA, mecC, OXA-48-like, CMY, and DHA. In some embodiments: (i) KPC is amplified in the presence of a forward primer comprising the nucleotide sequence TTTCTGCCACCGCGCTGAC (SEQ ID NO: 3) and a reverse primer comprising the nucleotide sequence GCAGCAAGAAAGCCCTTGAATG (SEQ ID NO: 4); (ii) CTX-M 14 is amplified in the presence of a forward primer comprising the nucleotide sequence ACGCTTTCCAATGTGCAGTACCAGTA (SEQ ID NO: 33) and a reverse primer comprising the nucleotide sequence TGCGATCCAGACGAAACGTCTCATCG (SEQ ID NO: 34); (iii) CTX-M 15 is amplified in the presence of a forward primer comprising the nucleotide sequence CCTCGGGCAATGGCGCAAAC (SEQ ID NO: 81) and a reverse primer comprising the nucleotide sequence ATCGCGACGGCTTTCTGCCTTA (SEQ ID NO: 82); (iv) NDM is amplified in the presence of a forward primer comprising the nucleotide sequence CCAGCTCGCACCGAATGTCT (SEQ ID NO: 1) and a reverse primer comprising the nucleotide sequence CATCTTGTCCTGATGCGCGTGAGTCA (SEQ ID NO: 2); (v) VIM is amplified in the presence of a forward primer comprising the nucleotide sequence CGTGCAGTCTCCACGCACT (SEQ ID NO: 7) and a reverse primer comprising the nucleotide sequence TCGAATGCGCAGCACCGGGATAG (SEQ ID NO: 8); (vi) IMP is amplified in the presence of a forward primer comprising the nucleotide sequence ACATTTCCATAGCGACAGCACGGGCGGAAT (SEQ ID NO: 5) and a reverse primer comprising the nucleotide sequence GGACTTTGGCCAAGCTTCTAAATTTGCGTC (SEQ ID NO: 6); (vii) vanA is amplified in the presence of a forward primer comprising the nucleotide sequence TATTCATCAGGAAGTCGAGCCGGA (SEQ ID NO: 85) and a reverse primer comprising the nucleotide sequence CAGTTCGGGAAGTGCAATACCTGCA (SEQ ID NO: 50); (viii) vanB is amplified in the presence of a forward primer comprising the nucleotide sequence AATTGAGCAAGCGATTTCGGGCTGT (SEQ ID NO: 51) and a reverse primer comprising the nucleotide sequence AAGATCAACACGGGCAAGCCCTCT (SEQ ID NO: 88); (ix) mecA is amplified in the presence of a forward primer comprising the nucleotide sequence ATGTTGGTCCCATTAACTCTGAAGAA (SEQ ID NO: 41) and a reverse primer comprising the nucleotide sequence CACCTGTTTGAGGGTGGATAGCAGTA (SEQ ID NO: 42); (x) mecC is amplified in the presence of a forward primer comprising the nucleotide sequence ATGTGGGTCCAATTAATTCTGACGAG (SEQ ID NO: 91) and a reverse primer comprising the nucleotide sequence CTCCAGTTTGGTTGTAATGCTGTA (SEQ ID NO: 92); (xi) OXA-48-like is amplified in the presence of a forward primer comprising the nucleotide sequence GGCTGTGTTTITGGTGGCATCGATTATC (SEQ ID NO: 9) and a reverse primer comprising the nucleotide sequence TCCCACTTAAAGACTTGGTGTTCATCC (SEQ ID NO: 10); (xii) CMY is amplified in the presence of a forward primer comprising the nucleotide sequence CCGCGGCGAAATTAAGCTCAGCGA (SEQ ID NO: 13) and a reverse primer comprising the nucleotide sequence CCAAACAGACCAATGCTGGAGTTAG (SEQ ID NO: 14); and/or (xiii) DHA is amplified in the presence of a forward primer comprising the nucleotide sequence ACGGGCCGGTAATGCGGATCTGGA (SEQ ID NO: 11) and a reverse primer comprising the nucleotide sequence TATTCGCCAGAATCACAATCGCCACCTGT (SEQ ID NO: 12).

In some embodiments: (i) the method comprises amplifying and/or detecting at least three, at least four, at least five, at least six, at least seven, at least eight, at least nine, or all ten antibiotic resistance genes selected from the group consisting of CTX-M 14, CTX-M 15, ermA, ermB, mecA, mefA, SHV, TEM, vanA, and vanB; and/or (ii) the multiplexed amplification reaction is configured to amplify at least three, at least four, at least five, at least six, at least seven, at least eight, at least nine, or all ten antibiotic resistance genes selected from the group consisting of CTX-M 14, CTX-M 15, ermA, ermB, mecA, mefA, SHV, TEM, vanA, and vanB. In some embodiments: (i) the method comprises amplifying and/or detecting CTX-M 14, CTX-M 15, ermA, ermB, mecA, mefA, SHV, TEM, vanA, and vanB; and/or (ii) the multiplexed amplification reaction is configured to amplify CTX-M 14, CTX-M 15, ermA, ermB, mecA, mefA, SHV, TEM, vanA, and vanB. In some embodiments: (i) CTX-M 14 is amplified in the presence of a forward primer comprising the nucleotide sequence ACGCTTTCCAATGTGCAGTACCAGTA (SEQ ID NO: 33) and a reverse primer comprising the nucleotide sequence TGCGATCCAGACGAAACGTCTCATCG (SEQ ID NO: 34); (ii) CTX-M 15 is amplified in the presence of a forward primer comprising the nucleotide sequence GTGATACCACTTCACCTCGGGCAA (SEQ ID NO: 35) and a reverse primer comprising the nucleotide sequence AATACATCGCGACGGCTTTCTGCC (SEQ ID NO: 36); (iii) ermA is amplified in the presence of a forward primer comprising the nucleotide sequence AGAATTACCTTTGAAAGTCAGGC (SEQ ID NO: 37) and a reverse primer comprising the nucleotide sequence GCTTCAAAGCCTGTCGGAATTGGTTT (SEQ ID NO: 38); (iv) ermB is amplified in the presence of a forward primer comprising the nucleotide sequence GGGCATTTAACGACGAAACTGGCTA (SEQ ID NO: 39) and a reverse primer comprising the nucleotide sequence GTGTTCGGTGAATATCCAAGGTACGC (SEQ ID NO: 40); (v) mecA is amplified in the presence of a forward primer comprising the nucleotide sequence ATGTTGGTCCCATTAACTCTGAAGAA (SEQ ID NO: 41) and a reverse primer comprising the nucleotide sequence CACCTGTTTGAGGGTGGATAGCAGTA (SEQ ID NO: 42); (vi) mefA is amplified in the presence of a forward primer comprising the nucleotide sequence GCAGGGCAAGCAGTATCATTAATCAC (SEQ ID NO: 43) and a reverse primer comprising the nucleotide sequence AATTAAATCAGCACCAATCATTATCTTCTTCC (SEQ ID NO: 44); (vii) SHV is amplified in the presence of a forward primer comprising the nucleotide sequence AAGCTGCTGACCAGCCAGCGTCTGA (SEQ ID NO: 45) and a reverse primer comprising the nucleotide sequence CGGCGATTTGCTGATTTCGCTCG (SEQ ID NO: 46); (viii) TEM is amplified in the presence of a forward primer comprising the nucleotide sequence TGCAGTGCTGCCATAACCATGAGTGA (SEQ ID NO: 47) and a reverse primer comprising the nucleotide sequence AGCGCAGAAGTGGTCCTGCAACTTT (SEQ ID NO: 48); (ix) vanA is amplified in the presence of a forward primer comprising the nucleotide sequence CAGTACGGAATCTTTCGTATTCATCAGGA (SEQ ID NO: 49) and a reverse primer comprising the nucleotide sequence CAGTTCGGGAAGTGCAATACCTGCA (SEQ ID NO: 50); and/or (x) vanB is amplified in the presence of a forward primer comprising the nucleotide sequence AATTGAGCAAGCGATTTCGGGCTGT (SEQ ID NO: 51) and a reverse primer comprising the nucleotide sequence CGTTTAGAACGATGCCGCCATCCT (SEQ ID NO: 52).

In some embodiments: amplifying step (c) further comprises amplifying a target nucleic acid characteristic of a bacterial pathogen, and step (i) further comprises detecting the amplified target nucleic acid characteristic of a bacterial pathogen to determine whether the bacterial pathogen is present; and/or the multiplexed amplification reaction is configured to amplify a target nucleic acid characteristic of a bacterial pathogen. In some embodiments, the target nucleic acid characteristic of a bacterial pathogen is characteristic of an Enterobacter spp., a Klebsiella spp., or Streptococcus pneumoniae. In some embodiments, the target nucleic acid characteristic of a bacterial pathogen is characteristic of Enterobacter spp. and Klebsiella spp. In some embodiments, the target nucleic acid characteristic of Enterobacter spp. and Klebsiella spp. is amplified in the presence of a forward primer comprising the nucleotide sequence ATTCGTTGCACTATCGTTAACTGAATACA (SEQ ID NO: 15) and a reverse primer comprising the nucleotide sequence CTGTACCGTCGGACTTTCCAGAC (SEQ ID NO: 16). In some embodiments, the target nucleic acid characteristic of a bacterial pathogen is characteristic of Streptococcus pneumoniae. In some embodiments, the target nucleic acid characteristic of Streptococcus pneumoniae is amplified in the presence of a forward primer comprising the nucleotide sequence CCTTGGACGGAAATGTAGCTGGCA (SEQ ID NO: 53) and a reverse primer comprising the nucleotide sequence AATCACATGGTTGACACCTGCTGTG (SEQ ID NO: 54).

In some embodiments: amplifying step (c) further comprises amplifying an IC target nucleic acid and step (i) further comprises detecting the amplified IC target nucleic acid; and/or the multiplexed amplification reaction is configured to amplify an amplified IC target nucleic acid.

In some embodiments, the magnetic particles of each population comprise two subpopulations, a first subpopulation bearing a first probe on its surface, and a second subpopulation bearing a second probe on its surface. In some embodiments, the magnetic particles of each population comprise two subpopulations, a first subpopulation bearing a first probe and a second probe on its surface, and a second subpopulation bearing a third probe and a fourth probe on its surface.

In some embodiments, the method comprises amplifying two or more antibiotic resistance genes selected from the group consisting of NDM, KPC, IMP, VIM, OXA-48-like, DHA, and/or CMY, and wherein: (i) a probe pair comprising a 5′ probe comprising the nucleotide sequence GCGACCGGCAGGTTGATCT (SEQ ID NO: 17) or GCGACCGGCAGGTTGATCTCCTGC (SEQ ID NO: 95) and a 3′ probe comprising the nucleotide sequence CATGTCGAGATAGGAAGTGTGCTGC (SEQ ID NO: 18) or CGGCATGTCGAGATAGGAAGTGTGCTGC (SEQ ID NO: 96) is used for detection of NDM; (ii) a probe pair comprising a 5′ probe comprising the nucleotide sequence CGGAACCATTCGCTAAACTC (SEQ ID NO: 19) and a 3′ probe comprising the nucleotide sequence AGGCGCAACTGTAAGTTACCG (SEQ ID NO: 20) is used for detection of KPC; (iii) a probe pair comprising a 5′ probe comprising the nucleotide sequence GCTTAATTCTCAATCTATCCCCACGTAT (SEQ ID NO: 21) and a 3′ probe comprising the nucleotide sequence CTCCAGATAACGTAGTGGTTTGGCTG (SEQ ID NO: 22) is used for detection of IMP; (iv) a probe pair comprising a 5′ probe comprising the nucleotide sequence CTTTCATGACGACCGCGTCGG (SEQ ID NO: 23) and a 3′ probe comprising the nucleotide sequence CTCTAGAAGGACTCTCATCGAGC (SEQ ID NO: 24) is used for detection of VIM; (v) a probe pair comprising a 5′ probe comprising the nucleotide sequence ATTTTAAAGGTAGATGCGGG (SEQ ID NO: 25) and a 3′ probe comprising the nucleotide sequence CGCCCTGTGATTTATGTTCA (SEQ ID NO: 26) is used for detection of OXA-48-like; (vi) a probe pair comprising a 5′ probe comprising the nucleotide sequence GTTTTATGCACCCAGGAAGC (SEQ ID NO: 27) and a 3′ probe comprising the nucleotide sequence TCTGCTGCGGCCAGTCATA (SEQ ID NO: 28) is used for detection of DHA; and/or (vii) a probe pair comprising a 5′ probe comprising the nucleotide sequence GCGGCTGCCAGTTTTGATAA (SEQ ID NO: 29) and a 3′ probe comprising the nucleotide sequence GTGGCTAAGTGCAGCAGGC (SEQ ID NO: 30) is used for detection of CMY. In some embodiments, a probe pair comprising a 5′ probe comprising the nucleotide sequence GCGACCGGCAGGTTGATCT (SEQ ID NO: 17) and a 3′ probe comprising the nucleotide sequence CATGTCGAGATAGGAAGTGTGCTGC (SEQ ID NO: 18) is used for detection of NDM. In other embodiments, a probe pair comprising a 5′ probe comprising the nucleotide sequence GCGACCGGCAGGTTGATCTCCTGC (SEQ ID NO: 95) and a 3′ probe comprising the nucleotide sequence CGGCATGTCGAGATAGGAAGTGTGCTGC (SEQ ID NO: 96) is used for detection of NDM.

In some embodiments, the method comprises amplifying a target nucleic acid characteristic of Enterobacter spp. and Klebsiella spp., and a probe pair comprising a 5′ probe comprising the nucleotide sequence CGTTCCACTAACACACAAGCTGATTCAG (SEQ ID NO: 31) and a 3′ probe comprising the nucleotide sequence ATCTCGGTTGATTTCTTTTCCTCGGG (SEQ ID NO: 32) is used for detection of the target nucleic acid characteristic of Enterobacter spp. and Klebsiella spp.

In some embodiments, the method comprises amplifying two or more antibiotic resistance genes selected from the group consisting of KPC, CTX-M 14, CTX-M 15, NDM, VIM, IMP, vanA, vanB, mecA, mecC, OXA-48-like, CMY, and DHA, and wherein: (i) a probe pair comprising a 5′ probe comprising the nucleotide sequence CGGAACCATTCGCTAAACTC (SEQ ID NO: 19) and a 3′ probe comprising the nucleotide sequence AGGCGCAACTGTAAGTTACCG (SEQ ID NO: 20) is used for detection of KPC; (ii) a probe pair comprising a 5′ probe comprising the nucleotide sequence CTGTCGAGATCAAGCCTGCCGA (SEQ ID NO: 57) and a 3′ probe comprising the nucleotide sequence ACAAATTGATTGCCCAGCTCGGT (SEQ ID NO: 58) is used for detection of CTX-M 14; (iii) a probe pair comprising a 5′ probe comprising the nucleotide sequence GGGCGCAGCTGGTGACAT (SEQ ID NO: 83) and a 3′ probe comprising the nucleotide sequence AAGATCGTGCGCCGCTGATT (SEQ ID NO: 84) is used for detection of CTX-M 15; (iv) a probe pair comprising a 5′ probe comprising the nucleotide sequence GCGACCGGCAGGTTGATCT (SEQ ID NO: 17) or GCGACCGGCAGGTTGATCTCCTGC (SEQ ID NO: 95) and a 3′ probe comprising the nucleotide sequence CATGTCGAGATAGGAAGTGTGCTGC (SEQ ID NO: 18) or CGGCATGTCGAGATAGGAAGTGTGCTGC (SEQ ID NO: 96) is used for detection of NDM; (v) a probe pair comprising a 5′ probe comprising the nucleotide sequence CTTTCATGACGACCGCGTCGG (SEQ ID NO: 23) and a 3′ probe comprising the nucleotide sequence CTCTAGAAGGACTCTCATCGAGC (SEQ ID NO: 24) is used for detection of VIM; (vi) a probe pair comprising a 5′ probe comprising the nucleotide sequence GCTTAATTCTCAATCTATCCCCACGTAT (SEQ ID NO: 21) and a 3′ probe comprising the nucleotide sequence CTCCAGATAACGTAGTGGTTTGGCTG (SEQ ID NO: 22) is used for detection of IMP; (vii) a probe pair comprising a 5′ probe comprising the nucleotide sequence GCAGTTATAACCGTTCCCGCAG (SEQ ID NO: 86) and a 3′ probe comprising the nucleotide sequence TAACGGCCGCATTGTACTGAACG (SEQ ID NO: 87) is used for detection of vanA; (viii) a probe pair comprising a 5′ probe comprising the nucleotide sequence GCACCCGATATACTTTCTTTGCC (SEQ ID NO: 75) and a 3′ probe comprising the nucleotide sequence CGCCGACAATCAAATCATCCT (SEQ ID NO: 76) is used for detection of vanB; (ix) a probe pair comprising a 5′ probe comprising the nucleotide sequence AAAGGCTATAAAGATGATGCAG (SEQ ID NO: 89) and a 3′ probe comprising the nucleotide sequence GAGTATTTATAACAACATGAAAAATGATT (SEQ ID NO: 90) is used for detection of mecA; (x) a probe pair comprising a 5′ probe comprising the nucleotide sequence GGCTTAGAACGCCTCTATGAT (SEQ ID NO: 93) and a 3′ probe comprising the nucleotide sequence AGAGTACAAGAAAGTATTTATAAACATATGA (SEQ ID NO: 94) is used for detection of mecC; (xi) a probe pair comprising a 5′ probe comprising the nucleotide sequence ATTTTAAAGGTAGATGCGGG (SEQ ID NO: 25) and a 3′ probe comprising the nucleotide sequence CGCCCTGTGATTTATGTTCA (SEQ ID NO: 26) is used for detection of OXA-48-like; (xii) a probe pair comprising a 5′ probe comprising the nucleotide sequence GCGGCTGCCAGTTTTGATAA (SEQ ID NO: 29) and a 3′ probe comprising the nucleotide sequence GTGGCTAAGTGCAGCAGGC (SEQ ID NO: 30) is used for detection of CMY; and/or (xiii) a probe pair comprising a 5′ probe comprising the nucleotide sequence GTTTTATGCACCCAGGAAGC (SEQ ID NO: 27) and a 3′ probe comprising the nucleotide sequence TCTGCTGCGGCCAGTCATA (SEQ ID NO: 28) is used for detection of DHA. In some embodiments, a probe pair comprising a 5′ probe comprising the nucleotide sequence GCGACCGGCAGGTTGATCT (SEQ ID NO: 17) and a 3′ probe comprising the nucleotide sequence CATGTCGAGATAGGAAGTGTGCTGC (SEQ ID NO: 18) is used for detection of NDM. In other embodiments, a probe pair comprising a 5′ probe comprising the nucleotide sequence GCGACCGGCAGGTTGATCTCCTGC (SEQ ID NO: 95) and a 3′ probe comprising the nucleotide sequence CGGCATGTCGAGATAGGAAGTGTGCTGC (SEQ ID NO: 96) is used for detection of NDM.

In some embodiments, the method comprises amplifying two or more antibiotic resistance genes selected from the group consisting of CTX-M 14, CTX-M 15, ermA, ermB, mecA, mefA, SHV, TEM, vanA, and vanB, and wherein: (i) a probe pair comprising a 5′ probe comprising the nucleotide sequence CTGTCGAGATCAAGCCTGCCGA (SEQ ID NO: 57) and a 3′ probe comprising the nucleotide sequence ACAAATTGATTGCCCAGCTCGGT (SEQ ID NO: 58) is used for detection of CTX-M 14; (ii) a probe pair comprising a 5′ probe comprising the nucleotide sequence AATCAGCGGCGCACGATCTTT (SEQ ID NO: 59) and a 3′ probe comprising the nucleotide sequence AATGCTCGCTGCACCGGTGGTAT (SEQ ID NO: 60) is used for detection of CTX-M 15; (iii) a probe pair comprising a 5′ probe comprising the nucleotide sequence AAATCTGCAACGAGCTTTGGG (SEQ ID NO: 61) and a 3′ probe comprising the nucleotide sequence GTTTATAAGTGGGTAAACCGTGAATATC (SEQ ID NO: 62) is used for detection of ermA; (iv) a probe pair comprising a 5′ probe comprising the nucleotide sequence TTCGTGTCACTTTAATTCACCAAGAT (SEQ ID NO: 63) and a 3′ probe comprising the nucleotide sequence AAAGCCATGCGTCTGACATCT (SEQ ID NO: 64) is used for detection of ermB; (v) a probe pair comprising a 5′ probe comprising the nucleotide sequence AAGCTCCAACATGAAGATGGCT (SEQ ID NO: 65) and a 3′ probe comprising the nucleotide sequence AGATGGCAAAGATATTCAACTAAC (SEQ ID NO: 66) is used for detection of mecA; (vi) a probe pair comprising a 5′ probe comprising the nucleotide sequence AGTGCCATCTTGCAAATGGCGAT (SEQ ID NO: 67) and a 3′ probe comprising the nucleotide sequence TGCAATTGGTGTGTTAGTGGATCGTCATGATA (SEQ ID NO: 68) is used for detection of mefA; (vii) a probe pair comprising a 5′ probe comprising the nucleotide sequence CAGTGGATGGTGGACGATCGGGT (SEQ ID NO: 69) and a 3′ probe comprising the nucleotide sequence TTGTGGTGATTTATCTGCGGGATACT (SEQ ID NO: 70) is used for detection of SHV; (viii) a probe pair comprising a 5′ probe comprising the nucleotide sequence CGCCAGTTAATAGTTTGCGCAACG (SEQ ID NO: 71) and a 3′ probe comprising the nucleotide sequence AAAGCGGTTAGCTCCTTCGGTCCT (SEQ ID NO: 72) is used for detection of TEM; (ix) a probe pair comprising a 5′ probe comprising the nucleotide sequence CGTTCAGTACAATGCGGCCGTTA (SEQ ID NO: 73) and a 3′ probe comprising the nucleotide sequence CTGCGGGAACGGTTATAACTGC (SEQ ID NO: 74) is used for detection of vanA; and/or (x) a probe pair comprising a 5′ probe comprising the nucleotide sequence GCACCCGATATACTTTCTTTGCC (SEQ ID NO: 75) and a 3′ probe comprising the nucleotide sequence CGCCGACAATCAAATCATCCT (SEQ ID NO: 76) is used for detection of vanB.

In some embodiments, the method comprises amplifying a target nucleic acid characteristic of Streptococcus pneumoniae, and a probe pair comprising a 5′ probe comprising the nucleotide sequence TTGACCAGTTCCGAGCAAATGGTA (SEQ ID NO: 77) and a 3′ probe comprising the nucleotide sequence GACAGTATCGATGTTCCAGCAGCT (SEQ ID NO: 78) is used for detection of the target nucleic acid characteristic of Streptococcus pneumoniae.

In some embodiments, an assay sample is contacted with 1×10⁶ to 1×10¹³ magnetic particles per milliliter of the biological sample.

In some embodiments, step (h) comprises measuring the T₂ relaxation response of the assay sample, and wherein increasing agglomeration in the assay sample produces an increase in the observed T₂ relaxation time of the assay sample.

In some embodiments, the magnetic particles have a mean diameter of from 600 nm to 1200 nm. In some embodiments, magnetic particles have a mean diameter of from 650 nm to 950 nm. In some embodiments, the magnetic particles have a mean diameter of from 670 nm to 890 nm.

In some embodiments, the magnetic particles have a T₂ relaxivity per particle of from 1×10⁹ to 1×10¹² mM⁻¹ s⁻¹.

In some embodiments, the magnetic particles are substantially monodisperse.

In some embodiments, the method further comprises sequencing the first and/or second amplified antibiotic resistance target nucleic acid.

In another aspect, the invention features a method for detecting the presence of an antibiotic resistance gene in a biological sample obtained from a subject, wherein the biological sample comprises subject-derived cells or cell debris, the method comprising: (a) amplifying in the biological sample or a fraction thereof one or more antibiotic resistance target nucleic acids in a multiplexed amplification reaction, wherein the multiplexed amplification reaction is configured to amplify target nucleic acids characteristic of two or more antibiotic resistance genes selected from the group consisting of NDM, KPC, IMP, VIM, OXA-48-like, DHA, CMY, FOX, mecA, mecC, vanA, vanB, CTX-M 14, CTX-M 15, mefA, mefE, ermA, ermB, SHV, and TEM, wherein the two or more antibiotic resistance genes comprises at least one of IMP, OXA-48-like, DHA, CMY, FOX, CTX-M 14, CTX-M 15, mecC, mefA, mefE, ermA, ermB, or TEM; and (b) sequencing the one or more amplified antibiotic resistance target nucleic acids to detect whether one or more of the antibiotic resistance genes is present in the biological sample, wherein the method is capable of detecting an antibiotic resistance gene of a pathogen present at a concentration of 10 cells/mL of biological sample or less.

In some embodiments: (i) the method comprises amplifying and/or sequencing at least three, at least four, at least five, at least six, or all seven antibiotic resistance genes selected from the group consisting of NDM, KPC, IMP, VIM, OXA-48-like, DHA, and CMY; and/or (ii) the multiplexed amplification reaction is configured to amplify at least three, at least four, at least five, at least six, or all seven antibiotic resistance genes selected from the group consisting of NDM, KPC, IMP, VIM, OXA-48-like, DHA, and CMY. In some embodiments: (i) the method comprises amplifying and/or sequencing NDM, KPC, IMP, VIM, OXA-48-like, DHA, and CMY; and/or (ii) the multiplexed amplification reaction is configured to amplify NDM, KPC, IMP, VIM, OXA-48-like, DHA, and CMY. In some embodiments: (i) NDM is amplified in the presence of a forward primer comprising the nucleotide sequence CCAGCTCGCACCGAATGTCT (SEQ ID NO: 1) and a reverse primer comprising the nucleotide sequence CATCTTGTCCTGATGCGCGTGAGTCA (SEQ ID NO: 2); (ii) KPC is amplified in the presence of a forward primer comprising the nucleotide sequence TTTCTGCCACCGCGCTGAC (SEQ ID NO: 3) and a reverse primer comprising the nucleotide sequence GCAGCAAGAAAGCCCTTGAATG (SEQ ID NO: 4); (iii) IMP is amplified in the presence of a forward primer comprising the nucleotide sequence ACATTTCCATAGCGACAGCACGGGCGGAAT (SEQ ID NO: 5) and a reverse primer comprising the nucleotide sequence GGACTTTGGCCAAGCTTCTAAATTTGCGTC (SEQ ID NO: 6); (iv) VIM is amplified in the presence of a forward primer comprising the nucleotide sequence CGTGCAGTCTCCACGCACT (SEQ ID NO: 7) and a reverse primer comprising the nucleotide sequence TCGAATGCGCAGCACCGGGATAG (SEQ ID NO: 8); (v) OXA-48-like is amplified in the presence of a forward primer comprising the nucleotide sequence GGCTGTGTTTTGGTGGCATCGATTATC (SEQ ID NO: 9) and a reverse primer comprising the nucleotide sequence TCCCACTTAAAGACTTGGTGTTCATCC (SEQ ID NO: 10); (vi) DHA is amplified in the presence of a forward primer comprising the nucleotide sequence ACGGGCCGGTAATGCGGATCTGGA (SEQ ID NO: 11) and a reverse primer comprising the nucleotide sequence TATTCGCCAGAATCACAATCGCCACCTGT (SEQ ID NO: 12); and/or (vii) CMY is amplified in the presence of a forward primer comprising the nucleotide sequence CCGCGGCGAAATTAAGCTCAGCGA (SEQ ID NO: 13) and a reverse primer comprising the nucleotide sequence CCAAACAGACCAATGCTGGAGTTAG (SEQ ID NO: 14).

In some embodiments: (i) the method comprises amplifying and/or sequencing at least three, at least four, at least five, at least six, at least seven, at least eight, at least nine, at least ten, at least eleven, at least twelve, or all thirteen antibiotic resistance genes selected from the group consisting of KPC, CTX-M 14, CTX-M 15, NDM, VIM, IMP, vanA, vanB, mecA, mecC, OXA-48-like, CMY, and DHA; and/or (ii) the multiplexed amplification reaction is configured to amplify at least three, at least four, at least five, at least six, at least seven, at least eight, at least nine, at least ten, at least eleven, at least twelve, or all thirteen antibiotic resistance genes selected from the group consisting of KPC, CTX-M 14, CTX-M 15, NDM, VIM, IMP, vanA, vanB, mecA, mecC, OXA-48-like, CMY, and DHA. In some embodiments: (i) the method comprises amplifying and/or sequencing KPC, CTX-M 14, CTX-M 15, NDM, VIM, IMP, vanA, vanB, mecA, mecC, OXA-48-like, CMY, and DHA; and/or (ii) the multiplexed amplification reaction is configured to amplify KPC, CTX-M 14, CTX-M 15, NDM, VIM, IMP, vanA, vanB, mecA, mecC, OXA-48-like, CMY, and DHA. In some embodiments: (i) KPC is amplified in the presence of a forward primer comprising the nucleotide sequence TTTCTGCCACCGCGCTGAC (SEQ ID NO: 3) and a reverse primer comprising the nucleotide sequence GCAGCAAGAAAGCCCTTGAATG (SEQ ID NO: 4); (ii) CTX-M 14 is amplified in the presence of a forward primer comprising the nucleotide sequence ACGCTTTCCAATGTGCAGTACCAGTA (SEQ ID NO: 33) and a reverse primer comprising the nucleotide sequence TGCGATCCAGACGAAACGTCTCATCG (SEQ ID NO: 34); (iii) CTX-M 15 is amplified in the presence of a forward primer comprising the nucleotide sequence CCTCGGGCAATGGCGCAAAC (SEQ ID NO: 81) and a reverse primer comprising the nucleotide sequence ATCGCGACGGCTTTCTGCCTTA (SEQ ID NO: 82); (iv) NDM is amplified in the presence of a forward primer comprising the nucleotide sequence CCAGCTCGCACCGAATGTCT (SEQ ID NO: 1) and a reverse primer comprising the nucleotide sequence CATCTTGTCCTGATGCGCGTGAGTCA (SEQ ID NO: 2); (v) VIM is amplified in the presence of a forward primer comprising the nucleotide sequence CGTGCAGTCTCCACGCACT (SEQ ID NO: 7) and a reverse primer comprising the nucleotide sequence TCGAATGCGCAGCACCGGGATAG (SEQ ID NO: 8); (vi) IMP is amplified in the presence of a forward primer comprising the nucleotide sequence ACATTTCCATAGCGACAGCACGGGCGGAAT (SEQ ID NO: 5) and a reverse primer comprising the nucleotide sequence GGACTTTGGCCAAGCTTCTAAATTTGCGTC (SEQ ID NO: 6); (vii) vanA is amplified in the presence of a forward primer comprising the nucleotide sequence TATTCATCAGGAAGTCGAGCCGGA (SEQ ID NO: 85) and a reverse primer comprising the nucleotide sequence CAGTTCGGGAAGTGCAATACCTGCA (SEQ ID NO: 50); (viii) vanB is amplified in the presence of a forward primer comprising the nucleotide sequence AATTGAGCAAGCGATTTCGGGCTGT (SEQ ID NO: 51) and a reverse primer comprising the nucleotide sequence AAGATCAACACGGGCAAGCCCTCT (SEQ ID NO: 88); (ix) mecA is amplified in the presence of a forward primer comprising the nucleotide sequence ATGTTGGTCCCATTAACTCTGAAGAA (SEQ ID NO: 41) and a reverse primer comprising the nucleotide sequence CACCTGTTTGAGGGTGGATAGCAGTA (SEQ ID NO: 42); (x) mecC is amplified in the presence of a forward primer comprising the nucleotide sequence ATGTGGGTCCAATTAATTCTGACGAG (SEQ ID NO: 91) and a reverse primer comprising the nucleotide sequence CTCCAGTTTTGGTTGTAATGCTGTA (SEQ ID NO: 92); (xi) OXA-48-like is amplified in the presence of a forward primer comprising the nucleotide sequence GGCTGTGTTTITGGTGGCATCGATTATC (SEQ ID NO: 9) and a reverse primer comprising the nucleotide sequence TCCCACTTAAAGACTTGGTGTTCATCC (SEQ ID NO: 10); (xii) CMY is amplified in the presence of a forward primer comprising the nucleotide sequence CCGCGGCGAAATTAAGCTCAGCGA (SEQ ID NO: 13) and a reverse primer comprising the nucleotide sequence CCAAACAGACCAATGCTGGAGTTAG (SEQ ID NO: 14); and/or (xiii) DHA is amplified in the presence of a forward primer comprising the nucleotide sequence ACGGGCCGGTAATGCGGATCTGGA (SEQ ID NO: 11) and a reverse primer comprising the nucleotide sequence TATTCGCCAGAATCACAATCGCCACCTGT (SEQ ID NO: 12).

In some embodiments: (i) the method comprises amplifying and/or sequencing at least three, at least four, at least five, at least six, at least seven, at least eight, at least nine, or all ten antibiotic resistance genes selected from the group consisting of CTX-M 14, CTX-M 15, ermA, ermB, mecA, mefA, SHV, TEM, vanA, and vanB; and/or (ii) the multiplexed amplification reaction is configured to amplify at least three, at least four, at least five, at least six, at least seven, at least eight, at least nine, or all ten antibiotic resistance genes selected from the group consisting of CTX-M 14, CTX-M 15, ermA, ermB, mecA, mefA, SHV, TEM, vanA, and vanB. In some embodiments: (i) the method comprises amplifying and/or sequencing CTX-M 14, CTX-M 15, ermA, ermB, mecA, mefA, SHV, TEM, vanA, and vanB; and/or (ii) the multiplexed amplification reaction is configured to amplify CTX-M 14, CTX-M 15, ermA, ermB, mecA, mefA, SHV, TEM, vanA, and vanB. In some embodiments: (i) CTX-M 14 is amplified in the presence of a forward primer comprising the nucleotide sequence ACGCTTTCCAATGTGCAGTACCAGTA (SEQ ID NO: 33) and a reverse primer comprising the nucleotide sequence TGCGATCCAGACGAAACGTCTCATCG (SEQ ID NO: 34); (ii) CTX-M 15 is amplified in the presence of a forward primer comprising the nucleotide sequence GTGATACCACTTCACCTCGGGCAA (SEQ ID NO: 35) and a reverse primer comprising the nucleotide sequence AATACATCGCGACGGCTTTCTGCC (SEQ ID NO: 36); (iii) ermA is amplified in the presence of a forward primer comprising the nucleotide sequence AGAATTACCTTTGAAAGTCAGGC (SEQ ID NO: 37) and a reverse primer comprising the nucleotide sequence GCTTCAAAGCCTGTCGGAATTGGTTT (SEQ ID NO: 38); (iv) ermB is amplified in the presence of a forward primer comprising the nucleotide sequence GGGCATTTAACGACGAAACTGGCTA (SEQ ID NO: 39) and a reverse primer comprising the nucleotide sequence GTGTTCGGTGAATATCCAAGGTACGC (SEQ ID NO: 40); (v) mecA is amplified in the presence of a forward primer comprising the nucleotide sequence ATGTTGGTCCCATTAACTCTGAAGAA (SEQ ID NO: 41) and a reverse primer comprising the nucleotide sequence CACCTGTTTGAGGGTGGATAGCAGTA (SEQ ID NO: 42); (vi) mefA is amplified in the presence of a forward primer comprising the nucleotide sequence GCAGGGCAAGCAGTATCATTAATCAC (SEQ ID NO: 43) and a reverse primer comprising the nucleotide sequence AATTAAATCAGCACCAATCATTATCTTCTTCC (SEQ ID NO: 44); (vii) SHV is amplified in the presence of a forward primer comprising the nucleotide sequence AAGCTGCTGACCAGCCAGCGTCTGA (SEQ ID NO: 45) and a reverse primer comprising the nucleotide sequence CGGCGATTTGCTGATTTCGCTCG (SEQ ID NO: 46); (viii) TEM is amplified in the presence of a forward primer comprising the nucleotide sequence TGCAGTGCTGCCATAACCATGAGTGA (SEQ ID NO: 47) and a reverse primer comprising the nucleotide sequence AGCGCAGAAGTGGTCCTGCAACTTT (SEQ ID NO: 48); (ix) vanA is amplified in the presence of a forward primer comprising the nucleotide sequence CAGTACGGAATCTTTCGTATTCATCAGGA (SEQ ID NO: 49) and a reverse primer comprising the nucleotide sequence CAGTTCGGGAAGTGCAATACCTGCA (SEQ ID NO: 50); and/or (x) vanB is amplified in the presence of a forward primer comprising the nucleotide sequence AATTGAGCAAGCGATTTCGGGCTGT (SEQ ID NO: 51) and a reverse primer comprising the nucleotide sequence CGTTTAGAACGATGCCGCCATCCT (SEQ ID NO: 52).

In some embodiments: (i) amplifying step (a) further comprises amplifying a target nucleic acid characteristic of a bacterial pathogen, and step (b) further comprises sequencing the amplified target nucleic acid characteristic of a bacterial pathogen to determine whether the bacterial pathogen is present; and/or (ii) the multiplexed amplification reaction is configured to amplify a target nucleic acid characteristic of a bacterial pathogen. In some embodiments, the target nucleic acid characteristic of a bacterial pathogen is characteristic of an Enterobacter spp., a Klebsiella spp., or Streptococcus pneumoniae. In some embodiments, the target nucleic acid characteristic of a bacterial pathogen is characteristic of Enterobacter spp. and Klebsiella spp. In some embodiments, the target nucleic acid characteristic of Enterobacter spp. and Klebsiella spp. is amplified in the presence of a forward primer comprising the nucleotide sequence ATTCGTTGCACTATCGTTAACTGAATACA (SEQ ID NO: 15) and a reverse primer comprising the nucleotide sequence CTGTACCGTCGGACTTTCCAGAC (SEQ ID NO: 16). In some embodiments, the target nucleic acid characteristic of a bacterial pathogen is characteristic of Streptococcus pneumoniae. In some embodiments, the target nucleic acid characteristic of Streptococcus pneumoniae is amplified in the presence of a forward primer comprising the nucleotide sequence CCTTGGACGGAAATGTAGCTGGCA (SEQ ID NO: 53) and a reverse primer comprising the nucleotide sequence AATCACATGGTTGACACCTGCTGTG (SEQ ID NO: 54).

In some embodiments, step (a) comprises amplifying the one or more antibiotic resistance target nucleic acids in a lysate produced by lysing cells in the biological sample. In some embodiments, the lysate has at least about a 2:1, a 5:1, a 10:1, a 20:1, a 40:1, or a 60:1 higher concentration of cell debris relative to the biological sample. In some embodiments, the cell debris is solid material.

In some embodiments, the biological sample has a volume of about 0.1 mL to about 5 mL. In some embodiments, the biological sample has a volume of about 2 mL.

In some embodiments, the biological sample is selected from the group consisting of blood, a bloody fluid, a tissue sample, bronchiolar lavage (BAL), urine, cerebrospinal fluid (CSF), synovial fluid (SF), and sputum. In some embodiments, the blood is whole blood, a crude blood lysate, serum, or plasma. In some embodiments, the whole blood is ethylenediaminetetraacetic acid (EDTA) whole blood, sodium citrate whole blood, sodium heparin whole blood, lithium heparin whole blood, or potassium oxylate/sodium fluoride whole blood. In some embodiments, the bloody fluid is wound exudate, wound aspirate, phlegm, or bile. In some embodiments, the tissue sample is a tissue sample from a transplant, a tissue biopsy (e.g., a skin biopsy, muscle biopsy, or lymph node biopsy), a homogenized tissue sample, or bone. In some embodiments, the biological sample is urine or BAL. In some embodiments, the biological sample is a swab.

In some embodiments, the method further comprises detecting the amplified target pathogen nucleic acid(s) using T2 magnetic resonance (T2MR).

In another aspect, the invention features a method for detecting the presence of an antibiotic resistance gene in a whole blood sample, the method comprising: (a) contacting a whole blood sample suspected of containing one or more pathogen cells with an erythrocyte lysis agent, thereby lysing red blood cells; (b) centrifuging the product of step (a) to form a supernatant and a pellet; (c) discarding some or all of the supernatant of step (b) and resuspending the pellet to form an extract, optionally washing the pellet one or more times prior to resuspending the pellet; (d) lysing the remaining cells in the extract of step (c) to form a lysate, the lysate containing both subject cell nucleic acid and pathogen nucleic acid; (e) amplifying in the lysate of step (d) one or more antibiotic resistance target nucleic acids in a multiplexed amplification reaction, wherein the multiplexed amplification reaction is configured to amplify target nucleic acids characteristic of two or more antibiotic resistance genes selected from the group consisting of NDM, KPC, IMP, VIM, OXA-48-like, DHA, CMY, FOX, mecA, mecC, vanA, vanB, CTX-M 14, CTX-M 15, mefA, mefE, ermA, ermB, SHV, and TEM, wherein the two or more antibiotic resistance genes comprises at least one of IMP, OXA-48-like, DHA, CMY, FOX, CTX-M 14, CTX-M 15, mecC, mefA, mefE, ermA, ermB, or TEM; and (f) detecting the one or more amplified target antibiotic resistance target nucleic acids, thereby detecting the presence of the one or more of the antibiotic resistance genes in the sample. In some embodiments: (i) the method comprises amplifying and/or detecting at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, or all nineteen antibiotic resistance genes selected from the group consisting of NDM, KPC, IMP, VIM, OXA-48-like, DHA, CMY, mecA, mecC, vanA, vanB, CTX-M 14, CTX-M 15, mefA, mefE, ermA, ermB, SHV, and TEM; and/or (ii) the multiplexed amplification reaction is configured to amplify at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, or all nineteen antibiotic resistance genes selected from the group consisting of NDM, KPC, IMP, VIM, OXA-48-like, DHA, CMY, mecA, mecC, vanA, vanB, CTX-M 14, CTX-M 15, mefA, mefE, ermA, ermB, SHV, and TEM.

In some embodiments: (i) the method comprises amplifying and/or detecting at least three, at least four, at least five, at least six, or all seven antibiotic resistance genes selected from the group consisting of NDM, KPC, IMP, VIM, OXA-48-like, DHA, and CMY; and/or (ii) the multiplexed amplification reaction is configured to amplify at least three, at least four, at least five, at least six, or all seven antibiotic resistance genes selected from the group consisting of NDM, KPC, IMP, VIM, OXA-48-like, DHA, and CMY. In some embodiments: (i) the method comprises amplifying and/or detecting NDM, KPC, IMP, VIM, OXA-48-like, DHA, and CMY; and/or (ii) the multiplexed amplification reaction is configured to amplify NDM, KPC, IMP, VIM, OXA-48-like, DHA, and CMY. In some embodiments: (i) NDM is amplified in the presence of a forward primer comprising the nucleotide sequence CCAGCTCGCACCGAATGTCT (SEQ ID NO: 1) and a reverse primer comprising the nucleotide sequence CATCTTGTCCTGATGCGCGTGAGTCA (SEQ ID NO: 2); (ii) KPC is amplified in the presence of a forward primer comprising the nucleotide sequence TTTCTGCCACCGCGCTGAC (SEQ ID NO: 3) and a reverse primer comprising the nucleotide sequence GCAGCAAGAAAGCCCTTGAATG (SEQ ID NO: 4); (iii) IMP is amplified in the presence of a forward primer comprising the nucleotide sequence ACATTTCCATAGCGACAGCACGGGCGGAAT (SEQ ID NO: 5) and a reverse primer comprising the nucleotide sequence GGACTTTGGCCAAGCTTCTAAATTTGCGTC (SEQ ID NO: 6); (iv) VIM is amplified in the presence of a forward primer comprising the nucleotide sequence CGTGCAGTCTCCACGCACT (SEQ ID NO: 7) and a reverse primer comprising the nucleotide sequence TCGAATGCGCAGCACCGGGATAG (SEQ ID NO: 8); (v) OXA-48-like is amplified in the presence of a forward primer comprising the nucleotide sequence GGCTGTGTTTTGGTGGCATCGATTATC (SEQ ID NO: 9) and a reverse primer comprising the nucleotide sequence TCCCACTTAAAGACTTGGTGTTCATCC (SEQ ID NO: 10); (vi) DHA is amplified in the presence of a forward primer comprising the nucleotide sequence ACGGGCCGGTAATGCGGATCTGGA (SEQ ID NO: 11) and a reverse primer comprising the nucleotide sequence TATTCGCCAGAATCACAATCGCCACCTGT (SEQ ID NO: 12); and/or (vii) CMY is amplified in the presence of a forward primer comprising the nucleotide sequence CCGCGGCGAAATTAAGCTCAGCGA (SEQ ID NO: 13) and a reverse primer comprising the nucleotide sequence CCAAACAGACCAATGCTGGAGTTAG (SEQ ID NO: 14).

In some embodiments: (i) the method comprises amplifying and/or detecting at least three, at least four, at least five, at least six, at least seven, at least eight, at least nine, at least ten, at least eleven, at least twelve, or all thirteen antibiotic resistance genes selected from the group consisting of KPC, CTX-M 14, CTX-M 15, NDM, VIM, IMP, vanA, vanB, mecA, mecC, OXA-48-like, CMY, and DHA; and/or (ii) the multiplexed amplification reaction is configured to amplify at least three, at least four, at least five, at least six, at least seven, at least eight, at least nine, at least ten, at least eleven, at least twelve, or all thirteen antibiotic resistance genes selected from the group consisting of KPC, CTX-M 14, CTX-M 15, NDM, VIM, IMP, vanA, vanB, mecA, mecC, OXA-48-like, CMY, and DHA. In some embodiments: (i) the method comprises amplifying and/or detecting KPC, CTX-M 14, CTX-M 15, NDM, VIM, IMP, vanA, vanB, mecA, mecC, OXA-48-like, CMY, and DHA; and/or (ii) the multiplexed amplification reaction is configured to amplify KPC, CTX-M 14, CTX-M 15, NDM, VIM, IMP, vanA, vanB, mecA, mecC, OXA-48-like, CMY, and DHA. In some embodiments: (i) KPC is amplified in the presence of a forward primer comprising the nucleotide sequence TTTCTGCCACCGCGCTGAC (SEQ ID NO: 3) and a reverse primer comprising the nucleotide sequence GCAGCAAGAAAGCCCTTGAATG (SEQ ID NO: 4); (ii) CTX-M 14 is amplified in the presence of a forward primer comprising the nucleotide sequence ACGCTTTCCAATGTGCAGTACCAGTA (SEQ ID NO: 33) and a reverse primer comprising the nucleotide sequence TGCGATCCAGACGAAACGTCTCATCG (SEQ ID NO: 34); (iii) CTX-M 15 is amplified in the presence of a forward primer comprising the nucleotide sequence CCTCGGGCAATGGCGCAAAC (SEQ ID NO: 81) and a reverse primer comprising the nucleotide sequence ATCGCGACGGCTTTCTGCCTTA (SEQ ID NO: 82); (iv) NDM is amplified in the presence of a forward primer comprising the nucleotide sequence CCAGCTCGCACCGAATGTCT (SEQ ID NO: 1) and a reverse primer comprising the nucleotide sequence CATCTTGTCCTGATGCGCGTGAGTCA (SEQ ID NO: 2); (v) VIM is amplified in the presence of a forward primer comprising the nucleotide sequence CGTGCAGTCTCCACGCACT (SEQ ID NO: 7) and a reverse primer comprising the nucleotide sequence TCGAATGCGCAGCACCGGGATAG (SEQ ID NO: 8); (vi) IMP is amplified in the presence of a forward primer comprising the nucleotide sequence ACATTTCCATAGCGACAGCACGGGCGGAAT (SEQ ID NO: 5) and a reverse primer comprising the nucleotide sequence GGACTTTGGCCAAGCTTCTAAATTTGCGTC (SEQ ID NO: 6); (vii) vanA is amplified in the presence of a forward primer comprising the nucleotide sequence TATTCATCAGGAAGTCGAGCCGGA (SEQ ID NO: 85) and a reverse primer comprising the nucleotide sequence CAGTTCGGGAAGTGCAATACCTGCA (SEQ ID NO: 50); (viii) vanB is amplified in the presence of a forward primer comprising the nucleotide sequence AATTGAGCAAGCGATTTCGGGCTGT (SEQ ID NO: 51) and a reverse primer comprising the nucleotide sequence AAGATCAACACGGGCAAGCCCTCT (SEQ ID NO: 88); (ix) mecA is amplified in the presence of a forward primer comprising the nucleotide sequence ATGTTGGTCCCATTAACTCTGAAGAA (SEQ ID NO: 41) and a reverse primer comprising the nucleotide sequence CACCTGTTTGAGGGTGGATAGCAGTA (SEQ ID NO: 42); (x) mecC is amplified in the presence of a forward primer comprising the nucleotide sequence ATGTGGGTCCAATTAATTCTGACGAG (SEQ ID NO: 91) and a reverse primer comprising the nucleotide sequence CTCCAGTTTTGGTTGTAATGCTGTA (SEQ ID NO: 92); (xi) OXA-48-like is amplified in the presence of a forward primer comprising the nucleotide sequence GGCTGTGTTTITGGTGGCATCGATTATC (SEQ ID NO: 9) and a reverse primer comprising the nucleotide sequence TCCCACTTAAAGACTTGGTGTTCATCC (SEQ ID NO: 10); (xii) CMY is amplified in the presence of a forward primer comprising the nucleotide sequence CCGCGGCGAAATTAAGCTCAGCGA (SEQ ID NO: 13) and a reverse primer comprising the nucleotide sequence CCAAACAGACCAATGCTGGAGTTAG (SEQ ID NO: 14); and/or (xiii) DHA is amplified in the presence of a forward primer comprising the nucleotide sequence ACGGGCCGGTAATGCGGATCTGGA (SEQ ID NO: 11) and a reverse primer comprising the nucleotide sequence TATTCGCCAGAATCACAATCGCCACCTGT (SEQ ID NO: 12).

In some embodiments: (i) the method comprises amplifying and/or detecting at least three, at least four, at least five, at least six, at least seven, at least eight, at least nine, or all ten antibiotic resistance genes selected from the group consisting of CTX-M 14, CTX-M 15, ermA, ermB, mecA, mefA, SHV, TEM, vanA, and vanB; and/or (ii) the multiplexed amplification reaction is configured to amplify at least three, at least four, at least five, at least six, at least seven, at least eight, at least nine, or all ten antibiotic resistance genes selected from the group consisting of CTX-M 14, CTX-M 15, ermA, ermB, mecA, mefA, SHV, TEM, vanA, and vanB. In some embodiments: (i) the method comprises amplifying and/or detecting CTX-M 14, CTX-M 15, ermA, ermB, mecA, mefA, SHV, TEM, vanA, and vanB; and/or (ii) the multiplexed amplification reaction is configured to amplify CTX-M 14, CTX-M 15, ermA, ermB, mecA, mefA, SHV, TEM, vanA, and vanB. In some embodiments: (i) CTX-M 14 is amplified in the presence of a forward primer comprising the nucleotide sequence ACGCTTTCCAATGTGCAGTACCAGTA (SEQ ID NO: 33) and a reverse primer comprising the nucleotide sequence TGCGATCCAGACGAAACGTCTCATCG (SEQ ID NO: 34); (ii) CTX-M 15 is amplified in the presence of a forward primer comprising the nucleotide sequence GTGATACCACTTCACCTCGGGCAA (SEQ ID NO: 35) and a reverse primer comprising the nucleotide sequence AATACATCGCGACGGCTTTCTGCC (SEQ ID NO: 36); (iii) ermA is amplified in the presence of a forward primer comprising the nucleotide sequence AGAATTACCTTTGAAAGTCAGGC (SEQ ID NO: 37) and a reverse primer comprising the nucleotide sequence GCTTCAAAGCCTGTCGGAATTGGTTT (SEQ ID NO: 38); (iv) ermB is amplified in the presence of a forward primer comprising the nucleotide sequence GGGCATTTAACGACGAAACTGGCTA (SEQ ID NO: 39) and a reverse primer comprising the nucleotide sequence GTGTTCGGTGAATATCCAAGGTACGC (SEQ ID NO: 40); (v) mecA is amplified in the presence of a forward primer comprising the nucleotide sequence ATGTTGGTCCCATTAACTCTGAAGAA (SEQ ID NO: 41) and a reverse primer comprising the nucleotide sequence CACCTGTTTGAGGGTGGATAGCAGTA (SEQ ID NO: 42); (vi) mefA is amplified in the presence of a forward primer comprising the nucleotide sequence GCAGGGCAAGCAGTATCATTAATCAC (SEQ ID NO: 43) and a reverse primer comprising the nucleotide sequence AATTAAATCAGCACCAATCATTATCTTCTTCC (SEQ ID NO: 44); (vii) SHV is amplified in the presence of a forward primer comprising the nucleotide sequence AAGCTGCTGACCAGCCAGCGTCTGA (SEQ ID NO: 45) and a reverse primer comprising the nucleotide sequence CGGCGATTTGCTGATTTCGCTCG (SEQ ID NO: 46); (viii) TEM is amplified in the presence of a forward primer comprising the nucleotide sequence TGCAGTGCTGCCATAACCATGAGTGA (SEQ ID NO: 47) and a reverse primer comprising the nucleotide sequence AGCGCAGAAGTGGTCCTGCAACTTT (SEQ ID NO: 48); (ix) vanA is amplified in the presence of a forward primer comprising the nucleotide sequence CAGTACGGAATCTTTCGTATTCATCAGGA (SEQ ID NO: 49) and a reverse primer comprising the nucleotide sequence CAGTTCGGGAAGTGCAATACCTGCA (SEQ ID NO: 50); and/or (x) vanB is amplified in the presence of a forward primer comprising the nucleotide sequence AATTGAGCAAGCGATTTCGGGCTGT (SEQ ID NO: 51) and a reverse primer comprising the nucleotide sequence CGTTTAGAACGATGCCGCCATCCT (SEQ ID NO: 52).

In some embodiments: (i) amplifying step (e) further comprises amplifying a target nucleic acid characteristic of a bacterial pathogen, and step (f) further comprises detecting the amplified target nucleic acid characteristic of a bacterial pathogen to determine whether the bacterial pathogen is present; and/or (ii) the multiplexed amplification reaction is configured to amplify a target nucleic acid characteristic of a bacterial pathogen. In some embodiments, the target nucleic acid characteristic of a bacterial pathogen is characteristic of an Enterobacter spp., a Klebsiella spp., or Streptococcus pneumoniae. In some embodiments, the target nucleic acid characteristic of a bacterial pathogen is characteristic of Enterobacter spp. and Klebsiella spp. In some embodiments, the target nucleic acid characteristic of 20 Enterobacter spp. and Klebsiella spp. is amplified in the presence of a forward primer comprising the nucleotide sequence ATTCGTTGCACTATCGTTAACTGAATACA (SEQ ID NO: 15) and a reverse primer comprising the nucleotide sequence CTGTACCGTCGGACTTTCCAGAC (SEQ ID NO: 16). In some embodiments, the target nucleic acid characteristic of a bacterial pathogen is characteristic of Streptococcus pneumoniae. In some embodiments, the target nucleic acid characteristic of Streptococcus pneumoniae is amplified in the presence of a forward primer comprising the nucleotide sequence CCTTGGACGGAAATGTAGCTGGCA (SEQ ID NO: 53) and a reverse primer comprising the nucleotide sequence AATCACATGGTTGACACCTGCTGTG (SEQ ID NO: 54).

In some embodiments, step (c) comprises washing the pellet one time prior to resuspending the pellet.

In some embodiments, the washing or resuspending is performed with a wash buffer solution. In some embodiments, the wash buffer solution is Tris-EDTA (TE) buffer. In some embodiments, the washing is performed with a wash buffer solution having a volume of about 100 μL to about 500 μL. In some embodiments, the volume is about 150 μL.

In some embodiments, the resuspending of step (c) is performed with a wash buffer solution having a volume of about 50 μL to about 150 μL. In some embodiments, the volume is about 100 μL. In some embodiments: (i) the amplifying further comprises amplifying an IC target nucleic acid and the method further comprises the amplified IC target nucleic acid; and/or (ii) the multiplexed amplification reaction is configured to amplify an amplified IC target nucleic acid. In some embodiments, the IC target nucleic acid is amplified in the presence of a forward primer comprising the nucleotide sequence GGAAATCTAACGAGAGAGCATGCT (SEQ ID NO: 55) and a reverse primer comprising the nucleotide sequence CGATGCGTGACACCCAGGC (SEQ ID NO: 56). In some embodiments, the wash buffer solution further comprises an IC nucleic acid.

In some embodiments, step (a) further comprises adding a total process control (TPC) to the whole blood sample. In some embodiments, the TPC is an engineered cell comprising a control target nucleic acid.

In some embodiments, amplifying is in the presence of whole blood proteins and non-target nucleic acids.

In some embodiments, lysing comprises mechanical lysis or heat lysis. In some embodiments, the mechanical lysis is beadbeating or sonicating.

In some embodiments, the steps of the method are completed within 5 hours. In some embodiments, the steps of the method are completed within 4 hours. In some embodiments, the steps of the method are completed within 3 hours.

In some embodiments, the detecting comprises T2MR.

In some embodiments, the detecting comprises sequencing.

In some embodiments, the amplifying comprises polymerase chain reaction (PCR), ligase chain reaction (LCR), multiple displacement amplification (MDA), strand displacement amplification (SDA), rolling circle amplification (RCA), loop mediated isothermal amplification (LAMP), nucleic acid sequence based amplification (NASBA), helicase dependent amplification, recombinase polymerase amplification, nicking enzyme amplification reaction, or ramification amplification (RAM). In some embodiments, the amplifying comprises PCR. In some embodiments, the PCR is symmetric PCR or asymmetric PCR.

In some embodiments, the sequencing comprises massively parallel sequencing, Sanger sequencing, or single-molecule sequencing. In some embodiments, the massively parallel sequencing comprises sequencing by synthesis or sequencing by ligation. In some embodiments, the massively parallel sequencing comprises sequencing by synthesis. In some embodiments, the sequencing by synthesis comprises ILLUMINA™ dye sequencing, ion semiconductor sequencing, or pyrosequencing. In some embodiments, the sequencing by synthesis comprises ILLUMINA™ dye sequencing. In some embodiments, the sequencing by ligation comprises sequencing by oligonucleotide ligation and detection (SOLiD™) sequencing or polony-based sequencing. In some embodiments, the single-molecule sequencing is nanopore sequencing, single-molecule real-time (SMRT™) sequencing, or Helicos™ sequencing.

In some embodiments, the pathogen is a gram negative bacterial pathogen or a gram positive bacterial pathogen.

In some embodiments, the method comprises detecting any of the panels set forth herein, e.g., in Tables 1-15, 19, or 22.

In another aspect, the invention features a method for identifying a patient infected with an antibiotic resistant pathogen, the method comprising: (a) providing a biological sample obtained from the subject; and (b) detecting the presence of an antibiotic resistance gene in the biological sample according to any one of the methods described herein, wherein the presence of an antibiotic resistance gene in the biological sample obtained from the subject identifies the subject as one who may be infected with an antibiotic resistant bacterial pathogen. In some embodiments, the method further comprises selecting an optimized anti-bacterial therapy for the patient based on the presence of the antibiotic resistance gene.

In some embodiments, the method further comprises selecting administering the optimized anti-bacterial therapy to the patient. In some embodiments, the method further comprises selecting optimized anti-bacterial therapy comprises one or more antibiotic agents. In some embodiments, the one or more antibiotic agents is selected from the group consisting of polymyxin B, colistin, tigecycline, ceftazidime-avibactam, meropenem-vaborbactam, aztreonam, and fosfomycin. In some embodiments, the antibiotic agent is administered as a monotherapy. In some embodiments, the antibiotic agent is administered as a combination therapy. In some embodiments, the combination therapy comprises one or more additional antibiotic agents selected from the group consisting of an aminoglycoside, colistin, tigecycline, fosfomycin, gentamicin, tobramycin, amikacin, plazomicin, rimfampin, meropenem, doripenem, ertapenem, and imipenem. In some embodiments, the optimized antibacterial therapy is administered to the patient orally, intravenously, intramuscularly, intra-arterially, subcutaneously, or intraperitoneally.

In another aspect, the invention features a magnetic particle conjugated to a nucleic acid probe, wherein the nucleic acid probe is specific for an antibiotic resistance gene selected from the group consisting of NDM, KPC, IMP, VIM, OXA-48-like, DHA, CMY, FOX, mecA, mecC, vanA, vanB, CTX-M 14, CTX-M 15, mefA, mefE, ermA, ermB, SHV, and TEM. In some embodiments, the magnetic particle further comprises an additional nucleic acid probe, wherein the second nucleic acid probe is specific for a second antibiotic resistance gene selected from the group consisting of NDM, KPC, IMP, VIM, OXA-48-like, DHA, CMY, FOX, mecA, mecC, vanA, vanB, CTX-M 14, CTX-M 15, mefA, mefE, ermA, ermB, SHV, and TEM, wherein the second antibiotic resistance gene is selected from IMP, OXA-48-like, DHA, CMY, FOX, CTX-M 14, CTX-M 15, mecC, mefA, mefE, ermA, ermB, or TEM. In some embodiments, the magnetic particle comprises a first nucleic acid probe specific for DHA, and a second nucleic acid probe specific for CMY.

In another aspect, the invention features a magnetic particle conjugated to one or more nucleic acid probes comprising a nucleic acid sequence selected from SEQ ID NOs: 17-32, 57-78, 83, 84, 86, 87, 89, 90, and 93-96, or a nucleic acid sequence having at least 95% sequence identity to any one of SEQ ID NOs: 17-32, 57-78, 83, 84, 86, 87, 89, 90, and 93-96.

In another aspect, the invention features a removable cartridge comprising a well comprising any of the magnetic particles described herein. In some embodiments, the removable cartridge further comprises one or more chambers for holding a plurality of reagent modules for holding one or more assay reagents. In some embodiments, the removable cartridge further comprises a chamber comprising beads for lysing cells. In some embodiments, the removable cartridge further comprises a chamber comprising a polymerase. In some embodiments, the removable cartridge further comprises a chamber comprising one or more primers. In some embodiments, the one or more primers comprising a nucleic acid sequence selected from SEQ ID NOs: 1-16, 33-54, 81, 82, 85, 88, 91, and 92, or a nucleic acid sequence having at least 95% sequence identity to any one of SEQ ID NOs: 1-16, 33-54, 81, 82, 85, 88, 91, and 92.

In another aspect, the invention features a system for the detection of two or more antibiotic resistance genes selected from the group consisting of NDM, KPC, IMP, VIM, OXA-48-like, DHA, CMY, FOX, mecA, mecC, vanA, vanB, CTX-M 14, CTX-M 15, mefA, mefE, ermA, ermB, SHV, and TEM, wherein the two or more antibiotic resistance genes comprises at least one of IMP, OXA-48-like, DHA, CMY, FOX, CTX-M 14, CTX-M 15, mecC, mefA, mefE, ermA, ermB, or TEM, the system comprising: (a) a first unit comprising (i) a permanent magnet defining a magnetic field; (ii) a support defining a well holding a liquid sample comprising magnetic particles having a mean particle diameter between 600 and 1200 nm, preferably between 650 and 950 nm, and the one or more antibiotic resistance genes and having an RF coil disposed about the well, the RF coil configured to detect a signal produced by exposing the liquid sample to a bias magnetic field created using the permanent magnet and an RF pulse sequence; and (iii) one or more electrical elements in communication with the RF coil, the electrical elements configured to amplify, rectify, transmit, and/or digitize the signal; and (b) a second unit comprising a removable cartridge sized to facilitate insertion into and removal from the system, wherein the removable cartridge is a modular cartridge comprising (i) a reagent module for holding one or more assay reagents, (ii) a detection module comprising a detection chamber for holding a liquid sample comprising the magnetic particles and the one or more analytes, and, optionally, (iii) a sterilizable inlet module, wherein the reagent module, the detection module, and, optionally, the sterilizable inlet module, can be assembled into the modular cartridge prior to use, and wherein the detection chamber is removable from the modular cartridge, preferably, wherein the system further comprises a system computer with processor for implementing an assay protocol and storing assay data, and wherein the removable cartridge further comprises (i) a readable label indicating the analyte to be detected, (ii) a readable label indicating the assay protocol to be implemented, (iii) a readable label indicating a patient identification number, (iv) a readable label indicating the position of assay reagents contained in the cartridge, or (v) a readable label comprising instructions for the programmable processor.

In another aspect, the invention features a nucleic acid probe comprising a nucleic acid sequence selected from SEQ ID NOs: 17-32, 57-78, 83, 84, 86, 87, 89, 90, and 93-96, or a nucleic acid sequence having at least 95% sequence identity to any one of SEQ ID NOs: 17-32, 57-78, 83, 84, 86, 87, 89, 90, and 93-96.

In another aspect, the invention features a nucleic acid primer comprising a nucleic acid sequence selected from SEQ ID NOs: 1-16, 33-54, 81, 82, 85, 88, 91, and 92, or a nucleic acid sequence having at least 95% sequence identity to any one of SEQ ID NOs: 1-16, 33-54, 81, 82, 85, 88, 91, and 92.

Other features and advantages of the invention will be apparent from the following detailed description, drawings, and the claims.

### BRIEF DESCRIPTION OF THE DRAWINGS

FIG. 1 is a table showing an exemplary panel of the invention.

FIG. 2 is a table showing variants of targets of the invention verified by lab or in silico testing.

FIG. 3 is a table showing average (ms), standard deviation (Std Dev), and coefficient of variation (% CV) of all T₂ signals obtained during an LoD study performed at a titer of 10 CFU/mL. Species, isolate, marker, and detection channels are specified. Positivity rate is the number of replicates in which the target was detected (numerator) compared to the total number of replicates (denominator).

FIG. 4 is a table showing average (ms), standard deviation (Std Dev), and coefficient of variation (% CV) of all T₂ signals obtained during an LoD study performed at a titer of 2 CFU/mL. Species, isolate, marker, and detection channels are specified. Positivity rate is the number of replicates in which the target was detected (numerator) compared to the total number of replicates (denominator).

FIG. 5 is a table summarizing the results of a cross-reactivity assay for the targets of the invention. All assays were performed at a titer of 1000 CFU/mL. Reported values are positivity rates expressed as the number of replicates in which the target was detected in a given channel (numerator) compared to the total number of replicates tested (denominator). Dashes indicate that assays were not performed.

FIG. 6 is a table summarizing the results of an assay for interfering substances. All assays were performed at 10 CFU/mL. Reported values are positivity rates expressed as the number of replicates in which the target was detected (numerator) compared to the total number of replicates tested (denominator).

FIG. 7 is a table showing the results of a competitive inhibition assay.

FIG. 8 is a table showing the results of a multi-spike experiment.

FIG. 9 is a table showing a comparison of targets detected in a T2 magnetic resonance (T2MR) assay performed on a T2DX® instrument and in a next-generation sequencing assay (NGS). (+) indicates that a target was detected; (−) indicates that a target was not detected.

FIG. 10 is a table showing exemplary panel configurations.

FIG. 11 is a table showing exemplary panel configurations.

### DETAILED DESCRIPTION OF EMBODIMENTS OF THE INVENTION

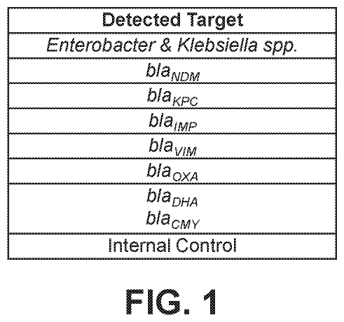

The invention provides, inter alia, methods, panels, systems, cartridges, and kits for detection of drug resistance markers (e.g., antibiotic resistance genes) in complex biological or environmental samples containing cells, cell debris (e.g., blood), or non-specific nucleic acids (e.g., subject (e.g., host) cell DNA). For example, the invention provides methods, panels, systems, cartridges, and kits for detection of Gram-negative carbapenem resistance genes, including targets listed in FIG. 1. In another example, the invention provides methods, systems, cartridges, and kits for detection of the T2Carba Resistance+panel described in Example 1 and FIG. 1. In another example, the invention provides methods, systems, cartridges, and kits for detection of the T2Resistance panel described in Example 3. In another example, the invention provides methods, systems, cartridges, and kits for detection of the T2ARx panel described in Example 4.

In some embodiments, detection of drug resistance markers (e.g., antibiotic resistance genes) allows for rapid, accurate, and high sensitivity detection and identification of a drug-resistant microbial pathogen present in a biological or environmental sample containing host cells, cell debris, and/or host cell nucleic acids (e.g., DNA), including but not limited to whole blood, processed whole blood (e.g., a crude whole blood lysate), serum, plasma, or other blood derivatives; bloody fluids such as wound exudate, phlegm, bile, and the like; bronchiolar lavage (BAL), urine, tissue samples (e.g., tissue biopsies); and sputum (e.g., purulent sputum and bloody sputum)), which may be used, for example, for diagnosis of a disease (e.g., sepsis, bloodstream infections (BSIs) (e.g., bacteremia, fungemia (e.g., Candidemia), and viremia), Lyme disease, septic shock, and diseases that may manifest with similar symptoms to diseases caused by or associated with drug resistant microbial pathogens, e.g., systemic inflammatory response syndrome (SIRS)).

In some embodiments, the methods, panels, systems, cartridges, and kits can involve T2MR detection of target nucleic acids. T2MR detection enables rapid and sensitive detection of drug resistance markers (e.g., antibiotic resistance genes) in complex samples. In some embodiments, the T2MR detection approaches employ magnetic particles. In some embodiments, the methods and systems employ an NMR unit, optionally one or more magnetic assisted agglomeration (MAA) units, optionally one or more incubation stations at different temperatures, optionally one or more vortexers, optionally one or more centrifuges, optionally a fluidic manipulation station, optionally a robotic system, and optionally one or more modular cartridges, as described in International Patent Application Publication No. WO 2012/054639, which is incorporated herein by reference in its entirety. In some embodiments, the methods of the invention are performed using a fully-automated system, e.g., which may contain a sequencing unit and, optionally, a NMR unit. T2MR approaches can be combined with sequencing. For example, in some embodiments, the T2MR detection can provide group-level information that is used to direct or narrow sequencing in a sample.

In some embodiments, the methods, systems, cartridges, kits, and panels can involve sequencing of target nucleic acids. Any suitable sequencing approach can be used, e.g., massively parallel sequencing (e.g., sequencing by synthesis (e.g., ILLUMINA™ dye sequencing, ion semiconductor sequencing, or pyrosequencing) or sequencing by ligation (e.g., oligonucleotide ligation and detection (SOLiD™) sequencing or polony-based sequencing)), long-read or single-molecule sequencing (e.g., Helicos™ sequencing, single-molecule real-time (SMRT™) sequencing, and nanopore sequencing) and/or Sanger sequencing.

Any of the methods, systems, cartridges, kits, and panels can involve detection of drug resistance markers (e.g., antibiotic resistance genes) as well as pathogen species, including any of the pathogen species described herein.

### Definitions

The terms “amplification” or “amplify” or derivatives thereof, as used herein, mean one or more methods known in the art for copying a target or template nucleic acid, thereby increasing the number of copies of a selected nucleic acid sequence. Amplification may be exponential or linear. A “target nucleic acid” refers to a nucleic acid or a portion thereof that is to be amplified, detected, and/or sequenced. A target or template nucleic acid may be any nucleic acid, including DNA or RNA. A target nucleic acid may include a drug resistance marker (e.g., an antibiotic resistance gene, e.g., an antibiotic resistance gene selected from the group consisting of NDM, KPC, IMP, VIM, OXA-48-like, DHA, CMY, FOX, mecA, mecC, vanA, vanB, CTX-M 14, CTX-M 15, mefA, mefE, ermA, ermB, SHV, and TEM) or a portion thereof that is to be amplified, detected, and/or sequenced. In the literature, the enzymes encoded by these genes are typically spelled in capital letters, while the gene names are italicized. For example, the enzyme NDM is encoded by the bla_{NDM} gene. This convention generally holds for all of the beta lactamase genes (e.g., NDM, KPC, IMP, VIM, DHA, CMY, FOX, CTX-M, SHV, TEM, and OXA-48-like). In the present application, these terms are used interchangeably, and the capitalized shorthand terms, e.g., “NDM” may be used to refer to a nucleic acid for simplicity. Other resistance genes are typically italicized in the literature (e.g., mecA, mecC, vanA, vanB, mefA, mefE, ermA, and ermB), but in the present application, it is to be understood that italicized and non-italicized versions of these names are used interchangeably. In other examples, a target nucleic acid may be characteristic of a pathogen species (for example, any of the pathogen species described herein). The sequences amplified in this manner form an “amplified target nucleic acid,” “amplified region,” or “amplicon,” which are used interchangeably herein. Primers and/or probes can be readily designed to target a specific template nucleic acid sequence. Exemplary amplification approaches include but are not limited to polymerase chain reaction (PCR), ligase chain reaction (LCR), multiple displacement amplification (MDA), strand displacement amplification (SDA), rolling circle amplification (RCA), loop mediated isothermal amplification (LAMP), nucleic acid sequence based amplification (NASBA), helicase dependent amplification, recombinase polymerase amplification, nicking enzyme amplification reaction, and ramification amplification (RAM).

The term “drug resistance” refers to the ability of a pathogen to resist one or more effects of a therapeutic agent. For example, “antimicrobial resistance” refers to the ability of a microbe (e.g., a bacterial or fungal pathogen) to resist one or more effects of an antimicrobial agent, and “antibiotic resistance” refers to the ability of a bacterium to resist one or more effects of an antibiotic agent. Drug-resistant pathogens can be more difficult to treat than drug-sensitive pathogens. Resistance can occur naturally in pathogens, or can arise via spontaneous mutation or by gene transfer between different species. A pathogen may be become to a therapeutic agent that previously was able to treat an infection caused by the pathogen. In some embodiments, a drug-resistant pathogen is able to survive or proliferate upon exposure to a concentration of a therapeutic agent that would kill or slow proliferation of a drug-sensitive pathogen.

A “drug resistance gene” or a “drug resistance target nucleic acid” refers to a gene that confers or facilitates drug resistance, or a portion thereof.

An “antibiotic resistance gene” or an “antibiotic resistance target nucleic acid” refers to a gene that confers or facilitates antibiotic resistance, or a portion thereof. Exemplary antibiotic (e.g., carbapenem) resistance genes include, but are not limited to, NDM, KPC, IMP, VIM, OXA-48-like, DHA, CMY, FOX, mecA, mecC, vanA, vanB, CTX-M 14, CTX-M 15, mefA, mefE, ermA, ermB, SHV, and TEM. Additional antibiotic resistance genes are described herein or are known in the art.

The terms “NDM” or “bla_{NDM}” refer to New Delhi metallo-beta-lactamase (e.g., NDM-1), as well as variants thereof, which may differ from NDM-1 by one or more amino acid substitutions, insertions, and/or deletions (including, e.g., NDM-2, NDM-3, NDM-4, NDM-5, NDM-6, NDM-7, NDM-8, NDM-9, NDM-10, NDM-11, NDM-12, NDM-13, NDM-14, NDM-15, NDM-16, NDM-17, NDM-18, NDM-19, NDM-20, NDM-21, NDM-22, NDM-23, NDM-24, and NDM-27).

The terms “KPC” or “bla_{KPC}” refer to K. pneumoniae carbapenemase (e.g., KPC-2), as well as variants thereof, which may differ from KPC-2 by one or more amino acid substitutions, insertions, and/or deletions (including, e.g., KPC-3, KPC-4, KPC-5, KPC-6, KPC-7, KPC-8, KPC-10, KPC-11, KPC-12, KPC-13, KPC-14, KPC-15, KPC-16, KPC-17, KPC-18, KPC-19, KPC-21, KPC-22, KPC-23, KPC-24, KPC-25, KPC-26, KPC-27, KPC-28, KPC-29, KPC-30, KPC-31, KPC-32, KPC-33, KPC-34, KPC-35, KPC-36, KPC-37, KPC-38, and KPC-39).

The terms “IMP” or “bla_{IMP}” refers to a metallo-beta-lactamase active on imipenem, including IMP-1, as well as variants thereof, which may differ from IMP-1 by one or more amino acid substitutions, insertions, and/or deletions (including, e.g., IMP-2, IMP-3, IMP-4, IMP-5, IMP-6, IMP-7, IMP-8, IMP-9, IMP-10, IMP-11, IMP-12, IMP-13, IMP-14, IMP-15, IMP-16, IMP-17, IMP-18, IMP-19, IMP-20, IMP-21, IMP-22, IMP-23, IMP-24, IMP-25, IMP-26, IMP-27, IMP-28, IMP-29, IMP-30, IMP-31, IMP-32, IMP-33, IMP-34, IMP-35, IMP-37, IMP-38, IMP-40, IMP-41, IMP-42, IMP-43, IMP-44, IMP-45, IMP-48, IMP-49, IMP-51, IMP-52, IMP-53, IMP-54, IMP-55, IMP-56, IMP-58, IMP-59, IMP-60, IMP-61, IMP-62, IMP-63, IMP-64, IMP-66, IMP-67, IMP-68, IMP-70, IMP-71, IMP-73, IMP-74, IMP-75, IMP-76, IMP-77, IMP-78, IMP-79, and IMP-80).

The terms “VIM” or “bla_{VIM}” refers to Verona integron-encoded metallo-beta-lactamase, also referred to as VIM-1, as well as variants thereof, which may differ from VIM-1 by one or more amino acid substitutions, insertions, and/or deletions (including, e.g., VIM-2, VIM-3, VIM-4, VIM-5, VIM-6, VIM-7, VIM-8, VIM-9, VIM-10, VIM-11, VIM-12, VIM-13, VIM-14, VIM-15, VIM-16, VIM-17, VIM-18, VIM-19, VIM-20, VIM-23, VIM-24, VIM-25, VIM-26, VIM-27, VIM-28, VIM-29, VIM-30, VIM-31, VIM-32, VIM-33, VIM-34, VIM-35, VIM-36, VIM-37, VIM-38, VIM-39, VIM-40, VIM-41, VIM-42, VIM-43, VIM-44, VIM-45, VIM-46, VIM-47, VIM-49, VIM-50, VIM-51, VIM-52, VIM-53, VIM-54, VIM-56, VIM-57, VIM-58, VIM-59, VIM-60, VIM-61, and VIM-62).

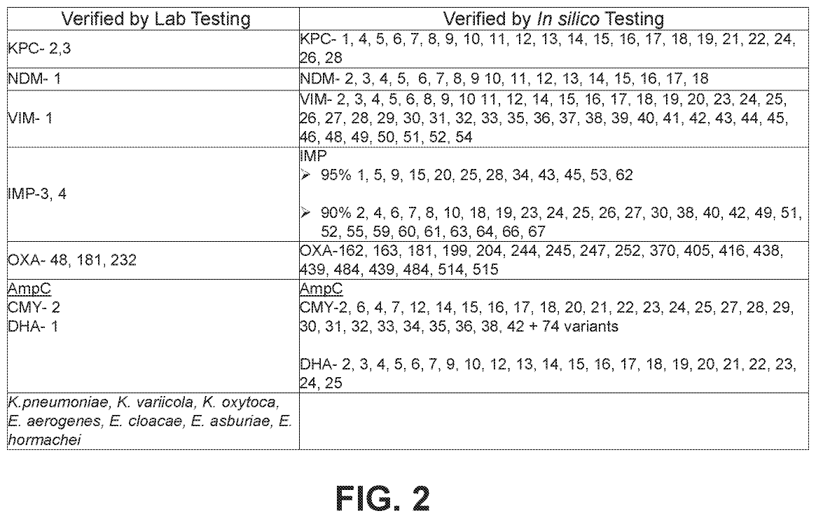

The term “OXA-48-like” refers to a group of carbapenem-hydroyzing class D beta lactamases. This group encompasses OXA-48 (also referred to as bla_{OXA-48}) as well as OXA-48-like variants, which may differ from OXA-48 by one or more amino acid substitutions, insertions, and/or deletions (including, e.g., OXA-162, OXA-163, OXA-181, OXA-199, OXA-204, OXA-232, OXA-244, OXA-245, OXA-247, OXA-252, OXA-370, OXA-405, OXA-416, OXA-438, OXA-439, OXA-484, OXA-505, OXA-514, OXA-515, OXA-517, OXA-519, OXA-538, OXA-546, OXA-547, OXA-566, OXA-567). A sequence alignment of OXA-48 and OXA-48-like variants is shown in FIG. 2 of Poirel et al. J. Antimicrob. Chemother. 67(7):1597-606, 2012).

The terms “DHA” or “bla_{DHA}” refer to plasmid-mediated Dhahran beta-lactamase, including DHA-1, as well as variants thereof, which may differ from DHA-1 by one or more amino acid substitutions, insertions, and/or deletions (including, e.g., DHA-2, DHA-3, DHA-4, DHA-5, DHA-6, DHA-7, DHA-10, DHA-12, DHA-13, DHA-14, DHA-15, DHA-16, DHA-17, DHA-18, DHA-19, DHA-20, DHA-21, DHA-22, DHA-23, DHA-24, DHA-25, DHA-26, DHA-27, and DHA-28).

The terms “CMY” or “bla_{CMY}” refers to a group of plasmid-mediated class C beta-lactamases that encode for resistance to antibiotics such as cephamycins, including CMY-2, as well as variants thereof, which may differ from CMY-2 by one or more amino acid substitutions, insertions, and/or deletions (including, e.g., CMY-4, CMY-5, CMY-6, CMY-7, CMY-12, CMY-13, CMY-14, CMY-15, CMY-16, CMY-17, CMY-18, CMY-20, CMY-21, CMY-22, CMY-23, CMY-24, CMY-25, CMY-26, CMY-27, CMY-28, CMY-29, CMY-30, CMY-31, CMY-32, CMY-33, CMY-34, CMY-35, CMY-36, CMY-37, CMY-38, CMY-39, CMY-40, CMY-41, CMY-42, CMY-43, CMY-44, CMY-45, CMY-46, CMY-47, CMY-48, CMY-49, CMY-50, CMY-51, CMY-53, CMY-54, CMY-55, CMY-56, CMY-57, CMY-58, CMY-59, CMY-60, CMY-61, CMY-62, CMY-63, CMY-64, CMY-65, CMY-66, CMY-67, CMY-68, CMY-69, CMY-70, CMY-71, CMY-72, CMY-73, CMY-74, CMY-75, CMY-76, CMY-77, CMY-78, CMY-79, CMY-80, CMY-81, CMY-82, CMY-83, CMY-84, CMY-85, CMY-86, CMY-87, CMY-89, CMY-90, CMY-93, CMY-94, CMY-95, CMY-96, CMY-97, CMY-99, CMY-100, CMY-101, CMY-102, CMY-103, CMY-104, CMY-105, CMY-106, CMY-107, CMY-108, CMY-109, CMY-110, CMY-111, CMY-112, CMY-113, CMY-114, CMY-115, CMY-116, CMY-117, CMY-118, CMY-119, CMY-121, CMY-122, CMY-124, CMY-125, CMY-127, CMY-128, CMY-129, CMY-130, CMY-131, CMY-132, CMY-133, CMY-134, CMY-135, CMY-138, CMY-139, CMY-140, CMY-141, CMY-142, CMY-143, CMY-144, CMY-145, CMY-146, CMY-147, CMY-148, CMY-149, CMY-150, CMY-151, CMY-152, CMY-153, CMY-154, CMY-155, CMY-156, CMY-158, CMY-159, CMY-160, CMY-161, CMY-162, CMY-163, and BIL-1).

The term “mecA” refers to a gene that confers resistance to antibiotics such as methicillin and other beta-lactam antibiotics. Methicillin-resistant S. aureus (MRSA) is a commonly known carrier of the mecA gene. An exemplary mecA gene is provided in the NCBI AMR database under accession number NG_047937.1.

The term “mecC” refers to a gene that confers resistance to antibiotics such as methicillin and other beta-lactam antibiotics. mecC is a divergent homologue of mecA, and is also known as mecA_{LGA251}.

The term “vanA” refers to a class of antibiotic resistance genes conferring resistance to antibiotics such as vancomycin.

The term “vanB” refers to a class of antibiotic resistance genes conferring resistance to antibiotics such as vancomycin.

The terms “CTX-M” or “bla_{CTX-M}” refer to a class of extended spectrum beta-lactamases active on cefotaxime and first discovered in Munich. For example, in some embodiments the CTX-M belongs to the “CTX-M 14” group (also referred to as the CTX-M 9 group), which includes CTX-M-9, CTX-M-13, CTX-M-14, CTX-M-16, CTX-M-17, CTX-M-19, CTX-M-21, CTX-M-24, CTX-M-27, CTX-M-46, CTX-M-47, CTX-M-48, CTX-M-49, CTX-M-50, CTX-M-64, CTX-M-73, CTX-M-81, CTX-M-87, CTX-M-90, CTX-M-93, CTX-M-98, CTX-M-102, CTX-M-104, CTX-M-121, CTX-M-125, CTX-M-148, CTX-M-168, CTX-M-198, CTX-M-199, CTX-M-201, CTX-M-214, CTX-M-221, and CTX-M-223. In other embodiments, the CTX-M belongs to the “CTX-M 15” group (also referred to as the CTX-M 1 group), which includes CTX-M-1, CTX-M-3, CTX-M-10, CTX-M-12, CTX-M-15, CTX-M-22, CTX-M-23, CTX-M-28, CTX-M-29, CTX-M-30, CTX-M-32, CTX-M-33, CTX-M-36, CTX-M-42, CTX-M-53, CTX-M-54, CTX-M-55, CTX-M-61, CTX-M-66, CTX-M-69, CTX-M-71, CTX-M-72, CTX-M-80, CTX-M-82, CTX-M-101, CTX-M-114, CTX-M-116, CTX-M-117, CTX-M-144, CTX-M-166, CTX-M-170, CTX-M-178, CTX-M-179, CTX-M-180, CTX-M-181, CTX-M-182, CTX-M-186, CTX-M-187, CTX-M-188, CTX-M-189, CTX-M-190, CTX-M-197, CTX-M-206, CTX-M-207, and CTX-M-222. Other CTX-M variants are known in the art and may be detected using the approaches described herein.

The term “mefA” refers to a gene conferring resistance to antibiotics such as macrolides by encoding drug efflux pumps. The term encompasses subclasses of mefA, including mefA and mefE.

The term “erm” refers to a class of genes conferring resistance to antibiotics such as the macrolide erythromycin. The term encompasses, for example, ermA and ermB.

The terms “SHV” or “bla_{SHV}” refers to a class of beta-lactamases. The term encompasses, for example, SHV-1, as well as variants thereof, which may differ from SHV-1 by one or more amino acid substitutions, insertions, and/or deletions (including, e.g., SHV-1, SHV-1b, SHV-2, SHV-2A, SHV-3, SHV-5, SHV-7, SHV-8, SHV-9, SHV-11, SHV-12, SHV-13, SHV-14, SHV-15, SHV-16, SHV-18, SHV-24, SHV-27, SHV-28, SHV-30, SHV-31, SHV-33, SHV-34, SHV-35, SHV-36, SHV-37, SHV-38, SHV-40, SHV-41, SHV-42, SHV-43, SHV-44, SHV-45, SHV-46, SHV-48, SHV-49, SHV-50, SHV-51, SHV-52, SHV-55, SHV-56, SHV-57, SHV-59, SHV-60, SHV-61, SHV-62, SHV-63, SHV-64, SHV-65, SHV-66, SHV-67, SHV-69, SHV-70, SHV-71, SHV-72, SHV-73, SHV-74, SHV-75, SHV-76, SHV-77, SHV-78, SHV-79, SHV-80, SHV-81, SHV-82, SHV-85, SHV-86, SHV-89, SHV-92, SHV-93, SHV-94, SHV-95, SHV-96, SHV-97, SHV-98, SHV-99, SHV-100, SHV-101, SHV-102, SHV-103, SHV-104, SHV-105, SHV-106, SHV-107, SHV-108, SHV-109, SHV-110, SHV-111, SHV-115, SHV-119, SHV-120, SHV-121, SHV-128, SHV-129, SHV-132, SHV-133, SHV-134, SHV-135, SHV-137, SHV-141, SHV-142, SHV-143, SHV-144, SHV-145, SHV-146, SHV-147, SHV-148, SHV-149, SHV-150, SHV-151, SHV-152, SHV-153, SHV-154, SHV-155, SHV-156, SHV-157, SHV-158, SHV-159, SHV-160, SHV-161, SHV-162, SHV-163, SHV-164, SHV-165, SHV-168, SHV-172, SHV-173, SHV-178, SHV-179, SHV-180, SHV-182, SHV-183, SHV-185, SHV-186, SHV-187, SHV-188, SHV-189, SHV-190, SHV-191, SHV-193, SHV-194, SHV-195, SHV-196, SHV-197, SHV-198, SHV-199, SHV-200, SHV-201, SHV-202, SHV-203, SHV-204, SHV-205, SHV-206, SHV-207, SHV-208, SHV-209, SHV-210, SHV-211, SHV-212, SHV-213, SHV-214, SHV-215, SHV-216, SHV-217, SHV-218, SHV-219, SHV-220, SHV-221, SHV-222, SHV-223, SHV-224, SHV-225, SHV-226, SHV-227, and SHV-228). For a review, see Liakoipolos et al. Front. Microbiol. 7:1374, 2016, which shows an alignment of SHV-type genes.

The terms “TEM” or “bla_{TEM}” refers to a class of beta-lactamases. The term encompasses, for example, TEM-1, as well as variants thereof, which may differ from TEM-1 by one or more amino acid substitutions, insertions, and/or deletions (including, e.g., TEM-2, TEM-3, TEM-4, TEM-6, TEM-8, TEM-9, TEM-10, TEM-11, TEM-12, TEM-15, TEM-16, TEM-17, TEM-19, TEM-20, TEM-21, TEM-22, TEM-24, TEM-26, TEM-28, TEM-29, TEM-30, TEM-32, TEM-33, TEM-34, TEM-35, TEM-36, TEM-40, TEM-43, TEM-45, TEM-47, TEM-48, TEM-49, TEM-52, TEM-53, TEM-54, TEM-55, TEM-56, TEM-57, TEM-60, TEM-63, TEM-67, TEM-68, TEM-70, TEM-71, TEM-72, TEM-76, TEM-77, TEM-78, TEM-79, TEM-80, TEM-81, TEM-82, TEM-83, TEM-84, TEM-85, TEM-86, TEM-87, TEM-88, TEM-90, TEM-91, TEM-92, TEM-93, TEM-94, TEM-95, TEM-96, TEM-97, TEM-98, TEM-99, TEM-101, TEM-102, TEM-104, TEM-105, TEM-106, TEM-107, TEM-108, TEM-109, TEM-110, TEM-111, TEM-112, TEM-113, TEM-114, TEM-115, TEM-116, TEM-120, TEM-121, TEM-122, TEM-123, TEM-124, TEM-125, TEM-126, TEM-127, TEM-128, TEM-129, TEM-130, TEM-131, TEM-132, TEM-133, TEM-134, TEM-135, TEM-136, TEM-137, TEM-138, TEM-139, TEM-141, TEM-142, TEM-143, TEM-144, TEM-145, TEM-146, TEM-147, TEM-148, TEM-149, TEM-150, TEM-151, TEM-152, TEM-153, TEM-154, TEM-155, TEM-156, TEM-157, TEM-158, TEM-159, TEM-160, TEM-162, TEM-163, TEM-164, TEM-166, TEM-167, TEM-168, TEM-169, TEM-171, TEM-176, TEM-177, TEM-178, TEM-181, TEM-182, TEM-183, TEM-184, TEM-185, TEM-186, TEM-187, TEM-188, TEM-189, TEM-190, TEM-191, TEM-193, TEM-194, TEM-195, TEM-196, TEM-197, TEM-198, TEM-201, TEM-205, TEM-206, TEM-207, TEM-208, TEM-209, TEM-210, TEM-211, TEM-212, TEM-213, TEM-214, TEM-215, TEM-216, TEM-217, TEM-219, TEM-220, TEM-224, TEM-225, TEM-226, TEM-227, TEM-229, TEM-230, TEM-231, TEM-233, TEM-234, TEM-236, and TEM-237).

As used herein, the terms “unit” or “units,” when used in reference to thermostable nucleic acid polymerases, refer to an amount of the thermostable nucleic acid polymerase (e.g., thermostable DNA polymerase). Typically a unit is defined as the amount of enzyme that will incorporate a particular amount of dNTPs (e.g., 10-20 nmol) into acid-insoluble material in 30-60 min at 65° C.-75° C. under particular assay conditions, although each manufacturer may define units differently. Unit definitions and assay conditions for commercially-available thermostable nucleic acid polymerases are known in the art. In some embodiments, one unit of thermostable nucleic acid polymerase (e.g., Taq DNA polymerase) may be the amount of enzyme that will incorporate 15 nmol of dNTP into acid-insoluble material in 30 min at 75° C. in an assay containing 1× ThermoPol® Reaction Buffer (New England Biosciences), 200 μM dNTPs including [H]-dTTP, and 15 nM primed M13 DNA.

The term “sequencing” refers to any method for determining the nucleotide order of a nucleic acid (e.g., DNA), such as a target nucleic acid or an amplified target nucleic acid. Exemplary sequencing approaches include but are not limited to massively parallel sequencing (e.g., sequencing by synthesis (e.g., ILLUMINA™ dye sequencing, ion semiconductor sequencing, or pyrosequencing) or sequencing by ligation (e.g., oligonucleotide ligation and detection (SOLiD™) sequencing or polony-based sequencing)), long-read or single-molecule sequencing (e.g., Helicos™ sequencing, single-molecule real-time (SMRT™) sequencing, and nanopore sequencing) and Sanger sequencing. Massively parallel sequencing is also referred to in the art as next-generation or second-generation sequencing, and typically involves parallel sequencing of a large number (e.g., thousands, millions, or billions) of spatially-separated, clonally amplified templates or single nucleic acid molecules. Short reads are often used in massively parallel sequencing. See, e.g., Metzker, Nature Reviews Genetics 11:31-36, 2010. Long-read sequencing and/or single-molecule sequencing are sometimes referred to as third-generation sequencing. Hybrid approaches (e.g., massively parallel and single molecule approaches or massively parallel and long-read approaches) can also be used. It is to be understood that some approaches may fall into more than one category, for example, some approaches may be considered both second-generation and third-generation approaches, and some sources refer to both second and third generation sequencing as “next-generation” sequencing.

By “analyte” is meant a substance or a constituent of a sample to be analyzed. Exemplary analytes include one or more species of one or more of the following: a nucleic acid (e.g., DNA or RNA (e.g., mRNA)), an oligonucleotide, a protein, a peptide, a polypeptide, an amino acid, an antibody, a carbohydrate, a polysaccharide, glucose, a lipid, a gas (e.g., oxygen or carbon dioxide), an electrolyte (e.g., sodium, potassium, chloride, bicarbonate, blood urea nitrogen (BUN), magnesium, phosphate, calcium, ammonia, lactate), a lipoprotein, cholesterol, a fatty acid, a glycoprotein, a proteoglycan, a lipopolysaccharide, a cell surface marker (e.g., a cell surface protein of a pathogen), a cytoplasmic marker (e.g., CD4/CD8 or CD4/viral load), a therapeutic agent, a metabolite of a therapeutic agent, a marker for the detection of a weapon (e.g., a chemical or biological weapon), an organism, a pathogen, a pathogen byproduct, a parasite (e.g., a protozoan or a helminth), a protist, a fungus (e.g., yeast (e.g., a Candida species (e.g., Candida albicans, Candida glabrata, Candida krusei, C. parapsilosis, Candida auris, Candida lusitaniae, Candida haemulonii, Candida duobushaemulonii, Candida pseudohaemulonii, Candida guilliermondii, and C. tropicalis)) or mold), a bacterium (e.g., Acinetobacter baumannii, Escherichia coli, Enterococcus faecalis, Enterococcus faecium, Kiebsiella pneumoniae, Pseudomonas aeruginosa, Staphylococcus aureus, Borrelia burgdoferi, Borrelia afzelii, Borrelia garinii, Rickettsia rickettsii, Anaplasma phagocytophilum, Coxiella bumeli, Ehrlichia chaffeensis, Ehrlichia ewingii, Francisella tularensis, Streptococcus pneumoniae, Enterobacter cloacae, Streptococcus pyogenes, Streptococcus mutans, Streptococcus sanguinis, Haemophilus influenzae, or Neisseria meningitides), an actinomycete, a cell (e.g., a whole cell, a tumor cell, a stem cell, a white blood cell, a T cell (e.g., displaying CD3, CD4, CD8, IL2R, CD35, or other surface markers), or another cell identified with one or more specific markers), a virus, a prion, a plant component, a plant by-product, algae, an algae by-product, plant growth hormone, an insecticide, a man-made toxin, an environmental toxin, an oil component, and components derived therefrom. In particular embodiments, the analyte is a nucleic acid (e.g., DNA or RNA (e.g., mRNA)), such as a target nucleic acid or an amplified target nucleic acid. In further particular embodiments, the analyte is a drug resistance marker (e.g., an antibiotic resistance gene, e.g., an antibiotic resistance gene selected from the group consisting of NDM, KPC, IMP, VIM, OXA-48-like, DHA, CMY, FOX, mecA, mecC, vanA, vanB, CTX-M 14, CTX-M 15, mefA, mefE, ermA, ermB, SHV, and TEM) or a portion thereof.

A “biological sample” is a sample obtained from a subject including but not limited to blood (e.g., whole blood, processed whole blood (e.g., a crude whole blood lysate), serum, plasma, and other blood derivatives), bloody fluids (e.g., wound exudate, phlegm, bile, and the like), urine, cerebrospinal fluid (CSF), synovial fluid (SF), breast milk, sweat, tears, saliva, semen, feces, vaginal fluid or tissue, sputum (e.g., purulent sputum and bloody sputum), nasopharyngeal aspirate or swab, lacrimal fluid, mucous, epithelial swab (e.g., a buccal swab, an axilla swab, a groin swab, an axilla/groin swab, or an ear swab), tissues (e.g., tissue biopsies (e.g., skin biopsies (e.g., from wounds, burns, or tick bites), muscle biopsies, or lymph node biopsies)), including tissue homogenates), organs, bones, teeth, or culture media (e.g., BHI, SABHI, SDA, LB, and the like), among others. In some embodiments, the biological sample is whole blood, which may contain an anticoagulant (e.g., EDTA, sodium citrate, sodium heparin, lithium heparin, and/or potassium oxylate/sodium fluoride). In several embodiments, the biological sample contains cells, cell debris, and/or nucleic acids (e.g., DNA) derived from the subject from which the sample was obtained. In particular embodiments, the subject is a host of a pathogen, and the biological sample obtained from the subject includes subject (host)-derived cells, cell debris, and nucleic acids (e.g., DNA), as well as one or more pathogen cells. The biological sample may be a swab sample, which may include a swab buffer diluent or swab transport medium. In some embodiments, the swab buffer diluent or swab transport medium is, without limitation, PBST, Amies Buffer, Amies Buffer+10% (v/v) 10×PBST, Cary Blair Media, or Liquid Stuart Swabs (which may include addition of 10% (v/v) 10×PBST). The biological sample may be a liquid sample.

As used herein, an “environmental sample” is a sample obtained from an environment, e.g., a surface swab sample, a sample from a building or a container, an air sample, a water sample, a soil sample, and the like. The environmental sample may contain any analyte described herein, e.g., a pathogen such as a bacterial (e.g., Acinetobacter baumannii, Escherichia coli, Enterococcus faecalis, Enterococcus faecium, Klebsiella pneumoniae, Pseudomonas aeruginosa, Staphylococcus aureus, Borrelia burgdorferi, Borrelia afzelii, Borrelia garinii, Rickettsia rickettsii, Anaplasma phagocytophilum, Coxiella burnetti, Ehrlichia chaffeensis, Ehrlichia ewingii, Francisella tularensis, Streptococcus pneumoniae, Enterobacter cloacae, Streptococcus pyogenes, Streptococcus mutans, Streptococcus sanguinis, Haemophilus influenzae, or Neisseria meningitides), fungal (e.g., a Candida species (e.g., Candida auris, Candida lusitaniae, Candida haemulonii, Candida duobushaemulonii, Candida pseudohaemulonii, Candida guilliermondii, Candida albicans, Candida glabrata, Candida krusei, C. parapsilosis, and/or C. tropicalis)), protozoan, or viral organism or pathogen. In some embodiments, an environmental sample is from a hospital or other healthcare facility. In some embodiments, the environmental sample is a swab, which may include a swab buffer diluent or swab transport medium. In some embodiments, the swab buffer diluent or swab transport medium is, without limitation, PBST, Amies Buffer, Amies Buffer+10% (v/v) 10×PBST, Cary Blair Media, or Liquid Stuart Swabs (which may include addition of 10% (v/v) 10×PBST). The environmental sample may be a liquid sample.

A “biomarker” is a biological substance that can be used as an indicator of a particular disease state or particular physiological state of an organism, generally a biomarker is a protein or other native compound measured in bodily fluid whose concentration reflects the presence or severity or staging of a disease state or dysfunction, can be used to monitor therapeutic progress of treatment of a disease or disorder or dysfunction, or can be used as a surrogate measure of clinical outcome or progression. In some embodiments, the biomarker is a nucleic acid (e.g., DNA or RNA (e.g., mRNA)).

The term “cell debris” refers to any materials released from cells that have been lysed or otherwise died. Cell debris may include any material that is contained within a cell, e.g., nucleic acids, proteins (e.g., hemoglobin), lipids, glycolipids, small molecules, carbohydrates, heme compounds, membranes, and the like. In several embodiments, the cell debris is or includes solid material, such as solid material that can be concentrated with a liquid-solid separation method (e.g., centrifugation or filtration). In some examples, the cell debris is the solid material present after centrifugation (such as solid material produced by the sample processing procedure described in Examples 1-6 of U.S. Provisional Patent Application No. 62/729,375).

As used herein, the term “cells/mL” indicates the number of cells per milliliter of a biological or environmental sample. The number of cells may be determined using any suitable method, for example, hemocytometer, quantitative PCR, and/or automated cell counting. It is to be understood that in some embodiments, cells/mL may indicate the number of colony-forming units (CFU) per milliliter of a biological or environmental sample.

As used herein, the term “copies/mL” indicates the number of copies of a nucleic acid (e.g., an antibiotic resistance gene, e.g., an antibiotic resistance gene selected from the group consisting of NDM, KPC, IMP, VIM, OXA-48-like, DHA, CMY, FOX, mecA, mecC, vanA, vanB, CTX-M 14, CTX-M 15, mefA, mefE, ermA, ermB, SHV, and TEM) or a portion thereof per milliliter of a biological or environmental sample.

A “group,” as used herein, refers to a grouping of organisms, including pathogens. In some embodiments, a group may be a taxonomic classification, for instance, a taxonomic domain, a taxonomic kingdom, a taxonomic phylum, a taxonomic class, a taxonomic order, a taxonomic family, or a taxonomic genus. In other embodiments, a group may be defined by any desired or suitable characteristics such as, for example, resistance to an antimicrobial agent or Gram staining (e.g., Gram negative or Gram positive). For example, the group may be pan-Gram positive or pan-Gram negative. It is to be understood that, in some instances, a pathogen may belong to more than one group.

A “group-level” identification refers to identification of an analyte (e.g., a target nucleic acid) that provides information regarding a group from which the analyte was obtained (e.g., a taxonomic classification, for instance, a taxonomic domain, a taxonomic kingdom, a taxonomic phylum, a taxonomic class, a taxonomic order, a taxonomic family, or a taxonomic genus). In some embodiments, a group-level identification does not provide species-level identification.

The term “species,” as used herein, refers to a basic unit of biological classification as well as a taxonomic rank. A skilled artisan appreciates that a species may be defined based on a number of criteria, including, for example, DNA similarity, morphology, and ecological niche. The term encompasses any suitable species concept, including evolutionary species, phylogenetic species, typological species, genetic species, and reproductive species. The term also encompasses subspecies or strains.

A “species-level” identification refers to identification of an analyte (e.g., a target nucleic acid) that provides information regarding the species from which the analyte was obtained. With respect to target nucleic acids, in some embodiments, species-level identification provides information regarding nucleic acid variants (e.g., a single nucleotide polymorphism (SNP), an insertion/deletion (indel), a repetitive element, or a microsatellite repeat), which is also referred to herein as a “variant-level” identification. In some embodiments, a species-level or variant-level identification also provides a group-level identification.

A “pathogen” means an agent causing disease or illness to its host, such as an organism or infectious particle, capable of producing a disease in another organism, and includes but is not limited to bacteria (e.g., Acinetobacter baumannii, Escherichia coli, Enterococcus faecalis, Enterococcus faecium, Klebsiella pneumoniae, Pseudomonas aeruginosa, Staphylococcus aureus, Borrelia burgdorferi, Borrelia afzelii, Borrelia garinii, Rickettsia rickettsii, Anaplasma phagocytophilum, Coxiella burnetii, Ehrlichia chaffeensis, Ehrlichia ewingii, Francisella tularensis, Streptococcus pneumoniae, Enterobacter cloacae, Streptococcus pyogenes, Streptococcus mutans, Streptococcus sanguinis, Haemophilus influenzae, or Neisseria meningiddes), viruses, protozoa, prions, fungi (e.g., yeast (e.g., Candida species)), or pathogen by-products. “Pathogen by-products” are those biological substances arising from the pathogen that can be deleterious to the host or stimulate an excessive host immune response, for example pathogen nucleic acids, antigen(s), metabolic substances, enzymes, biological substances, or toxins (e.g., Bacillus anthracis toxin genes protective antigen (pagA), edema factor (cya), and lethal factor (lef); enteropathogenic E. coli translocated intimin receptor (Tir); Clostridium difficile toxins TcdA and TcdB; and Clostridium botulinum toxins BoNT/A, BoNT/B, BoNT/C, BoNT/D, BoNT/E, BoNT/F, and BoNT/G). In some embodiments, the pathogen is a bacterial pathogen, e.g., a drug resistant bacterial pathogen, e.g., a bacterial pathogen that expresses one or more antibiotic resistance genes selected from the group consisting of NDM, KPC, IMP, VIM, OXA-48-like, DHA, CMY, FOX, mecA, mecC, vanA, vanB, CTX-M 14, CTX-M 15, mefA, mefE, ermA, ermB, SHV, and TEM.

By “pathogen-associated analyte” is meant an analyte characteristic of the presence of a pathogen (e.g., a bacterium, fungus, or virus) in a sample. The pathogen-associated analyte can be a particular substance derived from a pathogen (e.g., a nucleic acid (e.g., DNA or RNA (e.g., mRNA)), a protein, lipid, polysaccharide, or any other material produced by a pathogen) or a mixture derived from a pathogen (e.g., whole cells, or whole viruses). In certain instances, the pathogen-associated analyte is selected to be characteristic of the genus, species, or specific strain of pathogen being detected. Alternatively, the pathogen-associated analyte is selected to ascertain a property of the pathogen, such as resistance to a particular therapy. In some embodiments, a pathogen-associated analyte may be a target nucleic acid that has been amplified. In other embodiments, a pathogen-associated analyte may be a host antibody or other immune system protein that is expressed in response to an infection by a pathogen (e.g., an IgM antibody, an IgA antibody, an IgG antibody, or a major histocompatibility complex (MHC) protein).

A “subject” is an animal, preferably a mammal (including, for example, rodents (e.g., mice or rats), farm animals (e.g., cows, sheep, horses, and donkeys), pets (e.g., cats and dogs), or primates (e.g., humans and non-human primates (e.g., monkeys, chimpanzees, and gorillas)). In particular embodiments, the subject is a human. A subject may be a patient (e.g., a patient having or suspected of having a disease associated with or caused by a pathogen). In some embodiments, a subject is a host of one or more pathogens.

By “pharmaceutical composition” is meant any composition that contains a therapeutically or biologically active agent (e.g., an antifungal agent) that is suitable for administration to a subject. As used herein, by “administering” is meant a method of giving a dosage of a composition (e.g., a pharmaceutical composition) described herein (e.g., a composition comprising an antimicrobial agent (e.g., an antibiotic agent)) to a subject. The compositions utilized in the methods described herein can be administered by any suitable route, e.g., parenteral (for example, intravenous, intramuscular, intra-arterial, intracardiac, subcutaneous, or intraperitoneal), dermal, transdermal, ocular, inhalation, buccal, sublingual, perilingual, nasal, rectal, topical, and oral. The compositions utilized in the methods described herein can also be administered locally or systemically. The preferred method of administration can vary depending on various factors (e.g., the components of the composition being administered and the severity of the condition being treated).

As used herein, “linked” means attached or bound by covalent bonds, non-covalent bonds, and/or linked via Van der Waals forces, hydrogen bonds, and/or other intermolecular forces.

The term “magnetic particle” refers to particles including materials of high positive magnetic susceptibility such as paramagnetic compounds, superparamagnetic compounds, and magnetite, gamma ferric oxide, or metallic iron.

The terms “aggregation,” “agglomeration,” and “clustering” are used interchangeably in the context of the magnetic particles described herein and mean the binding of two or more magnetic particles to one another, for example, via a multivalent analyte, multimeric form of analyte, antibody, nucleic acid molecule, or other binding molecule or entity. In some instances, magnetic particle agglomeration is reversible. Such aggregation may lead to the formation of “aggregates,” which may include amplicons and magnetic particles bearing binding moieties.

As used herein, “nonspecific reversibility” refers to the colloidal stability and robustness of magnetic particles against non-specific aggregation in a liquid sample and can be determined by subjecting the particles to the intended assay conditions in the absence of a specific clustering moiety (i.e., an analyte or an agglomerator). For example, nonspecific reversibility can be determined by measuring the T₂ values of a solution of magnetic particles before and after incubation in a uniform magnetic field (defined as <5000 ppm) at 0.45 T for 3 minutes at 37° C. Magnetic particles are deemed to have nonspecific reversibility if the difference in T₂ values before and after subjecting the magnetic particles to the intended assay conditions vary by less than 10% (e.g., vary by less than 9%, 8%, 6%, 4%, 3%, 2%, or 1%). If the difference is greater than 10%, then the particles exhibit irreversibility in the buffer, diluents, and matrix tested, and manipulation of particle and matrix properties (e.g., coating and buffer formulation) may be required to produce a system in which the particles have nonspecific reversibility. In another example, the test can be applied by measuring the T₂ values of a solution of magnetic particles before and after incubation in a gradient magnetic field 1 Gauss/mm-10000 Gauss/mm.

As used herein, the term “NMR relaxation rate” refers to a measuring any of the following in a sample T₁, T₂, T₁/T₂ hybrid, T₁ᵣₕₒ, T₂ₕₒ, and T₂′. The systems and methods of the invention are designed to produce an NMR relaxation rate characteristic of whether an analyte is present in the liquid sample. In some instances the NMR relaxation rate is characteristic of the quantity of analyte present in the liquid sample.

As used herein, the term “T₁/T₂ hybrid” refers to any detection method that combines a T₁ and a T₂ measurement. For example, the value of a T₁/T₂ hybrid can be a composite signal obtained through the combination of, ratio, or difference between two or more different T₁ and T₂ measurements. The T₁/T₂ hybrid can be obtained, for example, by using a pulse sequence in which T₁ and T₂ are alternatively measured or acquired in an interleaved fashion. Additionally, the T₁/T₂ hybrid signal can be acquired with a pulse sequence that measures a relaxation rate that is comprised of both T₁ and T₂ relaxation rates or mechanisms.

By “pulse sequence” or “RF pulse sequence” is meant one or more radio frequency pulses to be applied to a sample and designed to measure, e.g., certain NMR relaxation rates, such as spin echo sequences. A pulse sequence may also include the acquisition of a signal following one or more pulses to minimize noise and improve accuracy in the resulting signal value.

As used herein, the term “signal” refers to an NMR relaxation rate, frequency shift, susceptibility measurement, diffusion measurement, or correlation measurements.

As used herein, reference to the “size” of a magnetic particle refers to the average diameter for a mixture of the magnetic particles as determined by microscopy, light scattering, or other methods.

As used herein, the term “substantially monodisperse” refers to a mixture of magnetic particles having a polydispersity in size distribution as determined by the shape of the distribution curve of particle size in light scattering measurements. The FWHM (full width half max) of the particle distribution curve less than 25% of the peak position is considered substantially monodisperse. In addition, only one peak should be observed in the light scattering experiments and the peak position should be within one standard deviation of a population of known monodisperse particles.

By “T₂ relaxivity per particle” is meant the average T₂ relaxivity per particle in a population of magnetic particles.

As used herein, “unfractionated” refers to an assay in which none of the components of the sample being tested are removed following the addition of magnetic particles to the sample and prior to the NMR relaxation measurement.

It is contemplated that units, methods, systems, cartridges, kits, panels, and processes of the claimed invention encompass variations and adaptations developed using information from the embodiments described herein. Throughout the description, where units, systems, cartridges, kits, or panels are described as having, including, or including specific components, or where processes and methods are described as having, including, or including specific steps, it is contemplated that, additionally, there are units, systems, cartridges, kits, or panels of the present invention that consist essentially of, or consist of, the recited components, and that there are processes and methods according to the present invention that consist essentially of, or consist of, the recited processing steps. It should be understood that the order of steps or order for performing certain actions is immaterial, unless otherwise specified, so long as the invention remains operable. Moreover, in many instances two or more steps or actions may be conducted simultaneously.

### Methods of Detecting Drug Resistance Markers in Complex Samples

The invention provides methods of detecting drug resistance markers (e.g., antibiotic resistance genes) in complex biological or environmental samples containing cells, cell debris (e.g., blood), or non-specific nucleic acids (e.g., subject (e.g., host) cell DNA).

In one example, provided herein is a method for detecting the presence of an antibiotic resistance gene in a biological sample, the method including: (a) amplifying in a biological sample or a fraction thereof one or more antibiotic resistance target nucleic acids in a multiplexed amplification reaction, wherein the multiplexed amplification reaction is configured to amplify target nucleic acids characteristic of two or more antibiotic resistance genes selected from the group consisting of NDM, KPC, IMP, VIM, OXA-48-like, DHA, CMY, FOX, mecA, mecC, vanA, vanB, CTX-M 14, CTX-M 15, mefA, mefE, ermA, ermB, SHV, and TEM; and (b) detecting the one or more amplified antibiotic resistance target nucleic acids to determine whether one or more of the antibiotic resistance genes is present in the biological sample, wherein the method individually detects an antibiotic resistance gene of a pathogen (e.g., a bacterial pathogen) present at a concentration of 10 cells/mL of biological sample or less (e.g., 10 cells/mL, 9 cells/mL, 8 cells/mL, 7 cells/mL, 6 cells/mL, 5 cells/mL, 4 cells/mL, 3 cells/mL, 2 cells/mL, or 1 cell/mL). In some embodiments, the two or more antibiotic resistance genes includes at least one of IMP, OXA-48-like, DHA, CMY, FOX, CTX-M 14, CTX-M 15, mecC, mefA, mefE, ermA, ermB, and TEM. In some embodiments, the method detects an antibiotic resistance gene of a pathogen (e.g., a bacterial pathogen) present at a concentration of 2 cells/mL of biological sample or less. In some embodiments, the method detects an antibiotic resistance gene of a pathogen (e.g., a bacterial pathogen) present at a concentration of 1 cells/mL of biological sample. In some embodiments, the two or more antibiotic resistance genes include NDM, KPC, IMP, VIM, OXA-48-like, DHA, CMY, mecA, mecC, vanA, vanB, CTX-M 14, CTX-M 15, mefA, mefE, ermA, ermB, SHV, and/or TEM.

In some embodiments, the method includes amplifying and/or detecting at least three, at least four, at least five, at least six, or all seven antibiotic resistance genes selected from the group consisting of NDM, KPC, IMP, VIM, OXA-48-like, DHA, and CMY. In some embodiments, the multiplexed amplification reaction is configured to amplify at least three, at least four, at least five, at least six, or all seven antibiotic resistance genes selected from the group consisting of NDM, KPC, IMP, VIM, OXA-48-like, DHA, and CMY. See, e.g., Tables 1-6 below. In some embodiments, the method includes amplifying and/or detecting NDM, KPC, IMP, VIM, OXA-48-like, DHA, and CMY. In some embodiments, the multiplexed amplification reaction is configured to amplify NDM, KPC, IMP, VIM, OXA-48-like, DHA, and CMY.

In some embodiments, (i) NDM is amplified in the presence of a forward primer comprising the nucleotide sequence CCAGCTCGCACCGAATGTCT (SEQ ID NO: 1) and a reverse primer comprising the nucleotide sequence CATCTTGTCCTGATGCGCGTGAGTCA (SEQ ID NO: 2); (ii) KPC is amplified in the presence of a forward primer comprising the nucleotide sequence TTTCTGCCACCGCGCTGAC (SEQ ID NO: 3) and a reverse primer comprising the nucleotide sequence GCAGCAAGAAAGCCCTTGAATG (SEQ ID NO: 4); (iii) IMP is amplified in the presence of a forward primer comprising the nucleotide sequence ACATTTCCATAGCGACAGCACGGGCGGAAT (SEQ ID NO: 5) and a reverse primer comprising the nucleotide sequence GGACTTTGGCCAAGCTTCTAAATTTGCGTC (SEQ ID NO: 6); (iv) VIM is amplified in the presence of a forward primer comprising the nucleotide sequence CGTGCAGTCTCCACGCACT (SEQ ID NO: 7) and a reverse primer comprising the nucleotide sequence TCGAATGCGCAGCACCGGGATAG (SEQ ID NO: 8); (v) OXA-48-like is amplified in the presence of a forward primer comprising the nucleotide sequence GGCTGTGTTTITGGTGGCATCGATTATC (SEQ ID NO: 9) and a reverse primer comprising the nucleotide sequence TCCCACTTAAAGACTTGGTGTTCATCC (SEQ ID NO: 10); (vi) DHA is amplified in the presence of a forward primer comprising the nucleotide sequence ACGGGCCGGTAATGCGGATCTGGA (SEQ ID NO: 11) and a reverse primer comprising the nucleotide sequence TATTCGCCAGAATCACAATCGCCACCTGT (SEQ ID NO: 12); and/or (vii) CMY is amplified in the presence of a forward primer comprising the nucleotide sequence CCGCGGCGAAATTAAGCTCAGCGA (SEQ ID NO: 13) and a reverse primer comprising the nucleotide sequence CCAAACAGACCAATGCTGGAGTTAG (SEQ ID NO: 14).

In another example, in some embodiments, (i) the method comprises amplifying and/or sequencing at least three, at least four, at least five, at least six, at least seven, at least eight, at least nine, at least ten, at least eleven, at least twelve, or all thirteen antibiotic resistance genes selected from the group consisting of KPC, CTX-M 14, CTX-M 15, NDM, VIM, IMP, vanA, vanB, mecA, mecC, OXA-48-like, CMY, and DHA; and/or (ii) the multiplexed amplification reaction is configured to amplify at least three, at least four, at least five, at least six, at least seven, at least eight, at least nine, at least ten, at least eleven, at least twelve, or all thirteen antibiotic resistance genes selected from the group consisting of KPC, CTX-M 14, CTX-M 15, NDM, VIM, IMP, vanA, vanB, mecA, mecC, OXA-48-like, CMY, and DHA.

In some embodiments, (i) the method comprises amplifying and/or sequencing KPC, CTX-M 14, CTX-M 15, NDM, VIM, IMP, vanA, vanB, mecA, mecC, OXA-48-like, CMY, and DHA; and/or (ii) the multiplexed amplification reaction is configured to amplify KPC, CTX-M 14, CTX-M 15, NDM, VIM, IMP, vanA, vanB, mecA, mecC, OXA-48-like, CMY, and DHA.

In some embodiments, (i) KPC is amplified in the presence of a forward primer comprising the nucleotide sequence TTTCTGCCACCGCGCTGAC (SEQ ID NO: 3) and a reverse primer comprising the nucleotide sequence GCAGCAAGAAAGCCCTTGAATG (SEQ ID NO: 4); (ii) CTX-M 14 is amplified in the presence of a forward primer comprising the nucleotide sequence ACGCTTTCCAATGTGCAGTACCAGTA (SEQ ID NO: 33) and a reverse primer comprising the nucleotide sequence TGCGATCCAGACGAAACGTCTCATCG (SEQ ID NO: 34); (iii) CTX-M 15 is amplified in the presence of a forward primer comprising the nucleotide sequence CCTCGGGCAATGGCGCAAAC (SEQ ID NO: 81) and a reverse primer comprising the nucleotide sequence ATCGCGACGGCTTTCTGCCTTA (SEQ ID NO: 82); (iv) NDM is amplified in the presence of a forward primer comprising the nucleotide sequence CCAGCTCGCACCGAATGTCT (SEQ ID NO: 1) and a reverse primer comprising the nucleotide sequence CATCTTGTCCTGATGCGCGTGAGTCA (SEQ ID NO: 2); (v) VIM is amplified in the presence of a forward primer comprising the nucleotide sequence CGTGCAGTCTCCACGCACT (SEQ ID NO: 7) and a reverse primer comprising the nucleotide sequence TCGAATGCGCAGCACCGGGATAG (SEQ ID NO: 8); (vi) IMP is amplified in the presence of a forward primer comprising the nucleotide sequence ACATTTCCATAGCGACAGCACGGGCGGAAT (SEQ ID NO: 5) and a reverse primer comprising the nucleotide sequence GGACTTTGGCCAAGCTTCTAAATTTGCGTC (SEQ ID NO: 6); (vii) vanA is amplified in the presence of a forward primer comprising the nucleotide sequence TATTCATCAGGAAGTCGAGCCGGA (SEQ ID NO: 85) and a reverse primer comprising the nucleotide sequence CAGTTCGGGAAGTGCAATACCTGCA (SEQ ID NO: 50); (viii) vanB is amplified in the presence of a forward primer comprising the nucleotide sequence AATTGAGCAAGCGATTTCGGGCTGT (SEQ ID NO: 51) and a reverse primer comprising the nucleotide sequence AAGATCAACACGGGCAAGCCCTCT (SEQ ID NO: 88); (ix) mecA is amplified in the presence of a forward primer comprising the nucleotide sequence ATGTTGGTCCCATTAACTCTGAAGAA (SEQ ID NO: 41) and a reverse primer comprising the nucleotide sequence CACCTGTTTGAGGGTGGATAGCAGTA (SEQ ID NO: 42); (x) mecC is amplified in the presence of a forward primer comprising the nucleotide sequence ATGTGGGTCCAATTAATTCTGACGAG (SEQ ID NO: 91) and a reverse primer comprising the nucleotide sequence CTCCAGTTTTGGTTGTAATGCTGTA (SEQ ID NO: 92); (xi) OXA-48-like is amplified in the presence of a forward primer comprising the nucleotide sequence GGCTGTGTTTITGGTGGCATCGATTATC (SEQ ID NO: 9) and a reverse primer comprising the nucleotide sequence TCCCACTTAAAGACTTGGTGTTCATCC (SEQ ID NO: 10); (xii) CMY is amplified in the presence of a forward primer comprising the nucleotide sequence CCGCGGCGAAATTAAGCTCAGCGA (SEQ ID NO: 13) and a reverse primer comprising the nucleotide sequence CCAAACAGACCAATGCTGGAGTTAG (SEQ ID NO: 14); and/or (xiii) DHA is amplified in the presence of a forward primer comprising the nucleotide sequence ACGGGCCGGTAATGCGGATCTGGA (SEQ ID NO: 11) and a reverse primer comprising the nucleotide sequence TATTCGCCAGAATCACAATCGCCACCTGT (SEQ ID NO: 12).

In another example, in some embodiments, (i) the method comprises amplifying and/or detecting at least three, at least four, at least five, at least six, at least seven, at least eight, at least nine, or all ten antibiotic resistance genes selected from the group consisting of CTX-M 14, CTX-M 15, ermA, ermB, mecA, mefA, SHV, TEM, vanA, and vanB; and/or (ii) the multiplexed amplification reaction is configured to amplify at least three, at least four, at least five, at least six, at least seven, at least eight, at least nine, or all ten antibiotic resistance genes selected from the group consisting of CTX-M 14, CTX-M 15, ermA, ermB, mecA, mefA, SHV, TEM, vanA, and vanB. See, e.g., Tables 7-15 below. In some embodiments, (i) the method comprises amplifying and/or detecting CTX-M 14, CTX-M 15, ermA, ermB, mecA, mefA, SHV, TEM, vanA, and vanB; and/or (ii) the multiplexed amplification reaction is configured to amplify CTX-M 14, CTX-M 15, ermA, ermB, mecA, mefA, SHV, TEM, vanA, and vanB.

In some embodiments, (i) CTX-M 14 is amplified in the presence of a forward primer comprising the nucleotide sequence ACGCTTTCCAATGTGCAGTACCAGTA (SEQ ID NO: 33) and a reverse primer comprising the nucleotide sequence TGCGATCCAGACGAAACGTCTCATCG (SEQ ID NO: 34); (ii) CTX-M 15 is amplified in the presence of a forward primer comprising the nucleotide sequence GTGATACCACTTCACCTCGGGCAA (SEQ ID NO: 35) and a reverse primer comprising the nucleotide sequence AATACATCGCGACGGCTTTCTGCC (SEQ ID NO: 36); (iii) ermA is amplified in the presence of a forward primer comprising the nucleotide sequence AGAATTACCTTTGAAAGTCAGGC (SEQ ID NO: 37) and a reverse primer comprising the nucleotide sequence GCTTCAAAGCCTGTCGGAATTGGTTT (SEQ ID NO: 38); (iv) ermB is amplified in the presence of a forward primer comprising the nucleotide sequence GGGCATTTAACGACGAAACTGGCTA (SEQ ID NO: 39) and a reverse primer comprising the nucleotide sequence GTGTTCGGTGAATATCCAAGGTACGC (SEQ ID NO: 40); (v) mecA is amplified in the presence of a forward primer comprising the nucleotide sequence ATGTTGGTCCCATTAACTCTGAAGAA (SEQ ID NO: 41) and a reverse primer comprising the nucleotide sequence CACCTGTTTGAGGGTGGATAGCAGTA (SEQ ID NO: 42); (vi) mefA is amplified in the presence of a forward primer comprising the nucleotide sequence GCAGGGCAAGCAGTATCATTAATCAC (SEQ ID NO: 43) and a reverse primer comprising the nucleotide sequence AATTAAATCAGCACCAATCATTATCTTCTTCC (SEQ ID NO: 44); (vii) SHV is amplified in the presence of a forward primer comprising the nucleotide sequence AAGCTGCTGACCAGCCAGCGTCTGA (SEQ ID NO: 45) and a reverse primer comprising the nucleotide sequence CGGCGATTTGCTGATTTCGCTCG (SEQ ID NO: 46); (viii) TEM is amplified in the presence of a forward primer comprising the nucleotide sequence TGCAGTGCTGCCATAACCATGAGTGA (SEQ ID NO: 47) and a reverse primer comprising the nucleotide sequence AGCGCAGAAGTGGTCCTGCAACTTT (SEQ ID NO: 48); (ix) vanA is amplified in the presence of a forward primer comprising the nucleotide sequence CAGTACGGAATCTTTCGTATTCATCAGGA (SEQ ID NO: 49) and a reverse primer comprising the nucleotide sequence CAGTTCGGGAAGTGCAATACCTGCA (SEQ ID NO: 50); and/or (x) vanB is amplified in the presence of a forward primer comprising the nucleotide sequence AATTGAGCAAGCGATTTCGGGCTGT (SEQ ID NO: 51) and a reverse primer comprising the nucleotide sequence CGTTTAGAACGATGCCGCCATCCT (SEQ ID NO: 52).

In some embodiments, the amplifying step (a) further comprises amplifying a target nucleic acid characteristic of a bacterial pathogen, and detecting step (b) further comprises detecting the amplified target nucleic acid characteristic of a bacterial pathogen to determine whether the bacterial pathogen is present; and/or the multiplexed amplification reaction is configured to amplify a target nucleic acid characteristic of a bacterial pathogen.

For example, in some embodiments, the amplifying step (a) further includes amplifying a target nucleic acid characteristic of a bacterial pathogen, and the detecting step (b) further includes detecting the amplified target nucleic acid characteristic of a bacterial pathogen to determine whether the bacterial pathogen is present. In some embodiments, the multiplexed amplification reaction is configured to amplify a target nucleic acid characteristic of a bacterial pathogen. In some embodiments, the target nucleic acid characteristic of a bacterial pathogen is characteristic of an Enterobacter spp., a Klebsiella spp., and/or Streptococcus pneumoniae In some embodiments, the target nucleic acid is characteristic of Enterobacter spp. and Klebsiella spp. In some embodiments, the target nucleic acid characteristic of Enterobacter spp. and Klebsiella spp. is amplified in the presence of a forward primer comprising the nucleotide sequence ATTCGTTGCACTATCGTTAACTGAATACA (SEQ ID NO: 15) and a reverse primer comprising the nucleotide sequence CTGTACCGTCGGACTTTCCAGAC (SEQ ID NO: 16). In other embodiments, the target nucleic acid characteristic of a bacterial pathogen is characteristic of Streptococcus pneumoniae. In some embodiments, the target nucleic acid characteristic of Streptococcus pneumoniae is amplified in the presence of a forward primer comprising the nucleotide sequence CCTTGGACGGAAATGTAGCTGGCA (SEQ ID NO: 53) and a reverse primer comprising the nucleotide sequence AATCACATGGTTGACACCTGCTGTG (SEQ ID NO: 54).

In some embodiments, the method includes amplifying and/or detecting NDM, KPC, IMP, VIM, OXA-48-like, DHA, CMY, and a target nucleic acid characteristic of Enterobacter spp. and Klebsiella spp.

In some embodiments, the multiplexed amplification reaction is configured to amplify NDM, KPC, IMP, VIM, OXA-48-like, DHA, CMY, and a target nucleic acid characteristic of Enterobacter spp. and Klebsiella spp.

In other embodiments, the method comprises amplifying and/or detecting KPC, CTX-M 14, CTX-M 15, NDM, VIM, IMP, vanA, vanB, and mecA, mecC, OXA-48-like, CMY, DHA, and a target nucleic acid characteristic of Enterobacter spp. and Klebsiella spp. In some embodiments, the multiplexed amplification reaction is configured to amplify KPC, CTX-M 14, CTX-M 15, NDM, VIM, IMP, vanA, vanB, and mecA, mecC, OXA-48-like, CMY, DHA, and a target nucleic acid characteristic of Enterobacter spp. and Klebsiella spp.

In yet other embodiments, the method comprises amplifying and/or detecting CTX-M 14, CTX-M 15, ermA, ermB, mecA, mefA, SHV, TEM, vanA, vanB, and a target nucleic acid characteristic of Streptococcus pneumoniae. In some embodiments, the multiplexed amplification reaction is configured to amplify CTX-M 14, CTX-M 15, ermA, ermB, mecA, mefA, SHV, TEM, vanA, vanB, and a target nucleic acid characteristic of Streptococcus pneumoniae.

In some embodiments of any of the preceding methods, the amplifying step (a) further includes amplifying an internal amplification control (IC) target nucleic acid and the detecting step (b) further includes detecting the amplified IC target nucleic acid. In some embodiments, the multiplexed amplification reaction is configured to amplify an amplified IC target nucleic acid. In some embodiments, the IC target nucleic acid is amplified in the presence of a forward primer comprising the nucleotide sequence of GGAAATCTAACGAGAGAGCATGCT (SEQ ID NO: 55) and a reverse primer comprising the oligonucleotide sequence CGATGCGTGACACCCAGGC (SEQ ID NO: 56).

The methods may utilize any suitable detection approach. In some embodiments of the preceding aspect, the detecting of step (b) includes magnetic, sequencing, optical, fluorescent, mass, density, chromatographic, and/or electrochemical detection. In some embodiments, the detecting of step (b) includes T2 magnetic resonance (T2MR). In some embodiments, the detecting of step (b) includes sequencing. In some embodiments, the detecting of step (b) includes T2MR and sequencing.

In another example, provided herein is a method for detecting the presence of an antibiotic resistance gene in a biological sample, the method including: (a) providing a biological sample; (b) lysing pathogen cells in the biological sample; (c) amplifying in the product of step (b) one or more antibiotic resistance target nucleic acids in a multiplexed amplification reaction to form an amplified biological sample, wherein the multiplexed amplification reaction is configured to amplify target nucleic acids characteristic of two or more antibiotic resistance genes selected from the group consisting of NDM, KPC, IMP, VIM, OXA-48-like, DHA, CMY, FOX, mecA, vanA, vanB, CTX-M 14, CTX-M 15, mefA, mecC, mefE, ermA, ermB, SHV, and TEM; (d) preparing a first assay sample by contacting a portion of the amplified biological sample with a first population of magnetic particles, wherein the magnetic particles of the first population have binding moieties characteristic of a first antibiotic resistance gene on their surface, the binding moieties operative to alter aggregation of the magnetic particles in the presence of a first amplified antibiotic resistance target nucleic acid; (e) preparing a second assay sample by contacting a portion of the amplified biological sample with a second population of magnetic particles, wherein the magnetic particles of the second population have binding moieties characteristic of a second antibiotic resistance gene on their surface, the binding moieties operative to alter aggregation of the magnetic particles in the presence of a second amplified antibiotic resistance target nucleic acid; (f) providing each assay sample in a detection tube within a device, the device including a support defining a well for holding the detection tube including the assay sample, and having an RF coil configured to detect a signal produced by exposing the mixture to a bias magnetic field created using one or more magnets and an RF pulse sequence; (g) exposing each assay sample to a bias magnetic field and an RF pulse sequence; (h) following step (g), measuring the signal produced by each assay sample; and (i) on the basis of the result of step (h), detecting whether one or more of the antibiotic resistance genes is present in the biological sample. In some embodiments, the two or more antibiotic resistance genes includes at least one of IMP, OXA-48-like, DHA, CMY, FOX, CTX-M 14, CTX-M 15, mecC, mefA, mefE, ermA, ermB, and TEM. In some embodiments, the two or more antibiotic resistance genes include NDM, KPC, IMP, VIM, OXA-48-like, DHA, CMY, mecA, mecC, vanA, vanB, CTX-M 14, CTX-M 15, mefA, mefE, ermA, ermB, SHV, and/or TEM.

In some embodiments, the method includes amplifying and/or detecting at least three, at least four, at least five, at least six, or all seven antibiotic resistance genes selected from the group consisting of NDM, KPC, IMP, VIM, OXA-48-like, DHA, and CMY. In some embodiments, the multiplexed amplification reaction is configured to amplify at least three, at least four, at least five, at least six, or all seven antibiotic resistance genes selected from the group consisting of NDM, KPC, IMP, VIM, OXA-48-like, DHA, and CMY. See, e.g., Tables 1-6 below. In some embodiments, the method includes amplifying and/or detecting NDM, KPC, IMP, VIM, OXA-48-like, DHA, and CMY. In some embodiments, the multiplexed amplification reaction is configured to amplify NDM, KPC, IMP, VIM, OXA-48-like, DHA, and CMY.

In some embodiments, (i) NDM is amplified in the presence of a forward primer comprising the nucleotide sequence CCAGCTCGCACCGAATGTCT (SEQ ID NO: 1) and a reverse primer comprising the nucleotide sequence CATCTTGTCCTGATGCGCGTGAGTCA (SEQ ID NO: 2); (ii) KPC is amplified in the presence of a forward primer comprising the nucleotide sequence TTTCTGCCACCGCGCTGAC (SEQ ID NO: 3) and a reverse primer comprising the nucleotide sequence GCAGCAAGAAAGCCCTTGAATG (SEQ ID NO: 4); (iii) IMP is amplified in the presence of a forward primer comprising the nucleotide sequence ACATTTCCATAGCGACAGCACGGGCGGAAT (SEQ ID NO: 5) and a reverse primer comprising the nucleotide sequence GGACTTTGGCCAAGCTTCTAAATTTGCGTC (SEQ ID NO: 6); (iv) VIM is amplified in the presence of a forward primer comprising the nucleotide sequence CGTGCAGTCTCCACGCACT (SEQ ID NO: 7) and a reverse primer comprising the nucleotide sequence TCGAATGCGCAGCACCGGGATAG (SEQ ID NO: 8); (v) OXA-48-like is amplified in the presence of a forward primer comprising the nucleotide sequence GGCTGTGTTTITGGTGGCATCGATTATC (SEQ ID NO: 9) and a reverse primer comprising the nucleotide sequence TCCCACTTAAAGACTTGGTGTTCATCC (SEQ ID NO: 10); (vi) DHA is amplified in the presence of a forward primer comprising the nucleotide sequence ACGGGCCGGTAATGCGGATCTGGA (SEQ ID NO: 11) and a reverse primer comprising the nucleotide sequence TATTCGCCAGAATCACAATCGCCACCTGT (SEQ ID NO: 12); and/or (vii) CMY is amplified in the presence of a forward primer comprising the nucleotide sequence CCGCGGCGAAATTAAGCTCAGCGA (SEQ ID NO: 13) and a reverse primer comprising the nucleotide sequence CCAAACAGACCAATGCTGGAGTTAG (SEQ ID NO: 14).

In another example, in some embodiments, (i) the method comprises amplifying and/or sequencing at least three, at least four, at least five, at least six, at least seven, at least eight, at least nine, at least ten, at least eleven, at least twelve, or all thirteen antibiotic resistance genes selected from the group consisting of KPC, CTX-M 14, CTX-M 15, NDM, VIM, IMP, vanA, vanB, mecA, mecC, OXA-48-like, CMY, and DHA; and/or (ii) the multiplexed amplification reaction is configured to amplify at least three, at least four, at least five, at least six, at least seven, at least eight, at least nine, at least ten, at least eleven, at least twelve, or all thirteen antibiotic resistance genes selected from the group consisting of KPC, CTX-M 14, CTX-M 15, NDM, VIM, IMP, vanA, vanB, mecA, mecC, OXA-48-like, CMY, and DHA.

In some embodiments, (i) the method comprises amplifying and/or sequencing KPC, CTX-M 14, CTX-M 15, NDM, VIM, IMP, vanA, vanB, mecA, mecC, OXA-48-like, CMY, and DHA; and/or (ii) the multiplexed amplification reaction is configured to amplify KPC, CTX-M 14, CTX-M 15, NDM, VIM, IMP, vanA, vanB, mecA, mecC, OXA-48-like, CMY, and DHA.

In some embodiments, (i) KPC is amplified in the presence of a forward primer comprising the nucleotide sequence TTTCTGCCACCGCGCTGAC (SEQ ID NO: 3) and a reverse primer comprising the nucleotide sequence GCAGCAAGAAAGCCCTTGAATG (SEQ ID NO: 4); (ii) CTX-M 14 is amplified in the presence of a forward primer comprising the nucleotide sequence ACGCTTTCCAATGTGCAGTACCAGTA (SEQ ID NO: 33) and a reverse primer comprising the nucleotide sequence TGCGATCCAGACGAAACGTCTCATCG (SEQ ID NO: 34); (iii) CTX-M 15 is amplified in the presence of a forward primer comprising the nucleotide sequence CCTCGGGCAATGGCGCAAAC (SEQ ID NO: 81) and a reverse primer comprising the nucleotide sequence ATCGCGACGGCTTTCTGCCTTA (SEQ ID NO: 82); (iv) NDM is amplified in the presence of a forward primer comprising the nucleotide sequence CCAGCTCGCACCGAATGTCT (SEQ ID NO: 1) and a reverse primer comprising the nucleotide sequence CATCTTGTCCTGATGCGCGTGAGTCA (SEQ ID NO: 2); (v) VIM is amplified in the presence of a forward primer comprising the nucleotide sequence CGTGCAGTCTCCACGCACT (SEQ ID NO: 7) and a reverse primer comprising the nucleotide sequence TCGAATGCGCAGCACCGGGATAG (SEQ ID NO: 8); (vi) IMP is amplified in the presence of a forward primer comprising the nucleotide sequence ACATTTCCATAGCGACAGCACGGGCGGAAT (SEQ ID NO: 5) and a reverse primer comprising the nucleotide sequence GGACTTTGGCCAAGCTTCTAAATTTGCGTC (SEQ ID NO: 6); (vii) vanA is amplified in the presence of a forward primer comprising the nucleotide sequence TATTCATCAGGAAGTCGAGCCGGA (SEQ ID NO: 85) and a reverse primer comprising the nucleotide sequence CAGTTCGGGAAGTGCAATACCTGCA (SEQ ID NO: 50); (viii) vanB is amplified in the presence of a forward primer comprising the nucleotide sequence AATTGAGCAAGCGATTTCGGGCTGT (SEQ ID NO: 51) and a reverse primer comprising the nucleotide sequence AAGATCAACACGGGCAAGCCCTCT (SEQ ID NO: 88); (ix) mecA is amplified in the presence of a forward primer comprising the nucleotide sequence ATGTTGGTCCCATTAACTCTGAAGAA (SEQ ID NO: 41) and a reverse primer comprising the nucleotide sequence CACCTGTTTGAGGGTGGATAGCAGTA (SEQ ID NO: 42); (x) mecC is amplified in the presence of a forward primer comprising the nucleotide sequence ATGTGGGTCCAATTAATTCTGACGAG (SEQ ID NO: 91) and a reverse primer comprising the nucleotide sequence CTCCAGTTTTGGTTGTAATGCTGTA (SEQ ID NO: 92); (xi) OXA-48-like is amplified in the presence of a forward primer comprising the nucleotide sequence GGCTGTGTTTITGGTGGCATCGATTATC (SEQ ID NO: 9) and a reverse primer comprising the nucleotide sequence TCCCACTTAAAGACTTGGTGTTCATCC (SEQ ID NO: 10); (xii) CMY is amplified in the presence of a forward primer comprising the nucleotide sequence CCGCGGCGAAATTAAGCTCAGCGA (SEQ ID NO: 13) and a reverse primer comprising the nucleotide sequence CCAAACAGACCAATGCTGGAGTTAG (SEQ ID NO: 14); and/or (xiii) DHA is amplified in the presence of a forward primer comprising the nucleotide sequence ACGGGCCGGTAATGCGGATCTGGA (SEQ ID NO: 11) and a reverse primer comprising the nucleotide sequence TATTCGCCAGAATCACAATCGCCACCTGT (SEQ ID NO: 12).

In another example, in some embodiments, (i) the method comprises amplifying and/or detecting at least three, at least four, at least five, at least six, at least seven, at least eight, at least nine, or all ten antibiotic resistance genes selected from the group consisting of CTX-M 14, CTX-M 15, ermA, ermB, mecA, mefA, SHV, TEM, vanA, and vanB; and/or (ii) the multiplexed amplification reaction is configured to amplify at least three, at least four, at least five, at least six, at least seven, at least eight, at least nine, or all ten antibiotic resistance genes selected from the group consisting of CTX-M 14, CTX-M 15, ermA, ermB, mecA, mefA, SHV, TEM, vanA, and vanB. In some embodiments, (i) the method comprises amplifying and/or detecting CTX-M 14, CTX-M 15, ermA, ermB, mecA, mefA, SHV, TEM, vanA, and vanB; and/or (ii) the multiplexed amplification reaction is configured to amplify CTX-M 14, CTX-M 15, ermA, ermB, mecA, mefA, SHV, TEM, vanA, and vanB.

In some embodiments, (i) CTX-M 14 is amplified in the presence of a forward primer comprising the nucleotide sequence ACGCTTTCCAATGTGCAGTACCAGTA (SEQ ID NO: 33) and a reverse primer comprising the nucleotide sequence TGCGATCCAGACGAAACGTCTCATCG (SEQ ID NO: 34); (ii) CTX-M 15 is amplified in the presence of a forward primer comprising the nucleotide sequence GTGATACCACTTCACCTCGGGCAA (SEQ ID NO: 35) and a reverse primer comprising the nucleotide sequence AATACATCGCGACGGCTTTCTGCC (SEQ ID NO: 36); (iii) ermA is amplified in the presence of a forward primer comprising the nucleotide sequence AGAATTACCTTTGAAAGTCAGGC (SEQ ID NO: 37) and a reverse primer comprising the nucleotide sequence GCTTCAAAGCCTGTCGGAATTGGTTT (SEQ ID NO: 38); (iv) ermB is amplified in the presence of a forward primer comprising the nucleotide sequence GGGCATTTAACGACGAAACTGGCTA (SEQ ID NO: 39) and a reverse primer comprising the nucleotide sequence GTGTTCGGTGAATATCCAAGGTACGC (SEQ ID NO: 40); (v) mecA is amplified in the presence of a forward primer comprising the nucleotide sequence ATGTTGGTCCCATTAACTCTGAAGAA (SEQ ID NO: 41) and a reverse primer comprising the nucleotide sequence CACCTGTTTGAGGGTGGATAGCAGTA (SEQ ID NO: 42); (vi) mefA is amplified in the presence of a forward primer comprising the nucleotide sequence GCAGGGCAAGCAGTATCATTAATCAC (SEQ ID NO: 43) and a reverse primer comprising the nucleotide sequence AATTAAATCAGCACCAATCATTATCTTCTTCC (SEQ ID NO: 44); (vii) SHV is amplified in the presence of a forward primer comprising the nucleotide sequence AAGCTGCTGACCAGCCAGCGTCTGA (SEQ ID NO: 45) and a reverse primer comprising the nucleotide sequence CGGCGATTTGCTGATTTCGCTCG (SEQ ID NO: 46); (viii) TEM is amplified in the presence of a forward primer comprising the nucleotide sequence TGCAGTGCTGCCATAACCATGAGTGA (SEQ ID NO: 47) and a reverse primer comprising the nucleotide sequence AGCGCAGAAGTGGTCCTGCAACTTT (SEQ ID NO: 48); (ix) vanA is amplified in the presence of a forward primer comprising the nucleotide sequence CAGTACGGAATCTTTCGTATTCATCAGGA (SEQ ID NO: 49) and a reverse primer comprising the nucleotide sequence CAGTTCGGGAAGTGCAATACCTGCA (SEQ ID NO: 50); and/or (x) vanB is amplified in the presence of a forward primer comprising the nucleotide sequence AATTGAGCAAGCGATTTCGGGCTGT (SEQ ID NO: 51) and a reverse primer comprising the nucleotide sequence CGTTTAGAACGATGCCGCCATCCT (SEQ ID NO: 52).

In some embodiments, the amplifying step (a) further comprises amplifying a target nucleic acid characteristic of a bacterial pathogen, and detecting step (b) further comprises detecting the amplified target nucleic acid characteristic of a bacterial pathogen to determine whether the bacterial pathogen is present; and/or the multiplexed amplification reaction is configured to amplify a target nucleic acid characteristic of a bacterial pathogen.

For example, in some embodiments of the preceding aspect, the amplifying step (c) further includes amplifying a target nucleic acid characteristic of a bacterial pathogen, and step (i) further includes detecting the amplified target nucleic acid characteristic of a bacterial pathogen to determine whether the bacterial pathogen is present. In some embodiments, the multiplexed amplification reaction is configured to amplify a target nucleic acid characteristic of a bacterial pathogen. In some embodiments, the target nucleic acid characteristic of a bacterial pathogen is characteristic of an Enterobacter spp., a Klebsiella spp., and/or Streptococcus pneumoniae. In some embodiments, the target nucleic acid is characteristic of Enterobacter spp. and Klebsiella spp. In some embodiments, the target nucleic acid characteristic of Enterobacter spp. and Klebsiella spp. is amplified in the presence of a forward primer comprising the nucleotide sequence ATTCGTTGCACTATCGTTAACTGAATACA (SEQ ID NO: 15) and a reverse primer comprising the nucleotide sequence CTGTACCGTCGGACTTTCCAGAC (SEQ ID NO: 16). In other embodiments, the target nucleic acid characteristic of a bacterial pathogen is characteristic of Streptococcus pneumoniae. In some embodiments, the target nucleic acid characteristic of Streptococcus pneumoniae is amplified in the presence of a forward primer comprising the nucleotide sequence CCTTGGACGGAAATGTAGCTGGCA (SEQ ID NO: 53) and a reverse primer comprising the nucleotide sequence AATCACATGGTTGACACCTGCTGTG (SEQ ID NO: 54).

In some embodiments, the method includes amplifying and/or detecting NDM, KPC, IMP, VIM, OXA-48-like, DHA, CMY, and a target nucleic acid characteristic of Enterobacter spp. and Klebsiella spp.

In some embodiments, the multiplexed amplification reaction is configured to amplify NDM, KPC, IMP, VIM, OXA-48-like, DHA, CMY, and a target nucleic acid characteristic of Enterobacter spp. and Klebsiella spp.

In other embodiments, the method comprises amplifying and/or detecting KPC, CTX-M 14, CTX-M 15, NDM, VIM, IMP, vanA, vanB, and mecA, mecC, OXA-48-like, CMY, DHA, and a target nucleic acid characteristic of Enterobacter spp. and Klebsiella spp. In some embodiments, the multiplexed amplification reaction is configured to amplify KPC, CTX-M 14, CTX-M 15, NDM, VIM, IMP, vanA, vanB, and mecA, mecC, OXA-48-like, CMY, DHA, and a target nucleic acid characteristic of Enterobacter spp. and Klebsiella spp.

In yet other embodiments, the method comprises amplifying and/or detecting CTX-M 14, CTX-M 15, ermA, ermB, mecA, mefA, SHV, TEM, vanA, vanB, and a target nucleic acid characteristic of Streptococcus pneumoniae. In some embodiments, the multiplexed amplification reaction is configured to amplify CTX-M 14, CTX-M 15, ermA, ermB, mecA, mefA, SHV, TEM, vanA, vanB, and a target nucleic acid characteristic of Streptococcus pneumoniae.

In some embodiments, the amplifying step (c) further includes amplifying an IC target nucleic acid and step (i) further includes detecting the amplified IC target nucleic acid. In some embodiments, the multiplexed amplification reaction is configured to amplify an amplified IC target nucleic acid. In some embodiments, the IC target nucleic acid is amplified in the presence of a forward primer comprising the nucleotide sequence of GGAAATCTAACGAGAGAGCATGCT (SEQ ID NO: 55) and a reverse primer comprising the oligonucleotide sequence CGATGCGTGACACCCAGGC (SEQ ID NO: 56).

The magnetic particles may be any of the magnetic particles described herein or in International Patent Application Publication Nos. WO 2012/054639, WO 2016/118766, WO 2017/127731, or in International Patent Application No. PCT/US2018/033278, each of which is incorporated herein by reference in its entirety.

In some embodiments, the magnetic particles of each population include two subpopulations, a first subpopulation bearing a first probe on its surface, and a second subpopulation bearing a second probe on its surface. In other embodiments, the magnetic particles of each population include two subpopulations, a first subpopulation bearing a first probe and a second probe on its surface, and a second subpopulation bearing a third probe and a fourth probe on its surface.

In some embodiments, the method comprises amplifying one or more (e.g., one, two, three, four, five, six, or all seven) antibiotic resistance genes selected from the group consisting of NDM, KPC, IMP, VIM, OXA-48-like, DHA, and/or CMY, and wherein: (i) a probe pair comprising a 5′ probe comprising the nucleotide sequence GCGACCGGCAGGTTGATCT (SEQ ID NO: 17) or GCGACCGGCAGGTTGATCTCCTGC (SEQ ID NO: 95) and a 3′ probe comprising the nucleotide sequence CATGTCGAGATAGGAAGTGTGCTGC (SEQ ID NO: 18) or CGGCATGTCGAGATAGGAAGTGTGCTGC (SEQ ID NO: 96) is used for detection of NDM; (ii) a probe pair comprising a 5′ probe comprising the nucleotide sequence CGGAACCATTCGCTAAACTC (SEQ ID NO: 19) and a 3′ probe comprising the nucleotide sequence AGGCGCAACTGTAAGTTACCG (SEQ ID NO: 20) is used for detection of KPC; (iii) a probe pair comprising a 5′ probe comprising the nucleotide sequence GCTTAATTCTCAATCTATCCCCACGTAT (SEQ ID NO: 21) and a 3′ probe comprising the nucleotide sequence CTCCAGATAACGTAGTGGTTTGGCTG (SEQ ID NO: 22) is used for detection of IMP; (iv) a probe pair comprising a 5′ probe comprising the nucleotide sequence CTTTCATGACGACCGCGTCGG (SEQ ID NO: 23) and a 3′ probe comprising the nucleotide sequence CTCTAGAAGGACTCTCATCGAGC (SEQ ID NO: 24) is used for detection of VIM; (v) a probe pair comprising a 5′ probe comprising the nucleotide sequence ATTTTAAAGGTAGATGCGGG (SEQ ID NO: 25) and a 3′ probe comprising the nucleotide sequence CGCCCTGTGATTTATGTTCA (SEQ ID NO: 26) is used for detection of OXA-48-like; (vi) a probe pair comprising a 5′ probe comprising the nucleotide sequence GTTTTATGCACCCAGGAAGC (SEQ ID NO: 27) and a 3′ probe comprising the nucleotide sequence TCTGCTGCGGCCAGTCATA (SEQ ID NO: 28) is used for detection of DHA; and/or (vii) a probe pair comprising a 5′ probe comprising the nucleotide sequence GCGGCTGCCAGTTTTGATAA (SEQ ID NO: 29) and a 3′ probe comprising the nucleotide sequence GTGGCTAAGTGCAGCAGGC (SEQ ID NO: 30) is used for detection of CMY. In some embodiments, the method comprises amplifying a target nucleic acid characteristic of Enterobacter spp. and Klebsiella spp., and a probe pair comprising a 5′ probe comprising the nucleotide sequence CGTTCCACTAACACACAAGCTGATTCAG (SEQ ID NO: 31) and a 3′ probe comprising the nucleotide sequence ATCTCGGTTGATTTCTTTTCCTCGGG (SEQ ID NO: 32) is used for detection of the target nucleic acid characteristic of Enterobacter spp. and Klebsiella spp.

In some embodiments, a probe pair comprising a 5′ probe comprising the nucleotide sequence GCGACCGGCAGGTTGATCT (SEQ ID NO: 17) and a 3′ probe comprising the nucleotide sequence CATGTCGAGATAGGAAGTGTGCTGC (SEQ ID NO: 18) is used for detection of NDM. For example, in some embodiments, the method comprises amplifying one or more (e.g., one, two, three, four, five, six, or all seven) antibiotic resistance genes selected from the group consisting of NDM, KPC, IMP, VIM, OXA-48-like, DHA, and/or CMY, and wherein: (i) a probe pair comprising a 5′ probe comprising the nucleotide sequence GCGACCGGCAGGTTGATCT (SEQ ID NO: 17) and a 3′ probe comprising the nucleotide sequence CATGTCGAGATAGGAAGTGTGCTGC (SEQ ID NO: 18) is used for detection of NDM; (ii) a probe pair comprising a 5′ probe comprising the nucleotide sequence CGGAACCATTCGCTAAACTC (SEQ ID NO: 19) and a 3′ probe comprising the nucleotide sequence AGGCGCAACTGTAAGTTACCG (SEQ ID NO: 20) is used for detection of KPC; (iii) a probe pair comprising a 5′ probe comprising the nucleotide sequence GCTTAATTCTCAATCTATCCCCACGTAT (SEQ ID NO: 21) and a 3′ probe comprising the nucleotide sequence CTCCAGATAACGTAGTGGTTTGGCTG (SEQ ID NO: 22) is used for detection of IMP; (iv) a probe pair comprising a 5′ probe comprising the nucleotide sequence CTTTCATGACGACCGCGTCGG (SEQ ID NO: 23) and a 3′ probe comprising the nucleotide sequence CTCTAGAAGGACTCTCATCGAGC (SEQ ID NO: 24) is used for detection of VIM; (v) a probe pair comprising a 5′ probe comprising the nucleotide sequence ATTTTAAAGGTAGATGCGGG (SEQ ID NO: 25) and a 3′ probe comprising the nucleotide sequence CGCCCTGTGATTTATGTTCA (SEQ ID NO: 26) is used for detection of OXA-48-like; (vi) a probe pair comprising a 5′ probe comprising the nucleotide sequence GTTTTATGCACCCAGGAAGC (SEQ ID NO: 27) and a 3′ probe comprising the nucleotide sequence TCTGCTGCGGCCAGTCATA (SEQ ID NO: 28) is used for detection of DHA; and/or (vii) a probe pair comprising a 5′ probe comprising the nucleotide sequence GCGGCTGCCAGTTTTGATAA (SEQ ID NO: 29) and a 3′ probe comprising the nucleotide sequence GTGGCTAAGTGCAGCAGGC (SEQ ID NO: 30) is used for detection of CMY. In some embodiments, the method comprises amplifying a target nucleic acid characteristic of Enterobacter spp. and Klebsiella spp., and a probe pair comprising a 5′ probe comprising the nucleotide sequence CGTTCCACTAACACACAAGCTGATTCAG (SEQ ID NO: 31) and a 3′ probe comprising the nucleotide sequence ATCTCGGTTGATTTCTTTTCCTCGGG (SEQ ID NO: 32) is used for detection of the target nucleic acid characteristic of Enterobacter spp. and Klebsiella spp.

In other embodiments, a probe pair comprising a 5′ probe comprising the nucleotide sequence GCGACCGGCAGGTTGATCTCCTGC (SEQ ID NO: 95) and a 3′ probe comprising the nucleotide sequence CGGCATGTCGAGATAGGAAGTGTGCTGC (SEQ ID NO: 96) is used for detection of NDM. For example, in some embodiments, the method comprises amplifying one or more (e.g., one, two, three, four, five, six, or all seven) antibiotic resistance genes selected from the group consisting of NDM, KPC, IMP, VIM, OXA-48-like, DHA, and/or CMY, and wherein: (i) a probe pair comprising a 5′ probe comprising the nucleotide sequence GCGACCGGCAGGTTGATCTCCTGC (SEQ ID NO: 95) and a 3′ probe comprising the nucleotide sequence CGGCATGTCGAGATAGGAAGTGTGCTGC (SEQ ID NO: 96) is used for detection of NDM; (ii) a probe pair comprising a 5′ probe comprising the nucleotide sequence CGGAACCATTCGCTAAACTC (SEQ ID NO: 19) and a 3′ probe comprising the nucleotide sequence AGGCGCAACTGTAAGTTACCG (SEQ ID NO: 20) is used for detection of KPC; (iii) a probe pair comprising a 5′ probe comprising the nucleotide sequence GCTTAATTCTCAATCTATCCCCACGTAT (SEQ ID NO: 21) and a 3′ probe comprising the nucleotide sequence CTCCAGATAACGTAGTGGTTTGGCTG (SEQ ID NO: 22) is used for detection of IMP; (iv) a probe pair comprising a 5′ probe comprising the nucleotide sequence CTTTCATGACGACCGCGTCGG (SEQ ID NO: 23) and a 3′ probe comprising the nucleotide sequence CTCTAGAAGGACTCTCATCGAGC (SEQ ID NO: 24) is used for detection of VIM; (v) a probe pair comprising a 5′ probe comprising the nucleotide sequence ATTTTAAAGGTAGATGCGGG (SEQ ID NO: 25) and a 3′ probe comprising the nucleotide sequence CGCCCTGTGATTTATGTTCA (SEQ ID NO: 26) is used for detection of OXA-48-like; (vi) a probe pair comprising a 5′ probe comprising the nucleotide sequence GTTTTATGCACCCAGGAAGC (SEQ ID NO: 27) and a 3′ probe comprising the nucleotide sequence TCTGCTGCGGCCAGTCATA (SEQ ID NO: 28) is used for detection of DHA; and/or (vii) a probe pair comprising a 5′ probe comprising the nucleotide sequence GCGGCTGCCAGTTTTGATAA (SEQ ID NO: 29) and a 3′ probe comprising the nucleotide sequence GTGGCTAAGTGCAGCAGGC (SEQ ID NO: 30) is used for detection of CMY. In some embodiments, the method comprises amplifying a target nucleic acid characteristic of Enterobacter spp. and Klebsiella spp., and a probe pair comprising a 5′ probe comprising the nucleotide sequence CGTTCCACTAACACACAAGCTGATTCAG (SEQ ID NO: 31) and a 3′ probe comprising the nucleotide sequence ATCTCGGTTGATTTCTTTTCCTCGGG (SEQ ID NO: 32) is used for detection of the target nucleic acid characteristic of Enterobacter spp. and Klebsiella spp.

In another example, the method comprises amplifying one or more (e.g., one, two, three, four, five, six, seven, eight, nine, ten, eleven, twelve, or all thirteen) antibiotic resistance genes selected from the group consisting of KPC, CTX-M 14, CTX-M 15, NDM, VIM, IMP, vanA, vanB, mecA, mecC, OXA-48-like, CMY, and DHA, and wherein: (i) a probe pair comprising a 5′ probe comprising the nucleotide sequence CGGAACCATTCGCTAAACTC (SEQ ID NO: 19) and a 3′ probe comprising the nucleotide sequence AGGCGCAACTGTAAGTTACCG (SEQ ID NO: 20) is used for detection of KPC; (ii) a probe pair comprising a 5′ probe comprising the nucleotide sequence CTGTCGAGATCAAGCCTGCCGA (SEQ ID NO: 57) and a 3′ probe comprising the nucleotide sequence ACAAATTGATTGCCCAGCTCGGT (SEQ ID NO: 58) is used for detection of CTX-M 14; (iii) a probe pair comprising a 5′ probe comprising the nucleotide sequence GGGCGCAGCTGGTGACAT (SEQ ID NO: 83) and a 3′ probe comprising the nucleotide sequence AAGATCGTGCGCCGCTGATT (SEQ ID NO: 84) is used for detection of CTX-M 15; (iv) a probe pair comprising a 5′ probe comprising the nucleotide sequence GCGACCGGCAGGTTGATCT (SEQ ID NO: 17) or GCGACCGGCAGGTTGATCTCCTGC (SEQ ID NO: 95) and a 3′ probe comprising the nucleotide sequence CATGTCGAGATAGGAAGTGTGCTGC (SEQ ID NO: 18) or CGGCATGTCGAGATAGGAAGTGTGCTGC (SEQ ID NO: 96) is used for detection of NDM; (v) a probe pair comprising a 5′ probe comprising the nucleotide sequence CTTTCATGACGACCGCGTCGG (SEQ ID NO: 23) and a 3′ probe comprising the nucleotide sequence CTCTAGAAGGACTCTCATCGAGC (SEQ ID NO: 24) is used for detection of VIM; (vi) a probe pair comprising a 5′ probe comprising the nucleotide sequence GCTTAATTCTCAATCTATCCCCACGTAT (SEQ ID NO: 21) and a 3′ probe comprising the nucleotide sequence CTCCAGATAACGTAGTGGTTTGGCTG (SEQ ID NO: 22) is used for detection of IMP; (vii) a probe pair comprising a 5′ probe comprising the nucleotide sequence GCAGTTATAACCGTTCCCGCAG (SEQ ID NO: 86) and a 3′ probe comprising the nucleotide sequence TAACGGCCGCATTGTACTGAACG (SEQ ID NO: 87) is used for detection of vanA; (viii) a probe pair comprising a 5′ probe comprising the nucleotide sequence GCACCCGATATACTTTCTTTGCC (SEQ ID NO: 75) and a 3′ probe comprising the nucleotide sequence CGCCGACAATCAAATCATCCT (SEQ ID NO: 76) is used for detection of vanB; (ix) a probe pair comprising a 5′ probe comprising the nucleotide sequence AAAGGCTATAAAGATGATGCAG (SEQ ID NO: 89) and a 3′ probe comprising the nucleotide sequence GAGTATTTATAACAACATGAAAAATGATT (SEQ ID NO: 90) is used for detection of mecA; (x) a probe pair comprising a 5′ probe comprising the nucleotide sequence GGCTTAGAACGCCTCTATGAT (SEQ ID NO: 93) and a 3′ probe comprising the nucleotide sequence AGAGTACAAGAAAGTATTTATAAACATATGA (SEQ ID NO: 94) is used for detection of mecC; (xi) a probe pair comprising a 5′ probe comprising the nucleotide sequence ATTTTAAAGGTAGATGCGGG (SEQ ID NO: 25) and a 3′ probe comprising the nucleotide sequence CGCCCTGTGATTTATGTTCA (SEQ ID NO: 26) is used for detection of OXA-48-like; (xii) a probe pair comprising a 5′ probe comprising the nucleotide sequence GCGGCTGCCAGTTTTGATAA (SEQ ID NO: 29) and a 3′ probe comprising the nucleotide sequence GTGGCTAAGTGCAGCAGGC (SEQ ID NO: 30) is used for detection of CMY; and/or (xiii) a probe pair comprising a 5′ probe comprising the nucleotide sequence GTTTTATGCACCCAGGAAGC (SEQ ID NO: 27) and a 3′ probe comprising the nucleotide sequence TCTGCTGCGGCCAGTCATA (SEQ ID NO: 28) is used for detection of DHA.

In some embodiments, a probe pair comprising a 5′ probe comprising the nucleotide sequence GCGACCGGCAGGTTGATCT (SEQ ID NO: 17) and a 3′ probe comprising the nucleotide sequence CATGTCGAGATAGGAAGTGTGCTGC (SEQ ID NO: 18) is used for detection of NDM. For example, in some embodiments, the method comprises amplifying one or more (e.g., one, two, three, four, five, six, seven, eight, nine, ten, eleven, twelve, or all thirteen) antibiotic resistance genes selected from the group consisting of KPC, CTX-M 14, CTX-M 15, NDM, VIM, IMP, vanA, vanB, mecA, mecC, OXA-48-like, CMY, and DHA, and wherein: (i) a probe pair comprising a 5′ probe comprising the nucleotide sequence CGGAACCATTCGCTAAACTC (SEQ ID NO: 19) and a 3′ probe comprising the nucleotide sequence AGGCGCAACTGTAAGTTACCG (SEQ ID NO: 20) is used for detection of KPC; (ii) a probe pair comprising a 5′ probe comprising the nucleotide sequence CTGTCGAGATCAAGCCTGCCGA (SEQ ID NO: 57) and a 3′ probe comprising the nucleotide sequence ACAAATTGATTGCCCAGCTCGGT (SEQ ID NO: 58) is used for detection of CTX-M 14; (iii) a probe pair comprising a 5′ probe comprising the nucleotide sequence GGGCGCAGCTGGTGACAT (SEQ ID NO: 83) and a 3′ probe comprising the nucleotide sequence AAGATCGTGCGCCGCTGATT (SEQ ID NO: 84) is used for detection of CTX-M 15; (iv) a probe pair comprising a 5′ probe comprising the nucleotide sequence GCGACCGGCAGGTTGATCT (SEQ ID NO: 17) and a 3′ probe comprising the nucleotide sequence CATGTCGAGATAGGAAGTGTGCTGC (SEQ ID NO: 18); (v) a probe pair comprising a 5′ probe comprising the nucleotide sequence CTTTCATGACGACCGCGTCGG (SEQ ID NO: 23) and a 3′ probe comprising the nucleotide sequence CTCTAGAAGGACTCTCATCGAGC (SEQ ID NO: 24) is used for detection of VIM; (vi) a probe pair comprising a 5′ probe comprising the nucleotide sequence GCTTAATTCTCAATCTATCCCCACGTAT (SEQ ID NO: 21) and a 3′ probe comprising the nucleotide sequence CTCCAGATAACGTAGTGGTTTGGCTG (SEQ ID NO: 22) is used for detection of IMP; (vii) a probe pair comprising a 5′ probe comprising the nucleotide sequence GCAGTTATAACCGTTCCCGCAG (SEQ ID NO: 86) and a 3′ probe comprising the nucleotide sequence TAACGGCCGCATTGTACTGAACG (SEQ ID NO: 87) is used for detection of vanA; (viii) a probe pair comprising a 5′ probe comprising the nucleotide sequence GCACCCGATATACTTTCTTTGCC (SEQ ID NO: 75) and a 3′ probe comprising the nucleotide sequence CGCCGACAATCAAATCATCCT (SEQ ID NO: 76) is used for detection of vanB; (ix) a probe pair comprising a 5′ probe comprising the nucleotide sequence AAAGGCTATAAAGATGATGCAG (SEQ ID NO: 89) and a 3′ probe comprising the nucleotide sequence GAGTATTTATAACAACATGAAAAATGATT (SEQ ID NO: 90) is used for detection of mecA; (x) a probe pair comprising a 5′ probe comprising the nucleotide sequence GGCTTAGAACGCCTCTATGAT (SEQ ID NO: 93) and a 3′ probe comprising the nucleotide sequence AGAGTACAAGAAAGTATTTATAAACATATGA (SEQ ID NO: 94) is used for detection of mecC; (xi) a probe pair comprising a 5′ probe comprising the nucleotide sequence ATTTTAAAGGTAGATGCGGG (SEQ ID NO: 25) and a 3′ probe comprising the nucleotide sequence CGCCCTGTGATTTATGTTCA (SEQ ID NO: 26) is used for detection of OXA-48-like; (xii) a probe pair comprising a 5′ probe comprising the nucleotide sequence GCGGCTGCCAGTTTTGATAA (SEQ ID NO: 29) and a 3′ probe comprising the nucleotide sequence GTGGCTAAGTGCAGCAGGC (SEQ ID NO: 30) is used for detection of CMY; and/or (xiii) a probe pair comprising a 5′ probe comprising the nucleotide sequence GTTTTATGCACCCAGGAAGC (SEQ ID NO: 27) and a 3′ probe comprising the nucleotide sequence TCTGCTGCGGCCAGTCATA (SEQ ID NO: 28) is used for detection of DHA.

In other embodiments, a probe pair comprising a 5′ probe comprising the nucleotide sequence GCGACCGGCAGGTTGATCTCCTGC (SEQ ID NO: 95) and a 3′ probe comprising the nucleotide sequence CGGCATGTCGAGATAGGAAGTGTGCTGC (SEQ ID NO: 96) is used for detection of NDM. For example, in some embodiments, the method comprises amplifying one or more (e.g., one, two, three, four, five, six, seven, eight, nine, ten, eleven, twelve, or all thirteen) antibiotic resistance genes selected from the group consisting of KPC, CTX-M 14, CTX-M 15, NDM, VIM, IMP, vanA, vanB, mecA, mecC, OXA-48-like, CMY, and DHA, and wherein: (i) a probe pair comprising a 5′ probe comprising the nucleotide sequence CGGAACCATTCGCTAAACTC (SEQ ID NO: 19) and a 3′ probe comprising the nucleotide sequence AGGCGCAACTGTAAGTTACCG (SEQ ID NO: 20) is used for detection of KPC; (ii) a probe pair comprising a 5′ probe comprising the nucleotide sequence CTGTCGAGATCAAGCCTGCCGA (SEQ ID NO: 57) and a 3′ probe comprising the nucleotide sequence ACAAATTGATTGCCCAGCTCGGT (SEQ ID NO: 58) is used for detection of CTX-M 14; (iii) a probe pair comprising a 5′ probe comprising the nucleotide sequence GGGCGCAGCTGGTGACAT (SEQ ID NO: 83) and a 3′ probe comprising the nucleotide sequence AAGATCGTGCGCCGCTGATT (SEQ ID NO: 84) is used for detection of CTX-M 15; (iv) a probe pair comprising a 5′ probe comprising the nucleotide sequence GCGACCGGCAGGTTGATCTCCTGC (SEQ ID NO: 95) and a 3′ probe comprising the nucleotide sequence CGGCATGTCGAGATAGGAAGTGTGCTGC (SEQ ID NO: 96) is used for detection of NDM; (v) a probe pair comprising a 5′ probe comprising the nucleotide sequence CTTTCATGACGACCGCGTCGG (SEQ ID NO: 23) and a 3′ probe comprising the nucleotide sequence CTCTAGAAGGACTCTCATCGAGC (SEQ ID NO: 24) is used for detection of VIM; (vi) a probe pair comprising a 5′ probe comprising the nucleotide sequence GCTTAATTCTCAATCTATCCCCACGTAT (SEQ ID NO: 21) and a 3′ probe comprising the nucleotide sequence CTCCAGATAACGTAGTGGTTTGGCTG (SEQ ID NO: 22) is used for detection of IMP; (vii) a probe pair comprising a 5′ probe comprising the nucleotide sequence GCAGTTATAACCGTTCCCGCAG (SEQ ID NO: 86) and a 3′ probe comprising the nucleotide sequence TAACGGCCGCATTGTACTGAACG (SEQ ID NO: 87) is used for detection of vanA; (viii) a probe pair comprising a 5′ probe comprising the nucleotide sequence GCACCCGATATACTTTCTTTGCC (SEQ ID NO: 75) and a 3′ probe comprising the nucleotide sequence CGCCGACAATCAAATCATCCT (SEQ ID NO: 76) is used for detection of vanB; (ix) a probe pair comprising a 5′ probe comprising the nucleotide sequence AAAGGCTATAAAGATGATGCAG (SEQ ID NO: 89) and a 3′ probe comprising the nucleotide sequence GAGTATTTATAACAACATGAAAAATGATT (SEQ ID NO: 90) is used for detection of mecA; (x) a probe pair comprising a 5′ probe comprising the nucleotide sequence GGCTTAGAACGCCTCTATGAT (SEQ ID NO: 93) and a 3′ probe comprising the nucleotide sequence AGAGTACAAGAAAGTATTTATAAACATATGA (SEQ ID NO: 94) is used for detection of mecC; (xi) a probe pair comprising a 5′ probe comprising the nucleotide sequence ATTTTAAAGGTAGATGCGGG (SEQ ID NO: 25) and a 3′ probe comprising the nucleotide sequence CGCCCTGTGATTTATGTTCA (SEQ ID NO: 26) is used for detection of OXA-48-like; (xii) a probe pair comprising a 5′ probe comprising the nucleotide sequence GCGGCTGCCAGTTTTGATAA (SEQ ID NO: 29) and a 3′ probe comprising the nucleotide sequence GTGGCTAAGTGCAGCAGGC (SEQ ID NO: 30) is used for detection of CMY; and/or (xiii) a probe pair comprising a 5′ probe comprising the nucleotide sequence GTTTTATGCACCCAGGAAGC (SEQ ID NO: 27) and a 3′ probe comprising the nucleotide sequence TCTGCTGCGGCCAGTCATA (SEQ ID NO: 28) is used for detection of DHA.

In another example, the method comprises amplifying one or more (e.g., one, two, three, four, five, six, seven, eight, nine, or all ten) antibiotic resistance genes selected from the group consisting of CTX-M 14, CTX-M 15, ermA, ermB, mecA, mefA, SHV, TEM, vanA, and vanB, and wherein: (i) a probe pair comprising a 5′ probe comprising the nucleotide sequence CTGTCGAGATCAAGCCTGCCGA (SEQ ID NO: 57) and a 3′ probe comprising the nucleotide sequence ACAAATTGATTGCCCAGCTCGGT (SEQ ID NO: 58) is used for detection of CTX-M 14; (ii) a probe pair comprising a 5′ probe comprising the nucleotide sequence AATCAGCGGCGCACGATCTTT (SEQ ID NO: 59) and a 3′ probe comprising the nucleotide sequence AATGCTCGCTGCACCGGTGGTAT (SEQ ID NO: 60) is used for detection of CTX-M 15; (iii) a probe pair comprising a 5′ probe comprising the nucleotide sequence AAATCTGCAACGAGCTTTGGG (SEQ ID NO: 61) and a 3′ probe comprising the nucleotide sequence GTTTATAAGTGGGTAAACCGTGAATATC (SEQ ID NO: 62) is used for detection of ermA; (iv) a probe pair comprising a 5′ probe comprising the nucleotide sequence TTCGTGTCACTTTAATTCACCAAGAT (SEQ ID NO: 63) and a 3′ probe comprising the nucleotide sequence AAAGCCATGCGTCTGACATCT (SEQ ID NO: 64) is used for detection of ermB; (v) a probe pair comprising a 5′ probe comprising the nucleotide sequence AAGCTCCAACATGAAGATGGCT (SEQ ID NO: 65) and a 3′ probe comprising the nucleotide sequence AGATGGCAAAGATATTCAACTAAC (SEQ ID NO: 66) is used for detection of mecA; (vi) a probe pair comprising a 5′ probe comprising the nucleotide sequence AGTGCCATCTTGCAAATGGCGAT (SEQ ID NO: 67) and a 3′ probe comprising the nucleotide sequence TGCAATTGGTGTGTTAGTGGATCGTCATGATA (SEQ ID NO: 68) is used for detection of mefA; (vii) a probe pair comprising a 5′ probe comprising the nucleotide sequence CAGTGGATGGTGGACGATCGGGT (SEQ ID NO: 69) and a 3′ probe comprising the nucleotide sequence TTGTGGTGATTTATCTGCGGGATACT (SEQ ID NO: 70) is used for detection of SHV; (viii) a probe pair comprising a 5′ probe comprising the nucleotide sequence CGCCAGTTAATAGTTTGCGCAACG (SEQ ID NO: 71) and a 3′ probe comprising the nucleotide sequence AAAGCGGTTAGCTCCTTCGGTCCT (SEQ ID NO: 72) is used for detection of TEM; (ix) a probe pair comprising a 5′ probe comprising the nucleotide sequence CGTTCAGTACAATGCGGCCGTTA (SEQ ID NO: 73) and a 3′ probe comprising the nucleotide sequence CTGCGGGAACGGTTATAACTGC (SEQ ID NO: 74) is used for detection of vanA; and/or (x) a probe pair comprising a 5′ probe comprising the nucleotide sequence GCACCCGATATACTTTCTTTGCC (SEQ ID NO: 75) and a 3′ probe comprising the nucleotide sequence CGCCGACAATCAAATCATCCT (SEQ ID NO: 76) is used for detection of vanB. In some embodiments, the method comprises amplifying a target nucleic acid characteristic of Streptococcus pneumoniae, and a probe pair comprising a 5′ probe comprising the nucleotide sequence TTGACCAGTTCCGAGCAAATGGTA (SEQ ID NO: 77) and a 3′ probe comprising the nucleotide sequence GACAGTATCGATGTTCCAGCAGCT (SEQ ID NO: 78) is used for detection of the target nucleic acid characteristic of Streptococcus pneumoniae.

Any suitable number of magnetic particles can be used in the methods, e.g., as described below. In some embodiments, an assay sample is contacted with 1×10⁶ to 1×10¹³ magnetic particles per milliliter of the biological sample.

In some embodiments, step (h) includes measuring the T₂ relaxation response of the assay sample, and increasing agglomeration in the assay sample produces an increase in the observed T₂ relaxation time of the assay sample.

The magnetic particles can have any suitable size, e.g., as described below. In some embodiments, the magnetic particles have a mean diameter of from 700 nm to 1200 nm. In some embodiments, the magnetic particles have a mean diameter of from 650 nm to 950 nm. In some embodiments, the magnetic particles have a mean diameter of from 670 nm to 890 nm.

The magnetic particles can have any suitable T₂ relaxivity per particle. In some embodiments, the magnetic particles have a T₂ relaxivity per particle of from 1×10⁹ to 1×10¹² mM⁻¹ s⁻¹.

In some embodiments, the magnetic particles are substantially monodisperse.

In some embodiments, the method further includes sequencing the first and/or second amplified antibiotic resistance target nucleic acid.

In yet another example, provided herein is a method for detecting the presence of an antibiotic resistance gene in a biological sample obtained from a subject, wherein the biological sample includes subject-derived cells or cell debris, the method including: (a) amplifying in the biological sample or a fraction thereof one or more antibiotic resistance target nucleic acids in a multiplexed amplification reaction, wherein the multiplexed amplification reaction is configured to amplify target nucleic acids characteristic of two or more antibiotic resistance genes selected from the group consisting of NDM, KPC, IMP, VIM, OXA-48-like, DHA, CMY, FOX, mecA, mecC, vanA, vanB, CTX-M 14, CTX-M 15, mefA, mefE, ermA, ermB, SHV, and TEM; and (b) sequencing the one or more amplified antibiotic resistance target nucleic acids to detect whether one or more of the antibiotic resistance genes is present in the biological sample, wherein the method is capable of detecting an antibiotic resistance gene of a pathogen present at a concentration of 10 cells/mL of biological sample or less (e.g., 10 cells/mL, 9 cells/mL, 8 cells/mL, 7 cells/mL, 6 cells/mL, 5 cells/mL, 4 cells/mL, 3 cells/mL, 2 cells/mL, or 1 cells/mL). In some embodiments, the two or more antibiotic resistance genes comprises at least one of IMP, OXA-48-like, DHA, CMY, FOX, CTX-M 14, CTX-M 15, mecC, mefA, mefE, ermA, ermB, and TEM. In some embodiments, the two or more antibiotic resistance genes include NDM, KPC, IMP, VIM, OXA-48-like, DHA, CMY, mecA, mecC, vanA, vanB, CTX-M 14, CTX-M 15, mefA, mefE, ermA, ermB, SHV, and/or TEM.

In some embodiments, the method includes amplifying and/or sequencing at least three, at least four, at least five, at least six, or all seven antibiotic resistance genes selected from the group consisting of NDM, KPC, IMP, VIM, OXA-48-like, DHA, and CMY. In some embodiments, the multiplexed amplification reaction is configured to amplify at least three, at least four, at least five, at least six, or all seven antibiotic resistance genes selected from the group consisting of NDM, KPC, IMP, VIM, OXA-48-like, DHA, and CMY. See, e.g., Tables 1-6 below. In some embodiments, the method includes amplifying and/or sequencing NDM, KPC, IMP, VIM, OXA-48-like, DHA, and CMY. In some embodiments, the multiplexed amplification reaction is configured to amplify NDM, KPC, IMP, VIM, OXA-48-like, DHA, and CMY.

In some embodiments, (i) NDM is amplified in the presence of a forward primer comprising the nucleotide sequence CCAGCTCGCACCGAATGTCT (SEQ ID NO: 1) and a reverse primer comprising the nucleotide sequence CATCTTGTCCTGATGCGCGTGAGTCA (SEQ ID NO: 2); (ii) KPC is amplified in the presence of a forward primer comprising the nucleotide sequence TTTCTGCCACCGCGCTGAC (SEQ ID NO: 3) and a reverse primer comprising the nucleotide sequence GCAGCAAGAAAGCCCTTGAATG (SEQ ID NO: 4); (iii) IMP is amplified in the presence of a forward primer comprising the nucleotide sequence ACATTTCCATAGCGACAGCACGGGCGGAAT (SEQ ID NO: 5) and a reverse primer comprising the nucleotide sequence GGACTTTGGCCAAGCTTCTAAATTTGCGTC (SEQ ID NO: 6); (iv) VIM is amplified in the presence of a forward primer comprising the nucleotide sequence CGTGCAGTCTCCACGCACT (SEQ ID NO: 7) and a reverse primer comprising the nucleotide sequence TCGAATGCGCAGCACCGGGATAG (SEQ ID NO: 8); (v) OXA-48-like is amplified in the presence of a forward primer comprising the nucleotide sequence GGCTGTGTTTITGGTGGCATCGATTATC (SEQ ID NO: 9) and a reverse primer comprising the nucleotide sequence TCCCACTTAAAGACTTGGTGTTCATCC (SEQ ID NO: 10); (vi) DHA is amplified in the presence of a forward primer comprising the nucleotide sequence ACGGGCCGGTAATGCGGATCTGGA (SEQ ID NO: 11) and a reverse primer comprising the nucleotide sequence TATTCGCCAGAATCACAATCGCCACCTGT (SEQ ID NO: 12); and/or (vii) CMY is amplified in the presence of a forward primer comprising the nucleotide sequence CCGCGGCGAAATTAAGCTCAGCGA (SEQ ID NO: 13) and a reverse primer comprising the nucleotide sequence CCAAACAGACCAATGCTGGAGTTAG (SEQ ID NO: 14).

In another example, in some embodiments, (i) the method comprises amplifying and/or sequencing at least three, at least four, at least five, at least six, at least seven, at least eight, at least nine, at least ten, at least eleven, at least twelve, or all thirteen antibiotic resistance genes selected from the group consisting of KPC, CTX-M 14, CTX-M 15, NDM, VIM, IMP, vanA, vanB, mecA, mecC, OXA-48-like, CMY, and DHA; and/or (ii) the multiplexed amplification reaction is configured to amplify at least three, at least four, at least five, at least six, at least seven, at least eight, at least nine, at least ten, at least eleven, at least twelve, or all thirteen antibiotic resistance genes selected from the group consisting of KPC, CTX-M 14, CTX-M 15, NDM, VIM, IMP, vanA, vanB, mecA, mecC, OXA-48-like, CMY, and DHA. In some embodiments, (i) the method comprises amplifying and/or sequencing KPC, CTX-M 14, CTX-M 15, NDM, VIM, IMP, vanA, vanB, mecA, mecC, OXA-48-like, CMY, and DHA; and/or (ii) the multiplexed amplification reaction is configured to amplify KPC, CTX-M 14, CTX-M 15, NDM, VIM, IMP, vanA, vanB, mecA, mecC, OXA-48-like, CMY, and DHA.

In some embodiments, (i) KPC is amplified in the presence of a forward primer comprising the nucleotide sequence TTTCTGCCACCGCGCTGAC (SEQ ID NO: 3) and a reverse primer comprising the nucleotide sequence GCAGCAAGAAAGCCCTTGAATG (SEQ ID NO: 4); (ii) CTX-M 14 is amplified in the presence of a forward primer comprising the nucleotide sequence ACGCTTTCCAATGTGCAGTACCAGTA (SEQ ID NO: 33) and a reverse primer comprising the nucleotide sequence TGCGATCCAGACGAAACGTCTCATCG (SEQ ID NO: 34); (iii) CTX-M 15 is amplified in the presence of a forward primer comprising the nucleotide sequence CCTCGGGCAATGGCGCAAAC (SEQ ID NO: 81) and a reverse primer comprising the nucleotide sequence ATCGCGACGGCTTTCTGCCTTA (SEQ ID NO: 82); (iv) NDM is amplified in the presence of a forward primer comprising the nucleotide sequence CCAGCTCGCACCGAATGTCT (SEQ ID NO: 1) and a reverse primer comprising the nucleotide sequence CATCTTGTCCTGATGCGCGTGAGTCA (SEQ ID NO: 2); (v) VIM is amplified in the presence of a forward primer comprising the nucleotide sequence CGTGCAGTCTCCACGCACT (SEQ ID NO: 7) and a reverse primer comprising the nucleotide sequence TCGAATGCGCAGCACCGGGATAG (SEQ ID NO: 8); (vi) IMP is amplified in the presence of a forward primer comprising the nucleotide sequence ACATTTCCATAGCGACAGCACGGGCGGAAT (SEQ ID NO: 5) and a reverse primer comprising the nucleotide sequence GGACTTTGGCCAAGCTTCTAAATTTGCGTC (SEQ ID NO: 6); (vii) vanA is amplified in the presence of a forward primer comprising the nucleotide sequence TATTCATCAGGAAGTCGAGCCGGA (SEQ ID NO: 85) and a reverse primer comprising the nucleotide sequence CAGTTCGGGAAGTGCAATACCTGCA (SEQ ID NO: 50); (viii) vanB is amplified in the presence of a forward primer comprising the nucleotide sequence AATTGAGCAAGCGATTTCGGGCTGT (SEQ ID NO: 51) and a reverse primer comprising the nucleotide sequence AAGATCAACACGGGCAAGCCCTCT (SEQ ID NO: 88); (ix) mecA is amplified in the presence of a forward primer comprising the nucleotide sequence ATGTTGGTCCCATTAACTCTGAAGAA (SEQ ID NO: 41) and a reverse primer comprising the nucleotide sequence CACCTGTTTGAGGGTGGATAGCAGTA (SEQ ID NO: 42); (x) mecC is amplified in the presence of a forward primer comprising the nucleotide sequence ATGTGGGTCCAATTAATTCTGACGAG (SEQ ID NO: 91) and a reverse primer comprising the nucleotide sequence CTCCAGTTTTGGTTGTAATGCTGTA (SEQ ID NO: 92); (xi) OXA-48-like is amplified in the presence of a forward primer comprising the nucleotide sequence GGCTGTGTTTITGGTGGCATCGATTATC (SEQ ID NO: 9) and a reverse primer comprising the nucleotide sequence TCCCACTTAAAGACTTGGTGTTCATCC (SEQ ID NO: 10); (xii) CMY is amplified in the presence of a forward primer comprising the nucleotide sequence CCGCGGCGAAATTAAGCTCAGCGA (SEQ ID NO: 13) and a reverse primer comprising the nucleotide sequence CCAAACAGACCAATGCTGGAGTTAG (SEQ ID NO: 14); and/or (xiii) DHA is amplified in the presence of a forward primer comprising the nucleotide sequence ACGGGCCGGTAATGCGGATCTGGA (SEQ ID NO: 11) and a reverse primer comprising the nucleotide sequence TATTCGCCAGAATCACAATCGCCACCTGT (SEQ ID NO: 12).

In another example, in some embodiments, (i) the method comprises amplifying and/or detecting at least three, at least four, at least five, at least six, at least seven, at least eight, at least nine, or all ten antibiotic resistance genes selected from the group consisting of CTX-M 14, CTX-M 15, ermA, ermB, mecA, mefA, SHV, TEM, vanA, and vanB; and/or (ii) the multiplexed amplification reaction is configured to amplify at least three, at least four, at least five, at least six, at least seven, at least eight, at least nine, or all ten antibiotic resistance genes selected from the group consisting of CTX-M 14, CTX-M 15, ermA, ermB, mecA, mefA, SHV, TEM, vanA, and vanB. In some embodiments, (i) the method comprises amplifying and/or detecting CTX-M 14, CTX-M 15, ermA, ermB, mecA, mefA, SHV, TEM, vanA, and vanB; and/or (ii) the multiplexed amplification reaction is configured to amplify CTX-M 14, CTX-M 15, ermA, ermB, mecA, mefA, SHV, TEM, vanA, and vanB.

In some embodiments, (i) CTX-M 14 is amplified in the presence of a forward primer comprising the nucleotide sequence ACGCTTTCCAATGTGCAGTACCAGTA (SEQ ID NO: 33) and a reverse primer comprising the nucleotide sequence TGCGATCCAGACGAAACGTCTCATCG (SEQ ID NO: 34); (ii) CTX-M 15 is amplified in the presence of a forward primer comprising the nucleotide sequence GTGATACCACTTCACCTCGGGCAA (SEQ ID NO: 35) and a reverse primer comprising the nucleotide sequence AATACATCGCGACGGCTTTCTGCC (SEQ ID NO: 36); (iii) ermA is amplified in the presence of a forward primer comprising the nucleotide sequence AGAATTACCTTTGAAAGTCAGGC (SEQ ID NO: 37) and a reverse primer comprising the nucleotide sequence GCTTCAAAGCCTGTCGGAATTGGTTT (SEQ ID NO: 38); (iv) ermB is amplified in the presence of a forward primer comprising the nucleotide sequence GGGCATTTAACGACGAAACTGGCTA (SEQ ID NO: 39) and a reverse primer comprising the nucleotide sequence GTGTTCGGTGAATATCCAAGGTACGC (SEQ ID NO: 40); (v) mecA is amplified in the presence of a forward primer comprising the nucleotide sequence ATGTTGGTCCCATTAACTCTGAAGAA (SEQ ID NO: 41) and a reverse primer comprising the nucleotide sequence CACCTGTTTGAGGGTGGATAGCAGTA (SEQ ID NO: 42); (vi) mefA is amplified in the presence of a forward primer comprising the nucleotide sequence GCAGGGCAAGCAGTATCATTAATCAC (SEQ ID NO: 43) and a reverse primer comprising the nucleotide sequence AATTAAATCAGCACCAATCATTATCTTCTTCC (SEQ ID NO: 44); (vii) SHV is amplified in the presence of a forward primer comprising the nucleotide sequence AAGCTGCTGACCAGCCAGCGTCTGA (SEQ ID NO: 45) and a reverse primer comprising the nucleotide sequence CGGCGATTTGCTGATTTCGCTCG (SEQ ID NO: 46); (viii) TEM is amplified in the presence of a forward primer comprising the nucleotide sequence TGCAGTGCTGCCATAACCATGAGTGA (SEQ ID NO: 47) and a reverse primer comprising the nucleotide sequence AGCGCAGAAGTGGTCCTGCAACTTT (SEQ ID NO: 48); (ix) vanA is amplified in the presence of a forward primer comprising the nucleotide sequence CAGTACGGAATCTTTCGTATTCATCAGGA (SEQ ID NO: 49) and a reverse primer comprising the nucleotide sequence CAGTTCGGGAAGTGCAATACCTGCA (SEQ ID NO: 50); and/or (x) vanB is amplified in the presence of a forward primer comprising the nucleotide sequence AATTGAGCAAGCGATTTCGGGCTGT (SEQ ID NO: 51) and a reverse primer comprising the nucleotide sequence CGTTTAGAACGATGCCGCCATCCT (SEQ ID NO: 52).

In some embodiments, the amplifying step (a) further comprises amplifying a target nucleic acid characteristic of a bacterial pathogen, and detecting step (b) further comprises detecting the amplified target nucleic acid characteristic of a bacterial pathogen to determine whether the pathogen is present; and/or the multiplexed amplification reaction is configured to amplify a target nucleic acid characteristic of a bacterial pathogen.

For example, in some embodiments, amplifying step (a) further includes amplifying a target nucleic acid characteristic of a bacterial pathogen, and step (b) further includes sequencing the amplified target nucleic acid characteristic of a bacterial pathogen to determine whether the bacterial pathogen is present. In some embodiments, the multiplexed amplification reaction is configured to amplify a target nucleic acid characteristic of a bacterial pathogen. In some embodiments, the target nucleic acid characteristic of a bacterial pathogen is characteristic of an Enterobacter spp., a Klebsiella spp., and/or Streptococcus pneumoniae. In some embodiments, the target nucleic acid characteristic of Enterobacter spp. and Klebsiella spp. is amplified in the presence of a forward primer comprising the nucleotide sequence ATTCGTTGCACTATCGTTAACTGAATACA (SEQ ID NO: 15) and a reverse primer comprising the nucleotide sequence CTGTACCGTCGGACTTTCCAGAC (SEQ ID NO: 16). In other embodiments, the target nucleic acid characteristic of a bacterial pathogen is characteristic of Streptococcus pneumoniae. In some embodiments, the target nucleic acid characteristic of Streptococcus pneumoniae is amplified in the presence of a forward primer comprising the nucleotide sequence CCTTGGACGGAAATGTAGCTGGCA (SEQ ID NO: 53) and a reverse primer comprising the nucleotide sequence AATCACATGGTTGACACCTGCTGTG (SEQ ID NO: 54).

In some embodiments, the method includes amplifying and/or detecting NDM, KPC, IMP, VIM, OXA-48-like, DHA, CMY, and a target nucleic acid characteristic of Enterobacter spp. and Klebsiella spp.

In some embodiments, the multiplexed amplification reaction is configured to amplify NDM, KPC, IMP, VIM, OXA-48-like, DHA, CMY, and a target nucleic acid characteristic of Enterobacter spp. and Klebsiella spp.

In other embodiments, the method comprises amplifying and/or detecting KPC, CTX-M 14, CTX-M 15, NDM, VIM, IMP, vanA, vanB, and mecA, mecC, OXA-48-like, CMY, DHA, and a target nucleic acid characteristic of Enterobacter spp. and Klebsiella spp. In some embodiments, the multiplexed amplification reaction is configured to amplify KPC, CTX-M 14, CTX-M 15, NDM, VIM, IMP, vanA, vanB, and mecA, mecC, OXA-48-like, CMY, DHA, and a target nucleic acid characteristic of Enterobacter spp. and Klebsiella spp.

In yet other embodiments, the method comprises amplifying and/or detecting CTX-M 14, CTX-M 15, ermA, ermB, mecA, mefA, SHV, TEM, vanA, vanB, and a target nucleic acid characteristic of Streptococcus pneumoniae. In some embodiments, the multiplexed amplification reaction is configured to amplify CTX-M 14, CTX-M 15, ermA, ermB, mecA, mefA, SHV, TEM, vanA, vanB, and a target nucleic acid characteristic of Streptococcus pneumoniae.

In some embodiments, step (a) includes amplifying the one or more antibiotic resistance target nucleic acids in a lysate produced by lysing cells in the biological sample. In some embodiments, the lysate has at least about a 2:1, a 5:1, a 10:1, a 20:1, a 40:1, or a 60:1 higher concentration of cell debris relative to the biological sample. In some embodiments, the cell debris is solid material.

In some embodiments of any of the preceding methods, the biological sample has a volume of about 0.1 mL to about 5 mL. In some embodiments, the biological sample has a volume of about 2 mL. In some embodiments of any of the preceding aspects, the biological sample is selected from the group consisting of blood, bloody fluids, tissue samples, bronchiolar lavage (BAL), urine, cerebrospinal fluid (CSF), synovial fluid (SF), and sputum. In some embodiments, the blood is whole blood, a crude blood lysate, serum, or plasma. In some embodiments, the whole blood is ethylenediaminetetraacetic acid (EDTA) whole blood, sodium citrate whole blood, sodium heparin whole blood, lithium heparin whole blood, or potassium oxylate/sodium fluoride whole blood. In some embodiments, the bloody fluid is wound exudate, wound aspirate, phlegm, or bile. In some embodiments, the tissue sample is a tissue sample from a transplant, a tissue biopsy (e.g., a skin biopsy, muscle biopsy, or lymph node biopsy), a homogenized tissue sample, or bone. In some embodiments, the biological sample is urine or BAL. In some embodiments of any of the preceding aspects, the biological sample is a swab.

In some embodiments, the method further includes detecting the amplified target pathogen nucleic acid(s) using T2 magnetic resonance (T2MR).

In another example, provided herein is a method for detecting the presence of an antibiotic resistance gene in a whole blood sample, the method including: (a) contacting a whole blood sample suspected of containing one or more pathogen cells with an erythrocyte lysis agent, thereby lysing red blood cells; (b) centrifuging the product of step (a) to form a supernatant and a pellet; (c) discarding some or all of the supernatant of step (b) and resuspending the pellet to form an extract, optionally washing the pellet one or more times prior to resuspending the pellet; (d) lysing the remaining cells in the extract of step (c) to form a lysate, the lysate containing both subject cell nucleic acid and pathogen nucleic acid; (e) amplifying in the lysate of step (d) one or more antibiotic resistance target nucleic acids in a multiplexed amplification reaction, wherein the multiplexed amplification reaction is configured to amplify target nucleic acids characteristic of two or more antibiotic resistance genes selected from the group consisting of NDM, KPC, IMP, VIM, OXA-48-like, DHA, CMY, FOX, mecA, mecC, vanA, vanB, CTX-M 14, CTX-M 15, mefA, mefE, ermA, ermB, SHV, and TEM; and (f) detecting the one or more amplified target antibiotic resistance target nucleic acids, thereby detecting the presence of the one or more of the antibiotic resistance genes in the sample. In some embodiments, the two or more antibiotic resistance genes comprises at least one of IMP, OXA-48-like, DHA, CMY, FOX, CTX-M 14, CTX-M 15, mecC, mefA, mefE, ermA, ermB, or TEM.

In some embodiments, the method includes amplifying and/or detecting at least three, at least four, at least five, at least six, or all seven antibiotic resistance genes selected from the group consisting of NDM, KPC, IMP, VIM, OXA-48-like, DHA, and CMY. In some embodiments, the multiplexed amplification reaction is configured to amplify at least three, at least four, at least five, at least six, or all seven antibiotic resistance genes selected from the group consisting of NDM, KPC, IMP, VIM, OXA-48-like, DHA, and CMY. See, e.g., Tables 1-6. In some embodiments of the previous aspect, the method includes amplifying and/or detecting NDM, KPC, IMP, VIM, OXA-48-like, DHA, and CMY. In some embodiments, the multiplexed amplification reaction is configured to amplify NDM, KPC, IMP, VIM, OXA-48-like, DHA, and CMY.

In some embodiments, (i) NDM is amplified in the presence of a forward primer comprising the nucleotide sequence CCAGCTCGCACCGAATGTCT (SEQ ID NO: 1) and a reverse primer comprising the nucleotide sequence CATCTTGTCCTGATGCGCGTGAGTCA (SEQ ID NO: 2); (ii) KPC is amplified in the presence of a forward primer comprising the nucleotide sequence TTTCTGCCACCGCGCTGAC (SEQ ID NO: 3) and a reverse primer comprising the nucleotide sequence GCAGCAAGAAAGCCCTTGAATG (SEQ ID NO: 4); (iii) IMP is amplified in the presence of a forward primer comprising the nucleotide sequence ACATTTCCATAGCGACAGCACGGGCGGAAT (SEQ ID NO: 5) and a reverse primer comprising the nucleotide sequence GGACTTTGGCCAAGCTTCTAAATTTGCGTC (SEQ ID NO: 6); (iv) VIM is amplified in the presence of a forward primer comprising the nucleotide sequence CGTGCAGTCTCCACGCACT (SEQ ID NO: 7) and a reverse primer comprising the nucleotide sequence TCGAATGCGCAGCACCGGGATAG (SEQ ID NO: 8); (v) OXA-48-like is amplified in the presence of a forward primer comprising the nucleotide sequence GGCTGTGTTTITGGTGGCATCGATTATC (SEQ ID NO: 9) and a reverse primer comprising the nucleotide sequence TCCCACTTAAAGACTTGGTGTTCATCC (SEQ ID NO: 10); (vi) DHA is amplified in the presence of a forward primer comprising the nucleotide sequence ACGGGCCGGTAATGCGGATCTGGA (SEQ ID NO: 11) and a reverse primer comprising the nucleotide sequence TATTCGCCAGAATCACAATCGCCACCTGT (SEQ ID NO: 12); and/or (vii) CMY is amplified in the presence of a forward primer comprising the nucleotide sequence CCGCGGCGAAATTAAGCTCAGCGA (SEQ ID NO: 13) and a reverse primer comprising the nucleotide sequence CCAAACAGACCAATGCTGGAGTTAG (SEQ ID NO: 14).

In another example, in some embodiments, (i) the method comprises amplifying and/or sequencing at least three, at least four, at least five, at least six, at least seven, at least eight, at least nine, at least ten, at least eleven, at least twelve, or all thirteen antibiotic resistance genes selected from the group consisting of KPC, CTX-M 14, CTX-M 15, NDM, VIM, IMP, vanA, vanB, mecA, mecC, OXA-48-like, CMY, and DHA; and/or (ii) the multiplexed amplification reaction is configured to amplify at least three, at least four, at least five, at least six, at least seven, at least eight, at least nine, at least ten, at least eleven, at least twelve, or all thirteen antibiotic resistance genes selected from the group consisting of KPC, CTX-M 14, CTX-M 15, NDM, VIM, IMP, vanA, vanB, mecA, mecC, OXA-48-like, CMY, and DHA. In some embodiments, (i) the method comprises amplifying and/or sequencing KPC, CTX-M 14, CTX-M 15, NDM, VIM, IMP, vanA, vanB, mecA, mecC, OXA-48-like, CMY, and DHA; and/or (ii) the multiplexed amplification reaction is configured to amplify KPC, CTX-M 14, CTX-M 15, NDM, VIM, IMP, vanA, vanB, mecA, mecC, OXA-48-like, CMY, and DHA.

In some embodiments, (i) KPC is amplified in the presence of a forward primer comprising the nucleotide sequence TTTCTGCCACCGCGCTGAC (SEQ ID NO: 3) and a reverse primer comprising the nucleotide sequence GCAGCAAGAAAGCCCTTGAATG (SEQ ID NO: 4); (ii) CTX-M 14 is amplified in the presence of a forward primer comprising the nucleotide sequence ACGCTTTCCAATGTGCAGTACCAGTA (SEQ ID NO: 33) and a reverse primer comprising the nucleotide sequence TGCGATCCAGACGAAACGTCTCATCG (SEQ ID NO: 34); (iii) CTX-M 15 is amplified in the presence of a forward primer comprising the nucleotide sequence CCTCGGGCAATGGCGCAAAC (SEQ ID NO: 81) and a reverse primer comprising the nucleotide sequence ATCGCGACGGCTTTCTGCCTTA (SEQ ID NO: 82); (iv) NDM is amplified in the presence of a forward primer comprising the nucleotide sequence CCAGCTCGCACCGAATGTCT (SEQ ID NO: 1) and a reverse primer comprising the nucleotide sequence CATCTTGTCCTGATGCGCGTGAGTCA (SEQ ID NO: 2); (v) VIM is amplified in the presence of a forward primer comprising the nucleotide sequence CGTGCAGTCTCCACGCACT (SEQ ID NO: 7) and a reverse primer comprising the nucleotide sequence TCGAATGCGCAGCACCGGGATAG (SEQ ID NO: 8); (vi) IMP is amplified in the presence of a forward primer comprising the nucleotide sequence ACATTTCCATAGCGACAGCACGGGCGGAAT (SEQ ID NO: 5) and a reverse primer comprising the nucleotide sequence GGACTTTGGCCAAGCTTCTAAATTTGCGTC (SEQ ID NO: 6); (vii) vanA is amplified in the presence of a forward primer comprising the nucleotide sequence TATTCATCAGGAAGTCGAGCCGGA (SEQ ID NO: 85) and a reverse primer comprising the nucleotide sequence CAGTTCGGGAAGTGCAATACCTGCA (SEQ ID NO: 50); (viii) vanB is amplified in the presence of a forward primer comprising the nucleotide sequence AATTGAGCAAGCGATTTCGGGCTGT (SEQ ID NO: 51) and a reverse primer comprising the nucleotide sequence AAGATCAACACGGGCAAGCCCTCT (SEQ ID NO: 88); (ix) mecA is amplified in the presence of a forward primer comprising the nucleotide sequence ATGTTGGTCCCATTAACTCTGAAGAA (SEQ ID NO: 41) and a reverse primer comprising the nucleotide sequence CACCTGTTTGAGGGTGGATAGCAGTA (SEQ ID NO: 42); (x) mecC is amplified in the presence of a forward primer comprising the nucleotide sequence ATGTGGGTCCAATTAATTCTGACGAG (SEQ ID NO: 91) and a reverse primer comprising the nucleotide sequence CTCCAGTTTGGTTGTAATGCTGTA (SEQ ID NO: 92); (xi) OXA-48-like is amplified in the presence of a forward primer comprising the nucleotide sequence GGCTGTGTTTITGGTGGCATCGATTATC (SEQ ID NO: 9) and a reverse primer comprising the nucleotide sequence TCCCACTTAAAGACTTGGTGTTCATCC (SEQ ID NO: 10); (xii) CMY is amplified in the presence of a forward primer comprising the nucleotide sequence CCGCGGCGAAATTAAGCTCAGCGA (SEQ ID NO: 13) and a reverse primer comprising the nucleotide sequence CCAAACAGACCAATGCTGGAGTTAG (SEQ ID NO: 14); and/or (xiii) DHA is amplified in the presence of a forward primer comprising the nucleotide sequence ACGGGCCGGTAATGCGGATCTGGA (SEQ ID NO: 11) and a reverse primer comprising the nucleotide sequence TATTCGCCAGAATCACAATCGCCACCTGT (SEQ ID NO: 12).

In another example, in some embodiments, (i) the method comprises amplifying and/or detecting at least three, at least four, at least five, at least six, at least seven, at least eight, at least nine, or all ten antibiotic resistance genes selected from the group consisting of CTX-M 14, CTX-M 15, ermA, ermB, mecA, mefA, SHV, TEM, vanA, and vanB; and/or (ii) the multiplexed amplification reaction is configured to amplify at least three, at least four, at least five, at least six, at least seven, at least eight, at least nine, or all ten antibiotic resistance genes selected from the group consisting of CTX-M 14, CTX-M 15, ermA, ermB, mecA, mefA, SHV, TEM, vanA, and vanB. In some embodiments, (i) the method comprises amplifying and/or detecting CTX-M 14, CTX-M 15, ermA, ermB, mecA, mefA, SHV, TEM, vanA, and vanB; and/or (ii) the multiplexed amplification reaction is configured to amplify CTX-M 14, CTX-M 15, ermA, ermB, mecA, mefA, SHV, TEM, vanA, and vanB.

In some embodiments, (i) CTX-M 14 is amplified in the presence of a forward primer comprising the nucleotide sequence ACGCTTTCCAATGTGCAGTACCAGTA (SEQ ID NO: 33) and a reverse primer comprising the nucleotide sequence TGCGATCCAGACGAAACGTCTCATCG (SEQ ID NO: 34); (ii) CTX-M 15 is amplified in the presence of a forward primer comprising the nucleotide sequence GTGATACCACTTCACCTCGGGCAA (SEQ ID NO: 35) and a reverse primer comprising the nucleotide sequence AATACATCGCGACGGCTTTCTGCC (SEQ ID NO: 36); (iii) ermA is amplified in the presence of a forward primer comprising the nucleotide sequence AGAATTACCTTTGAAAGTCAGGC (SEQ ID NO: 37) and a reverse primer comprising the nucleotide sequence GCTTCAAAGCCTGTCGGAATTGGTTT (SEQ ID NO: 38); (iv) ermB is amplified in the presence of a forward primer comprising the nucleotide sequence GGGCATTTAACGACGAAACTGGCTA (SEQ ID NO: 39) and a reverse primer comprising the nucleotide sequence GTGTTCGGTGAATATCCAAGGTACGC (SEQ ID NO: 40); (v) mecA is amplified in the presence of a forward primer comprising the nucleotide sequence ATGTTGGTCCCATTAACTCTGAAGAA (SEQ ID NO: 41) and a reverse primer comprising the nucleotide sequence CACCTGTTTGAGGGTGGATAGCAGTA (SEQ ID NO: 42); (vi) mefA is amplified in the presence of a forward primer comprising the nucleotide sequence GCAGGGCAAGCAGTATCATTAATCAC (SEQ ID NO: 43) and a reverse primer comprising the nucleotide sequence AATTAAATCAGCACCAATCATTATCTTCTTCC (SEQ ID NO: 44); (vii) SHV is amplified in the presence of a forward primer comprising the nucleotide sequence AAGCTGCTGACCAGCCAGCGTCTGA (SEQ ID NO: 45) and a reverse primer comprising the nucleotide sequence CGGCGATTTGCTGATTTCGCTCG (SEQ ID NO: 46); (viii) TEM is amplified in the presence of a forward primer comprising the nucleotide sequence TGCAGTGCTGCCATAACCATGAGTGA (SEQ ID NO: 47) and a reverse primer comprising the nucleotide sequence AGCGCAGAAGTGGTCCTGCAACTTT (SEQ ID NO: 48); (ix) vanA is amplified in the presence of a forward primer comprising the nucleotide sequence CAGTACGGAATCTTTCGTATTCATCAGGA (SEQ ID NO: 49) and a reverse primer comprising the nucleotide sequence CAGTTCGGGAAGTGCAATACCTGCA (SEQ ID NO: 50); and/or (x) vanB is amplified in the presence of a forward primer comprising the nucleotide sequence AATTGAGCAAGCGATTTCGGGCTGT (SEQ ID NO: 51) and a reverse primer comprising the nucleotide sequence CGTTTAGAACGATGCCGCCATCCT (SEQ ID NO: 52).

In some embodiments, the amplifying step (e) further comprises amplifying a target nucleic acid characteristic of a bacterial pathogen, and step (f) further comprises detecting the amplified target nucleic acid characteristic of a bacterial pathogen to determine whether the bacterial pathogen is present; and/or the multiplexed amplification reaction is configured to amplify a target nucleic acid characteristic of a bacterial pathogen.

For example, in some embodiments of the preceding methods, the amplifying step (e) further includes amplifying a target nucleic acid characteristic of a bacterial pathogen, and step (f) further includes detecting the amplified target nucleic acid characteristic of a bacterial pathogen to determine whether the bacterial pathogen is present. In some embodiments, the multiplexed amplification reaction is configured to amplify a target nucleic acid characteristic of a bacterial pathogen. In some embodiments, the target nucleic acid characteristic of a bacterial pathogen is characteristic of an Enterobacter spp., a Klebsiella spp., and/or Streptococcus pneumoniae. In some embodiments, the target nucleic acid is characteristic of Enterobacter spp. and Klebsiella spp. In some embodiments, the target nucleic acid characteristic of Enterobacter spp. and Klebsiella spp. is amplified in the presence of a forward primer comprising the nucleotide sequence ATTCGTTGCACTATCGTTAACTGAATACA (SEQ ID NO: 15) and a reverse primer comprising the nucleotide sequence CTGTACCGTCGGACTTTCCAGAC (SEQ ID NO: 16). In other embodiments, the target nucleic acid characteristic of a bacterial pathogen is characteristic of Streptococcus pneumoniae. In some embodiments, the target nucleic acid characteristic of Streptococcus pneumoniae is amplified in the presence of a forward primer comprising the nucleotide sequence CCTTGGACGGAAATGTAGCTGGCA (SEQ ID NO: 53) and a reverse primer comprising the nucleotide sequence AATCACATGGTTGACACCTGCTGTG (SEQ ID NO: 54).

In some embodiments, the method includes amplifying and/or detecting NDM, KPC, IMP, VIM, OXA-48-like, DHA, CMY, and a target nucleic acid characteristic of Enterobacter spp. and Klebsiella spp.

In some embodiments, the multiplexed amplification reaction is configured to amplify NDM, KPC, IMP, VIM, OXA-48-like, DHA, CMY, and a target nucleic acid characteristic of Enterobacter spp. and Klebsiella spp.

In other embodiments, the method comprises amplifying and/or detecting KPC, CTX-M 14, CTX-M 15, NDM, VIM, IMP, vanA, vanB, and mecA, mecC, OXA-48-like, CMY, DHA, and a target nucleic acid characteristic of Enterobacter spp. and Klebsiella spp. In some embodiments, the multiplexed amplification reaction is configured to amplify KPC, CTX-M 14, CTX-M 15, NDM, VIM, IMP, vanA, vanB, and mecA, mecC, OXA-48-like, CMY, DHA, and a target nucleic acid characteristic of Enterobacter spp. and Klebsiella spp.

In yet other embodiments, the method comprises amplifying and/or detecting CTX-M 14, CTX-M 15, ermA, ermB, mecA, mefA, SHV, TEM, vanA, vanB, and a target nucleic acid characteristic of Streptococcus pneumoniae. In some embodiments, the multiplexed amplification reaction is configured to amplify CTX-M 14, CTX-M 15, ermA, ermB, mecA, mefA, SHV, TEM, vanA, vanB, and a target nucleic acid characteristic of Streptococcus pneumoniae.

In some embodiments of the preceding methods, step (c) includes washing the pellet one time prior to resuspending the pellet. In some embodiments, washing or resuspending is performed with a wash buffer solution. In some embodiments, the wash buffer solution is Tris-EDTA (TE) buffer. In some embodiments, the washing is performed with a wash buffer solution having a volume of about 100 μL to about 500 μL. In some embodiments, the volume is about 150 μL. In some embodiments, resuspending of step (c) is performed with a wash buffer solution having a volume of about 50 μL to about 150 μL. In some embodiments, the volume is about 100 μL.

In some embodiments of any of the preceding methods, the amplifying further includes amplifying an IC target nucleic acid and the method further includes the amplified IC target nucleic acid. In some embodiments, the multiplexed amplification reaction is configured to amplify an amplified IC target nucleic acid. In some embodiments, the IC target nucleic acid is amplified in the presence of a forward primer comprising the nucleotide sequence of GGAAATCTAACGAGAGAGCATGCT (SEQ ID NO: 55) and a reverse primer comprising the oligonucleotide sequence CGATGCGTGACACCCAGGC (SEQ ID NO: 56). In some embodiments, the wash buffer solution further includes an IC nucleic acid. In some embodiments of the preceding methods, step (a) further includes adding a total process control (TPC) to the whole blood sample. In some embodiments, the TPC is an engineered cell includes a control target nucleic acid.

In some embodiments of any of the preceding methods, amplifying is in the presence of whole blood proteins and non-target nucleic acids.

Any suitable lysis approach can be used. In some embodiments of any of the preceding methods, lysing includes mechanical lysis or heat lysis. In some embodiments, the mechanical lysis is beadbeating or sonicating.

In some embodiments of any of the preceding methods, the steps of the method are completed within 5 hours. In some embodiments, the steps of the method are completed within 4 hours. In some embodiments, the steps of the method are completed within 3 hours.

In some embodiments of the preceding methods, the detecting includes T2MR. In some embodiments, the detecting includes sequencing. In some embodiments, the detecting includes T2MR and sequencing.

Any suitable amplification approach can be used, e.g., any amplification approach described herein or known in the art. In some embodiments of any of the preceding aspects, the amplifying includes polymerase chain reaction (PCR), ligase chain reaction (LCR), multiple displacement amplification (MDA), strand displacement amplification (SDA), rolling circle amplification (RCA), loop mediated isothermal amplification (LAMP), nucleic acid sequence based amplification (NASBA), helicase dependent amplification, recombinase polymerase amplification, nicking enzyme amplification reaction, or ramification amplification (RAM). In some embodiments, the amplifying includes PCR. In some embodiments, the PCR is symmetric PCR or asymmetric PCR.

Any suitable sequencing approach can be used, e.g., any sequencing approach described herein or known in the art. In some embodiments of any of the preceding aspects, the sequencing includes massively parallel sequencing, Sanger sequencing, or single-molecule sequencing. In some embodiments, the massively parallel sequencing includes sequencing by synthesis or sequencing by ligation. In some embodiments, the massively parallel sequencing includes sequencing by synthesis. In some embodiments, the sequencing by synthesis includes ILLUMINA™ dye sequencing, ion semiconductor sequencing, or pyrosequencing. In some embodiments, the sequencing by synthesis includes ILLUMINA™ dye sequencing. In some embodiments, the sequencing by ligation includes sequencing by oligonucleotide ligation and detection (SOLiD™) sequencing or polony-based sequencing.

In some embodiments, the single-molecule sequencing is nanopore sequencing, single-molecule real-time (SMRT™) sequencing, or Helicos™ sequencing.

In some embodiments of any of the preceding aspects, the bacterial pathogen is a gram negative bacterial pathogen.

In some embodiments, the method comprises detecting any of the panels described herein, e.g., any panel set forth in any one of Tables 1-15, 19, or 22.

### Additional Sequencing-Based Methods of Detecting and Analyzing Drug Resistance Markers in Complex Samples

The invention provides methods for sequencing target nucleic acids associated with drug resistance (e.g., antibiotic resistance genes) in complex samples (e.g., biological or environmental samples) containing cells, cell debris (e.g., solid material), or nucleic acids (e.g., DNA or RNA (e.g., mRNA), e.g., non-target and/or subject-derived nucleic acids). In several embodiments, the sample contains cells and/or cell debris derived from a mammalian host subject and one or more pathogen cells. In several embodiments, the sample contains nucleic acids (e.g., DNA or RNA (e.g., mRNA)) derived from a mammalian host subject and one or more pathogen cells.

For example, provided herein is a method for detecting a drug resistance (e.g., antibiotic resistance) target nucleic acid (e.g., NDM, KPC, IMP, VIM, OXA-48-like, DHA, CMY, FOX, mecA, mecC, vanA, vanB, CTX-M 14, CTX-M 15, mefA, mefE, ermA, ermB, SHV, or TEM) in an environmental sample or a biological sample obtained from a subject, wherein the environmental or biological sample includes cells (e.g., subject-derived cells) or cell debris, the method including one or two of the following steps: (a) amplifying a target nucleic acid in the biological sample to form an amplified solution including an amplified target nucleic acid; and (b) sequencing the amplified target nucleic acid to detect whether the target nucleic acid is present in the biological sample, wherein the method is capable of detecting a concentration of about 10 copies/mL (or less, e.g., 1, 2, 3, 4, 5, 6, 7, 8, or 9 copies/mL) of the target nucleic acid in the biological sample.

Also provided herein is a method for determining the sequence of a drug resistance (e.g., antibiotic resistance) target nucleic acid (e.g., NDM, KPC, IMP, VIM, OXA-48-like, DHA, CMY, FOX, mecA, mecC, vanA, vanB, CTX-M 14, CTX-M 15, mefA, mefE, ermA, ermB, SHV, or TEM) in an environmental sample or a biological sample obtained from a subject, wherein the environmental or biological sample includes cells (e.g., subject-derived cells) or cell debris, the method including one or two of the following steps: (a) amplifying a target nucleic acid in the biological sample to form an amplified solution including an amplified target nucleic acid; and (b) sequencing the amplified target nucleic acid to detect whether the target nucleic acid is present in the biological sample, wherein the method is capable of determining the sequence of the target nucleic acid at a concentration of about 10 copies/mL (or less, e.g., 1, 2, 3, 4, 5, 6, 7, 8, or 9 copies/mL) of the target nucleic acid in the biological sample.

Further provided herein is a method for detecting a drug resistance (e.g., antibiotic resistance) target nucleic acid (e.g., NDM, KPC, IMP, VIM, OXA-48-like, DHA, CMY, FOX, mecA, mecC, vanA, vanB, CTX-M 14, CTX-M 15, mefA, mefE, ermA, ermB, SHV, or TEM) in an environmental sample or a biological sample obtained from a subject, wherein the environmental or biological sample includes cells (e.g., subject-derived cells) or cell debris, the method including sequencing an amplified target nucleic acid to detect whether the target nucleic acid is present in the biological sample, wherein the amplified target nucleic acid has been amplified in the environmental or biological sample, and the method is capable of detecting a concentration of about 10 copies/mL (or less, e.g., 1, 2, 3, 4, 5, 6, 7, 8, or 9 copies/mL) of the target nucleic acid in the biological sample.

Also provided herein is a method for determining the sequence of a drug resistance (e.g., antibiotic resistance) target nucleic acid (e.g., NDM, KPC, IMP, VIM, OXA-48-like, DHA, CMY, FOX, mecA, mecC, vanA, vanB, CTX-M 14, CTX-M 15, mefA, mefE, ermA, ermB, SHV, or TEM) in an environmental sample or a biological sample obtained from a subject, wherein the environmental or biological sample includes cells (e.g., subject-derived cells) or cell debris, the method including sequencing an amplified target nucleic acid to detect whether the target nucleic acid is present in the biological sample, wherein the amplified target nucleic acid has been amplified in the environmental or biological sample, and the method is capable of detecting a concentration of about 10 copies/mL (or less, e.g., 1, 2, 3, 4, 5, 6, 7, 8, or 9 copies/mL) of the target nucleic acid in the biological sample. In any of the preceding methods, the environmental sample or biological sample may contain nucleic acids (e.g., DNA or RNA (e.g., mRNA)), such as host-derived nucleic acids or non-target nucleic acids present in the sample.

Further provided herein is a method for detecting a drug resistance (e.g., antibiotic resistance) target nucleic acid (e.g., NDM, KPC, IMP, VIM, OXA-48-like, DHA, CMY, FOX, mecA, mecC, vanA, vanB, CTX-M 14, CTX-M 15, mefA, mefE, ermA, ermB, SHV, or TEM) in an environmental sample or a biological sample obtained from a subject, wherein the environmental or biological sample also includes non-target nucleic acids (e.g., DNA or RNA (e.g., mRNA)), the method including one or two of the following steps: (a) amplifying a target nucleic acid in the biological sample to form an amplified solution including an amplified target nucleic acid; and (b) sequencing the amplified target nucleic acid to detect whether the target nucleic acid is present in the biological sample, wherein the method is capable of detecting a concentration of about 10 copies/mL (or less, e.g., 1, 2, 3, 4, 5, 6, 7, 8, or 9 copies/mL) of the target nucleic acid in the biological sample.

Also provided herein is a method for determining the sequence of a drug resistance (e.g., antibiotic resistance) target nucleic acid (e.g., NDM, KPC, IMP, VIM, OXA-48-like, DHA, CMY, FOX, mecA, mecC, vanA, vanB, CTX-M 14, CTX-M 15, mefA, mefE, ermA, ermB, SHV, or TEM) in an environmental sample or a biological sample obtained from a subject, wherein the environmental or biological sample also includes non-target nucleic acids (e.g., DNA or RNA (e.g., mRNA)), the method including one or two of the following steps: (a) amplifying a target nucleic acid in the biological sample to form an amplified solution including an amplified target nucleic acid; and (b) sequencing the amplified target nucleic acid to detect whether the target nucleic acid is present in the biological sample, wherein the method is capable of determining the sequence of the target nucleic acid at a concentration of about 10 copies/mL (or less, e.g., 1, 2, 3, 4, 5, 6, 7, 8, or 9 copies/mL) of the target nucleic acid in the biological sample.

In yet another example, provided herein is a method for detecting a drug resistance (e.g., antibiotic resistance) target nucleic acid (e.g., NDM, KPC, IMP, VIM, OXA-48-like, DHA, CMY, FOX, mecA, mecC, vanA, vanB, CTX-M 14, CTX-M 15, mefA, mefE, ermA, ermB, SHV, or TEM) in an environmental sample or a biological sample obtained from a subject, wherein the environmental or biological sample also includes non-target nucleic acids (e.g., DNA or RNA (e.g., mRNA)), the method including sequencing an amplified target nucleic acid to detect whether the target nucleic acid is present in the biological sample, wherein the amplified target nucleic acid has been amplified in the environmental or biological sample, and the method is capable of detecting a concentration of about 10 copies/mL (or less, e.g., 1, 2, 3, 4, 5, 6, 7, 8, or 9 copies/mL) of the target nucleic acid in the biological sample. Further provided herein is a method for determining the sequence of a drug resistance (e.g., antibiotic resistance) target nucleic acid (e.g., NDM, KPC, IMP, VIM, OXA-48-like, DHA, CMY, FOX, mecA, mecC, vanA, vanB, CTX-M 14, CTX-M 15, mefA, mefE, ermA, ermB, SHV, or TEM) in an environmental sample or a biological sample obtained from a subject, wherein the environmental or biological sample also includes non-target nucleic acids (e.g., DNA or RNA (e.g., mRNA)), the method including sequencing an amplified target nucleic acid to detect whether the target nucleic acid is present in the biological sample, wherein the amplified target nucleic acid has been amplified in the environmental or biological sample, and the method is capable of detecting a concentration of about 10 copies/mL (or less, e.g., 1, 2, 3, 4, 5, 6, 7, 8, or 9 copies/mL) of the target nucleic acid in the biological sample. In any of the methods described herein, the environmental or biological sample (e.g., blood (e.g., whole blood (e.g., ethylenediaminetetraacetic acid (EDTA) whole blood, sodium citrate whole blood, sodium heparin whole blood, lithium heparin whole blood, and/or potassium oxylate/sodium fluoride whole blood), a crude blood lysate, serum, or plasma)) can have any suitable volume. For example, in some embodiments, the environmental or biological sample has a volume of about 0.2 mL to about 50 mL, e.g., about 0.2 mL, about 0.3 mL, about 0.4 mL, about 0.5 mL, about 0.6 mL, about 0.7 mL, about 0.8 mL, about 0.9 mL, about 1 mL, about 2 mL, about 3 mL, about 4 mL, about 5 mL, about 6 mL, about 7 mL, about 8 mL, about 9 mL, about 10 mL, about 15 mL, about 20 mL, about 25 mL, about 30 mL, about 35 mL, about 40 mL, about 45 mL, or about 50 mL. In some embodiments, the environmental or biological sample has a volume of about 0.2 mL to about 20 mL, about 0.2 mL to about 15 mL, about 0.2 mL to about 10 mL, about 0.2 mL to about 5 mL, about 0.2 mL to about 2 mL, about 0.4 mL to about 20 mL, about 0.4 mL to about 15 mL, about 0.4 mL to about 10 mL, about 0.4 mL to about 5 mL, about 0.4 mL to about 2 mL, about 0.6 mL to about 20 mL, about 0.6 mL to about 15 mL, about 0.6 mL to about 10 mL, about 0.6 mL to about 5 mL, about 0.6 mL to about 2 mL, about 0.8 mL to about 20 mL, about 0.8 mL to about 15 mL, about 0.8 mL to about 10 mL, about 0.8 mL to about 5 mL, about 0.8 mL to about 2 mL, about 1 mL to about 20 mL, about 1 mL to about 15 mL, about 1 mL to about 10 mL, about 1 mL to about 5 mL, about 1 mL to about 4 mL, about 1 mL to about 3 mL, about 1 mL to about 2 mL, about 1.5 mL to about 20 mL, about 1.5 mL to about 15 mL, about 1.5 mL to about 10 mL, about 1.5 mL to about 5 mL, about 1.5 mL to about 4 mL, about 1.5 mL to about 3 mL, about 1.5 mL to about 2 mL, about 2 mL to about 20 mL, about 2 mL to about 15 mL, about 2 mL to about 10 mL, about 2 mL to about 5 mL, about 2 mL to about 4 mL, about 2 mL to about 3 mL, about 3 mL to about 20 mL, about 3 mL to about 15 mL, about 3 mL to about 10 mL, about 3 mL to about 5 mL, about 3 mL to about 4 mL, about 4 mL to about 20 mL, about 4 mL to about 15 mL, about 4 mL to about 10 mL, about 4 mL to about 5 mL, about 5 mL to about 20 mL, about 5 mL to about 15 mL, about 5 mL to about 10 mL, about 6 mL to about 20 mL, about 6 mL to about 15 mL, about 6 mL to about 10 mL, about 7 mL to about 20 mL, about 7 mL to about 15 mL, about 7 mL to about 10 mL, about 8 mL to about 20 mL, about 8 mL to about 15 mL, about 8 mL to about 10 mL, about 9 mL to about 20 mL, about 9 mL to about 15 mL, about 9 mL to about 10 mL, about 10 mL to about 20 mL, or about 10 mL to about 15 mL. In some embodiments, the environmental or biological sample has a volume of about 0.2 mL to about 20 mL, about 0.2 mL to about 15 mL, about 0.2 mL to about 10 mL, about 0.2 mL to about 5 mL, or about 0.2 mL to about 2 mL. In some embodiments, the environmental or biological sample has a volume of about 2 mL.

Any suitable environmental or biological sample can be used. For example, the biological sample can be selected from the group consisting of blood (e.g., whole blood (e.g., ethylenediaminetetraacetic acid (EDTA) whole blood, sodium citrate whole blood, sodium heparin whole blood, lithium heparin whole blood, and/or potassium oxylate/sodium fluoride whole blood), a crude blood lysate, serum, or plasma), bloody fluids (e.g., wound exudate, phlegm, or bile), tissue samples (e.g., a tissue biopsy such as a skin biopsy, muscle biopsy, or lymph node biopsy), urine, bronchiolar lavage (BAL), cerebrospinal fluid (CSF), synovial fluid (SF), and sputum. In some embodiments, the biological sample is blood (e.g., whole blood (e.g., EDTA whole blood, sodium citrate whole blood, sodium heparin whole blood, lithium heparin whole blood, and/or potassium oxylate/sodium fluoride whole blood), a crude blood lysate, serum, or plasma). In some embodiments, the tissue sample is a homogenized tissue sample. The environmental sample may be an environmental swab, e.g., a surface swab. In some embodiments, the swab buffer diluent or swab transport medium is, without limitation, phospho-buffered saline-TWEEN® (PBST), Amies Buffer, Amies Buffer+10% (v/v) 10×PBST, Cary Blair Media, or Liquid Stuart Swabs (which may include addition of 10% (v/v) 10×PBST).

In some embodiments of any of the preceding methods, the target nucleic acid is characteristic of a drug resistance marker, e.g., an antibiotic resistance gene. In some embodiments, the antibiotic resistance gene is selected from the group consisting of NDM, KPC, IMP, VIM, OXA-48-like, DHA, CMY, FOX, mecA, mecC, vanA, vanB, CTX-M 14, CTX-M 15, mefA, mefE, ermA, ermB, SHV, and TEM. In some embodiments, the antibiotic resistance gene is selected from the group consisting of NDM, KPC, IMP, VIM, OXA-48-like, DHA, CMY, mecA, mecC, vanA, vanB, CTX-M 14, CTX-M 15, mefA, mefE, ermA, ermB, SHV, and TEM.

In some embodiments of any of the methods described herein, the method (e.g., step (a) of the method) includes amplifying the target nucleic acid in a lysate produced by lysing cells in the environmental or biological sample. The lysate may contain a concentration of cells, cell debris, and/or non-target or subject-cell derived nucleic acids (e.g., DNA) relative to the original environmental or biological sample. As an example, 2 mL of a biological sample concentrated down to 0.1 mL in a lysate corresponds to a 20:1 higher concentration of debris compared to the original sample. If the lysate is diluted 1:1 for amplification, the amplification is performed in a lysate that represents a 10:1 concentration of the debris in the original sample. In some embodiments, the lysate has at least about a 1.5:1 higher concentration of cell debris relative to the environmental or biological sample, e.g., about 1.5:1, about 2:1, about 3:1, about 4:1, about 5:1, about 6:1, about 7:1, about 8:1, about 9:1, about 10:1, about 11:1, about 12:1, about 13:1, about 14:1, about 15:1, about 16:1, about 18:1, about 19:1, about 20:1, about 21:1, about 22:1, about 23:1, about 24:1, about 25:1, about 30:1, about 35:1, about 40:1, about 45:1, about 50:1, about 55:1, about 60:1, about 65:1, about 70:1, about 75:1, about 80:1, about 90:1, about 100:1, about 120:1, about 140:1, about 160:1, about 180:1, about 200:1, about 300:1, about 400:1, about 500:1, about 600:1, about 700:1, about 800:1, about 900:1, about 1000:1, or higher concentration of cell debris relative to the environmental or biological sample. In some embodiments, the lysate is not diluted prior to amplification. In other embodiments, the lysate is diluted to produce a diluted lysate (e.g., for use in amplification), and the diluted lysate has at least about a 1.5:1 higher concentration of cell debris relative to the environmental or biological sample, e.g., about 1.5:1, about 2:1, about 3:1, about 4:1, about 5:1, about 6:1, about 7:1, about 8:1, about 9:1, about 10:1, about 11:1, about 12:1, about 13:1, about 14:1, about 15:1, about 16:1, about 18:1, about 19:1, about 20:1, about 21:1, about 22:1, about 23:1, about 24:1, about 25:1, about 30:1, about 35:1, about 40:1, about 45:1, about 50:1, about 55:1, about 60:1, about 65:1, about 70:1, about 75:1, about 80:1, about 90:1, about 100:1, about 120:1, about 140:1, about 160:1, about 180:1, about 200:1, about 300:1, about 400:1, about 500:1, about 600:1, about 700:1, about 800:1, about 900:1, about 1000:1, or higher concentration of cell debris relative to the environmental or biological sample. In some embodiments, the lysate or the diluted lysate has about a 10:1 higher concentration of cell debris relative to the environmental or biological sample. In other embodiments, the lysate or the diluted lysate has about a 20:1 higher concentration of cell debris relative to the environmental or biological sample. In some embodiments, the cell debris is solid material (e.g., solid material that can be concentrated with a liquid-solid separation method (e.g., centrifugation or filtration). In some embodiments, the lysate or the amplified lysate solution is a super-saturated solution of cell debris (e.g., solid material).

Also provided herein is a method for detecting a drug resistance (e.g., antibiotic resistance) target pathogen nucleic acid (e.g., NDM, KPC, IMP, VIM, OXA-48-like, DHA, CMY, FOX, mecA, mecC, vanA, vanB, CTX-M 14, CTX-M 15, mefA, mefE, ermA, ermB, SHV, or TEM) in a whole blood sample, the method including the following steps: (a) contacting a whole blood sample suspected of containing one or more pathogen cells with an erythrocyte lysis agent, thereby lysing red blood cells; (b) centrifuging the product of step (a) to form a supernatant and a pellet; (c) discarding some or all of the supernatant of step (b) and resuspending the pellet to form an extract, optionally washing the pellet one or more times prior to resuspending the pellet; (d) lysing the remaining cells in the extract of step (c) to form a lysate, the lysate containing both subject cell nucleic acid and pathogen nucleic acid; (e) amplifying pathogen nucleic acids in the lysate of step (d) to form an amplified lysate solution including an amplified target pathogen nucleic acid; and (f) sequencing the amplified target pathogen nucleic acid, thereby detecting the target pathogen nucleic acid in the sample.

Further provided herein is a method for determining the sequence of a drug resistance (e.g., antibiotic resistance) target pathogen nucleic acid (e.g., NDM, KPC, IMP, VIM, OXA-48-like, DHA, CMY, FOX, mecA, mecC, vanA, vanB, CTX-M 14, CTX-M 15, mefA, mefE, ermA, ermB, SHV, or TEM) in a whole blood sample, the method including the following steps: (a) contacting a whole blood sample suspected of containing one or more pathogen cells with an erythrocyte lysis agent, thereby lysing red blood cells; (b) centrifuging the product of step (a) to form a supernatant and a pellet; (c) discarding some or all of the supernatant of step (b) and resuspending the pellet to form an extract, optionally washing the pellet one or more times prior to resuspending the pellet; (d) lysing the remaining cells in the extract of step (c) to form a lysate, the lysate containing both subject cell nucleic acid and pathogen nucleic acid; (e) amplifying pathogen nucleic acids in the lysate of step (d) to form an amplified lysate solution including an amplified target pathogen nucleic acid; and (f) sequencing the amplified target pathogen nucleic acid, thereby determining the sequence of the target pathogen nucleic acid in the sample.

In some embodiments of any of the preceding methods, step (c) can include washing the pellet one time prior to resuspending the pellet. In other embodiments, step (c) can include washing the pellet more than one time prior to resuspending the pellet, e.g., two, three, four, five, six, seven, eight, nine, or ten times. In some embodiments, the washing or resuspending is performed with a wash buffer solution (e.g., Tris-EDTA (TE) buffer). The wash buffer solution may have any suitable volume, e.g., about 25 μL to about 1 mL, e.g., about 25 μL, about 30 μL, about 35 μL, about 40 μL, about 45 μL, about 50 μL, about 55 μL, about 60 μL, about 65 μL, about 70 μL, about 75 μL, about 80 μL, about 85 μL, about 90 μL, about 100 μL, about 110 μL, about 120 μL, about 130 μL, about 140 μL, about 150 μL, about 160 μL, about 170 μL, about 180 μL, about 190 μL, about 200 μL, about 225 μL, about 250 μL, about 275 μL, about 300 μL, about 350 μL, about 400 μL, about 450 μL, about 500 μL, about 550 μL, about 600 μL, about 650 μL, about 700 μL, about 800 μL, about 850 μL, about 900 μL, about 950 μL, or about 1 mL. In some embodiments, the washing is performed with a wash buffer solution having a volume of about 100 μL to about 500 μL. In some embodiments, the washing is performed with a wash buffer solution having a volume of about 100 μL to about 200 μL. In some embodiments, the volume is about 150 μL. The resuspending of step (c) can be performed with a wash buffer solution having any suitable volume, e.g., about 25 μL to about 1 mL, e.g., about 25 μL, about 30 μL, about 35 μL, about 40 μL, about 45 μL, about 50 μL, about 55 μL, about 60 μL, about 65 μL, about 70 μL, about 75 μL, about 80 μL, about 85 μL, about 90 μL, about 100 μL, about 110 μL, about 120 μL, about 130 μL, about 140 μL, about 150 μL, about 160 μL, about 170 μL, about 180 μL, about 190 μL, about 200 μL, about 225 μL, about 250 μL, about 275 μL, about 300 μL, about 350 μL, about 400 μL, about 450 μL, about 500 μL, about 550 μL, about 600 μL, about 650 μL, about 700 μL, about 800 μL, about 850 μL, about 900 μL, about 950 μL, or about 1 mL. In some embodiments, the resuspending is performed with a wash buffer solution having a volume of about 100 μL to about 500 μL or about 100 μL to about 200 μL. In some embodiments, the volume is about 100 μL.

In some embodiments of any of the preceding methods, the wash buffer solution further includes an amplification control nucleic acid. In some embodiments of any of the preceding methods, step (a) further includes adding a total process control (TPC) to the whole blood sample, e.g., an engineered cell including a control target nucleic acid.

In another example, provided herein is a method for detecting a drug resistance (e.g., antibiotic resistance) target pathogen nucleic acid in a whole blood sample, the method including the following steps: (a) providing an amplified lysate solution that has been produced by: (i) contacting a whole blood sample suspected of containing one or more pathogen cells with an erythrocyte lysis agent, thereby lysing red blood cells; (ii) centrifuging the product of step (a)(i) to form a supernatant and a pellet; (iii) discarding some or all of the supernatant of step (a)(ii) and resuspending the pellet to form an extract, optionally washing the pellet one or more times prior to resuspending the pellet; (iv) lysing the remaining cells in the extract of step (a)(iii) to form a lysate, the lysate containing both subject cell nucleic acid and pathogen nucleic acid; (v) amplifying pathogen nucleic acids in the lysate of step (a)(iv) to form an amplified lysate solution including an amplified target pathogen nucleic acid; and (b) sequencing the amplified target pathogen nucleic acid, thereby detecting the target pathogen nucleic acid in the sample. In a further example, provided herein is a method for determining the sequence of a drug resistance (e.g., antibiotic resistance) target pathogen nucleic acid in a whole blood sample, the method including the following steps: (a) providing an amplified lysate solution that has been produced by: (i) contacting a whole blood sample suspected of containing one or more pathogen cells with an erythrocyte lysis agent, thereby lysing red blood cells; (ii) centrifuging the product of step (a)(i) to form a supernatant and a pellet; (iii) discarding some or all of the supernatant of step (a)(ii) and resuspending the pellet to form an extract, optionally washing the pellet one or more times prior to resuspending the pellet; (iv) lysing the remaining cells in the extract of step (a)(iii) to form a lysate, the lysate containing both subject cell nucleic acid and pathogen nucleic acid; (v) amplifying pathogen nucleic acids in the lysate of step (a)(iv) to form an amplified lysate solution including an amplified target pathogen nucleic acid; and (b) sequencing the amplified target pathogen nucleic acid, thereby determining the sequence of the target pathogen nucleic acid in the sample.

In some embodiments of any of the preceding methods, step (a)(iii) includes washing the pellet one time prior to resuspending the pellet. In other embodiments, step (a)(iii) includes washing the pellet more than one time prior to resuspending the pellet, e.g., two, three, four, five, six, seven, eight, nine, or ten times.

In some embodiments, the washing or resuspending is performed with a wash buffer solution (e.g., Tris-EDTA (TE) buffer). The wash buffer solution may have any suitable volume, e.g., about 25 μL to about 1 mL, e.g., about 25 μL, about 30 μL, about 35 μL, about 40 μL, about 45 μL, about 50 μL, about 55 μL, about 60 μL, about 65 μL, about 70 μL, about 75 μL, about 80 μL, about 85 μL, about 90 μL, about 100 μL, about 110 μL, about 120 μL, about 130 μL, about 140 μL, about 150 μL, about 160 μL, about 170 μL, about 180 μL, about 190 μL, about 200 μL, about 225 μL, about 250 μL, about 275 μL, about 300 μL, about 350 μL, about 400 μL, about 450 μL, about 500 μL, about 550 μL, about 600 μL, about 650 μL, about 700 μL, about 800 μL, about 850 μL, about 900 μL, about 950 μL, or about 1 mL. In some embodiments, the washing is performed with a wash buffer solution having a volume of about 100 μL to about 500 μL. In some embodiments, the washing is performed with a wash buffer solution having a volume of about 100 μL to about 200 μL. In some embodiments, the volume is about 150 μL. The resuspending of step (a)(iii) can be performed with a wash buffer solution having any suitable volume, e.g., about 25 μL to about 1 mL, e.g., about 25 μL, about 30 μL, about 35 μL, about 40 μL, about 45 μL, about 50 μL, about 55 μL, about 60 μL, about 65 μL, about 70 μL, about 75 μL, about 80 μL, about 85 μL, about 90 μL, about 100 μL, about 110 μL, about 120 μL, about 130 μL, about 140 μL, about 150 μL, about 160 μL, about 170 μL, about 180 μL, about 190 μL, about 200 μL, about 225 μL, about 250 μL, about 275 μL, about 300 μL, about 350 μL, about 400 μL, about 450 μL, about 500 μL, about 550 μL, about 600 μL, about 650 μL, about 700 μL, about 800 μL, about 850 μL, about 900 μL, about 950 μL, or about 1 mL. In some embodiments, the resuspending is performed with a wash buffer solution having a volume of about 100 μL to about 500 μL or about 100 μL to about 200 μL. In some embodiments, the volume is about 100 μL.

In some embodiments of any of the preceding methods, the wash buffer solution further includes an amplification control nucleic acid. In some embodiments of any of the preceding methods, step (a)(i) further includes adding a total process control (TPC) to the whole blood sample, e.g., an engineered cell including a control target nucleic acid.

In a still further example, provided herein is a method for detecting a drug resistance (e.g., antibiotic resistance) target pathogen nucleic acid (e.g., NDM, KPC, IMP, VIM, OXA-48-like, DHA, CMY, FOX, mecA, mecC, vanA, vanB, CTX-M 14, CTX-M 15, mefA, mefE, ermA, ermB, SHV, or TEM) in a whole blood sample, the method including the following steps: (a) contacting a whole blood sample suspected of containing one or more pathogen cells with an erythrocyte lysis agent, thereby lysing red blood cells; (b) centrifuging the product of step (a) to form a supernatant and a pellet; (c) discarding some or all of the supernatant of step (b) and washing the pellet once; (d) centrifuging the product of step (c) to form a supernatant and a pellet; (e) discarding some or all of the supernatant of step (d) and mixing the pellet of (d) with a buffer solution; (f) combining the product of step (e) with beads to form a mixture and agitating the mixture to form a lysate, said lysate containing both subject cell nucleic acid and pathogen nucleic acid; (g) amplifying pathogen nucleic acids in the lysate of step (f) to form an amplified lysate solution including an amplified target pathogen nucleic acid; and (h) sequencing the amplified target pathogen nucleic acid, thereby detecting the target pathogen nucleic acid in the sample.

In yet another example, provided herein is a method for determining the sequence of a drug resistance (e.g., antibiotic resistance) target pathogen nucleic acid (e.g., NDM, KPC, IMP, VIM, OXA-48-like, DHA, CMY, FOX, mecA, mecC, vanA, vanB, CTX-M 14, CTX-M 15, mefA, mefE, ermA, ermB, SHV, or TEM) in a whole blood sample, the method including the following steps: (a) contacting a whole blood sample suspected of containing one or more pathogen cells with an erythrocyte lysis agent, thereby lysing red blood cells; (b) centrifuging the product of step (a) to form a supernatant and a pellet; (c) discarding some or all of the supernatant of step (b) and washing the pellet once; (d) centrifuging the product of step (c) to form a supernatant and a pellet; (e) discarding some or all of the supernatant of step (d) and mixing the pellet of (d) with a buffer solution; (f) combining the product of step (e) with beads to form a mixture and agitating the mixture to form a lysate, said lysate containing both subject cell nucleic acid and pathogen nucleic acid; (g) amplifying pathogen nucleic acids in the lysate of step (f) to form an amplified lysate solution including an amplified target pathogen nucleic acid; and (h) sequencing the amplified target pathogen nucleic acid, thereby determining the sequence of the target pathogen nucleic acid in the sample.

In some embodiments of any of the preceding methods, the washing of step (c) is performed with a wash buffer solution (e.g., TE buffer). The wash buffer solution may have any suitable volume, e.g., about 25 μL to about 1 mL, e.g., about 25 μL, about 30 μL, about 35 μL, about 40 μL, about 45 μL, about 50 μL, about 55 μL, about 60 μL, about 65 μL, about 70 μL, about 75 μL, about 80 μL, about 85 μL, about 90 μL, about 100 μL, about 110 μL, about 120 μL, about 130 μL, about 140 μL, about 150 μL, about 160 μL, about 170 μL, about 180 μL, about 190 μL, about 200 μL, about 225 μL, about 250 μL, about 275 μL, about 300 μL, about 350 μL, about 400 μL, about 450 μL, about 500 μL, about 550 μL, about 600 μL, about 650 μL, about 700 μL, about 800 μL, about 850 μL, about 900 μL, about 950 μL, or about 1 mL. In some embodiments, the washing is performed with a wash buffer solution having a volume of about 100 μL to about 500 μL. In some embodiments, the washing is performed with a wash buffer solution having a volume of about 100 μL to about 200 μL. In some embodiments, the volume is about 150 μL. In some embodiments of any of the preceding methods, step (e) includes mixing the pellet with a buffer solution. The buffer solution (e.g., TE buffer) may have any suitable volume, e.g., about 25 μL to about 1 mL, e.g., about 25 μL, about 30 μL, about 35 μL, about 40 μL, about 45 μL, about 50 μL, about 55 μL, about 60 μL, about 65 μL, about 70 μL, about 75 μL, about 80 μL, about 85 μL, about 90 μL, about 100 μL, about 110 μL, about 120 μL, about 130 μL, about 140 μL, about 150 μL, about 160 μL, about 170 μL, about 180 μL, about 190 μL, about 200 μL, about 225 μL, about 250 μL, about 275 μL, about 300 μL, about 350 μL, about 400 μL, about 450 μL, about 500 μL, about 550 μL, about 600 μL, about 650 μL, about 700 μL, about 800 μL, about 850 μL, about 900 μL, about 950 μL, or about 1 mL. In some embodiments, the buffer solution has a volume of about 100 μL to about 200 μL. In some embodiments, the volume is about 100 μL.

In some embodiments of any of the preceding methods, the buffer solution and/or the wash buffer solution further includes an amplification control nucleic acid. In some embodiments of any of the preceding methods, step (a) further includes adding a TPC to the whole blood sample, e.g., an engineered cell including a control target nucleic acid.

In any of the method described herein, the lysate or the amplified lysate solution may contain a concentration of cells, cell debris, and/or non-target or subject-cell derived nucleic acids (e.g., DNA) relative to the original environmental or biological sample. As an example, 2 mL of a biological sample concentrated down to 0.1 mL in a lysate corresponds to a 20:1 higher concentration of debris compared to the original sample. If the lysate is diluted 1:1 for amplification, the amplification is performed in a lysate (e.g., an amplified lysate solution) that represents a 10:1 concentration of the debris in the original sample. In some embodiments of any of the preceding methods, the lysate or the amplified lysate solution has at least about a 1.5:1 higher concentration of subject cell DNA and/or cell debris relative to the whole blood sample, e.g., about 1.5:1. about 2:1, about 3:1, about 4:1, about 5:1, about 6:1, about 7:1, about 8:1, about 9:1, about 10:1, about 11:1, about 12:1, about 13:1, about 14:1, about 15:1, about 16:1, about 18:1, about 19:1, about 20:1, about 21:1, about 22:1, about 23:1, about 24:1, about 25:1, about 30:1, about 35:1, about 40:1, about 45:1, about 50:1, about 55:1, about 60:1, about 65:1, about 70:1, about 75:1, about 80:1, about 90:1, about 100:1, about 120:1, about 140:1, about 160:1, about 180:1, about 200:1, about 300:1, about 400:1, about 500:1, about 600:1, about 700:1, about 800:1, about 900:1, about 1000:1, or higher concentration of subject cell DNA and/or cell debris relative to the whole blood sample. In some embodiments, the lysate or the amplified lysate solution has about a 10:1 higher concentration of subject cell DNA and/or cell debris relative to the whole blood sample. In some embodiments, the lysate or the amplified lysate solution has a 20:1 higher concentration of subject cell DNA and/or cell debris relative to the whole blood sample. In some embodiments, the lysate has about a 20:1 higher concentration of subject cell DNA and/or cell debris relative to the whole blood sample. In some embodiments, the amplified lysate solution has about a 10:1 higher concentration of subject cell DNA and/or cell debris relative to the whole blood sample. In some embodiments, the cell debris is solid material (e.g., solid material that can be concentrated with a liquid-solid separation method (e.g., centrifugation or filtration)). In some embodiments, the lysate or the amplified lysate solution is a super-saturated solution of cell debris (e.g., solid material).

In some embodiments of any of the methods described herein, an amplified drug resistance (e.g., antibiotic resistance) target nucleic acid is produced in the presence of at least 1 μg of subject DNA, e.g., at least 1 μg of subject DNA, at least 5 μg of subject DNA, at least 10 μg of subject DNA, at least 15 μg of subject DNA, at least 20 μg of subject DNA, at least 25 μg of subject DNA, at least 30 μg of subject DNA, at least 35 μg of subject DNA, at least 40 μg of subject DNA, at least 45 μg of subject DNA, at least 50 μg of subject DNA, at least 55 μg of subject DNA, at least 60 μg of subject DNA, at least 80 μg of subject DNA, at least 90 μg of subject DNA, at least 100 μg of subject DNA, or more. For example, in some embodiments, the amplifying is performed in the presence of from about 0.5 μg to about 100 μg or about 20 to about 80 μg of subject cell DNA. In some embodiments, the amplifying is performed in the presence of about 60 μg of subject cell DNA. In some embodiments, at least a portion of the subject DNA is from white blood cells of the subject.

Any suitable amplification approach can be used. For example, in some embodiments, the amplifying includes polymerase chain reaction (PCR), ligase chain reaction (LCR), multiple displacement amplification (MDA), strand displacement amplification (SDA), rolling circle amplification (RCA), loop mediated isothermal amplification (LAMP), nucleic acid sequence based amplification (NASBA), helicase dependent amplification, recombinase polymerase amplification, nicking enzyme amplification reaction, or ramification amplification (RAM). In some embodiments, the amplifying includes PCR (e.g., symmetric PCR or asymmetric PCR).

In some embodiments of any of the preceding methods, the amplifying includes the following steps: (i) adding to the lysate an amplification buffer solution including a buffering agent to form a reaction mixture; (ii) heating the reaction mixture to form a denatured reaction mixture; and (iii) adding a thermostable nucleic acid polymerase to the denatured reaction mixture under conditions and for a time sufficient for amplification of the target nucleic acid. The method can further include centrifuging the denatured reaction mixture to form a pellet and a supernatant prior to step (iii). In some embodiments, step (iii) includes adding the thermostable nucleic acid polymerase to the supernatant. In other embodiments of any of the preceding methods, the amplifying includes the following steps: (i) adding to the lysate an amplification buffer solution including a buffering agent and a thermostable nucleic acid polymerase to form a reaction mixture under conditions and for a time sufficient for amplification of the target nucleic acid; (ii) heating the reaction mixture to form a denatured reaction mixture; and (iii) centrifuging the denatured reaction mixture to form a pellet and a supernatant. In some embodiments, the final concentration of the thermostable nucleic acid polymerase in step (i) is at least about 0.02 units per microliter of the reaction mixture. In some embodiments, step (i) comprises adding at least about 2.4×10⁻⁵ micrograms of a thermostable nucleic acid polymerase per microliter of reaction mixture. In some embodiments of any of the preceding methods, the amplification buffer solution has a moderately alkaline pH at ambient temperature. In some embodiments of any of the preceding methods, the moderately alkaline pH at ambient temperature is from about pH 7.1 to about pH 11.5 or higher (e.g., about pH 7.1, about pH 7.2, about pH 7.3, about pH 7.4, about pH 7.5, about pH 7.6, about pH 7.7, about pH 7.8, about pH 7.9, about pH 8.0, about pH 8.1, about pH 8.2, about pH 8.3, about pH 8.4, about pH 8.5, about pH 8.6, about pH 8.7, about pH 8.8, about pH 8.9, about pH 9.0, about pH 9.1, about pH 9.2, about pH 9.3, about pH 9.4, about pH 9.5, about pH 9.6, about pH 9.7, about pH 9.8, about pH 9.9, about pH 10.0, about pH 10.1, about pH 10.2, about pH 10.3, about pH 10.4, about pH 10.5, about pH 10.6, about pH 10.7, about pH 10.8, about pH 10.9, about pH 11, about pH 11.1, about pH 11.2, about pH 11.3, about pH 11.3, about pH 11.4, about pH 11.5, or higher. In some embodiments, the moderately alkaline pH at ambient temperature is from about pH 7.1 to about pH 11.5, about pH 7.1 to about pH 11.0, about pH 7.1 to about pH 10.5, about pH 7.1 to about pH 10.0, about pH 7.1 to about pH 9.5, about pH 7.1 to about pH 9.0, about pH 7.1 to about pH 8.5, about pH 7.1 to about pH 8, about pH 7.1 to about pH 7.5, about pH 7.5 to about pH 11.5, about pH 7.5 to about pH 11.0, about pH 7.5 to about pH 10.5, about pH 7.5 to about pH 10.0, about pH 7.5 to about pH 9.5, about pH 7.5 to about pH 9.0, about pH 7.5 to about pH 8.5, about pH 7.5 to about pH 8.0, about pH 8.0 to about pH 11.5, about pH 8.0 to about pH 11.0, about pH 8.0 to about pH 10.5, about pH 8.0 to about pH 10.0, about pH 8.0 to about pH 9.5, about pH 8.0 to about pH 9.0, about pH 8.0 to about pH 9.0, about pH 8.0 to about pH 8.5, about pH 8.5 to about pH 11.5, about pH 8.5 to about pH 11.0, about pH 8.5 to about pH 10.0, about pH 8.5 to about pH 9.5, about pH 8.5 to about pH 9.0, about pH 9.0 to about pH 11.5, about pH 9.0 to about pH 11.0, about pH 9.0 to about pH 10.5, about pH 9.0 to about pH 10.0, about pH 9.0 to about pH 9.5, about pH 9.5 to about pH 11.5, about pH 9.5 to about pH 11.0, about pH 9.5 to about pH 10.5, or about pH 9.5 to about pH 10.0. In some embodiments, the moderately alkaline pH at ambient temperature is about pH 8.7. In some embodiments, ambient temperature is about 25° C. (e.g., about 20° C., about 21° C., about 22° C., about 23° C., about 24° C., about 25° C., about 26° C., about 27° C., about 28° C., about 29° C., about 30° C.). In some embodiments, ambient temperature is about 20° C. to about 30° C., about 20° C. to about 29° C., about 20° C. to about 28° C., about 20° C. to about 27° C., about 20° C. to about 26° C., about 20° C. to about 25° C., about 20° C. to about 24° C., about 20° C. to about 23° C., about 20° C. to about 22° C., about 20° C. to about 21° C., about 22° C. to about 30° C., about 22° C. to about 29° C., about 22° C. to about 28° C., about 22° C. to about 27° C., about 22° C. to about 26° C., about 22° C. to about 25° C., about 22° C. to about 24° C., about 22° C. to about 23° C., about 24° C. to about 30° C., about 24° C. to about 29° C., about 24° C. to about 28° C., about 24° C. to about 27° C., about 24° C. to about 26° C., or about 24° C. to about 25° C.

In some embodiments of any of the preceding methods, the pH of the buffer solution remains approximately at or above a neutral pH at 95° C. In some embodiments, the pH of the buffer solution is about pH 6.5 to about pH 10 (e.g., about pH 6.5, about pH 6.6, about pH 6.7, about pH 6.8, about pH 6.9, about pH 7.0, about pH 7.1, about pH 7.2, about pH 7.3, about pH 7.4, about pH 7.5, about pH 7.6, about pH 7.7, about pH 7.8, about pH 7.9, about pH 8.0, about pH 8.1, about pH 8.2, about pH 8.3, about pH 8.4, about pH 8.5, about pH 8.6, about pH 8.7, about pH 8.8, about pH 8.9, about pH 9.0, about pH 9.1, about pH 9.2, about pH 9.3, about pH 9.4, about pH 9.5, about pH 9.6, about pH 9.7, about pH 9.8, about pH 9.9, or about pH 10.0) at 95° C. For example, in some embodiments, the pH of the buffer solution at 95° C. is from about pH 6.5 to about pH 10.0, about pH 6.5 to about pH 9.5, about pH 6.5 to about pH 9.0, about pH 6.5 to about pH 8.5, about pH 6.5 to about pH 8.0, about pH 6.5 to about pH 7.5, about pH 7.0 to about pH 10.0, about pH 7.0 to about pH 9.5, about pH 7.0 to about pH 9.0, about pH 7.0 to about pH 8.5, about pH 7.0 to about pH 8.0, about pH 7.0 to about pH 7.5, about pH 7.5 to about pH 10.0, about pH 7.5 to about pH 9.5, about pH 7.5 to about pH 9.0, about pH 7.5 to about pH 8.5, about pH 7.5 to about pH 8.0, about pH 8.0 to about pH 10.0, about pH 8.0 to about pH 9.5, about pH 8.0 to about pH 9.0, about pH 8.0 to about pH 8.5, about pH 8.5 to about pH 10.0, about pH 8.5 to about pH 9.5, about pH 8.5 to about pH 9.0, about pH 9.0 to about pH 10.0, or about pH 9.5 to about pH 10.0. For example, in some embodiments, the pH of the buffer solution is about 6.5 to about 9.0, about 7.0 to about 8.5, or about 7.0 to about 7.5 at 95° C. In some embodiments, the pH of the buffer solution is about 7.2 at 95° C.

In some embodiments of any of the preceding methods, the final concentration of the thermostable nucleic acid polymerase in step (iii) is at least about 0.01 units (e.g., about 0.01 units, about 0.02 units, about 0.03 units, about 0.04 units, about 0.05 units, about 0.06 units, about 0.07 units, about 0.08 units, about 0.09 units, about 0.10 units, about 0.15 units about 0.2 units, about 0.25 units, about 0.3 units, about 0.35 units, about 0.4 units, about 0.45 units, about 0.5 units, about 0.6 units, about 0.65 units, about 0.7 units, about 0.8 units, about 0.9 units, about 1 unit, or more) per microliter of the denatured reaction mixture. In some embodiments, the final concentration of the thermostable nucleic acid polymerase may range from about 0.01 units to about 1 unit (e.g., about 0.01 units to about 1 unit, about 0.01 units to about 0.9 units, about 0.01 units to about 0.8 units, about 0.01 units to about 0.7 units, about 0.01 units to about 0.6 units, about 0.01 units to about 0.5 units, about 0.01 units to about 0.4 units, about 0.01 units to about 0.3 units, about 0.01 units to about 0.25 units, about 0.01 units to about 0.2 units, about 0.01 units to about 0.1 unit, about 0.02 units to about 1 unit, about 0.02 units to about 0.9 units, about 0.02 units to about 0.8 units, about 0.02 units to about 0.7 units, about 0.02 units to about 0.6 units, about 0.02 units to about 0.5 units, about 0.02 units to about 0.4 units, about 0.02 units to about 0.3 units, about 0.02 units to about 0.25 units, about 0.02 units to about 0.2 units, about 0.02 units to about 0.1 units, about 0.04 units to about 1 unit, about 0.04 unit to about 0.9 units, about 0.04 units to about 0.8 units, about 0.04 units to about 0.7 units, about 0.04 units to about 0.6 units, about 0.04 units to about 0.5 units, about 0.04 units to about 0.4 units, about 0.04 units to about 0.3 units, about 0.04 units to about 0.25 units, about 0.04 units to about 0.2 units, about 0.04 units to about 0.1 units, about 0.06 units to about 1 unit, about 0.06 units to about 0.9 units, about 0.06 units to about 0.8 units, about 0.06 units to about 0.7 units, about 0.06 units to about 0.6 units, about 0.06 units to about 0.5 units, about 0.06 units to about 0.4 units, about 0.06 units to about 0.3 units, about 0.06 units to about 0.25 units, about 0.06 units to about 0.2 units, about 0.06 units to about 0.1 units, about 0.08 units to about 1 unit, about 0.08 units to about 0.9 units, about 0.08 units to about 0.8 units, about 0.08 units to about 0.7 units, about 0.08 units to about 0.6 units, about 0.08 units to about 0.5 units, about 0.08 units to about 0.4 units, about 0.08 units to about 0.3 units, about 0.08 units to about 0.25 units, about 0.08 units to about 0.2 units, about 0.08 units to about 0.1 units, about 0.1 units to about 1 unit, about 0.1 units to about 0.9 units, about 0.1 units to about 0.8 units, about 0.1 units to about 0.7 units, about 0.1 units to about 0.6 units, about 0.1 units to about 0.5 units, about 0.1 units to about 0.4 units, about 0.1 units to about 0.3 units, about 0.1 units to about 0.25 units, about 0.1 units to about 0.2 units, about 0.2 units to about 1 unit, about 0.2 units to about 0.9 units, about 0.2 units to about 0.8 units, about 0.2 units to about 0.7 units, about 0.2 units to about 0.6 units, about 0.2 units to about 0.5 units, about 0.2 units to about 0.4 units, about 0.2 units to about 0.3 units, about 0.2 units to about 0.25 units, about 0.3 units to about 1 unit, about 0.3 units to about 0.9 units, about 0.3 units to about 0.8 units, about 0.3 units to about 0.7 units, about 0.3 units to about 0.6 units, about 0.3 units to about 0.5 units, about 0.3 units to about 0.4 units, about 0.4 units to about 1 unit, about 0.4 units to about 0.9 units, about 0.4 units to about 0.8 units, about 0.4 units to about 0.7 units, about 0.4 units to about 0.6 units, about 0.4 units to about 0.5 units, about 0.5 units to about 1 unit, about 0.5 units to about 0.9 units, about 0.5 units to about 0.8 units, about 0.5 units to about 0.7 units, about 0.5 units to about 0.6 units, about 0.6 units to about 1 unit, about 0.6 units to about 0.9 units, about 0.6 units to about 0.8 units, about 0.6 units to about 0.7 units, about 0.6 units to about 0.6 units, about 0.7 units to about 1 unit, about 0.7 units to about 0.9 units, about 0.7 units to about 0.8 units, about 0.8 units to about 1 unit, or about 0.8 units to about 0.9 units) per microliter of the mixture. In some embodiments, the final concentration of the thermostable nucleic acid polymerase ranges from about 0.125 to about 0.5 units/μL. In some embodiments, the final concentration of the thermostable nucleic acid ranges from about 0.125 to about 0.25 units/μL.

In some embodiments of any of the preceding methods, step (iii) includes adding to the denatured reaction mixture at least about 1×10⁻⁵ micrograms (e.g., about 1×10⁻⁵ micrograms, about 1.5×10⁻⁵ micrograms, about 2×10⁻⁵ micrograms, about 2.4×10⁻⁵ micrograms, about 2.5×10⁻⁵ micrograms, about 3×10⁻⁵ micrograms, about 4×10⁻⁵ micrograms, about 5×10⁻⁵ micrograms, about 6×10⁻⁵ micrograms, about 7×10⁻⁵ micrograms, about 8×10⁻⁵ micrograms, about 9×10⁻⁵ micrograms, about 1×10⁻⁴ micrograms, about 2×10⁻⁴ micrograms, about 3×10⁻⁴ micrograms, about 4×10⁻⁴ micrograms, about 5×10⁻⁴ micrograms, about 6×10⁻⁴ micrograms, about 7×10⁻⁴ micrograms, about 8×10⁻⁴ micrograms, about 9×10⁻⁴ micrograms, about 1×10⁻⁴ micrograms, about 2×10⁻⁴ micrograms, 3×10⁻⁴ micrograms, about 4×10⁻⁴ micrograms, about 5×10⁻⁴ micrograms, about 6×10⁻⁴ micrograms, about 7×10⁻⁴ micrograms, about 8×10⁻⁴ micrograms, about 9×10⁻³ micrograms, about 0.01 micrograms, about 0.02 micrograms, about 0.03 micrograms, about 0.04 micrograms, about 0.05 micrograms, or more) of a thermostable nucleic acid polymerase per microliter of denatured reaction mixture. For example, In some embodiments of any of the preceding methods, the mixture includes from about 1×10⁻⁵ micrograms to about 0.05 micrograms (e.g., about 1×10⁻⁵ micrograms to about 0.05 micrograms, about 1×10⁻⁵ micrograms to about 0.025 micrograms, about 1×10⁻⁵ micrograms to about 0.01 micrograms, about 1×10⁻⁵ micrograms to about 0.0075 micrograms, about 1×10⁻⁵ micrograms to about 0.005 micrograms, about 1×10⁻⁵ micrograms to about 0.0025 micrograms, about 1×10⁻⁵ micrograms to about 0.001 micrograms, about 1×10⁻⁵ micrograms to about 1×10⁻⁴ micrograms, about 2×10⁻⁵ micrograms to about 0.05 micrograms, about 2×10⁻⁵ micrograms to about 0.025 micrograms, about 2×10⁻⁵ micrograms to about 0.01 micrograms, about 2×10⁻⁵ micrograms to about 0.0075 micrograms, about 2×10⁻⁵ micrograms to about 0.005 micrograms, about 2×10⁻⁵ micrograms to about 0.0025 micrograms, about 2×10⁻⁵ micrograms to about 0.001 micrograms, about 2×10⁻⁵ micrograms to about 1×10⁻⁴ micrograms, about 2.4×10⁻⁵ micrograms to about 0.05 micrograms, about 2.4×10⁻⁵ micrograms to about 0.025 micrograms, about 2.4×10⁻⁵ micrograms to about 0.01 micrograms, about 2.4×10⁻⁵ micrograms to about 0.0075 micrograms, about 2.4×10⁻⁵ micrograms to about 0.005 micrograms, about 2.4×10⁻⁵ micrograms to about 0.0025 micrograms, about 2.4×10⁻⁵ micrograms to about 0.001 micrograms, about 2.4×10⁻⁵ micrograms to about 1×10⁻⁴ micrograms, about 5×10⁻⁵ micrograms to about 0.05 micrograms, about 5×10⁻⁵ micrograms to about 0.025 micrograms, about 5×10⁻⁵ micrograms to about 0.01 micrograms, about 5×10⁻⁵ micrograms to about 0.0075 micrograms, about 5×10⁻⁵ micrograms to about 0.005 micrograms, about 5×10⁻⁵ micrograms to about 0.0025 micrograms, about 5×10⁻⁵ micrograms to about 0.001 micrograms, about 5×10⁻⁵ micrograms to about 1×10⁻⁴ micrograms, about 8×10⁻⁵ micrograms to about 0.05 micrograms, about 8×10⁻⁵ micrograms to about 0.025 micrograms, about 8×10⁻⁵ micrograms to about 0.01 micrograms, about 8×10⁻⁵ micrograms to about 0.0075 micrograms, about 8×10⁻⁵ micrograms to about 0.005 micrograms, about 8×10⁻⁵ micrograms to about 0.0025 micrograms, about 8×10⁻⁵ micrograms to about 0.001 micrograms, about 8×10⁻⁵ micrograms to about 1×10⁻⁴ micrograms, about 1×10⁻⁴ micrograms to about 0.05 micrograms, about 1×10⁻⁴ micrograms to about 0.025 micrograms, about 1×10⁻⁴ micrograms to about 0.01 micrograms, about 1×10⁻⁴ micrograms to about 0.0075 micrograms, about 1×10⁻⁴ micrograms to about 0.005 micrograms, about 1×10⁻⁴ micrograms to about 0.0025 micrograms, about 1×10⁻⁴ micrograms to about 0.001 micrograms, about 5×10⁻⁴ micrograms to about 0.05 micrograms, about 5×10⁻⁴ micrograms to about 0.025 micrograms, about 5×10⁻⁴ micrograms to about 0.01 micrograms, about 5×10⁻⁴ micrograms to about 0.0075 micrograms, about 5×10⁻⁴ micrograms to about 0.005 micrograms, about 5×10⁻⁴ micrograms to about 0.0025 micrograms, about 5×10⁻⁴ micrograms to about 0.001 micrograms, about 1×10⁻³ micrograms to about 0.05 micrograms, about 1×10⁻³ micrograms to about 0.025 micrograms, about 1×10⁻³ micrograms to about 0.01 micrograms, about 1×10⁻³ micrograms to about 0.0075 micrograms, about 1×10⁻³ micrograms to about 0.005 micrograms, or about 1×10⁻³ micrograms to about 0.0025 micrograms) of the thermostable nucleic acid polymerase per microliter of the mixture. In some embodiments, the final concentration of thermostable nucleic acid polymerase is from about 2.4×10⁻⁵ micrograms to about 0.01 micrograms per microliter of denatured reaction mixture or reaction mixture. In some embodiments, the final concentration of thermostable nucleic acid polymerase is from about 2.4×10⁻⁵ micrograms to about 0.0001 micrograms per microliter of denatured reaction mixture or reaction mixture.

In some embodiments of any of the preceding methods, step (ii) may include heating the reaction mixture to greater than about 55° C., e.g., 55° C., 60° C., 65° C., 70° C., 75° C., 80° C., 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., or 100° C. In some embodiments, the temperature is about 95° C.

In some embodiments of any of the methods described herein, the thermostable nucleic acid polymerase is a thermostable DNA polymerase. Any suitable thermostable DNA polymerase may be used in the methods of the invention, for example, commercially available thermostable DNA polymerases, or any thermostable DNA polymerase described herein and/or known in the art. In some embodiments, the thermostable DNA polymerase is a wild-type thermostable DNA polymerase, e.g., Thermus aquaticus (Taq) DNA polymerase (see, e.g., U.S. Pat. No. 4,889,818), Thermus thermophilus (Tth) DNA polymerase (see, e.g., U.S. Pat. Nos. 5,192,674; 5,242,818; and 5,413,926), Thermus filiformis (Tfi) DNA polymerase, Thermus flavus (Tfl) DNA polymerase, Thermococcus litoralis (Tli) DNA polymerase (see, e.g., U.S. Pat. No. 5,332,785), Thermatoga maritima (Tma) DNA polymerase, Thermus spp. Z05 DNA polymerase, Tsp sps17 DNA polymerase derived from Thermus species spsl 7, now called Thermus oshimai(see, e.g. U.S. Pat. No. 5,405,774), Bacillus stearothermophilus (Bst) DNA polymerase (see, e.g., U.S. Pat. No. 5,747,298), an archaeal polymerase (e.g., thermostable DNA polymerases from hyperthermophylic archaeons Pyrococcus furiosus (e.g., Pfu; see, e.g., U.S. Pat. No. 5,948,663), KOD DNA polymerase derived from Pyrococcus sp. KOD1 (e.g., U.S. Pat. No. 6,033,859), Thermococcus litoralis (e.g., VENTR® (NEB)), and 9°N™ (NEB)), or a mutant, derivative, or fragment thereof having DNA polymerase activity (e.g., mutant DNA polymerases that include point mutations compared to a reference thermostable DNA polymerase sequence, e.g., Taq A271 F667Y, Tth A273 F668Y, and Taq A271 F667Y E681W; truncation mutants, e.g., KIenTAQ®, an N-terminal deletion variant of Taq lacking the first 280 amino acids; and mutants that include truncations and point mutations, e.g., Hemo KlenTaq®, an N-terminal deletion variant of Taq lacking the first 280 amino acids containing three internal point mutations that make it resistant to inhibitors in whole blood). For example, suitable DNA polymerases include, but are not limited to, Taq, Hemo KlenTaq®, Hawk Z05, APTATAQ™, Pfu, VENTR®, or higher fidelity DNA polymerases such as PHUSION® (Thermo Scientific), Q50 (NEB), KAPA HiFi™ (Roche), PfuUltra (Agilent), KOD XTREME™ (Millipore), HotStar HiFidelity (Qiagen), ACCUPRIME™ Pfx (Invitrogen), and PLATINUM™ Taq (Invitrogen).

In some embodiments, the thermostable DNA polymerase is a mutant thermostable DNA polymerase. In some embodiments, the mutant thermostable DNA polymerase is listed in Table B. In some embodiments, the mutant thermostable DNA polymerase is selected from the group consisting of Klentaq®1, Klentaq® LA, Cesium Klentaq® AC, Cesium Klentaq® AC LA, Cesium Klentaq® C, Cesium Klentaq® C LA, Omni Klentaq®, Omni Klentaq® 2, Omni Klentaq® LA, Omni Taq, OmniTaq LA, Omni Taq 2, Omni Taq 3, Hemo KlenTaq®, KAPA Blood DNA polymerase, KAPA3G Plant DNA polymerase, KAPA 3G Robust DNA polymerase, MyTaq™ Blood, PHUSION® Blood II DNA polymerase, AmpliTaq® (Taq G46D), AmpliTaq® Gold, RealTaq, ExcelTaq™, and BioReady Taq. In some embodiments, the thermostable DNA polymerase is a hot start thermostable DNA polymerase (e.g., APTATAQ™, Hawk Z05, or PHUSION® Blood II DNA polymerase).

In some embodiments, the thermostable nucleic acid polymerase (e.g., thermostable DNA polymerase) is inhibited by the presence of subject-derived cells or cell debris under normal reaction conditions. In some embodiments, the thermostable nucleic acid polymerase (e.g., thermostable DNA polymerase) is inhibited by whole blood under normal reaction conditions. In some embodiments, the thermostable nucleic acid polymerase (e.g., thermostable DNA polymerase) is inhibited by 1% (v/v) whole blood under normal reaction conditions. In some embodiments, the thermostable nucleic acid polymerase (e.g., thermostable DNA polymerase) is inhibited by 8% (v/v) whole blood under normal reaction conditions. In some embodiments, the normal reaction conditions are the reaction conditions recommended by the manufacturer of the thermostable DNA polymerase or reaction conditions that are commonly used in the art.

In some embodiments of any of the preceding methods, the method further includes amplifying one or more additional target nucleic acids in a multiplexed PCR reaction to generate one or more additional amplicons. In some embodiments, the multiplexed PCR reaction amplifies 2, 3, 4, 5, 6, 7, 8, 9, 10, 11, 12, 15, 20, 25, 30, 35, 40, 45, 50, 60, or more target nucleic acids.

In some embodiments of any of the preceding methods, the method further includes adding deoxynucleotide triphosphates (dNTPs), magnesium, one or more forward primers (e.g., 1, 2, 3, 4, 5, 6, 7, 8, 9, 10, 11, or 12 forward primers), and/or one or more reverse primers (e.g., 1, 2, 3, 4, 5, 6, 7, 8, 9, 10, 11, or 12 reverse primers) during step (i) or during step (iii).

In some embodiments of any of the preceding methods, the whole blood sample is about 0.2 mL to about 5 mL (e.g., about 0.2 mL, about 0.3 mL, about 0.4 mL, about 0.5 mL, about 0.6 mL, about 0.7 mL, about 0.8 mL, about 0.9 mL, about 1 mL, about 1.1 mL, about 1.2 mL, about 1.3 mL, about 1.4 mL, about 1.5 mL, about 1.6 mL, about 1.7 mL, about 1.8 mL, about 1.9 mL, about 2 mL, about 2.5 mL, about 3 mL, about 3.5 mL, about 4 mL, about 5 mL).

The invention allows use of a concentrated lysate prepared from a larger volume of whole blood. In some embodiments, a lysate produced from a whole blood sample of about 0.2 mL to about 10 mL has a volume of about 10 μL to about 1000 μL (e.g., about 10 μL, about 20 μL about 30 μL, about 40 μL, about 50 μL, about 60 μL, about 70 μL, about 80 μL, about 90 μL, about 100 μL, about 125 μL, about 150 μL, about 175 μL, about 200 μL, about 225 μL, about 250 μL, about 275 μL, about 300 μL, about 325 μL, about 350 μL, about 375 μL, about 400 μL, about 425 μL, about 450 μL, about 475 μL, about 500 μL, about 525 μL, about 550 μL, about 600 μL, about 625 μL, about 650 μL, about 675 μL, about 700 μL, about 725 μL, about 750 μL, about 775 μL, about 800 μL, about 825 μL, about 850 μL, about 875 μL, about 900 μL, about 925 μL, about 950 μL, about 975 μL, or about 1000 μL). In some embodiments, the lysate produced from the whole blood sample has a volume of about 25 μL to about 200 μL. In some embodiments, the lysate produced from the whole blood sample has a volume of about 50 μL. In some embodiments, the lysate is concentrated at least 2-fold, 3-fold, 4-fold, 5-fold, 6-fold, 7-fold, 8-fold, 9-fold, 10-fold, 20-fold, 30-fold, 40-fold, 50-fold, or more compared to the whole blood sample. In some embodiments, the reaction mixture of step (i) contains about 1% to about 80% lysate (e.g., about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, or about 80% crude blood lysate In some embodiments, the reaction mixture of step (i) contains about 50% lysate.

In some embodiments of any of the preceding methods, the denatured reaction mixture has a volume ranging from about 0.1 μL to about 250 μL or more, e.g., about 1 μL, about 10 μL, about 20 μL, about 30 μL, about 40 μL, about 50 μL, about 50 μL, about 60 μL, about 70 μL, about 80 μL, about 90 μL, about 100 μL, about 110 μL, about 120 μL, about 130 μL, about 140 μL, about 150 μL, about 160 μL, about 170 μL, about 180 μL, about 190 μL, about 200 μL, or more. In some embodiments, the volume of the denatured reaction mixture is about 100 μL.

In some embodiments of any of the preceding methods, the method does not include extracting or purifying the amplified drug resistance (e.g., antibiotic resistance) target nucleic acid prior to the sequencing. In some embodiments, the extracting includes chloroform or phenol/chloroform extraction, nuclease digestion, salting out, ion exchange extraction, binding to silica or other solid phase materials, or gel extraction.

In some embodiments of any of the preceding methods, the method further includes cleaning up the amplified drug resistance (e.g., antibiotic resistance) target nucleic acid prior to the sequencing. In some embodiments, the cleaning up includes magnetic bead purification, enzymatic clean-up, or column clean-up. In other embodiments, no clean-up step is performed.

Any suitable sequencing approach can be used in the methods described herein. For example, in some embodiments, the sequencing includes massively parallel sequencing (e.g., sequencing by synthesis (e.g., ILLUMINA™ dye sequencing, ion semiconductor sequencing, or pyrosequencing) or sequencing by ligation (e.g., oligonucleotide ligation and detection (SOLiD™) sequencing or polony-based sequencing)), single molecule sequencing (e.g., Helicos™ sequencing, single-molecule real-time (SMRT™) sequencing, and nanopore sequencing) and Sanger sequencing.

In some embodiments of any of the preceding methods, the method further includes amplifying one or more additional target nucleic acids in a multiplexed amplification (e.g., multiplexed PCR) reaction to generate one or more additional amplicons. Such additional target nucleic acids can be used, for example, to detect members of any of the panels described herein.

In some embodiments of any of the preceding methods, the method identifies the genus from which the drug resistance (e.g., antibiotic resistance) target nucleic acid is derived. In some embodiments of any of the preceding methods, the method identifies the species of the pathogen from which the drug resistance (e.g., antibiotic resistance) target nucleic acid is derived.

Any of the methods described above may include detecting the amplified drug resistance (e.g., antibiotic resistance) target nucleic acid using T2 magnetic resonance (T2MR). The detecting by T2MR can occur prior to, after, or concurrent with the sequencing. In particular embodiments, the detecting by T2MR occurs prior to the sequencing. For example, in some embodiments, the method includes the following steps: (i) adding magnetic particles to a portion of the amplified solution or amplified lysate solution to form a detection mixture, wherein the magnetic particles have binding moieties on their surface, the binding moieties operative to alter aggregation of the magnetic particles in the presence of the amplified target nucleic acid, and (ii) detecting the presence of the amplified target nucleic acid by measuring the aggregation of the magnetic particles using T2MR. In some embodiments, step (ii) includes the following steps: (a) providing the detection mixture in a detection tube within a device, the device including a support defining a well for holding the detection tube including the mixture, and having an RF coil configured to detect a signal produced by exposing the mixture to a bias magnetic field created using one or more magnets and an RF pulse sequence; (b) exposing the detection mixture to a bias magnetic field and an RF pulse sequence; (c) following step (b), measuring the signal from the detection tube; and (d) on the basis of the result of step (c), detecting the amplified target nucleic acid.

The magnetic particles may be any of the magnetic particles described herein or in International Patent Application Publication Nos. WO 2012/054639, WO 2016/118766, WO 2017/127731, or in International Patent Application No. PCT/US2018/033278, each of which is incorporated herein by reference in its entirety. In some embodiments, the magnetic particles include a first population of magnetic particles conjugated to a first probe, and a second population of magnetic particles conjugated to a second probe, the first probe operative to bind to a first segment of the amplified target nucleic acid and the second probe operative to bind to a second segment of the amplified target nucleic acid, wherein the magnetic particles form aggregates in the presence of the amplified target nucleic acid. The magnetic particles may be substantially monodisperse.

The magnetic particles may have any suitable size, for example, any size described below, or in International Patent Application Publication Nos. WO 2012/054639, WO 2016/118766, WO 2017/127731, or in International Patent Application No. PCT/US2018/033278. In some embodiments, the magnetic particles have a mean diameter of from 650 nm to 950 nm. In some embodiments, the magnetic particles have a mean diameter of from about 670 nm to about 890 nm.

The magnetic particles may have any T₂ relaxivity per particle, for example, T₂ relaxivity per particle described below. In some embodiments, the magnetic particles have a T₂ relaxivity per particle of from 1×10⁹ to 1×10¹² mM⁻¹ s⁻¹.

Any suitable amount of magnetic particles can be added to the sample, for example, any amount described below. In some embodiments, from 1×10⁶ to 1×10¹³ magnetic particles are added per milliliter of the sample or the amplified solution.

In another example, provided herein is a method for detecting a drug resistance (e.g., antibiotic resistance) target nucleic acid in a biological sample obtained from a subject, wherein the biological sample includes subject-derived cells or cell debris, the method including the following steps: (a) amplifying a target nucleic acid in the biological sample to form an amplified solution including an amplified target nucleic acid; (b) detecting the amplified target nucleic acid using T2MR to provide a group-level identification of the target nucleic acid; and (c) sequencing the amplified target nucleic acid to provide a species-level or variant-level identification of the target nucleic acid, wherein the method is capable of detecting a concentration of about 10 copies/mL of the target nucleic acid in the biological sample.

In any of the preceding methods, detecting the amplified drug resistance (e.g., antibiotic resistance) target nucleic acid using T2MR can result in a group-level identification of the target drug resistance (e.g., antibiotic resistance) nucleic acid by T2MR. The detecting can provide group-level information for any species described herein. In some embodiments, the group-level identification identifies the organism from which the target drug resistance (e.g., antibiotic resistance) nucleic acid is obtained as pan-Gram positive, pan-Gram negative, Enterobacteriaceae, an Enterobacter spp., an Enterobacter cloacae complex, a Citrobacter spp., an Enterococcus spp., a Streptococcus spp., a Staphylococcus spp. (e.g., a coagulase-negative Staphylococcus spp.), an Acinetobacter spp., a Corynebacterium spp., a Mycobacterium spp., pan-fungal, a Candida spp., or a biothreat species (e.g., Bacillus anthracis, Francisella tularensis, Burkholderia spp. (e.g., B. mallei or B. pseudomallei), Yersinia pestis, or Rickettsia prowazekii). In some embodiments, the group-level identification identifies the organism from which the target drug resistance (e.g., antibiotic resistance) nucleic acid is obtained as a Gram positive bacterium (pan Gram positive), a Gram negative bacterium (pan Gram negative), an Enterobacteriaceae spp., an Enterobacter spp., a Kiebsiella spp., an Escherichia spp., a Klebsiella spp., an Enterobacter cloacae complex, a Citrobacter spp., a Pseudomonas spp., an Enterococcus spp., a Streptococcus spp., a viridans group Streptococcus, Staphylococcus spp., a coagulase-negative 35 Staphylococcus spp., an Acinetobacter spp., an Cornybacterium spp., a Mycobacterium spp., a Haemophilus spp., a Salmonella spp., a Clostridium spp., a Neisseria spp., a Serratia spp., a Proteus spp., a Stenotrophomonas spp., or an anaerobe (e.g., Clostridium spp. and/or Bacteroides spp). In other embodiments, the group-level identification identifies the organism from which the target drug resistance (e.g., antibiotic resistance) nucleic acid is obtained as a fungal pathogen (pan Fungal), a Candida spp., an Aspergillus spp., or a Cryptococcus spp.

In some embodiments, the group-level identification identifies the target nucleic acid as including a sequence of an antimicrobial resistance gene or a toxin gene or a fragment thereof. In some embodiments, detecting the amplified target nucleic acid using T2MR results in the identification of a sequence of an antimicrobial resistance gene or a fragment thereof. Non-limiting examples of antimicrobial resistance genes include bla_{KPC}, blaZ, bla_{NDM}, bla_{IMP}, bla_{VIM}, bla_{OXA}, bla_{CMY}, bla_{DHA}, bla_{TEM}, bla_{SHV}, bla_{CTX-M}, bla_{SME}, bla_{FOX}, bla_{MIR}, femA, femB, mecA, mecC, macB, fosA, vanA, vanB, vanC, vanD, vanE, vanG, mefA, mefE, ermA, ermB, tetA, tetB, tetX, tetR, qnrA, qnrB, or qnrS, or variants thereof.

In any of the preceding methods, sequencing the amplified target nucleic acid can result in a species-level or variant-level identification of the target drug resistance (e.g., antibiotic resistance) nucleic acid. In some embodiments, the species level is a taxonomic species, a taxonomic subspecies, or a strain. In some embodiments, the variant-level identification is a nucleic acid variant (e.g., a single nucleotide polymorphism (SNP), an insertion/deletion (indel), a repetitive element, or a microsatellite repeat).

In some embodiments, the group-level identification by T2MR is an antimicrobial resistance gene, and the species-level identification by sequencing is a nucleic acid variant of the antimicrobial resistance gene (e.g., bla_{KPC}, blaZ, bla_{NDM}, bla_{IMP}, bla_{VIM}, bla_{OXA}, bla_{CMY}, bla_{DHA}, bla_{TEM}, bla_{SHV}, bla_{CTX-M}, bla_{SME}, bla_{FOX}, bla_{MIR}, femA, femB, mecA, mecC, macB, fosA, vanA, vanB, vanC, vanD, vanE, vanG, mefA, mefE, ermA, ermB, tetA, tetB, tetX, tetR, qnrA, qnrB, or qnrS). For example, any of the variants listed in FIG. 2 can be identified, e.g., by sequencing. For example, in some embodiments, (i) the identification by T2MR is bla_{KPC}, and the variant-level identification by sequencing is KPC-1, KPC-2, KPC-3, KPC-4, KPC-5, KPC-6, KPC-7, KPC-8, KPC-10, KPC-11, KPC-12, KPC-13, KPC-14, KPC-15, KPC-16, KPC-17, KPC-18, KPC-19, KPC-21, KPC-22, KPC-23, KPC-24, KPC-25, KPC-26, KPC-27, KPC-28, KPC-29, KPC-30, KPC-31, KPC-32, KPC-33, KPC-34, or KPC-35; (ii) the identification by T2MR is bla_{CTX-M}, and the variant-level identification by sequencing is CTX-M-1, CTX-M-2, CTX-M-3, CTX-M-4, CTX-M-5, CTX-M-6, CTX-M-7, CTX-M-8, CTX-M-9, CTX-M-10, CTX-M-12, CTX-M-13, CTX-M-14, CTX-M-15, CTX-M-16, CTX-M-17, CTX-M-19, CTX-M-20, CTX-M-21, CTX-M-22, CTX-M-23, CTX-M-24, CTX-M-25, CTX-M-26, CTX-M-27, CTX-M-28, CTX-M-29, CTX-M-30, CTX-M-31, CTX-M-32, CTX-M-33, CTX-M-34, CTX-M-35, CTX-M-36, CTX-M-37, CTX-M-38, CTX-M-39, CTX-M-40, CTX-M-41, CTX-M-42, CTX-M-43, CTX-M-44, CTX-M-46, CTX-M-47, CTX-M-48, CTX-M-49, CTX-M-50, CTX-M-51, CTX-M-52, CTX-M-53, CTX-M-54, CTX-M-55, CTX-M-56, CTX-M-58, CTX-M-59, CTX-M-60, CTX-M-61, CTX-M-62, CTX-M-63, CTX-M-64, CTX-M-65, CTX-M-66, CTX-M-67, CTX-M-68, CTX-M-69, CTX-M-71, CTX-M-72, CTX-M-73, CTX-M-74, CTX-M-75, CTX-M-76, CTX-M-77, CTX-M-78, CTX-M-79, CTX-M-80, CTX-M-81, CTX-M-82, CTX-M-83, CTX-M-84, CTX-M-85, CTX-M-86, CTX-M-87, CTX-M-88, CTX-M-89, CTX-M-90, CTX-M-91, CTX-M-92, CTX-M-93, CTX-M-94, CTX-M-95, CTX-M-96, CTX-M-97, CTX-M-98, CTX-M-99, CTX-M-100, CTX-M-101, CTX-M-102, CTX-M-103, CTX-M-104, CTX-M-105, CTX-M-110, CTX-M-111, CTX-M-112, CTX-M-113, CTX-M-114, CTX-M-115, CTX-M-116, CTX-M-117, CTX-M-121, CTX-M-122, CTX-M-123, CTX-M-124, CTX-M-125, CTX-M-126, CTX-M-127, CTX-M-129, CTX-M-130, CTX-M-131, CTX-M-132, CTX-M-134, CTX-M-136, CTX-M-137, CTX-M-138, CTX-M-139, CTX-M-141, CTX-M-142, CTX-M-144, CTX-M-146, CTX-M-147, CTX-M-148, CTX-M-150, CTX-M-151, CTX-M-152, CTX-M-155, CTX-M-156, CTX-M-157, CTX-M-158, CTX-M-159, CTX-M-160, CTX-M-161, CTX-M-162, CTX-M-163, CTX-M-164, CTX-M-165, CTX-M-166, CTX-M-167, CTX-M-168, CTX-M-169, CTX-M-170, CTX-M-171, CTX-M-172, CTX-M-173, CTX-M-174, CTX-M-175, CTX-M-176, CTX-M-177, CTX-M-178, CTX-M-179, CTX-M-180, CTX-M-181, CTX-M-182, CTX-M-183, CTX-M-184, CTX-M-185, CTX-M-186, CTX-M-187, CTX-M-188, CTX-M-189, CTX-M-190, CTX-M-191, CTX-M-192, CTX-M-193, CTX-M-194, CTX-M-195, CTX-M-196, CTX-M-197, CTX-M-198, CTX-M-199, CTX-M-200, CTX-M-201, CTX-M-202, CTX-M-203, CTX-M-204, CTX-M-205, CTX-M-206, CTX-M-207, CTX-M-208, CTX-M-209, CTX-M-210, CTX-M-211, CTX-M-212, CTX-M-213, CTX-M-214, CTX-M-216, CTX-M-217, CTX-M-218, CTX-M-219, or CTX-M-220; or (iii) the identification by T2MR is bla_{NDM}, and the variant-level identification by sequencing is NDM-1, NDM-2, NDM-3, NDM-4, NDM-5, NDM-6, NDM-7, NDM-8, NDM-9, NDM-10, NDM-11, NDM-12, NDM-13, NDM-14, NDM-15, NDM-16, NDM-17, NDM-18, NDM-19, NDM-20, NDM-21, NDM-22, NDM-23, or NDM-24.

In any of the preceding methods, the detecting by T2MR can be completed within 5 hours of amplifying the target nucleic acid, e.g., within about 5, 4, 3, 2, or 1 hour.

Any suitable pathogen can be detected and/or sequenced using any of the methods described herein. For example, in some embodiments, the pathogen is a bacterial pathogen. In some embodiments, the pathogen is a drug-resistant pathogen (e.g., an antibiotic-resistant bacterium (e.g., an antibiotic-resistant Gram negative pathogen)).

Any suitable bacterial pathogen may be detected, including any described herein. In some embodiments, the amplifying includes amplifying a pan-bacterial amplicon (e.g., a 16S rRNA amplicon). Any suitable primer pair described herein or known in the art can be used In some embodiments, the bacterial pathogen is a Gram positive bacterium, a Gram negative bacterium, an Enterobacteriaceae family bacterium, an Enterobacter spp., a Citrobacter spp., a Enterococcus spp., a Streptococcus spp. (e.g., a viridans Streptococcus), a Staphylococcus spp. (e.g., a coagulase-negative Staphylococcus spp.), an Acinetobacter spp., a Corynebacterium spp., Enterobacter cloacae complex, or a Mycobacterium spp. In some embodiments, the bacterial pathogen is selected from the group consisting of Acinetobacter baumannii, Escherichia coli, Enterococcus faecalis, Enterococcus faecium, Kiebsiella pneumoniae, Pseudomonas aeruginosa, Staphylococcus aureus, Borrelia burgdoferi, Borrelia afzelii, Borrelia garinii, Rickettsia rickettsii, Anaplasma phagocytophilum, Coxiella burnetii, Ehrlichia chaffeensis, Ehrlichia ewingii, Francisella tularensis, Streptococcus pneumoniae, Enterobacter cloacae, Streptococcus pyogenes, Streptococcus mutans, Streptococcus sanguinis, Haemophilus influenzae, and Neisseria meningitides. In some embodiments, the bacterial pathogen is selected from the group consisting of Acinetobacter baumannii, Enterococcus faecium, Staphylococcus aureus, Klebsiella pneumoniae, Pseudomonas aeruginosa, and Escherichia coli. In other embodiments, the bacterial pathogen is selected from Borrelia burgdorferi, Borrelia afzelii, and Borrelia garinii.

In some embodiments of any of the preceding methods, the target nucleic acid may be an antimicrobial (e.g., antibiotic) resistance gene. Any suitable antimicrobial resistance gene may be detected and/or sequenced using the methods described herein. Exemplary antimicrobial resistance genes include but are not limited to bla_{KPC}, blaZ, bla_{NDM}, bla_{IMP}, bla_{VIM}, bla_{OXA}, bla_{CMY}, bla_{DHA}, bla_{TEM}, bla_{SHV}, bla_{CTX-M}, bla_{SME}, bla_{FOX}, bla_{MIR}, femA, femB, mecA, macB, fosA, vanA, vanB, vanC, vanD, vanE, vanG, mefA, mefE, ermA, ermB, tetA, tetB, tetX, tetR, qnrA, qnrB, or qnrS.

Any of the methods described herein may further include diagnosing the subject based on the detection of the target drug resistance (e.g., antibiotic resistance) nucleic acid, or the nucleotide sequence of the target drug resistance (e.g., antibiotic resistance) nucleic acid, wherein the presence or sequence of the target nucleic indicates that the subject is suffering from a disease associated with a drug-resistant pathogen. The method may further include administering to the subject a suitable therapy, e.g., a therapy tailored to the drug resistance profile of the pathogen based on the sequence of the target nucleic acid.

In another example, in some embodiments, the invention provides a method for sequencing a drug resistance (e.g., antibiotic resistance) target nucleic acid in a sample including unprocessed whole blood, the method including: (a) providing a mixture including a buffer solution including a buffering agent, dNTPs, magnesium, a forward primer, a reverse primer, and a thermostable nucleic acid polymerase, wherein the buffer solution has a moderately alkaline pH at ambient temperature, and wherein the final concentration of the thermostable nucleic acid polymerase is at least about 0.01 units (e.g., about 0.01 units, about 0.02 units, about 0.03 units, about 0.04 units, about 0.05 units, about 0.06 units, about 0.07 units, about 0.08 units, about 0.09 units, about 0.10 units, about 0.15 units about 0.2 units, about 0.25 units, about 0.3 units, about 0.35 units, about 0.4 units, about 0.45 units, about 0.5 units, about 0.6 units, about 0.65 units, about 0.7 units, about 0.8 units, about 0.9 units, about 1 unit, or more) per microliter of the mixture; (b) adding to the mixture a portion of a whole blood sample obtained from a subject to form a reaction mixture; (c) amplifying the target nucleic acid to form an amplified solution including an amplicon; and (d) sequencing the amplicon. In some embodiments, the reaction mixture contains from about 1% to about 70% (v/v) whole blood, e.g., about 1%, about 2%, about 3%, about 4%, about 5%, about 10%, about 15%, about 20%, about 25%, about 30%, about 35%, about 40%, about 45%, about 50%, about 55%, about 60%, about 65%, or about 70% (v/v) whole blood). In some embodiments, the reaction mixture contains more than about 1%, more than about 2%, more than about 3%, more than about 4%, more than about 5%, more than about 10%, more than about 15%, more than about 20%, more than about 25%, more than about 30%, more than about 35%, more than about 40%, more than about 45%, more than about 50%, more than about 55%, more than about 60%, more than about 65%, or more than about 70% (v/v) whole blood.

In a still further example, in some embodiments, the invention provides a method for sequencing a drug resistance (e.g., antibiotic resistance) target nucleic acid in a sample including whole blood, the method including: (a) providing a mixture, wherein the mixture includes a buffer solution including a buffering agent, dNTPs, magnesium, a forward primer, a reverse primer, and a thermostable nucleic acid polymerase, wherein the buffer solution has a moderately alkaline pH at ambient temperature, and wherein the mixture contains about at least about 1×10⁻⁵ micrograms (e.g., about 1×10⁻⁵ micrograms, about 1.5×10⁻⁵ micrograms, about 2×10⁻⁵ micrograms, about 2.4×10⁻⁵ micrograms, about 2.5×10⁻⁵ micrograms, about 3×10⁻⁵ micrograms, about 4×10⁻⁵ micrograms, about 5×10⁻⁵ micrograms, about 6×10⁻⁵ micrograms, about 7×10⁻⁵ micrograms, about 8×10⁻⁵ micrograms, about 9×10⁻⁵ micrograms, about 1×10⁻⁴ micrograms, about 2×10⁻⁴ micrograms, about 3×10⁻⁴ micrograms, about 4×10⁻⁴ micrograms, about 5×10⁻⁴ micrograms, about 6×10⁻⁴ micrograms, about 7×10⁻⁴ micrograms, about 8×10⁻⁴ micrograms, about 9×10⁻⁴ micrograms, about 1×10⁻³ micrograms, about 2×10⁻³ micrograms, 3×10⁻³ micrograms, about 4×10⁻³ micrograms, about 5×10⁻³ micrograms, about 6×10⁻³ micrograms, about 7×10⁻³ micrograms, about 8×10⁻³ micrograms, about 9×10⁻³ micrograms, about 0.01 micrograms, about 0.02 micrograms, about 0.03 micrograms, about 0.04 micrograms, about 0.05 micrograms, or more) of the thermostable nucleic acid polymerase per microliter of the mixture of the thermostable nucleic acid polymerase; (b) adding to the mixture a portion of a whole blood sample obtained from a subject to form a reaction mixture; (c) amplifying the target nucleic acid to form an amplified solution including an amplicon; and (d) sequencing the amplicon. In some embodiments, the reaction mixture contains from about 1% to about 70% (v/v) whole blood, e.g., about 1%, about 2%, about 3%, about 4%, about 5%, about 10%, about 15%, about 20%, about 25%, about 30%, about 35%, about 40%, about 45%, about 50%, about 55%, about 60%, about 65%, or about 70% (v/v) whole blood).

Any suitable buffering agent may be used in the methods of the invention. For example, in some embodiments, any buffer with a pKa ranging from about 7.0 to about 9.2 (e.g., about 7.0 to about 7.6; from about 7.6 to about 8.2; or about 8.2 to about 9.2) may be used. Exemplary buffering agents with a pKa ranging from about 7.0 to about 7.6 include but are not limited to: MOPS, BES, phosphoric acid, TES, HEPES, and DIPSO. Exemplary buffering agents with a pKa ranging from about 7.6 to about 8.2 include but are not limited to: TAPSO, TEA, n-ethylmorpholine, POPSO, EPPS, HEPPSO, Tris, and Tricine. Exemplary buffering agents with a pKa ranging from about 8.2 to about 9.2 include but are not limited to: glycyiglycine, Bicine, TAPS, morpholine, n-methyldiethanolamine, AMPD (2-amino-2-methyl-1,3-propanediol), diethanolamine, and AMPSO. In some embodiments, a buffering agent with a pKa greater than 9.2 may be used. Exemplary buffering agents with a pKa greater than 9.2 include but are not limited to boric acid, CHES, glycine, CAPSO, ethanolamine, AMP (2-amino-2-methyl-1-propanol), piperazine, CAPS, 1,3-diaminopropane, CABS, and piperadine.

In some embodiments of any of the preceding methods, the method results in the production of at least 10⁵ copies of the amplicon, e.g., at least 10⁵ copies, at least 10⁶ copies, at least 10⁷ copies, at least 10⁸ copies, at least 10⁹ copies, at least 10¹⁰ copies, at least 10¹¹ copies, at least 10¹² copies, at least 10¹³ copies, or at least 10¹⁴ copies of the amplicon. For example, in some embodiments, the method results in the production of at least 10⁸ copies of the amplicon. In some embodiments, the method results in the production of at least 10⁹ copies of the amplicon.

Any of the preceding methods can further include detecting one or more (e.g., 1, 2, 3, 4, 5, 6, 7, 8, 9, 10, or more) additional analytes (e.g., nucleic acids (e.g., DNA or RNA (e.g., mRNA)), proteins, cells, or the like. The detecting may be by any suitable approach, e.g., sequencing (e.g., massively-parallel, long-read, and/or Sanger sequencing), optical, fluorescent, mass, density, magnetic, chromatographic, and/or electrochemical measurement. In some embodiments, the detecting is performed by T2MR. Further provided herein are systems for performing any of the methods described herein, as described further below.

### Sample Preparation and Cell Lysis

The methods and systems of the invention may involve sample preparation and/or cell lysis. For example, an organism (e.g., a drug-resistant pathogen) present in a biological sample containing cells, cell debris, and/or nucleic acids (e.g., DNA or RNA (e.g., mRNA)), including but not limited to blood (e.g., whole blood, a crude whole blood lysate, serum, or plasma), bloody fluids (e.g., wound exudate, phlegm, bile, and the like), tissue samples (e.g., tissue biopsies, including homogenized tissue samples), urine, BAL, CSF, SF, or sputum may be lysed prior to amplification of a target nucleic acid. Suitable lysis methods for lysing cells (e.g., pathogen cells) in a biological sample include, for example, mechanical lysis (e.g., beadbeating and sonication), heat lysis, and alkaline lysis.

In some embodiments, the lysis method is beadbeating. In some embodiments, beadbeating may be performed by adding glass beads (e.g., 0.5 mm glass beads, 0.6 mm glass beads, 0.7 mm glass beads, 0.8 mm glass beads, or 0.9 mm glass beads) to a biological sample to form a mixture and agitating the mixture. As an example, the sample preparation and cell lysis (e.g., beadbeating) may be performed using any of the approaches and methods described in WO 2012/054639. Following lysis, the sample may include cell debris or nucleic acids derived from mammalia n host cells and/or from the pathogen cell(s) present in the sample.

In some embodiments, the methods of the invention may include preparing a tissue homogenate. Any suitable method or approach known in the art and/or described herein may be used, including but not limited to grinding (e.g., mortar and pestle grinding, cryogenic mortar and pestle grinding, or glass homogenizer), shearing (e.g., blender, rotor-stator, dounce homogenizer, or French press), beating (e.g., beadbeating), or sonication. In some embodiments, several approaches may be combined to prepare a tissue homogenate.

In some embodiments, the methods of the invention involve detection of one or more antibiotic resistance genes (e.g., one or more antibiotic resistance genes selected from the group consisting of NDM, KPC, IMP, VIM, OXA-48-like, DHA, CMY, FOX, mecA, mecC, vanA, vanB, CTX-M 14, CTX-M 15, mefA, mefE, ermA, ermB, SHV, and TEM) in a whole blood sample. In some embodiments, the methods may involve disruption of red blood cells (erythrocytes). In some embodiments, the disruption of the red blood cells can be carried out using an erythrocyte lysis agent (i.e., a lysis buffer, an isotonic lysis agent, or a nonionic detergent). Erythrocyte lysis buffers which can be used in the methods of the invention include, without limitation, isotonic solutions of ammonium chloride (optionally including carbonate buffer and/or EDTA), and hypotonic solutions. The basic mechanism of hemolysis using isotonic ammonium chloride is by diffusion of ammonia across red blood cell membranes. This influx of ammonium increases the intracellular concentration of hydroxyl ions, which in turn reacts with CO₂ to form hydrogen carbonate. Erythrocytes exchange excess hydrogen carbonate with chloride which is present in blood plasma via anion channels and subsequently increase in intracellular ammonium chloride concentrations. The resulting swelling of the cells eventually causes loss of membrane integrity.

Alternatively, the erythrocyte lysis agent can be an aqueous solution of nonionic detergents (e.g., nonyl phenoxypolyethoxylethanol (NP-40), 4-octylphenol polyethoxylate (TRITON™ X-100), BRIJ 58, or related nonionic surfactants, and mixtures thereof). The erythrocyte lysis agent disrupts at least some of the red blood cells, allowing a large fraction of certain components of whole blood (e.g., certain whole blood proteins) to be separated (e.g., as supernatant following centrifugation) from the white blood cells or other cells (e.g., pathogen cells (e.g., bacterial cells and/or fungal cells)) present in the whole blood sample. Following erythrocyte lysis and centrifugation, the resulting pellet may be lysed, for example, as described above.

In some embodiments, the methods provided herein may include (a) providing a whole blood sample from a subject; (b) mixing the whole blood sample with an erythrocyte lysis agent solution to produce disrupted red blood cells; (c) following step (b), centrifuging the sample to form a supernatant and a pellet, discarding some or all of the supernatant, and resuspending the pellet to form an extract, (d) lysing cells of the extract (which may include white blood cells and/or pathogen cells) to form a lysate. In some embodiments, the method further comprises amplifying one or more target drug resistance (e.g., antibiotic resistance) nucleic acids in the lysate. In some embodiments, the method further comprises sequencing one or more target drug resistance (e.g., antibiotic resistance) nucleic acids in the lysate. In some embodiments, the sample of whole blood is from about 0.5 to about 10 mL of whole blood, for example, 0.5 mL, 1 mL, 2 mL, 3 mL, 4 mL, 5 mL, 6 mL, 7 mL, 8 mL, 9 mL, or 10 mL of whole blood. In some embodiments, the method may include washing the pellet (e.g., with a buffer such as TE buffer) prior to resuspending the pellet and optionally repeating step (c). In some embodiments, step (c) does not involve resuspending the pellet but instead includes adding a buffer solution to the pellet to form the extract. In some embodiments, the method may include 1, 2, 3, 4, 5, or more wash steps. In other embodiments, the method is performed without performing any wash step. In some embodiments, the amplifying is in the presence of whole blood proteins, non-target nucleic acids, or both. In some embodiments, the amplifying may be in the presence of from about 0.5 μg to about 200 μg (e.g., about 0.5 μg, 1 μg, 5 μg, 10 μg, 15 μg, 20 μg, 25 μg, 30 μg, 35 μg, 40 μg, 45 μg, 50 μg, 55 μg, 60 μg, 70 μg, 80 μg, 90 μg, 100 μg, 110 μg, 120 μg, 130 μg, 140 μg, 150 μg, 160 μg, 170 μg, 180 μg, 190 μg, or 200 μg) of subject (i.e., host) DNA. In some embodiments, the amplifying may be in the presence of more than about 1 μg (e.g., more than about 10 μg, 15 μg, 20 μg, 25 μg, 30 μg, 35 μg, 40 μg, 45 μg, 50 μg, 55 μg, 60 μg, 70 μg, 80 μg, 90 μg, 100 μg, 110 μg, 120 μg, 130 μg, 140 μg, 150 μg, 160 μg, 170 μg, 180 μg, 190 μg, or 200 μg) of subject (i.e., host) DNA. In some embodiments, at least a portion of the subject (i.e., host) DNA is from white blood cells of the subject. In some embodiments, the subject (i.e., host) DNA is from white blood cells of the subject.

### Amplification Approaches

In several embodiments, the methods and systems of the invention involve amplification of one or more nucleic acids. Amplification may be exponential or linear. A target or template nucleic acid may be any suitable nucleic acid (e.g., one or more antibiotic resistance genes selected from the group consisting of NDM, KPC, IMP, VIM, OXA-48-like, DHA, CMY, FOX, mecA, mecC, vanA, vanB, CTX-M 14, CTX-M 15, mefA, mefE, ermA, ermB, SHV, and TEM, or a portion thereof). In some embodiments, the target nucleic acid is DNA or RNA (e.g., mRNA). The sequences amplified in this manner form an amplified target nucleic acid (also referred to herein as an amplicon). Primers and probes can be readily designed by those skilled in the art to target a specific template nucleic acid sequence. In certain preferred embodiments, resulting amplicons are short to allow for rapid cycling and generation of copies. The size of the amplicon can vary as needed, for example, to provide the ability to discriminate target nucleic acids from non-target nucleic acids. For example, amplicons can be less than about 1,000 nucleotides in length. In some embodiments, the amplicons are from 100 to 500 nucleotides in length (e.g., 100 to 200, 150 to 250, 300 to 400, 350 to 450, or 400 to 500 nucleotides in length). In other embodiments, the amplicons are greater than about 1,000 nucleotides in length, e.g., about 1,000, about 2,000, about 3,000, about 4,000, about 5,000, about 6,000, about 7,000, about 8,000, about 9,000, about 10,000, or more nucleotides in length. In some embodiments, more than one (e.g., 1, 2, 3, 4, 5, 6, 7, 8, 9, 10, or more than 10) target nucleic acids may be amplified in one reaction. In other embodiments, a single target nucleic acid may be amplified in one reaction. In some embodiments, the invention provides amplification-based nucleic acid detection assays conducted in complex samples containing cells and/or cell debris, including but not limited to blood (e.g., whole blood, a crude whole blood lysate, serum, or plasma), bloody fluids (e.g., wound exudate, phlegm, bile, and the like), tissue samples (e.g., tissue biopsies (e.g., skin biopsies, muscle biopsies, or lymph node biopsies), including homogenized tissue samples), urine, CSF, BAL, SF, or sputum (e.g., purulent sputum or bloody sputum). In several embodiments, the method provides methods for amplifying target nucleic acids in a biological sample that includes cells, cell debris, and/or nucleic acids (e.g., DNA or RNA (e.g., mRNA)) derived from both a host mammalian subject and from a microbial organism, particularly a microbial pathogen. The resulting amplified target nucleic acids, or portions or fragments thereof, can be sequenced according to any of the sequencing approaches known in the art and/or described herein.

Sample preparation typically involves removing or providing resistance for common PCR inhibitors found in complex samples containing cells and/or cell debris. Common inhibitors are listed in Table A (see also Wilson, Appl. Environ. Microbiol., 63:3741 (1997)). The “facilitators” in Table A indicate methodologies or compositions that may be used to reduce or overcome inhibition. Any of the facilitators may be used in the methods described herein. Inhibitors typically act by either prevention of cell lysis, degradation or sequestering a target nucleic acid, and/or inhibition of a polymerase activity. The most commonly employed polymerase, Taq, is typically inhibited by the presence of 0.1% blood in a reaction. Mutant Taq polymerases have been engineered that are resistant to common inhibitors (e.g., hemoglobin and/or humic acid) found in blood (see, e.g., Kermekchiev et al., Nucl. Acid. Res., 37(5): e40, (2009)). Manufacturer recommendations indicate these mutations enable direct amplification from up to 20% blood.

TABLE A
PCR inhibitors and facilitators for overcoming inhibition

| Sample or | | | |
|---|---|---|---|
| Specimen Type | Target | Inhibitor | Facilitator |
| feces | Escherichia coli | >10³ bacterial cells | ion-exchange column |
| CSF | Treponema | Cell debris causing nonspecific | nested primers |
| | pallidum | amplification | |
| whole blood | mammalian | >4 μl of blood/100-ml reaction | 1-2% blood per reaction |
| | tissue | mix (hemoglobin) | |
| feces | Rotavirus | unknown dilution | cellulose fiber |
| clinical | Cytomegalovirus | unidentified components | glass bead extraction |
| specimens | | | |
| human blood | human genes | DNA binding proteins | thermophilic protease from |
| and tissue | | | Thermus strain rt44A |
| mammalian | Mammalian | thermal cycler variations | formamide |
| tissue | tissue genetics | | |
| mammalian | Mammalian | thermal cycler variations | DMSO, glycerol, PEG, |
| tissue | tissue genetics | | organic solvents |
| clinical | Treponema | unknown factors | Various substrate-specific |
| specimens | pallidum | | physicochemical methods |
| forensic semen | Sperm | Genotyping errors; | |
| samples | | selective/total PCR inhibition by | |
| | | vaginal microorganisms | |
| feces | Salmonella | various body fluids | immunomagnetic |
| | enterica | | separation |
| feces | Various enteric | unknown | size exclusion |
| | viruses | | chromatography, |
| | | | physicochemical extraction |
| clinical | Herpes simplex | endogenous inhibitors, random | repurification, coamplified |
| specimens | virus | effects | positive control |
| feces | Escherichia | nonspecific inhibitors, urea, | additional primers and |
| | coli | hemoglobin, heparin, phenol, | reaction cyclers, booster |
| | | SDS | PCR |
| tissue culture | Cytomegalovirus | glove powder | |
| | HIV | | |
| suspensions, | Mycobacterium | mercury-based fixatives, | reduced fixation times, |
| skin biopsies | leprae | neutral buffered formaline | ethanol fixation |
| clinical | Mycobacterium | unknown inhibitors in pus, | physicochemical extraction |
| specimens | tuberculosis | tissue biopsies, sputum, pleural | |
| | | fluid | |
| mammalian | mammalian | unknown contaminant of | additional DNA |
| tissue | tissue genetics | reverse transcriptase | |
| formalin-fixed | Hepatitis C virus | ribonucleotide vanadyl | phenol/chloroform |
| paraffin tissue | | complexes | extraction |
| nasopharyngeal | Bordetella | unknown inhibitors | phenol/chloroform |
| aspirates and | pertussis | | extraction |
| swabs | | | |
| human | HIV type 1 | detergents | mineral oil |
| mononuclear | | | |
| blood cells | | | |
| bloodstain | human mitochondrial | unidentified heme compound, | BSA |
| | DNA | hemin | |
| blood | various | heparin | alternative polymerases |
| | | | and buffers, chelex, |
| | | | spermine, [Mg2+], glycerol, |
| | | | BSA, heparinase |
| sputum | Mycoplasma | N-acetyl-L-cysteine, | |
| | pneumoniae | dithiothreitol, mucolytic agents | |
| human tissue | HLA-DRB1 | pollen, glove powder, impure | |
| | genotyping | DNA, heparin, hemoglobin | |
| clinical | Mycobacterium | unknown | competitive internal control |
| specimens | tuberculosis | | |
| dental plaque | many | unknown | diatomaceous earth, |
| | | | guanidium isothiocyante, |
| | | | ethanol, acetone |
| ancient | Cytochrome b | unknown | ammonium acetate, |
| mammalian | gene | | ethidium bromide |
| tissues | | | |

Polymerase chain reaction amplification of DNA or cDNA is a tried and trusted methodology; however, as discussed above, polymerases are inhibited by agents contained in complex biological samples containing cells, cell debris, and/or nucleic acids (e.g., DNA or RNA)), including but not limited to commonly used anticoagulants and hemoglobin. Recently, mutant Taq polymerases have been engineered to harbor resistance to common inhibitors found in blood and soil. Currently available polymerases, e.g., HemoKlenTaq® (New England BioLabs, Inc., Ipswich, Mass.) as well as OmniTaq® and OmniKlenTaq® (DNA Polymerase Technology, Inc., St. Louis, Mo.) are mutant (e.g., N-terminal truncation and/or point mutations) Taq polymerase that render them capable of amplifying DNA in the presence of up to 10%, 20% or 25% whole blood, depending on the product and reaction conditions (See, e.g., Kermekchiev et al. Nucl. Acids Res. 31:6139 (2003); and Kermekchiev et al., Nucl. Acid. Res., 37:e40 (2009); and see U.S. Pat. No. 7,462,475). Additionally, PHUSION® Blood Direct PCR Kits (Finnzymes Oy, Espoo, Finland), include a unique fusion DNA polymerase enzyme engineered to incorporate a double-stranded DNA binding domain, which allows amplification under conditions which are typically inhibitory to conventional polymerases such as Taq or Pfu, and allow for amplification of DNA in the presence of up to about 40% whole blood under certain reaction conditions. See Wang et al., Nucl. Acids Res. 32:1197 (2004); and see U.S. Pat. Nos. 5,352,778 and 5,500,363. Furthermore, Kapa Blood PCR Mixes (Kapa Biosystems, Woburn, Mass.), provide a genetically engineered DNA polymerase enzyme which allows for direct amplification of whole blood at up to about 20% of the reaction volume under certain reaction conditions. Despite these breakthroughs, direct optical detection of generated amplicons is typically not possible with existing methods since fluorescence, absorbance, and other light based methods yield signals that are quenched by the presence of blood. See Kermekchiev et al., Nucl. Acid. Res., 37:e40 (2009).

Table B shows a list of mutant thermostable DNA polymerases that are compatible with many types of interfering substances and that may be used in the methods of the invention for amplification of target nucleic acids in biological samples containing cells and/or cell debris. In certain embodiments, the invention features the use of enzymes compatible with whole blood, e.g., mutant thermostable DNA polymerases including but not limited to NEB HemoKlenTag™, DNAP OmniKlenTaq™, Kapa Biosystems whole blood enzyme, Thermo-Fisher Finnzymes PHUSION® enzyme, or any of the mutant thermostable DNA polymerases shown in Table B.

TABLE B
Exemplary mutant thermostable DNA polymerases

| Polymerase | Reference |
|---|---|
| Klentaq ®1 | Barnes, Proc Natl Acad Sci USA. 91 (6): |
| | 2216-2220, 1994. |
| Klentaq ® LA | Barnes, Proc Natl Acad Sci USA. 91 (6): |
| | 2216-2220, 1994. |
| Cesium Klentaq ® AC | Kermekchiev et al., Nuc. Acids Res. 31(21): |
| | 6139-6147, 2003. |
| Cesium Klentaq ® AC LA | Kermekchiev et al., Nuc. Acids Res. 31(21): |
| | 6139-6147, 2003. |
| Cesium Klentaq ® C | Kermekchiev et al., Nuc. Acids Res. 31(21): |
| | 6139-6147, 2003. |
| Cesium Klentaq ® C LA | Kermekchiev et al., Nuc. Acids Res. 31(21): |
| | 6139-6147, 2003. |
| Omni Klentaq ® | Kermekchiev et al. Nuc. Acids Res. 37(5): |
| | e40, 2009. |
| Omni Klentaq ® 2 | Kermekchiev et al. Nuc. Acids Res. 37(5): |
| | e40, 2009. |
| Omni Klentaq ® LA | Kermekchiev et al. Nuc. Acids Res. 37(5): |
| | e40, 2009. |
| Omni Taq | Kermekchiev et al. Nuc. Acids Res. 37(5): |
| | e40, 2009. |
| Omni Taq LA | Kermekchiev et al. Nuc. Acids Res. 37(5): |
| | e40, 2009. |
| Omni Taq 2 | Kermekchiev et al. Nuc. Acids Res. 37(5): |
| | e40, 2009. |
| Omni Taq 3 | Kermekchiev et al. Nuc. Acids Res. 37(5): |
| | e40, 2009. |
| Hemo KlenTaq ® | Kermekchiev et al. Nuc. Acids Res. 37(5): |
| | e40, 2009. |
| KAPA Blood DNA | KAPA Biosystems |
| Polymerase | |
| KAPA3G Plant DNA | KAPA Biosystems |
| Polymerase | |
| KAPA2G Robust DNA | KAPA Biosystems |
| Polymerase | |
| MyTaq ™ Blood-PCR Kit | Bioline |
| Phusion ® Blood DNA Kit | Thermo Scientific Manage et al., |
| with Hot Start Phusion II | Microfluid. Nanofluid. 10, 697-702, 2011. |

As described above, a variety of impurities and components of whole blood can be inhibitory to the polymerase and primer annealing. These inhibitors can sometimes lead to generation of false positives and low sensitivities. To reduce the generation of false positives and low sensitivities when amplifying and detecting nucleic acids in complex samples, it is desirable to utilize a thermal stable polymerase not inhibited by whole blood samples, for example as described above, and include one or more internal PCR assay controls (see Rosenstraus et al. J. Clin Microbiol. 36:191 (1998) and Hoofar et al., J. Clin. Microbiol. 42:1863 (2004)).

For example, the assay can include an internal control nucleic acid that contains primer binding regions identical to those of the target sequence to assure that clinical specimens are successfully amplified and detected. In some embodiments, the target nucleic acid and internal control can be selected such that each has a unique probe binding region that differentiates the internal control from the target nucleic acid. The internal control is, optionally, employed in combination with a processing positive control, a processing negative control, and a reagent control for the safe and accurate determination and identification of an infecting organism in, e.g., a whole blood clinical sample. The internal control can be an inhibition control that is designed to co-amplify with the nucleic acid target being detected. Failure of the internal inhibition control to be amplified is evidence of a reagent failure or process error. Universal primers can be designed such that the target sequence and the internal control sequence are amplified in the same reaction tube. Thus, using this format, if the target DNA is amplified but the internal control is not it is then assumed that the target DNA is present in a proportionally greater amount than the internal control and the positive result is valid as the internal control amplification is unnecessary. If, on the other hand, neither the internal control nor the target is amplified it is then assumed that inhibition of the PCR reaction has occurred and the test for that particular sample is not valid.

The assays of the invention can include one or more positive processing controls in which one or more target nucleic acids is included in the assay (e.g., each included with one or more cartridges) at 3× to 5× the limit of detection. If detected by T2MR, the measured T₂ for each of the positive processing controls must be above the pre-determined threshold indicating the presence of the target nucleic acid. The positive processing controls can detect all reagent failures in each step of the process (e.g., lysis, PCR, and T2MR detection), and can be used for quality control of the system. The assays of the invention can include one or more negative processing controls consisting of a solution free of target nucleic acid (e.g., buffer alone). If detected by T2MR, the T₂ measurements for the negative processing control should be below the threshold indicating a negative result while the T₂ measured for the internal control is above the decision threshold indicating an internal control positive result. The purpose of the negative control is to detect carry-over contamination and/or reagent contamination. The assays of the invention can include one or more reagent controls. The reagent control will detect reagent failures in the PCR stage of the reaction (i.e. incomplete transfer of master mix to the PCR tubes). The reagent controls can also detect gross failures in reagent transfer prior to T₂ detection.

The methods of the invention can also include use of a total process control (TPC), for example, an engineered cell (e.g., an engineered bacterium or fungus (e.g., yeast)) comprising a control target nucleic acid that has a known and defined sequence. The TPC may be added to the sample (e.g., environmental or biological sample) as a control to monitor steps including cell lysis, amplification, and sequencing.

In some embodiments, complex samples, which may be a liquid sample (including, for example, a biological sample containing cells and/or cell debris including but not limited to blood (e.g., whole blood, a crude whole blood lysate, serum, or plasma), bloody fluids (e.g., wound exudate, phlegm, bile, and the like), tissue samples (e.g., tissue biopsies, including homogenized tissue samples), urine, BAL, CSF, SF, or sputum) can be directly amplified using about 5%, about 10%, about 20%, about 25%, about 30%, about 25%, about 40%, about 45%, about 50%, about 55%, about 60%, about 65%, about 70%, or more complex liquid sample in amplification reactions, and that the resulting amplicons can be directly detected from amplification reaction using, for example, sequencing (e.g., massively parallel, long-read, and/or Sanger sequencing) and/or magnetic resonance (MR) relaxation measurements (e.g., T2MR) upon the addition of conjugated magnetic particles bound to oligonucleotides complementary to the target nucleic acid sequence. Alternatively, the magnetic particles can be added to the sample prior to amplification. Thus, provided are methods for the use of nucleic acid amplification in a complex dirty sample, sequencing and/or hybridization of the resulting amplicon to paramagnetic particles, which may be followed by direct detection of hybridized magnetic particle conjugate and target amplicons using magnetic particle based detection systems. In some embodiments, the detection is by sequencing only. In other embodiments, direct detection of hybridized magnetic particle conjugates and amplicons is via MR relaxation measurements (e.g., T₂, T₁, T₁/T₂ hybrid, T₂, and the like). Further provided are methods which are kinetic, in order to quantify the original nucleic acid copy number within the sample (e.g., sampling and nucleic acid detection at pre-defined cycle numbers, comparison of endogenous internal control nucleic acid, use of exogenous spiked homologous competitive control nucleic acid). In some embodiments, the resulting amplicons are detected using a non-MR-based approach, for example, optical, fluorescent, mass, density, chromatographic, and/or electrochemical measurement.

While the exemplary methods described hereinafter relate to amplification using PCR, numerous other methods are known in the art for amplification of nucleic acids (e.g., isothermal methods, rolling circle methods, etc.). Those skilled in the art will understand that these other methods may be used either in place of, or together with, PCR methods. See, e.g., Saiki, “Amplification of Genomic DNA” in PCR Protocols, Innis et al., Eds., Academic Press, San Diego, Calif., pp 13-20 (1990); Wharam et al., Nucleic Acids Res. 29:E54 (2001); Hafner et al., Biotechniques, 30:852 (2001). Further amplification methods suitable for use with the present methods include, for example, reverse transcription PCR (RT-PCR), ligase chain reaction (LCR), multiple displacement amplification (MDA), strand displacement amplification (SDA), rolling circle amplification (RCA), loop mediated isothermal amplification (LAMP), nucleic acid sequence based amplification (NASBA), helicase dependent amplification, recombinase polymerase amplification, nicking enzyme amplification reaction, ramification amplification (RAM), transcription based amplification system (TAS), transcription mediated amplification (TMA), the isothermal and chimeric primer-initiated amplification of nucleic acid (ICAN) method, and the smart amplification system (SMAP) method. These methods, as well as others are well known in the art and can be adapted for use in conjunction with provided methods of detection of amplified nucleic acid.

The PCR method is a technique for making many copies of a specific template DNA sequence. The PCR process is disclosed, for example, in U.S. Pat. Nos. 4,683,195; 4,683,202; and 4,965,188. One set of primers complementary to a template DNA are designed, and a region flanked by the primers is amplified by DNA polymerase in a reaction including multiple amplification cycles. Each amplification cycle includes an initial denaturation, and up to 50 cycles of annealing, strand elongation (or extension) and strand separation (denaturation). In each cycle of the reaction, the DNA sequence between the primers is copied. Primers can bind to the copied DNA as well as the original template sequence, so the total number of copies increases exponentially with time. PCR can be performed as according to Whelan et al., Journal of Clinical Microbiology 33:556 (1995). Various modified PCR methods are available and well known in the art. Various modifications such as the “RT-PCR” method, in which DNA is synthesized from RNA using a reverse transcriptase before performing PCR; and the “TaqMan® PCR” method, in which only a specific allele is amplified and detected using a fluorescently labeled TaqMan® probe, and Taq DNA polymerase, are known to those skilled in the art. RT-PCR and variations thereof have been described, for example, in U.S. Pat. Nos. 5,804,383; 5,407,800; 5,322,770; and 5,310,652, and references described therein; and TaqMan® PCR and related reagents for use in the method have been described, for example, in U.S. Pat. Nos. 5,210,015; 5,876,930; 5,538,848; 6,030,787; and 6,258,569.

In some embodiments, asymmetric PCR is performed to preferentially amplify one strand of a double-stranded DNA (dsDNA) template. Asymmetric PCR typically involves addition of an excess of the primer for the strand targeted for amplification. An exemplary asymmetric PCR condition is 300 nM of the excess primer and 75 nM of the limiting primer to favor single strand amplification. In other embodiments, 400 nM of the excess primer and 100 nM of the limiting primer may be used to favor single strand amplification. In other embodiments, symmetric PCR is performed.

In some embodiments, including embodiments that employ multiplexed PCR reactions, hot start PCR conditions may be used to reduce mis-priming, primer-dimer formation, improve yield, and/or and ensure high PCR specificity and sensitivity. A variety of approaches may be employed to achieve hot start PCR conditions, including hot start DNA polymerases (e.g., hot start DNA polymerases with aptamer-based inhibitors or with mutations that limit activity at lower temperatures) as well as hot start dNTPs (e.g., CLEANAMP™ dNTPs, TriLink Biotechnologies).

In some embodiments, a PCR reaction may include from about 20 cycles to about 55 cycles or more (e.g., about 20, 25, 30, 35, 40, 45, 50, or 55 cycles).

LCR is a method of DNA amplification similar to PCR, except that it uses four primers instead of two and uses the enzyme ligase to ligate or join two segments of DNA. Amplification can be performed in a thermal cycler (e.g., LCx of Abbott Labs, North Chicago, Ill.). LCR can be performed for example, as according to Moore et al., Journal of Clinical Microbiology 36:1028 (1998). LCR methods and variations have been described, for example, in European Patent Application Publication No. EP0320308, and U.S. Pat. No. 5,427,930.

The TAS method is a method for specifically amplifying a target RNA in which a transcript is obtained from a template RNA by a cDNA synthesis step and an RNA transcription step. In the cDNA synthesis step, a sequence recognized by a DNA-dependent RNA polymerase (i.e., a polymerase-binding sequence or PBS) is inserted into the cDNA copy downstream of the target or marker sequence to be amplified using a two-domain oligonucleotide primer. In the second step, an RNA polymerase is used to synthesize multiple copies of RNA from the cDNA template. Amplification using TAS requires only a few cycles because DNA-dependent RNA transcription can result in 10-1000 copies for each copy of cDNA template. TAS can be performed according to Kwoh et al., PNAS 86:1173 (1989). The TAS method has been described, for example, in International Patent Application Publication No. WO1988/010315.

Transcription mediated amplification (TMA) is a transcription-based isothermal amplification reaction that uses RNA transcription by RNA polymerase and DNA transcription by reverse transcriptase to produce an RNA amplicon from target nucleic acid. TMA methods are advantageous in that they can produce 100 to 1000 copies of amplicon per amplification cycle, as opposed to PCR or LCR methods that produce only 2 copies per cycle. TMA has been described, for example, in U.S. Pat. No. 5,399,491. NASBA is a transcription-based method which for specifically amplifying a target RNA from either an RNA or DNA template. NASBA is a method used for the continuous amplification of nucleic acids in a single mixture at one temperature. A transcript is obtained from a template RNA by a DNA-dependent RNA polymerase using a forward primer having a sequence identical to a target RNA and a reverse primer having a sequence complementary to the target RNA a on the 3′ side and a promoter sequence that recognizes T7 RNA polymerase on the 5′ side. A transcript is further synthesized using the obtained transcript as template. This method can be performed as according to Heim, et al., Nucleic Acids Res., 262250 (1998). The NASBA method has been described in U.S. Pat. No. 5,130,238.

The SDA method is an isothermal nucleic acid amplification method in which target DNA is amplified using a DNA strand substituted with a strand synthesized by a strand substitution type DNA polymerase lacking 5′->3′ exonuclease activity by a single stranded nick generated by a restriction enzyme as a template of the next replication. A primer containing a restriction site is annealed to template, and then amplification primers are annealed to 5′ adjacent sequences (forming a nick). Amplification is initiated at a fixed temperature. Newly synthesized DNA strands are nicked by a restriction enzyme and the polymerase amplification begins again, displacing the newly synthesized strands. SDA can be performed according to Walker, et al., PNAS, 89:392 (1992). SDA methods have been described in U.S. Pat. Nos. 5,455,166 and 5,457,027.

The LAMP method is an isothermal amplification method in which a loop is always formed at the 3′ end of a synthesized DNA, primers are annealed within the loop, and specific amplification of the target DNA is performed isothermally. LAMP can be performed according to Nagamine et al., Clinical Chemistry. 47:1742 (2001). LAMP methods have been described in U.S. Pat. Nos. 6,410,278; 6,974,670; and 7,175,985.

The ICAN method is anisothermal amplification method in which specific amplification of a target DNA is performed isothermally by a strand substitution reaction, a template exchange reaction, and a nick introduction reaction, using a chimeric primer including RNA-DNA and DNA polymerase having a strand substitution activity and RNase H. ICAN can be performed according to Mukai et al., J. Biochem. 142: 273 (2007). The ICAN method has been described in U.S. Pat. No. 6,951,722.

The SMAP (MITANI) method is a method in which a target nucleic acid is continuously synthesized under isothermal conditions using a primer set including two kinds of primers and DNA or RNA as a template. The first primer included in the primer set includes, in the 3′ end region thereof, a sequence (Ac′) hybridizable with a sequence (A) in the 3′ end region of a target nucleic acid sequence as well as, on the 5′ side of the above-mentioned sequence (Ac′), a sequence (B′) hybridizable with a sequence (Bc) complementary to a sequence (B) existing on the 5′ side of the above-mentioned sequence (A) in the above-mentioned target nucleic acid sequence. The second primer includes, in the 3′ end region thereof, a sequence (Cc′) hybridizable with a sequence (C) in the 3′ end region of a sequence complementary to the above-mentioned target nucleic acid sequence as well as a loopback sequence (D-Dc′) including two nucleic acid sequences hybridizable with each other on an identical strand on the 5′ side of the above-mentioned sequence (Cc′). SMAP can be performed according to Mitani et al., Nat. Methods, 4(3): 257 (2007). SMAP methods have been described in U.S. Patent Application Publication Nos. 2006/0160084, 2007/0190531 and 2009/0042197.

The amplification reaction can be designed to produce a specific type of amplified product, such as nucleic acids that are double stranded; single stranded; double stranded with 3′ or 5′ overhangs; or double stranded with chemical ligands on the 5′ and 3′ ends. The amplified PCR product can be detected by: (i) sequencing; (ii) hybridization of the amplified product to magnetic particle bound complementary oligonucleotides, where two different oligonucleotides are used that hybridize to the amplified product such that the nucleic acid serves as an interparticle tether promoting particle agglomeration; (iii) hybridization mediated detection where the DNA of the amplified product must first be denatured; (iv) hybridization mediated detection where the particles hybridize to 5′ and 3′ overhangs of the amplified product; and/or (v) binding of the particles to the chemical or biochemical ligands on the termini of the amplified product, such as streptavidin functionalized particles binding to biotin functionalized amplified product.

### Analytes

Embodiments of the invention include methods and systems for detecting and/or measuring the concentration of one or more analytes (e.g., analytes associated with drug resistance (e.g., antibiotic resistance), e.g., one or more antibiotic resistance genes selected from the group consisting of NDM, KPC, IMP, VIM, OXA-48-like, DHA, CMY, FOX, mecA, mecC, vanA, vanB, CTX-M 14, CTX-M 15, mefA, mefE, ermA, ermB, SHV, or TEM) in a complex biological sample containing cells and/or cell debris, including but not limited to blood (e.g., whole blood, a crude whole blood lysate, serum, or plasma), bloody fluids (e.g., wound exudate, phlegm, bile, and the like), tissue samples (e.g., a tissue biopsy (e.g., a skin biopsy, muscle biopsy, or lymph node biopsy), including homogenized tissue samples), urine, cerebrospinal fluid (CSF), synovial fluid (SF), or sputum. In several embodiments, the analyte may be a nucleic acid derived from an organism. In some embodiments, the nucleic acid is a target nucleic acid derived from the organism that has been amplified to form an amplicon. In some embodiments, the organism is a plant, a mammal, or a microbial species. The nucleic acid can be detected by sequencing. In some embodiments, the nucleic acid may further be detected by other approaches, including T2MR.

In some embodiments, the analyte is an antimicrobial resistance marker. Exemplary non-limiting antimicrobial resistance markers include, e.g., bla_{KPC}, blaZ, bla_{NDM}, bla_{IMP}, bla_{VIM}, bla_{OXA}, bla_{CMY}, bla_{DHA}, bla_{TEM}, bla_{SHV}, bla_{CTX-M}, bla_{SME}, bla_{FOX}, bla_{MIR}, femA, femB, mecA, macB, fosA, vanA, vanB, vanC, vanD, vanE, vanG, mefA, mefE, ermA, ermB, tetA, tetB, tetX, tetR, qnrA, qnrB, or qnrS.

In particular embodiments, the analyte includes an antibiotic resistance gene selected from the group consisting of NDM, KPC, IMP, VIM, OXA-48-like, DHA, CMY, FOX, mecA, mecC, vanA, vanB, CTX-M 14, CTX-M 15, mefA, mefE, ermA, ermB, SHV, and TEM.

In some embodiments, the analyte may be derived from a microbial pathogen. In such embodiments, the biological sample may include cells and/or cell debris from the host mammalian subject as well as one or more microbial pathogen cells. Exemplary analytes are described herein, e.g., in FIGS. 1 and 2. For example, in some embodiments, the analyte is derived from a Gram-negative bacterium, a Gram-positive bacterium, a fungal pathogen (e.g., a yeast (e.g., Candida spp.) or Aspergillus spp.), a protozoan pathogen, or a viral pathogen. In some embodiments, the analyte is derived from a bacterial pathogen, including Acinetobacter spp. (e.g., Acinetobacter baumannii, Acinetobacter pittii, and Acinetobacter nosocomialis), Enterobacteriaceae spp., Enterococcus spp. (e.g., Enterococcus faecium (including E. faecium with resistance marker vanA/B) and Enterococcus faecalis), Klebsiella spp. (e.g., Klebsiella pneumoniae (e.g., K. pneumoniae with resistance marker KPC) and Klebsiella oxytoca), Pseudomonas spp. (e.g., Pseudomonas aeruginosa), Staphylococcus spp. (e.g., Staphylococcus aureus (e.g., S. aureus with resistance marker mecA), Staphylococcus haemolyticus, Staphylococcus lugdunensis, Staphylococcus maltophilia, Staphylococcus saprophytcus, coagulase-positive Staphylococcus species, and coagulase-negative (CoNS) Staphylococcus species), Streptococcus spp. (e.g., Streptococcus mitis, Streptococcus pneumoniae, Streptococcus agalactiae, Streptococcus anginosa, Streptococcus bovis, Streptococcus dysgalactiae, Streptococcus mutans, Streptococcus sanguinis, and Streptococcus pyogenes), Escherichia spp. (e.g., Escherichia coli), Stenotrophomonas spp. (e.g., Stenotrophomonas maltophilia), Proteus spp. (e.g., Proteus mirabilis and Proteus vulgaris), Serratia spp. (e.g., Serrata marcescens), Citrobacter spp. (e.g., Citrobacter freundii and Citrobacter koseri), Haemophilus spp. (e.g., Haemophilus influenzae), Listeria spp. (e.g., Listeria monocytogenes), Neisseria spp. (e.g., Neisseria meningitidis), Bacteroides spp. (e.g., Bacteroides fragilis), Burkholderia spp. (e.g., Burkholderia cepacia), Campylobacter (e.g., Campylobacter jejuni and Campylobacter coli), Clostridium spp. (e.g., Clostridium perfringens), Kingella spp. (e.g., Kingella kingae), Morganella spp. (e.g., Morganella morgana), Prevotella spp. (e.g., Prevotella buccae, Prevotella intermedia, and Prevotella melaninogenica), Propionibacterium spp. (e.g., Propionibacterium acnes), Salmonella spp. (e.g., Salmonella enterica), Shigella spp. (e.g., Shigella dysenteriae and Shigella flexneri), Borrelia spp., (e.g., Borrelia burgdorferi sensu lato (Borrelia burgdorferi, Borrelia afzelii, and Borrelia garinii) species), Rickettsia spp. (including Rickettsia rickettsii and Rickettsia parker), Ehrlichia spp. (including Ehrlichia chaffeensis, Ehrlichia ewingii, and Ehrlichia muris-like), Coxiella spp. (including Coxiella burnetii), Anaplasma spp. (including Anaplasma phagocytophilum), Francisella spp., (including Francisella tularensis (including Francisella tularensis subspp. holarctica, mediasiatica, and novicida) and Enterobacter spp. (e.g., Enterobacter aerogenes and Enterobacter cloacae).

In some embodiments, a pathogen-associated analyte may be a nucleic acid derived from any of the organisms described above, for example, DNA or RNA (e.g., mRNA). In some embodiments, the nucleic acid is a target nucleic acid derived from the organism that has been amplified to form an amplicon. In some embodiments, the target nucleic acid may be a multi-copy locus. Use of a target nucleic acid derived from a multi-copy locus, in particular in methods involving amplification, may lead to an increase in sensitivity in the assay. Exemplary multi-copy loci may include, for example, ribosomal DNA (rDNA) operons and multi-copy plasmids. In other embodiments, the target nucleic acid may be a single-copy locus. In particular embodiments, the target nucleic acid may be derived from an essential locus, for example, an essential house-keeping gene. In particular embodiments, the target nucleic acid may be derived from a locus that is involved in virulence (e.g., a virulence gene). In any of the above embodiments, a locus may include a gene and/or an intragenic region, for example, an internally transcribed sequence (ITS) between rRNA genes (e.g., ITS1, between the 16S and 23S rRNA genes, or ITS2, between the 5S and 23S rRNA genes). In some embodiments, the target nucleic acid is a 16S rRNA target nucleic acid.

In some embodiments, a target nucleic acid may be (a) species-specific, (b) species-inclusive (in other words, present in all strains or subspecies of a given species), (c) compatible with an amplification/detection protocol, and/or (d) present in multiple copies. In some embodiments, a target nucleic acid may be group-specific or group-inclusive, e.g., genus-specific or genus inclusive. In particular embodiments, a target nucleic acid is chromosomally-encoded.

### Panels

The methods and compositions (e.g., systems, devices, kits, or cartridges) described herein can be configured to detect and/or sequence target nucleic acids from a predetermined panel of targets. The panels can be configured to detect and/or sequence drug resistance markers (e.g., antibiotic resistance genes). Any of the antibiotic resistance genes described herein can be detected and/or sequenced using the panels described herein. In some embodiments, the panels may be configured to detect one or more of NDM, KPC, IMP, VIM, OXA-48-like, DHA, CMY, FOX, mecA, mecC, vanA, vanB, CTX-M 14, CTX-M 15, mefA, mefE, ermA, ermB, SHV, and TEM. In some embodiments, the panel may be configured to detect one or more of IMP, OXA-48-like, DHA, CMY, FOX, CTX-M 14, CTX-M 15, mecC, mefA, mefE, ermA, ermB, and TEM. In some embodiments, the panel may be configured to detect one or more of IMP, OXA-48-like, DHA, CMY, FOX, CTX-M 14, CTX-M 15, mecC, mefA, mefE, ermA, ermB, and TEM.

For example, provided herein is a panel is configured to individually detect one or more (e.g., 1, 2, 3, 4, 5, 6, or 7) antibiotic resistance genes selected from the group consisting of NDM, KPC, IMP, VIM, OXA-48-like, DHA, and CMY. In some embodiments, the panel is further configured to detect Enterobacter and Klebsiella spp. Thus, in some embodiments, the panel is configured to detect NDM, KPC, IMP, VIM, OXA-48-like, DHA, CMY, and Enterobacter and Klebsiella spp.

For example, the panel may contain any of the combinations set forth in Tables 1-6 below. In some embodiments, the panel is configured to individually detect NDM, KPC, IMP, VIM, OXA-48-like, DHA, and CMY (see, e.g., FIG. 1).

TABLE 1
Two-target combinations of EK
(Enterobacter and Klebsiella), NDM, KPC, IMP,
VIM, OXA-48-like, DHA, and CMY

EK and NDM
EK and KPC
EK and IMP
EK and VIM
EK and OXA-48-like
EK and DHA
EK and CMY
NDM and KPC
NDM and IMP
NDM and VIM
NDM and OXA-48-like
NDM and DHA
NDM and CMY
KPC and IMP
KPC and VIM
KPC and OXA-48-like
KPC and DHA
KPC and CMY
IMP and VIM
IMP and OXA-48-like
IMP and DHA
IMP and CMY
VIM and OXA-48-like
VIM and DHA
VIM and CMY
OXA-48-like and DHA
OXA-48-like and CMY
DHA and CMY

TABLE 2
Three-target combinations of EK
(Enterobacter and Klebsiella), NDM, KPC, IMP,
VIM, OXA-48-like, DHA, and CMY

EK, NDM, and KPC
EK, NDM, and IMP
EK, NDM, and VIM
EK, NDM, and OXA-48-like
EK, NDM, and DHA
EK, NDM, and CMY
EK, KPC, and IMP
EK, KPC, and VIM
EK, KPC, and OXA-48-like
EK, KPC, and DHA
EK, KPC, and CMY
EK, IMP, and VIM
EK, IMP, and OXA-48-like
EK, IMP, and DHA
EK, IMP, and CMY
EK, VIM, and OXA-48-like
EK, VIM, and DHA
EK, VIM, and CMY
EK, OXA-48-like, and DHA
EK, OXA-48-like, and CMY
EK, DHA, and CMY
NDM, KPC, and IMP
NDM, KPC, and VIM
NDM, KPC, and OXA-48-like
NDM, KPC, and DHA
NDM, KPC, and CMY
NDM, IMP, and VIM
NDM, IMP, and OXA-48-like
NDM, IMP, and DHA
NDM, IMP, and CMY
NDM, VIM, and OXA-48-like
NDM, VIM, and DHA
NDM, VIM, and CMY
NDM, OXA-48-like, and DHA
NDM, OXA-48-like, and CMY
NDM, DHA, and CMY
KPC, IMP, and VIM
KPC, IMP, and OXA-48-like
KPC, IMP, and DHA
KPC, IMP, and CMY
KPC, VIM, and OXA-48-like
KPC, VIM, and DHA
KPC, VIM, and CMY
KPC, OXA-48-like, and DHA
KPC, OXA-48-like, and CMY
KPC, DHA, and CMY
IMP, VIM, and OXA-48-like
IMP, VIM, and DHA
IMP, VIM, and CMY
IMP, OXA-48-like, and DHA
IMP, OXA-48-like, and CMY
IMP, DHA, and CMY
VIM, OXA-48-like, and DHA
VIM, OXA-48-like, and CMY
VIM, DHA, and CMY
OXA-48-like, DHA, and CMY

TABLE 3
Four-target combinations of EK
(Enterobacter and Klebsiella), NDM, KPC, IMP,
VIM, OXA-48-like, DHA, and CMY

EK, NDM, KPC, and IMP
EK, NDM, KPC, and VIM
EK, NDM, KPC, and OXA-48-like
EK, NDM, KPC, and DHA
EK, NDM, KPC, and CMY
EK, NDM, IMP, and VIM
EK, NDM, IMP, and OXA-48-like
EK, NDM, IMP, and DHA
EK, NDM, IMP, and CMY
EK, NDM, VIM, and OXA-48-like
EK, NDM, VIM, and DHA
EK, NDM, VIM, and CMY
EK, NDM, OXA-48-like, and DHA
EK, NDM, OXA-48-like, and CMY
EK, NDM, DHA, and CMY
EK, KPC, IMP, and VIM
EK, KPC, IMP, and OXA-48-like
EK, KPC, IMP, and DHA
EK, KPC, IMP, and CMY
EK, KPC, VIM, and OXA-48-like
EK, KPC, VIM, and DHA
EK, KPC, VIM, and CMY
EK, KPC, OXA-48-like, and DHA
EK, KPC, OXA-48-like, and CMY
EK, KPC, DHA, and CMY
EK, IMP, VIM, and OXA-48-like
EK, IMP, VIM, and DHA
EK, IMP, VIM, and CMY
EK, IMP, OXA-48-like, and DHA
EK, IMP, OXA-48-like, and CMY
EK, IMP, DHA, and CMY
EK, VIM, OXA-48-like, and DHA
EK, VIM, OXA-48-like, and CMY
EK, VIM, DHA, and CMY
EK, OXA-48-like, DHA, and CMY
NDM, KPC, IMP, and VIM
NDM, KPC, IMP, and OXA-48-like
NDM, KPC, IMP, and DHA
NDM, KPC, IMP, and CMY
NDM, KPC, VIM, and OXA-48-like
NDM, KPC, VIM, and DHA
NDM, KPC, VIM, and CMY
NDM, KPC, OXA-48-like, and DHA
NDM, KPC, OXA-48-like, and CMY
NDM, KPC, DHA, and CMY
NDM, IMP, VIM, and OXA-48-like
NDM, IMP, VIM, and DHA
NDM, IMP, VIM, and CMY
NDM, IMP, OXA-48-like, and DHA
NDM, IMP, OXA-48-like, and CMY
NDM, IMP, DHA, and CMY
NDM, VIM, OXA-48-like, and DHA
NDM, VIM, OXA-48-like, and CMY
NDM, VIM, DHA, and CMY
NDM, OXA-48-like, DHA, and CMY
KPC, IMP, VIM, and OXA-48-like
KPC, IMP, VIM, and DHA
KPC, IMP, VIM, and CMY
KPC, IMP, OXA-48-like, and DHA
KPC, IMP, OXA-48-like, and CMY
KPC, IMP, DHA, and CMY
KPC, VIM, OXA-48-like, and DHA
KPC, VIM, OXA-48-like, and CMY
KPC, VIM, DHA, and CMY
KPC, OXA-48-like, DHA, and CMY
IMP, VIM, OXA-48-like, and DHA
IMP, VIM, OXA-48-like, and CMY
IMP, VIM, DHA, and CMY
IMP, OXA-48-like, DHA, and CMY
VIM, OXA-48-like, DHA, and CMY

TABLE 4
Five-target combinations of EK
(Enterobacter and Klebsiella), NDM, KPC, IMP,
VIM, OXA-48-like, DHA, and CMY

EK, NDM, KPC, IMP, and VIM
EK, NDM, KPC, IMP, and OXA-48-like
EK, NDM, KPC, IMP, and DHA
EK, NDM, KPC, IMP, and CMY
EK, NDM, KPC, VIM, and OXA-48-like
EK, NDM, KPC, VIM, and DHA
EK, NDM, KPC, VIM, and CMY
EK, NDM, KPC, OXA-48-like, and DHA
EK, NDM, KPC, OXA-48-like, and CMY
EK, NDM, KPC, DHA, and CMY
EK, NDM, IMP, VIM, and OXA-48-like
EK, NDM, IMP, VIM, and DHA
EK, NDM, IMP, VIM, and CMY
EK, NDM, IMP, OXA-48-like, and DHA
EK, NDM, IMP, OXA-48-like, and CMY
EK, NDM, IMP, DHA, and CMY
EK, NDM, VIM, OXA-48-like, and DHA
EK, NDM, VIM, OXA-48-like, and CMY
EK, NDM, VIM, DHA, and CMY
EK, NDM, OXA-48-like, DHA, and CMY
EK, KPC, IMP, VIM, and OXA-48-like
EK, KPC, IMP, VIM, and DHA
EK, KPC, IMP, VIM, and CMY
EK, KPC, IMP, OXA-48-like, and DHA
EK, KPC, IMP, OXA-48-like, and CMY
EK, KPC, IMP, DHA, and CMY
EK, KPC, VIM, OXA-48-like, and DHA
EK, KPC, VIM, OXA-48-like, and CMY
EK, KPC, VIM, DHA, and CMY
EK, KPC, OXA-48-like, DHA, and CMY
EK, IMP, VIM, OXA-48-like, and DHA
EK, IMP, VIM, OXA-48-like, and CMY
EK, IMP, VIM, DHA, and CMY
EK, IMP, OXA-48-like, DHA, and CMY
EK, VIM, OXA-48-like, DHA, and CMY
NDM, KPC, IMP, VIM, and OXA-48-like
NDM, KPC, IMP, VIM, and DHA
NDM, KPC, IMP, VIM, and CMY
NDM, KPC, IMP, OXA-48-like, and DHA
NDM, KPC, IMP, OXA-48-like, and CMY
NDM, KPC, IMP, DHA, and CMY
NDM, KPC, VIM, OXA-48-like, and DHA
NDM, KPC, VIM, OXA-48-like, and CMY
NDM, KPC, VIM, DHA, and CMY
NDM, KPC, OXA-48-like, DHA, and CMY
NDM, IMP, VIM, OXA-48-like, and DHA
NDM, IMP, VIM, OXA-48-like, and CMY
NDM, IMP, VIM, DHA, and CMY
NDM, IMP, OXA-48-like, DHA, and CMY
NDM, VIM, OXA-48-like, DHA, and CMY
KPC, IMP, VIM, OXA-48-like, and DHA
KPC, IMP, VIM, OXA-48-like, and CMY
KPC, IMP, VIM, DHA, and CMY
KPC, IMP, OXA-48-like, DHA, and CMY
KPC, VIM, OXA-48-like, DHA, and CMY
IMP, VIM, OXA-48-like, DHA, and CMY

TABLE 5
Six-target combinations of EK
(Enterobacter and Klebsiella), NDM, KPC, IMP,
VIM, OXA-48-like, DHA, and CMY

EK, NDM, KPC, IMP, VIM, and OXA-48-like
EK, NDM, KPC, IMP, VIM, and DHA
EK, NDM, KPC, IMP, VIM, and CMY
EK, NDM, KPC, IMP, OXA-48-like, and DHA
EK, NDM, KPC, IMP, OXA-48-like, and CMY
EK, NDM, KPC, IMP, DHA, and CMY
EK, NDM, KPC, VIM, OXA-48-like, and DHA
EK, NDM, KPC, VIM, OXA-48-like, and CMY
EK, NDM, KPC, VIM, DHA, and CMY
EK, NDM, KPC, OXA-48-like, DHA, and CMY
EK, NDM, IMP, VIM, OXA-48-like, and DHA
EK, NDM, IMP, VIM, OXA-48-like, and CMY
EK, NDM, IMP, VIM, DHA, and CMY
EK, NDM, IMP, OXA-48-like, DHA, and CMY
EK, NDM, VIM, OXA-48-like, DHA, and CMY
EK, KPC, IMP, VIM, OXA-48-like, and DHA
EK, KPC, IMP, VIM, OXA-48-like, and CMY
EK, KPC, IMP, VIM, DHA, and CMY
EK, KPC, IMP, OXA-48-like, DHA, and CMY
EK, KPC, VIM, OXA-48-like, DHA, and CMY
EK, IMP, VIM, OXA-48-like, DHA, and CMY
NDM, KPC, IMP, VIM, OXA-48-like, and DHA
NDM, KPC, IMP, VIM, OXA-48-like, and CMY
NDM, KPC, IMP, VIM, DHA, and CMY
NDM, KPC, IMP, OXA-48-like, DHA, and CMY
NDM, KPC, VIM, OXA-48-like, DHA, and CMY
NDM, IMP, VIM, OXA-48-like, DHA, and CMY
KPC, IMP, VIM, OXA-48-like, DHA, and CMY

TABLE 6
Seven-target combinations of EK
(Enterobacter and Klebsiella), NDM, KPC, IMP,
VIM, OXA-48-like, DHA, and CMY

EK, NDM, KPC, IMP, VIM, OXA-48-like, and DHA
EK, NDM, KPC, IMP, VIM, OXA-48-like, and CMY
EK, NDM, KPC, IMP, VIM, DHA, and CMY
EK, NDM, KPC, IMP, OXA-48-like, DHA, and CMY
EK, NDM, KPC, VIM, OXA-48-like, DHA, and CMY
EK, NDM, IMP, VIM, OXA-48-like, DHA, and CMY
EK, KPC, IMP, VIM, OXA-48-like, DHA, and CMY
NDM, KPC, IMP, VIM, OXA-48-like, DHA, and CMY

In another example, provided herein is a panel is configured to individually detect one or more (e.g., 1, 2, 3, 4, 5, 6, 7, 8, 9, 10, 11, 12, or 13) antibiotic resistance genes selected from the group consisting of KPC, CTX-M 14, CTX-M 15, NDM, VIM, IMP, vanA, vanB, mecA, mecC, OXA-48-like, CMY, and DHA. In some embodiments, the panel is configured to individually detect at least two, at least three, at least four, at least five, at least six, at least seven, at least eight, at least nine, at least ten, at least eleven, at least twelve, or all thirteen antibiotic resistance genes selected from the group consisting of KPC, CTX-M 14, CTX-M 15, NDM, VIM, IMP, vanA, vanB, mecA, mecC, OXA-48-like, CMY, and DHA. In some embodiments, the panel is further configured to detect Enterobacter and Klebsiella spp. Thus, in some embodiments, the panel is configured to detect KPC, CTX-M 14, CTX-M 15, NDM, VIM, IMP, vanA, vanB, mecA, mecC, OXA-48-like, CMY, DHA, and Enterobacter and Klebsiella spp.

In another example, provided herein is a panel is configured to individually detect one or more (e.g., 1, 2, 3, 4, 5, 6, 7, 8, 9, or 10) antibiotic resistance genes selected from the group consisting of CTX-M 14, CTX-M 15, ermA, ermB, mecA, mefA, SHV, TEM, vanA, and vanB. In some embodiments, the panel is configured to individually detect at least two, at least three, at least four, at least five, at least six, at least seven, at least eight, at least nine, or all ten antibiotic resistance genes selected from the group consisting of CTX-M 14, CTX-M 15, ermA, ermB, mecA, mefA, SHV, TEM, vanA, and vanB. In some embodiments, the panel is further configured to detect Streptococcus pneumoniae. For example, the panel may contain any of the combinations set forth in Tables 7-15 below. In some embodiments, the panel is configured to detect CTX-M 14, CTX-M 15, ermA, ermB, mecA, mefA, SHV, TEM, vanA, vanB, and Streptococcus pneumoniae.

TABLE 7
Two-target combinations of Sp
(Streptococcus pneumoniae), CTX-M 14, CTX-M 15, ermA,
ermB, mecA, mefA, SHV, TEM, vanA, and vanB

CTX-M 14 and CTX-M 15
CTX-M 14 and ermA
CTX-M 14 and ermB
CTX-M 14 and mecA
CTX-M 14 and mefA
CTX-M 14 and SHV
CTX-M 14 and TEM
CTX-M 14 and vanA
CTX-M 14 and vanB
CTX-M 14 and Sp
CTX-M 15 and ermA
CTX-M 15 and ermB
CTX-M 15 and mecA
CTX-M 15 and mefA
CTX-M 15 and SHV
CTX-M 15 and TEM
CTX-M 15 and vanA
CTX-M 15 and vanB
CTX-M 15 and Sp
ermA and ermB
ermA and mecA
ermA and mefA
ermA and SHV
ermA and TEM
ermA and vanA
ermA and vanB
ermA and Sp
ermB and mecA
ermB and mefA
ermB and SHV
ermB and TEM
ermB and vanA
ermB and vanB
ermB and Sp
mecA and mefA
mecA and SHV
mecA and TEM
mecA and vanA
mecA and vanB
mecA and Sp
mefA and SHV
mefA and TEM
mefA and vanA
mefA and vanB
mefA and Sp
SHV and TEM
SHV and vanA
SHV and vanB
SHV and Sp
TEM and vanA
TEM and vanB
TEM and Sp
vanA and vanB
vanA and Sp
vanB and Sp

TABLE 8
Three-target combinations of Sp
(Streptococcus pneumoniae), CTX-M 14, CTX-M 15,
ermA, ermB, mecA, mefA, SHV, TEM, vanA, and vanB

CTX-M 14, CTX-M 15, and ermA
CTX-M 14, CTX-M 15, and ermB
CTX-M 14, CTX-M 15, and mecA
CTX-M 14, CTX-M 15, and mefA
CTX-M 14, CTX-M 15, and SHV
CTX-M 14, CTX-M 15, and TEM
CTX-M 14, CTX-M 15, and vanA
CTX-M 14, CTX-M 15, and vanB
CTX-M 14, CTX-M 15, and Sp
CTX-M 14, ermA, and ermB
CTX-M 14, ermA, and mecA
CTX-M 14, ermA, and mefA
CTX-M 14, ermA, and SHV
CTX-M 14, ermA, and TEM
CTX-M 14, ermA, and vanA
CTX-M 14, ermA, and vanB
CTX-M 14, ermA, and Sp
CTX-M 14, ermB, and mecA
CTX-M 14, ermB, and mefA
CTX-M 14, ermB, and SHV
CTX-M 14, ermB, and TEM
CTX-M 14, ermB, and vanA
CTX-M 14, ermB, and vanB
CTX-M 14, ermB, and Sp
CTX-M 14, mecA, and mefA
CTX-M 14, mecA, and SHV
CTX-M 14, mecA, and TEM
CTX-M 14, mecA, and vanA
CTX-M 14, mecA, and vanB
CTX-M 14, mecA, and Sp
CTX-M 14, mefA, and SHV
CTX-M 14, mefA, and TEM
CTX-M 14, mefA, and vanA
CTX-M 14, mefA, and vanB
CTX-M 14, mefA, and Sp
CTX-M 14, SHV, and TEM
CTX-M 14, SHV, and vanA
CTX-M 14, SHV, and vanB
CTX-M 14, SHV, and Sp
CTX-M 14, TEM, and vanA
CTX-M 14, TEM, and vanB
CTX-M 14, TEM, and Sp
CTX-M 14, vanA, and vanB
CTX-M 14, vanA, and Sp
CTX-M 14, vanB, and Sp
CTX-M 15, ermA, and ermB
CTX-M 15, ermA, and mecA
CTX-M 15, ermA, and mefA
CTX-M 15, ermA, and SHV
CTX-M 15, ermA, and TEM
CTX-M 15, ermA, and vanA
CTX-M 15, ermA, and vanB
CTX-M 15, ermA, and Sp
CTX-M 15, ermB, and mecA
CTX-M 15, ermB, and mefA
CTX-M 15, ermB, and SHV
CTX-M 15, ermB, and TEM
CTX-M 15, ermB, and vanA
CTX-M 15, ermB, and vanB
CTX-M 15, ermB, and Sp
CTX-M 15, mecA, and mefA
CTX-M 15, mecA, and SHV
CTX-M 15, mecA, TEM
CTX-M 15, mecA, and vanA
CTX-M 15, mecA, and vanB
CTX-M 15, mecA, and Sp
CTX-M 15, mefA, and SHV
CTX-M 15, mefA, and TEM
CTX-M 15, mefA, and vanA
CTX-M 15, mefA, and vanB
CTX-M 15, mefA, and Sp
CTX-M 15, SHV, and TEM
CTX-M 15, SHV, and vanA
CTX-M 15, SHV, and vanB
CTX-M 15, SHV, and Sp
CTX-M 15, TEM, and vanA
CTX-M 15, TEM, and vanB
CTX-M 15, TEM, and Sp
CTX-M 15, vanA, and vanB
CTX-M 15, vanA, and Sp
CTX-M 15, vanB, and Sp
ermA, ermB, and mecA
ermA, ermB, and mefA
ermA, ermB, and SHV
ermA, ermB, and TEM
ermA, ermB, and vanA
ermA, ermB, and vanB
ermA, ermB, and Sp
ermA, mecA, and mefA
ermA, mecA, and SHV
ermA, mecA, and TEM
ermA, mecA, and vanA
ermA, mecA, and vanB
ermA, mecA, and Sp
ermA, mefA, and SHV
ermA, mefA, and TEM
ermA, mefA, and vanA
ermA, mefA, and vanB
ermA, mefA, and Sp
ermA, SHV, and TEM
ermA, SHV, and vanA
ermA, SHV, and vanB
ermA, SHV, and Sp
ermA, TEM, and vanA
ermA, TEM, and vanB
ermA, TEM, and Sp
ermA, vanA, and vanB
ermA, vanA, and Sp
ermA, vanB, and Sp
ermB, mecA, and mefA
ermB, mecA, and SHV
ermB, mecA, and TEM
ermB, mecA, and vanA
ermB, mecA, and vanB
ermB, mecA, and Sp
ermB, mefA, and SHV
ermB, mefA, and TEM
ermB, mefA, and vanA
ermB, mefA, and vanB
ermB, mefA, and Sp
ermB, SHV, and TEM
ermB, SHV, and vanA
ermB, SHV, and vanB
ermB, SHV, and Sp
ermB, TEM, and vanA
ermB, TEM, and vanB
ermB, TEM, and Sp
ermB, vanA, and vanB
ermB, vanA, and Sp
ermB, vanB, and Sp
mecA, mefA, and SHV
mecA, mefA, and TEM
mecA, mefA, and vanA
mecA, mefA, and vanB
mecA, mefA, and Sp
mecA, SHV, and TEM
mecA, SHV, and vanA
mecA, SHV, and vanB
mecA, SHV, and Sp
mecA, TEM, and vanA
mecA, TEM, and vanB
mecA, TEM, and Sp
mecA, vanA, and vanB
mecA, vanA, and Sp
mecA, vanB, and Sp
mefA, SHV, and TEM
mefA, SHV, and vanA
mefA, SHV, and vanB
mefA, SHV, and Sp
mefA, TEM, and vanA
mefA, TEM, and vanB
mefA, TEM, and Sp
mefA, vanA, and vanB
mefA, vanA, and Sp
mefA, vanB, and Sp
SHV, TEM, and vanA
SHV, TEM, and vanB
SHV, TEM, and Sp
SHV, vanA, and vanB
SHV, vanA, and Sp
SHV, vanB, and Sp
TEM, vanA, and vanB
TEM, vanA, and Sp
TEM, vanB, and Sp
vanA, vanB, and Sp

TABLE 9
Four-target combinations of Sp
(Streptococcus pneumoniae), CTX-M 14, CTX-M 15, ermA,
ermB, mecA, mefA, SHV, TEM, vanA, and vanB

CTX-M 14, CTX-M 15, ermA, and ermB
CTX-M 14, CTX-M 15, ermA, and mecA
CTX-M 14, CTX-M 15, ermA, and mefA
CTX-M 14, CTX-M 15, ermA, and SHV
CTX-M 14, CTX-M 15, ermA, and TEM
CTX-M 14, CTX-M 15, ermA, and vanA
CTX-M 14, CTX-M 15, ermA, and vanB
CTX-M 14, CTX-M 15, ermA, and Sp
CTX-M 14, CTX-M 15, ermB, and mecA
CTX-M 14, CTX-M 15, ermB, and mefA
CTX-M 14, CTX-M 15, ermB, and SHV
CTX-M 14, CTX-M 15, ermB, and TEM
CTX-M 14, CTX-M 15, ermB, and vanA
CTX-M 14, CTX-M 15, ermB, and vanB
CTX-M 14, CTX-M 15, ermB, and Sp
CTX-M 14, CTX-M 15, mecA, and mefA
CTX-M 14, CTX-M 15, mecA, and SHV
CTX-M 14, CTX-M 15, mecA, and TEM
CTX-M 14, CTX-M 15, mecA, and vanA
CTX-M 14, CTX-M 15, mecA, and vanB
CTX-M 14, CTX-M 15, mecA, and Sp
CTX-M 14, CTX-M 15, mefA, and SHV
CTX-M 14, CTX-M 15, mefA, and TEM
CTX-M 14, CTX-M 15, mefA, and vanA
CTX-M 14, CTX-M 15, mefA, and vanB
CTX-M 14, CTX-M 15, mefA, and Sp
CTX-M 14, CTX-M 15, SHV, and TEM
CTX-M 14, CTX-M 15, SHV, and vanA
CTX-M 14, CTX-M 15, SHV, and vanB
CTX-M 14, CTX-M 15, SHV, and Sp
CTX-M 14, CTX-M 15, TEM, and vanA
CTX-M 14, CTX-M 15, TEM, and vanB
CTX-M 14, CTX-M 15, TEM, and Sp
CTX-M 14, CTX-M 15, vanA, and vanB
CTX-M 14, CTX-M 15, vanA, and Sp
CTX-M 14, CTX-M 15, vanB, and Sp
CTX-M 14, ermA, ermB, and mecA
CTX-M 14, ermA, ermB, and mefA
CTX-M 14, ermA, ermB, and SHV
CTX-M 14, ermA, ermB, and TEM
CTX-M 14, ermA, ermB, and vanA
CTX-M 14, ermA, ermB, and vanB
CTX-M 14, ermA, ermB, and Sp
CTX-M 14, ermA, mecA, and mefA
CTX-M 14, ermA, mecA, and SHV
CTX-M 14, ermA, mecA, and TEM
CTX-M 14, ermA, mecA, and vanA
CTX-M 14, ermA, mecA, and vanB
CTX-M 14, ermA, mecA, and Sp
CTX-M 14, ermA, mefA, and SHV
CTX-M 14, ermA, mefA, and TEM
CTX-M 14, ermA, mefA, and vanA
CTX-M 14, ermA, mefA, and vanB
CTX-M 14, ermA, mefA, and Sp
CTX-M 14, ermA, SHV, and TEM
CTX-M 14, ermA, SHV, and vanA
CTX-M 14, ermA, SHV, and vanB
CTX-M 14, ermA, SHV, and Sp
CTX-M 14, ermA, TEM, and vanA
CTX-M 14, ermA, TEM, and vanB
CTX-M 14, ermA, TEM, and Sp
CTX-M 14, ermA, vanA, and vanB
CTX-M 14, ermA, vanA, and Sp
CTX-M 14, ermA, vanB, and Sp
CTX-M 14, ermB, mecA, and mefA
CTX-M 14, ermB, mecA, and SHV
CTX-M 14, ermB, mecA, and TEM
CTX-M 14, ermB, mecA, and vanA
CTX-M 14, ermB, mecA, and vanB
CTX-M 14, ermB, mecA, and Sp
CTX-M 14, ermB, mefA, and SHV
CTX-M 14, ermB, mefA, and TEM
CTX-M 14, ermB, mefA, and vanA
CTX-M 14, ermB, mefA, and vanB
CTX-M 14, ermB, mefA, and Sp
CTX-M 14, ermB, SHV, and TEM
CTX-M 14, ermB, SHV, and vanA
CTX-M 14, ermB, SHV, and vanB
CTX-M 14, ermB, SHV, and Sp
CTX-M 14, ermB, TEM, and vanA
CTX-M 14, ermB, TEM, and vanB
CTX-M 14, ermB, TEM, and Sp
CTX-M 14, ermB, vanA, and vanB
CTX-M 14, ermB, vanA, and Sp
CTX-M 14, ermB, vanB, and Sp
CTX-M 14, mecA, mefA, and SHV
CTX-M 14, mecA, mefA, and TEM
CTX-M 14, mecA, mefA, and vanA
CTX-M 14, mecA, mefA, and vanB
CTX-M 14, mecA, mefA, and Sp
CTX-M 14, mecA, SHV, and TEM
CTX-M 14, mecA, SHV, and vanA
CTX-M 14, mecA, SHV, and vanB
CTX-M 14, mecA, SHV, and Sp
CTX-M 14, mecA, TEM, and vanA
CTX-M 14, mecA, TEM, and vanB
CTX-M 14, mecA, TEM, and Sp
CTX-M 14, mecA, vanA, and vanB
CTX-M 14, mecA, vanA, and Sp
CTX-M 14, mecA, vanB, and Sp
CTX-M 14, mefA, SHV, and TEM
CTX-M 14, mefA, SHV, and vanA
CTX-M 14, mefA, SHV, and vanB
CTX-M 14, mefA, SHV, and Sp
CTX-M 14, mefA, TEM, and vanA
CTX-M 14, mefA, TEM, and vanB
CTX-M 14, mefA, TEM, and Sp
CTX-M 14, mefA, vanA, and vanB
CTX-M 14, mefA, vanA, and Sp
CTX-M 14, mefA, vanB, and Sp
CTX-M 14, SHV, TEM, and vanA
CTX-M 14, SHV, TEM, and vanB
CTX-M 14, SHV, TEM, and Sp
CTX-M 14, SHV, vanA, and vanB
CTX-M 14, SHV, vanA, and Sp
CTX-M 14, SHV, vanB, and Sp
CTX-M 14, TEM, vanA, and vanB
CTX-M 14, TEM, vanA, and Sp
CTX-M 14, TEM, vanB, and Sp
CTX-M 14, vanA, vanB, and Sp,
CTX-M 15, ermA, ermB, and mecA
CTX-M 15, ermA, ermB, and mefA
CTX-M 15, ermA, ermB, and SHV
CTX-M 15, ermA, ermB, and TEM
CTX-M 15, ermA, ermB, and vanA
CTX-M 15, ermA, ermB, and vanB
CTX-M 15, ermA, ermB, and Sp
CTX-M 15, ermA, mecA, and mefA
CTX-M 15, ermA, mecA, and SHV
CTX-M 15, ermA, mecA, and TEM
CTX-M 15, ermA, mecA, and vanA
CTX-M 15, ermA, mecA, and vanB
CTX-M 15, ermA, mecA, and Sp
CTX-M 15, ermA, mefA, and SHV
CTX-M 15, ermA, mefA, and TEM
CTX-M 15, ermA, mefA, and vanA
CTX-M 15, ermA, mefA, and vanB
CTX-M 15, ermA, mefA, and Sp
CTX-M 15, ermA, SHV, and TEM
CTX-M 15, ermA, SHV, and vanA
CTX-M 15, ermA, SHV, and vanB
CTX-M 15, ermA, SHV, and Sp
CTX-M 15, ermA, TEM, and vanA
CTX-M 15, ermA, TEM, and vanB
CTX-M 15, ermA, TEM, and Sp
CTX-M 15, ermA, vanA, and vanB
CTX-M 15, ermA, vanA, and Sp
CTX-M 15, ermA, vanB, and Sp
CTX-M 15, ermB, mecA, and mefA
CTX-M 15, ermB, mecA, and SHV
CTX-M 15, ermB, mecA, and TEM
CTX-M 15, ermB, mecA, and vanA
CTX-M 15, ermB, mecA, and vanB
CTX-M 15, ermB, mecA, and Sp
CTX-M 15, ermB, mefA, and SHV
CTX-M 15, ermB, mefA, and TEM
CTX-M 15, ermB, mefA, and vanA
CTX-M 15, ermB, mefA, and vanB
CTX-M 15, ermB, mefA, and Sp
CTX-M 15, ermB, SHV, and TEM
CTX-M 15, ermB, SHV, and vanA
CTX-M 15, ermB, SHV, and vanB
CTX-M 15, ermB, SHV, and Sp
CTX-M 15, ermB, TEM, and vanA
CTX-M 15, ermB, TEM, and vanB
CTX-M 15, ermB, TEM, and Sp
CTX-M 15, ermB, vanA, and vanB
CTX-M 15, ermB, vanA, and Sp
CTX-M 15, ermB, vanB, and Sp
CTX-M 15, mecA, mefA, and SHV
CTX-M 15, mecA, mefA, and TEM
CTX-M 15, mecA, mefA, and vanA
CTX-M 15, mecA, mefA, and vanB
CTX-M 15, mecA, mefA, and Sp
CTX-M 15, mecA, SHV, and TEM
CTX-M 15, mecA, SHV, and vanA
CTX-M 15, mecA, SHV, and vanB
CTX-M 15, mecA, SHV, and Sp
CTX-M 15, mecA, TEM, and vanA
CTX-M 15, mecA, TEM, and vanB
CTX-M 15, mecA, TEM, and Sp
CTX-M 15, mecA, vanA, and vanB
CTX-M 15, mecA, vanA, and Sp
CTX-M 15, mecA, vanB, and Sp
CTX-M 15, mefA, SHV, and TEM
CTX-M 15, mefA, SHV, and vanA
CTX-M 15, mefA, SHV, and vanB
CTX-M 15, mefA, SHV, and Sp
CTX-M 15, mefA, TEM, and vanA
CTX-M 15, mefA, TEM, and vanB
CTX-M 15, mefA, TEM, and Sp
CTX-M 15, mefA, vanA, and vanB
CTX-M 15, mefA, vanA, and Sp
CTX-M 15, mefA, vanB, and Sp
CTX-M 15, SHV, TEM, and vanA
CTX-M 15, SHV, TEM, and vanB
CTX-M 15, SHV, TEM, and Sp
CTX-M 15, SHV, vanA, and vanB
CTX-M 15, SHV, vanA, and Sp
CTX-M 15, SHV, vanB, and Sp
CTX-M 15, TEM, vanA, and vanB
CTX-M 15, TEM, vanA, and Sp
CTX-M 15, TEM, vanB, and Sp
CTX-M 15, vanA, vanB, and Sp
ermA, ermB, mecA, and mefA
ermA, ermB, mecA, and SHV
ermA, ermB, mecA, and TEM
ermA, ermB, mecA, and vanA
ermA, ermB, mecA, and vanB
ermA, ermB, mecA, and Sp
ermA, ermB, mefA, and SHV
ermA, ermB, mefA, and TEM
ermA, ermB, mefA, and vanA
ermA, ermB, mefA, and vanB
ermA, ermB, mefA, and Sp
ermA, ermB, SHV, and TEM
ermA, ermB, SHV, and vanA
ermA, ermB, SHV, and vanB
ermA, ermB, SHV, and Sp
ermA, ermB, TEM, and vanA
ermA, ermB, TEM, and vanB
ermA, ermB, TEM, and Sp
ermA, ermB, vanA, and vanB
ermA, ermB, vanA, and Sp
ermA, ermB, vanB, and Sp
ermA, mecA, mefA, and SHV
ermA, mecA, mefA, and TEM
ermA, mecA, mefA, and vanA
ermA, mecA, mefA, and vanB
ermA, mecA, mefA, and Sp
ermA, mecA, SHV, and TEM
ermA, mecA, SHV, and vanA
ermA, mecA, SHV, and vanB
ermA, mecA, SHV, and Sp
ermA, mecA, TEM, and vanA
ermA, mecA, TEM, and vanB
ermA, mecA, TEM, and Sp
ermA, mecA, vanA, and vanB
ermA, mecA, vanA, and Sp
ermA, mecA, vanB, and Sp
ermA, mefA, SHV, and TEM
ermA, mefA, SHV, and vanA
ermA, mefA, SHV, and vanB
ermA, mefA, SHV, and Sp
ermA, mefA, TEM, and vanA
ermA, mefA, TEM, and vanB
ermA, mefA, TEM, and Sp
ermA, mefA, vanA, and vanB
ermA, mefA, vanA, and Sp
ermA, mefA, vanB, and Sp
ermA, SHV, TEM, and vanA
ermA, SHV, TEM, and vanB
ermA, SHV, TEM, and Sp
ermA, SHV, vanA, and vanB
ermA, SHV, vanA, and Sp
ermA, SHV, vanB, and Sp
ermA, TEM, vanA, and vanB
ermA, TEM, vanA, and Sp
ermA, TEM, vanB, and Sp
ermA, vanA, vanB, and Sp
ermB, mecA, mefA, and SHV
ermB, mecA, mefA, and TEM
ermB, mecA, mefA, and vanA
ermB, mecA, mefA, and vanB
ermB, mecA, mefA, and Sp
ermB, mecA, SHV, and TEM
ermB, mecA, SHV, and vanA
ermB, mecA, SHV, and vanB
ermB, mecA, SHV, and Sp
ermB, mecA, TEM, and vanA
ermB, mecA, TEM, and vanB
ermB, mecA, TEM, and Sp
ermB, mecA, vanA, and vanB
ermB, mecA, vanA, and Sp
ermB, mecA, vanB, and Sp
ermB, mefA, SHV, and TEM
ermB, mefA, SHV, and vanA
ermB, mefA, SHV, and vanB
ermB, mefA, SHV, and Sp
ermB, mefA, TEM, and vanA
ermB, mefA, TEM, and vanB
ermB, mefA, TEM, and Sp
ermB, mefA, vanA, and vanB
ermB, mefA, vanA, and Sp
ermB, mefA, vanB, and Sp
ermB, SHV, TEM, and vanA
ermB, SHV, TEM, and vanB
ermB, SHV, TEM, and Sp
ermB, SHV, vanA, and vanB
ermB, SHV, vanA, and Sp
ermB, SHV, vanB, and Sp
ermB, TEM, vanA, and vanB
ermB, TEM, vanA, and Sp
ermB, TEM, vanB, and Sp
ermB, vanA, vanB, and Sp
mecA, mefA, SHV, and TEM
mecA, mefA, SHV, and vanA
mecA, mefA, SHV, and vanB
mecA, mefA, SHV, and Sp
mecA, mefA, TEM, and vanA
mecA, mefA, TEM, and vanB
mecA, mefA, TEM, and Sp
mecA, mefA, vanA, and vanB
mecA, mefA, vanA, and Sp
mecA, mefA, vanB, and Sp
mecA, SHV, TEM, and vanA
mecA, SHV, TEM, and vanB
mecA, SHV, TEM, and Sp
mecA, SHV, vanA, and vanB
mecA, SHV, vanA, and Sp
mecA, SHV, vanB, and Sp
mecA, TEM, vanA, and vanB
mecA, TEM, vanA, and Sp
mecA, TEM, vanB, and Sp
mecA, vanA, vanB, and Sp
mefA, SHV, TEM, and vanA
mefA, SHV, TEM, and vanB
mefA, SHV, TEM, and Sp
mefA, SHV, vanA, and vanB
mefA, SHV, vanA, and Sp
mefA, SHV, vanB, and Sp
mefA, TEM, vanA, and vanB
mefA, TEM, vanA, and Sp
mefA, TEM, vanB, and Sp
mefA, vanA, vanB, and Sp
SHV, TEM, vanA, and vanB
SHV, TEM, vanA, and Sp
SHV, TEM, vanB, and Sp
SHV, vanA, vanB, and Sp
TEM, vanA, vanB, and Sp

TABLE 10
Five-target combinations of Sp
(Streptococcus pneumoniae), CTX-M 14, CTX-M 15, ermA,
ermB, mecA, mefA, SHV, TEM, vanA, and vanB

CTX-M 14, CTX-M 15, ermA, ermB, and mecA
CTX-M 14, CTX-M 15, ermA, ermB, and mefA
CTX-M 14, CTX-M 15, ermA, ermB, and SHV
CTX-M 14, CTX-M 15, ermA, ermB, and TEM
CTX-M 14, CTX-M 15, ermA, ermB, and vanA
CTX-M 14, CTX-M 15, ermA, ermB, and vanB
CTX-M 14, CTX-M 15, ermA, ermB, and Sp
CTX-M 14, CTX-M 15, ermA, mecA, and mefA
CTX-M 14, CTX-M 15, ermA, mecA, and SHV
CTX-M 14, CTX-M 15, ermA, mecA, and TEM
CTX-M 14, CTX-M 15, ermA, mecA, and vanA
CTX-M 14, CTX-M 15, ermA, mecA, and vanB
CTX-M 14, CTX-M 15, ermA, mecA, and Sp
CTX-M 14, CTX-M 15, ermA, mefA, and SHV
CTX-M 14, CTX-M 15, ermA, mefA, and TEM
CTX-M 14, CTX-M 15, ermA, mefA, and vanA
CTX-M 14, CTX-M 15, ermA, mefA, and vanB
CTX-M 14, CTX-M 15, ermA, mefA, and Sp
CTX-M 14, CTX-M 15, ermA, SHV, and TEM
CTX-M 14, CTX-M 15, ermA, SHV, and vanA
CTX-M 14, CTX-M 15, ermA, SHV, and vanB
CTX-M 14, CTX-M 15, ermA, SHV, and Sp
CTX-M 14, CTX-M 15, ermA, TEM, and vanA
CTX-M 14, CTX-M 15, ermA, TEM, and vanB
CTX-M 14, CTX-M 15, ermA, TEM, and Sp
CTX-M 14, CTX-M 15, ermA, vanA, and vanB
CTX-M 14, CTX-M 15, ermA, vanA, and Sp
CTX-M 14, CTX-M 15, ermA, vanB, and Sp
CTX-M 14, CTX-M 15, ermB, mecA, and mefA
CTX-M 14, CTX-M 15, ermB, mecA, and SHV
CTX-M 14, CTX-M 15, ermB, mecA, and TEM
CTX-M 14, CTX-M 15, ermB, mecA, and vanA
CTX-M 14, CTX-M 15, ermB, mecA, and vanB
CTX-M 14, CTX-M 15, ermB, mecA, and Sp
CTX-M 14, CTX-M 15, ermB, mefA, and SHV
CTX-M 14, CTX-M 15, ermB, mefA, and TEM
CTX-M 14, CTX-M 15, ermB, mefA, and vanA
CTX-M 14, CTX-M 15, ermB, mefA, and vanB
CTX-M 14, CTX-M 15, ermB, mefA, and Sp
CTX-M 14, CTX-M 15, ermB, SHV, and TEM
CTX-M 14, CTX-M 15, ermB, SHV, and vanA
CTX-M 14, CTX-M 15, ermB, SHV, and vanB
CTX-M 14, CTX-M 15, ermB, SHV, and Sp
CTX-M 14, CTX-M 15, ermB, TEM, and vanA
CTX-M 14, CTX-M 15, ermB, TEM, and vanB
CTX-M 14, CTX-M 15, ermB, TEM, and Sp
CTX-M 14, CTX-M 15, ermB, vanA, and vanB
CTX-M 14, CTX-M 15, ermB, vanA, and Sp
CTX-M 14, CTX-M 15, ermB, vanB, and Sp
CTX-M 14, CTX-M 15, mecA, mefA, and SHV
CTX-M 14, CTX-M 15, mecA, mefA, and TEM
CTX-M 14, CTX-M 15, mecA, mefA, and vanA
CTX-M 14, CTX-M 15, mecA, mefA, and vanB
CTX-M 14, CTX-M 15, mecA, mefA, and Sp
CTX-M 14, CTX-M 15, mecA, SHV, and TEM
CTX-M 14, CTX-M 15, mecA, SHV, and vanA
CTX-M 14, CTX-M 15, mecA, SHV, and vanB
CTX-M 14, CTX-M 15, mecA, SHV, and Sp
CTX-M 14, CTX-M 15, mecA, TEM, and vanA
CTX-M 14, CTX-M 15, mecA, TEM, and vanB
CTX-M 14, CTX-M 15, mecA, TEM, and Sp
CTX-M 14, CTX-M 15, mecA, vanA, and vanB
CTX-M 14, CTX-M 15, mecA, vanA, and Sp
CTX-M 14, CTX-M 15, mecA, vanB, and Sp
CTX-M 14, CTX-M 15, mefA, SHV, and TEM
CTX-M 14, CTX-M 15, mefA, SHV, and vanA
CTX-M 14, CTX-M 15, mefA, SHV, and vanB
CTX-M 14, CTX-M 15, mefA, SHV, and Sp
CTX-M 14, CTX-M 15, mefA, TEM, and vanA
CTX-M 14, CTX-M 15, mefA, TEM, and vanB
CTX-M 14, CTX-M 15, mefA, TEM, and Sp
CTX-M 14, CTX-M 15, mefA, vanA, and vanB
CTX-M 14, CTX-M 15, mefA, vanA, and Sp
CTX-M 14, CTX-M 15, mefA, vanB, and Sp
CTX-M 14, CTX-M 15, SHV, TEM, and vanA
CTX-M 14, CTX-M 15, SHV, TEM, and vanB
CTX-M 14, CTX-M 15, SHV, TEM, and Sp
CTX-M 14, CTX-M 15, SHV, vanA, and vanB
CTX-M 14, CTX-M 15, SHV, vanA, and Sp
CTX-M 14, CTX-M 15, SHV, vanB, and Sp
CTX-M 14, CTX-M 15, TEM, vanA, and vanB
CTX-M 14, CTX-M 15, TEM, vanA, and Sp
CTX-M 14, CTX-M 15, TEM, vanB, and Sp
CTX-M 14, CTX-M 15, vanA, vanB, and Sp
CTX-M 14, ermA, ermB, mecA, and mefA
CTX-M 14, ermA, ermB, mecA, and SHV
CTX-M 14, ermA, ermB, mecA, and TEM
CTX-M 14, ermA, ermB, mecA, and vanA
CTX-M 14, ermA, ermB, mecA, and vanB
CTX-M 14, ermA, ermB, mecA, and Sp
CTX-M 14, ermA, ermB, mefA, and SHV
CTX-M 14, ermA, ermB, mefA, and TEM
CTX-M 14, ermA, ermB, mefA, and vanA
CTX-M 14, ermA, ermB, mefA, and vanB
CTX-M 14, ermA, ermB, mefA, and Sp
CTX-M 14, ermA, ermB, SHV, and TEM
CTX-M 14, ermA, ermB, SHV, and vanA
CTX-M 14, ermA, ermB, SHV, and vanB
CTX-M 14, ermA, ermB, SHV, and Sp
CTX-M 14, ermA, ermB, TEM, and vanA
CTX-M 14, ermA, ermB, TEM, and vanB
CTX-M 14, ermA, ermB, TEM, and Sp
CTX-M 14, ermA, ermB, vanA, and vanB
CTX-M 14, ermA, ermB, vanA, and Sp
CTX-M 14, ermA, ermB, vanB, and Sp
CTX-M 14, ermA, mecA, mefA, and SHV
CTX-M 14, ermA, mecA, mefA, and TEM
CTX-M 14, ermA, mecA, mefA, and vanA
CTX-M 14, ermA, mecA, mefA, and vanB
CTX-M 14, ermA, mecA, mefA, and Sp
CTX-M 14, ermA, mecA, SHV, and TEM
CTX-M 14, ermA, mecA, SHV, and vanA
CTX-M 14, ermA, mecA, SHV, and vanB
CTX-M 14, ermA, mecA, SHV, and Sp
CTX-M 14, ermA, mecA, TEM, and vanA
CTX-M 14, ermA, mecA, TEM, and vanB
CTX-M 14, ermA, mecA, TEM, and Sp
CTX-M 14, ermA, mecA, vanA, and vanB
CTX-M 14, ermA, mecA, vanA, and Sp
CTX-M 14, ermA, mecA, vanB, and Sp
CTX-M 14, ermA, mefA, SHV, and TEM
CTX-M 14, ermA, mefA, SHV, and vanA
CTX-M 14, ermA, mefA, SHV, and vanB
CTX-M 14, ermA, mefA, SHV, and Sp
CTX-M 14, ermA, mefA, TEM, and vanA
CTX-M 14, ermA, mefA, TEM, and vanB
CTX-M 14, ermA, mefA, TEM, and Sp
CTX-M 14, ermA, mefA, vanA, and vanB
CTX-M 14, ermA, mefA, vanA, and Sp
CTX-M 14, ermA, mefA, vanB, and Sp
CTX-M 14, ermA, SHV, TEM, and vanA
CTX-M 14, ermA, SHV, TEM, and vanB
CTX-M 14, ermA, SHV, TEM, and Sp
CTX-M 14, ermA, SHV, vanA, and vanB
CTX-M 14, ermA, SHV, vanA, and Sp
CTX-M 14, ermA, SHV, vanB, and Sp
CTX-M 14, ermA, TEM, vanA, and vanB
CTX-M 14, ermA, TEM, vanA, and Sp
CTX-M 14, ermA, TEM, vanB, and Sp
CTX-M 14, ermA, vanA, vanB, and Sp
CTX-M 14, ermB, mecA, mefA, and SHV
CTX-M 14, ermB, mecA, mefA, and TEM
CTX-M 14, ermB, mecA, mefA, and vanA
CTX-M 14, ermB, mecA, mefA, and vanB
CTX-M 14, ermB, mecA, mefA, and Sp
CTX-M 14, ermB, mecA, SHV, and TEM
CTX-M 14, ermB, mecA, SHV, and vanA
CTX-M 14, ermB, mecA, SHV, and vanB
CTX-M 14, ermB, mecA, SHV, and Sp
CTX-M 14, ermB, mecA, TEM, and vanA
CTX-M 14, ermB, mecA, TEM, and vanB
CTX-M 14, ermB, mecA, TEM, and Sp
CTX-M 14, ermB, mecA, vanA, and vanB
CTX-M 14, ermB, mecA, vanA, and Sp
CTX-M 14, ermB, mecA, vanB, and Sp
CTX-M 14, ermB, mefA, SHV, and TEM
CTX-M 14, ermB, mefA, SHV, and vanA
CTX-M 14, ermB, mefA, SHV, and vanB
CTX-M 14, ermB, mefA, SHV, and Sp
CTX-M 14, ermB, mefA, TEM, and vanA
CTX-M 14, ermB, mefA, TEM, and vanB
CTX-M 14, ermB, mefA, TEM, and Sp
CTX-M 14, ermB, mefA, vanA, and vanB
CTX-M 14, ermB, mefA, vanA, and Sp
CTX-M 14, ermB, mefA, vanB, and Sp
CTX-M 14, ermB, SHV, TEM, and vanA
CTX-M 14, ermB, SHV, TEM, and vanB
CTX-M 14, ermB, SHV, TEM, and Sp
CTX-M 14, ermB, SHV, vanA, and vanB
CTX-M 14, ermB, SHV, vanA, and Sp
CTX-M 14, ermB, SHV, vanB, and Sp
CTX-M 14, ermB, TEM, vanA, and vanB
CTX-M 14, ermB, TEM, vanA, and Sp
CTX-M 14, ermB, TEM, vanB, and Sp
CTX-M 14, ermB, vanA, vanB, and Sp
CTX-M 14, mecA, mefA, SHV, and TEM
CTX-M 14, mecA, mefA, SHV, and vanA
CTX-M 14, mecA, mefA, SHV, and vanB
CTX-M 14, mecA, mefA, SHV, and Sp
CTX-M 14, mecA, mefA, TEM, and vanA
CTX-M 14, mecA, mefA, TEM, and vanB
CTX-M 14, mecA, mefA, TEM, and Sp
CTX-M 14, mecA, mefA, vanA, and vanB
CTX-M 14, mecA, mefA, vanA, and Sp
CTX-M 14, mecA, mefA, vanB, and Sp
CTX-M 14, mecA, SHV, TEM, and vanA
CTX-M 14, mecA, SHV, TEM, and vanB
CTX-M 14, mecA, SHV, TEM, and Sp
CTX-M 14, mecA, SHV, vanA, and vanB
CTX-M 14, mecA, SHV, vanA, and Sp
CTX-M 14, mecA, SHV, vanB, and Sp
CTX-M 14, mecA, TEM, vanA, and vanB
CTX-M 14, mecA, TEM, vanA, and Sp
CTX-M 14, mecA, TEM, vanB, and Sp
CTX-M 14, mecA, vanA, vanB, and Sp
CTX-M 14, mefA, SHV, TEM, and vanA
CTX-M 14, mefA, SHV, TEM, and vanB
CTX-M 14, mefA, SHV, TEM, and Sp
CTX-M 14, mefA, SHV, vanA, and vanB
CTX-M 14, mefA, SHV, vanA, and Sp
CTX-M 14, mefA, SHV, vanB, and Sp
CTX-M 14, mefA, TEM, vanA, and vanB
CTX-M 14, mefA, TEM, vanA, and Sp
CTX-M 14, mefA, TEM, vanB, and Sp
CTX-M 14, mefA, vanA, vanB, and Sp
CTX-M 14, SHV, TEM, vanA, and vanB
CTX-M 14, SHV, TEM, vanA, and Sp
CTX-M 14, SHV, TEM, vanB, and Sp
CTX-M 14, SHV, vanA, vanB, and Sp
CTX-M 14, TEM, vanA, vanB, and Sp
CTX-M 15, ermA, ermB, mecA, and mefA
CTX-M 15, ermA, ermB, mecA, and SHV
CTX-M 15, ermA, ermB, mecA, and TEM
CTX-M 15, ermA, ermB, mecA, and vanA
CTX-M 15, ermA, ermB, mecA, and vanB
CTX-M 15, ermA, ermB, mecA, and Sp
CTX-M 15, ermA, ermB, mefA, and SHV
CTX-M 15, ermA, ermB, mefA, and TEM
CTX-M 15, ermA, ermB, mefA, and vanA
CTX-M 15, ermA, ermB, mefA, and vanB
CTX-M 15, ermA, ermB, mefA, and Sp
CTX-M 15, ermA, ermB, SHV, and TEM
CTX-M 15, ermA, ermB, SHV, and vanA
CTX-M 15, ermA, ermB, SHV, and vanB
CTX-M 15, ermA, ermB, SHV, and Sp
CTX-M 15, ermA, ermB, TEM, and vanA
CTX-M 15, ermA, ermB, TEM, and vanB
CTX-M 15, ermA, ermB, TEM, and Sp
CTX-M 15, ermA, ermB, vanA, and vanB
CTX-M 15, ermA, ermB, vanA, and Sp
CTX-M 15, ermA, ermB, vanB, and Sp
CTX-M 15, ermA, mecA, mefA, and SHV
CTX-M 15, ermA, mecA, mefA, and TEM
CTX-M 15, ermA, mecA, mefA, and vanA
CTX-M 15, ermA, mecA, mefA, and vanB
CTX-M 15, ermA, mecA, mefA, and Sp
CTX-M 15, ermA, mecA, SHV, and TEM
CTX-M 15, ermA, mecA, SHV, and vanA
CTX-M 15, ermA, mecA, SHV, and vanB
CTX-M 15, ermA, mecA, SHV, and Sp
CTX-M 15, ermA, mecA, TEM, and vanA
CTX-M 15, ermA, mecA, TEM, and vanB
CTX-M 15, ermA, mecA, TEM, and Sp
CTX-M 15, ermA, mecA, vanA, and vanB
CTX-M 15, ermA, mecA, vanA, and Sp
CTX-M 15, ermA, mecA, vanB, and Sp
CTX-M 15, ermA, mefA, SHV, and TEM
CTX-M 15, ermA, mefA, SHV, and vanA
CTX-M 15, ermA, mefA, SHV, and vanB
CTX-M 15, ermA, mefA, SHV, and Sp
CTX-M 15, ermA, mefA, TEM, and vanA
CTX-M 15, ermA, mefA, TEM, and vanB
CTX-M 15, ermA, mefA, TEM, and Sp
CTX-M 15, ermA, mefA, vanA, and vanB
CTX-M 15, ermA, mefA, vanA, and Sp
CTX-M 15, ermA, mefA, vanB, and Sp
CTX-M 15, ermA, SHV, TEM, and vanA
CTX-M 15, ermA, SHV, TEM, and vanB
CTX-M 15, ermA, SHV, TEM, and Sp
CTX-M 15, ermA, SHV, vanA, and vanB
CTX-M 15, ermA, SHV, vanA, and Sp
CTX-M 15, ermA, SHV, vanB, and Sp
CTX-M 15, ermA, TEM, vanA, and vanB
CTX-M 15, ermA, TEM, vanA, and Sp
CTX-M 15, ermA, TEM, vanB, and Sp
CTX-M 15, ermA, vanA, vanB, and Sp
CTX-M 15, ermB, mecA, mefA, and SHV
CTX-M 15, ermB, mecA, mefA, and TEM
CTX-M 15, ermB, mecA, mefA, and vanA
CTX-M 15, ermB, mecA, mefA, and vanB
CTX-M 15, ermB, mecA, mefA, and Sp
CTX-M 15, ermB, mecA, SHV, and TEM
CTX-M 15, ermB, mecA, SHV, and vanA
CTX-M 15, ermB, mecA, SHV, and vanB
CTX-M 15, ermB, mecA, SHV, and Sp
CTX-M 15, ermB, mecA, TEM, and vanA
CTX-M 15, ermB, mecA, TEM, and vanB
CTX-M 15, ermB, mecA, TEM, and Sp
CTX-M 15, ermB, mecA, vanA, and vanB
CTX-M 15, ermB, mecA, vanA, and Sp
CTX-M 15, ermB, mecA, vanB, and Sp
CTX-M 15, ermB, mefA, SHV, and TEM
CTX-M 15, ermB, mefA, SHV, and vanA
CTX-M 15, ermB, mefA, SHV, and vanB
CTX-M 15, ermB, mefA, SHV, and Sp
CTX-M 15, ermB, mefA, TEM, and vanA
CTX-M 15, ermB, mefA, TEM, and vanB
CTX-M 15, ermB, mefA, TEM, and Sp
CTX-M 15, ermB, mefA, vanA, and vanB
CTX-M 15, ermB, mefA, vanA, and Sp
CTX-M 15, ermB, mefA, vanB, and Sp
CTX-M 15, ermB, SHV, TEM, and vanA
CTX-M 15, ermB, SHV, TEM, and vanB
CTX-M 15, ermB, SHV, TEM, and Sp
CTX-M 15, ermB, SHV, vanA, and vanB
CTX-M 15, ermB, SHV, vanA, and Sp
CTX-M 15, ermB, SHV, vanB, and Sp
CTX-M 15, ermB, TEM, vanA, and vanB
CTX-M 15, ermB, TEM, vanA, and Sp
CTX-M 15, ermB, TEM, vanB, and Sp
CTX-M 15, ermB, vanA, vanB, and Sp
CTX-M 15, mecA, mefA, SHV, and TEM
CTX-M 15, mecA, mefA, SHV, and vanA
CTX-M 15, mecA, mefA, SHV, and vanB
CTX-M 15, mecA, mefA, SHV, and Sp
CTX-M 15, mecA, mefA, TEM, and vanA
CTX-M 15, mecA, mefA, TEM, and vanB
CTX-M 15, mecA, mefA, TEM, and Sp
CTX-M 15, mecA, mefA, vanA, and vanB
CTX-M 15, mecA, mefA, vanA, and Sp
CTX-M 15, mecA, mefA, vanB, and Sp
CTX-M 15, mecA, SHV, TEM, and vanA
CTX-M 15, mecA, SHV, TEM, and vanB
CTX-M 15, mecA, SHV, TEM, and Sp
CTX-M 15, mecA, SHV, vanA, and vanB
CTX-M 15, mecA, SHV, vanA, and Sp
CTX-M 15, mecA, SHV, vanB, and Sp
CTX-M 15, mecA, TEM, vanA, and vanB
CTX-M 15, mecA, TEM, vanA, and Sp
CTX-M 15, mecA, TEM, vanB, and Sp
CTX-M 15, mecA, vanA, vanB, and Sp
CTX-M 15, mefA, SHV, TEM, and vanA
CTX-M 15, mefA, SHV, TEM, and vanB
CTX-M 15, mefA, SHV, TEM, and Sp
CTX-M 15, mefA, SHV, vanA, and vanB
CTX-M 15, mefA, SHV, vanA, and Sp
CTX-M 15, mefA, SHV, vanB, and Sp
CTX-M 15, mefA, TEM, vanA, and vanB
CTX-M 15, mefA, TEM, vanA, and Sp
CTX-M 15, mefA, TEM, vanB, and Sp
CTX-M 15, mefA, vanA, vanB, and Sp
CTX-M 15, SHV, TEM, vanA, and vanB
CTX-M 15, SHV, TEM, vanA, and Sp
CTX-M 15, SHV, TEM, vanB, and Sp
CTX-M 15, SHV, vanA, vanB, and Sp
CTX-M 15, TEM, vanA, vanB, and Sp
ermA, ermB, mecA, mefA, and SHV
ermA, ermB, mecA, mefA, and TEM
ermA, ermB, mecA, mefA, and vanA
ermA, ermB, mecA, mefA, and vanB
ermA, ermB, mecA, mefA, and Sp
ermA, ermB, mecA, SHV, and TEM
ermA, ermB, mecA, SHV, and vanA
ermA, ermB, mecA, SHV, and vanB
ermA, ermB, mecA, SHV, and Sp
ermA, ermB, mecA, TEM, and vanA
ermA, ermB, mecA, TEM, and vanB
ermA, ermB, mecA, TEM, and Sp
ermA, ermB, mecA, vanA, and vanB
ermA, ermB, mecA, vanA, and Sp
ermA, ermB, mecA, vanB, and Sp
ermA, ermB, mefA, SHV, and TEM
ermA, ermB, mefA, SHV, and vanA
ermA, ermB, mefA, SHV, and vanB
ermA, ermB, mefA, SHV, and Sp
ermA, ermB, mefA, TEM, and vanA
ermA, ermB, mefA, TEM, and vanB
ermA, ermB, mefA, TEM, and Sp
ermA, ermB, mefA, vanA, and vanB
ermA, ermB, mefA, vanA, and Sp
ermA, ermB, mefA, vanB, and Sp
ermA, ermB, SHV, TEM, and vanA
ermA, ermB, SHV, TEM, and vanB
ermA, ermB, SHV, TEM, and Sp
ermA, ermB, SHV, vanA, and vanB
ermA, ermB, SHV, vanA, and Sp
ermA, ermB, SHV, vanB, and Sp
ermA, ermB, TEM, vanA, and vanB
ermA, ermB, TEM, vanA, and Sp
ermA, ermB, TEM, vanB, and Sp
ermA, ermB, vanA, vanB, and Sp
ermA, mecA, mefA, SHV, and TEM
ermA, mecA, mefA, SHV, and vanA
ermA, mecA, mefA, SHV, and vanB
ermA, mecA, mefA, SHV, and Sp
ermA, mecA, mefA, TEM, and vanA
ermA, mecA, mefA, TEM, and vanB
ermA, mecA, mefA, TEM, and Sp
ermA, mecA, mefA, vanA, and vanB
ermA, mecA, mefA, vanA, and Sp
ermA, mecA, mefA, vanB, and Sp
ermA, mecA, SHV, TEM, and vanA
ermA, mecA, SHV, TEM, and vanB
ermA, mecA, SHV, TEM, and Sp
ermA, mecA, SHV, vanA, and vanB
ermA, mecA, SHV, vanA, and Sp
ermA, mecA, SHV, vanB, and Sp
ermA, mecA, TEM, vanA, and vanB
ermA, mecA, TEM, vanA, and Sp
ermA, mecA, TEM, vanB, and Sp
ermA, mecA, vanA, vanB, and Sp
ermA, mefA, SHV, TEM, and vanA
ermA, mefA, SHV, TEM, and vanB
ermA, mefA, SHV, TEM, and Sp
ermA, mefA, SHV, vanA, and vanB
ermA, mefA, SHV, vanA, and Sp
ermA, mefA, SHV, vanB, and Sp
ermA, mefA, TEM, vanA, and vanB
ermA, mefA, TEM, vanA, and Sp
ermA, mefA, TEM, vanB, and Sp
ermA, mefA, vanA, vanB, and Sp
ermA, SHV, TEM, vanA, and vanB
ermA, SHV, TEM, vanA, and Sp
ermA, SHV, TEM, vanB, and Sp
ermA, SHV, vanA, vanB, and Sp
ermA, TEM, vanA, vanB, and Sp
ermB, mecA, mefA, SHV, and TEM
ermB, mecA, mefA, SHV, and vanA
ermB, mecA, mefA, SHV, and vanB
ermB, mecA, mefA, SHV, and Sp
ermB, mecA, mefA, TEM, and vanA
ermB, mecA, mefA, TEM, and vanB
ermB, mecA, mefA, TEM, and Sp
ermB, mecA, mefA, vanA, and vanB
ermB, mecA, mefA, vanA, and Sp
ermB, mecA, mefA, vanB, and Sp
ermB, mecA, SHV, TEM, and vanA
ermB, mecA, SHV, TEM, and vanB
ermB, mecA, SHV, TEM, and Sp
ermB, mecA, SHV, vanA, and vanB
ermB, mecA, SHV, vanA, and Sp
ermB, mecA, SHV, vanB, and Sp
ermB, mecA, TEM, vanA, and vanB
ermB, mecA, TEM, vanA, and Sp
ermB, mecA, TEM, vanB, and Sp
ermB, mecA, vanA, vanB, and Sp
ermB, mefA, SHV, TEM, and vanA
ermB, mefA, SHV, TEM, and vanB
ermB, mefA, SHV, TEM, and Sp
ermB, mefA, SHV, vanA, and vanB
ermB, mefA, SHV, vanA, and Sp
ermB, mefA, SHV, vanB, Sp
ermB, mefA, TEM, vanA, and vanB
ermB, mefA, TEM, vanA, and Sp
ermB, mefA, TEM, vanB, and Sp
ermB, mefA, vanA, vanB, and Sp
ermB, SHV, TEM, vanA, and vanB
ermB, SHV, TEM, vanA, and Sp
ermB, SHV, TEM, vanB, and Sp
ermB, SHV, vanA, vanB, and Sp
ermB, TEM, vanA, vanB, and Sp
mecA, mefA, SHV, TEM, and vanA
mecA, mefA, SHV, TEM, and vanB
mecA, mefA, SHV, TEM, and Sp
mecA, mefA, SHV, vanA, and vanB
mecA, mefA, SHV, vanA, and Sp
mecA, mefA, SHV, vanB, and Sp
mecA, mefA, TEM, vanA, and vanB
mecA, mefA, TEM, vanA, and Sp
mecA, mefA, TEM, vanB, and Sp
mecA, mefA, vanA, vanB, and Sp
mecA, SHV, TEM, vanA, and vanB
mecA, SHV, TEM, vanA, and Sp
mecA, SHV, TEM, vanB, and Sp
mecA, SHV, vanA, vanB, and Sp
mecA, TEM, vanA, vanB, and Sp
mefA, SHV, TEM, vanA, and vanB
mefA, SHV, TEM, vanA, and Sp
mefA, SHV, TEM, vanB, and Sp
mefA, SHV, vanA, vanB, and Sp
mefA, TEM, vanA, vanB, and Sp
SHV, TEM, vanA, vanB, and Sp

TABLE 11
Six-target combinations of Sp
(Streptococcus pneumoniae), CTX-M 14, CTX-M 15, ermA,
ermB, mecA, mefA, SHV, TEM, vanA, and vanB

CTX-M 14, CTX-M 15, ermA, ermB, mecA, and mefA
CTX-M 14, CTX-M 15, ermA, ermB, mecA, and SHV
CTX-M 14, CTX-M 15, ermA, ermB, mecA, and TEM
CTX-M 14, CTX-M 15, ermA, ermB, mecA, and vanA
CTX-M 14, CTX-M 15, ermA, ermB, mecA, and vanB
CTX-M 14, CTX-M 15, ermA, ermB, mecA, and Sp
CTX-M 14, CTX-M 15, ermA, ermB, mefA, and SHV
CTX-M 14, CTX-M 15, ermA, ermB, mefA, and TEM
CTX-M 14, CTX-M 15, ermA, ermB, mefA, and vanA
CTX-M 14, CTX-M 15, ermA, ermB, mefA, and vanB
CTX-M 14, CTX-M 15, ermA, ermB, mefA, and Sp
CTX-M 14, CTX-M 15, ermA, ermB, SHV, and TEM
CTX-M 14, CTX-M 15, ermA, ermB, SHV, and vanA
CTX-M 14, CTX-M 15, ermA, ermB, SHV, and vanB
CTX-M 14, CTX-M 15, ermA, ermB, SHV, and Sp
CTX-M 14, CTX-M 15, ermA, ermB, TEM, and vanA
CTX-M 14, CTX-M 15, ermA, ermB, TEM, and vanB
CTX-M 14, CTX-M 15, ermA, ermB, TEM, and Sp
CTX-M 14, CTX-M 15, ermA, ermB, vanA, and vanB
CTX-M 14, CTX-M 15, ermA, ermB, vanA, and Sp
CTX-M 14, CTX-M 15, ermA, ermB, vanB, and Sp
CTX-M 14, CTX-M 15, ermA, mecA, mefA, and SHV
CTX-M 14, CTX-M 15, ermA, mecA, mefA, and TEM
CTX-M 14, CTX-M 15, ermA, mecA, mefA, and vanA
CTX-M 14, CTX-M 15, ermA, mecA, mefA, and vanB
CTX-M 14, CTX-M 15, ermA, mecA, mefA, and Sp
CTX-M 14, CTX-M 15, ermA, mecA, SHV, and TEM
CTX-M 14, CTX-M 15, ermA, mecA, SHV, and vanA
CTX-M 14, CTX-M 15, ermA, mecA, SHV, and vanB
CTX-M 14, CTX-M 15, ermA, mecA, SHV, and Sp
CTX-M 14, CTX-M 15, ermA, mecA, TEM, and vanA
CTX-M 14, CTX-M 15, ermA, mecA, TEM, and vanB
CTX-M 14, CTX-M 15, ermA, mecA, TEM, and Sp
CTX-M 14, CTX-M 15, ermA, mecA, vanA, and vanB
CTX-M 14, CTX-M 15, ermA, mecA, vanA, and Sp
CTX-M 14, CTX-M 15, ermA, mecA, vanB, and Sp
CTX-M 14, CTX-M 15, ermA, mefA, SHV, and TEM
CTX-M 14, CTX-M 15, ermA, mefA, SHV, and vanA
CTX-M 14, CTX-M 15, ermA, mefA, SHV, and vanB
CTX-M 14, CTX-M 15, ermA, mefA, SHV, and Sp
CTX-M 14, CTX-M 15, ermA, mefA, TEM, and vanA
CTX-M 14, CTX-M 15, ermA, mefA, TEM, and vanB
CTX-M 14, CTX-M 15, ermA, mefA, TEM, and Sp
CTX-M 14, CTX-M 15, ermA, mefA, vanA, and vanB
CTX-M 14, CTX-M 15, ermA, mefA, vanA, and Sp
CTX-M 14, CTX-M 15, ermA, mefA, vanB, and Sp
CTX-M 14, CTX-M 15, ermA, SHV, TEM, and vanA
CTX-M 14, CTX-M 15, ermA, SHV, TEM, and vanB
CTX-M 14, CTX-M 15, ermA, SHV, TEM, and Sp
CTX-M 14, CTX-M 15, ermA, SHV, vanA, vanB
CTX-M 14, CTX-M 15, ermA, SHV, vanA, and Sp
CTX-M 14, CTX-M 15, ermA, SHV, vanB, and Sp
CTX-M 14, CTX-M 15, ermA, TEM, vanA, and vanB
CTX-M 14, CTX-M 15, ermA, TEM, vanA, and Sp
CTX-M 14, CTX-M 15, ermA, TEM, vanB, and Sp
CTX-M 14, CTX-M 15, ermA, vanA, vanB, and Sp
CTX-M 14, CTX-M 15, ermB, mecA, mefA and, SHV
CTX-M 14, CTX-M 15, ermB, mecA, mefA, and TEM
CTX-M 14, CTX-M 15, ermB, mecA, mefA, and vanA
CTX-M 14, CTX-M 15, ermB, mecA, mefA, and vanB
CTX-M 14, CTX-M 15, ermB, mecA, mefA, and Sp
CTX-M 14, CTX-M 15, ermB, mecA, SHV, and TEM
CTX-M 14, CTX-M 15, ermB, mecA, SHV, and vanA
CTX-M 14, CTX-M 15, ermB, mecA, SHV, and vanB
CTX-M 14, CTX-M 15, ermB, mecA, SHV, and Sp
CTX-M 14, CTX-M 15, ermB, mecA, TEM, and vanA
CTX-M 14, CTX-M 15, ermB, mecA, TEM, and vanB
CTX-M 14, CTX-M 15, ermB, mecA, TEM, and Sp
CTX-M 14, CTX-M 15, ermB, mecA, vanA, and vanB
CTX-M 14, CTX-M 15, ermB, mecA, vanA, and Sp
CTX-M 14, CTX-M 15, ermB, mecA, vanB, and Sp
CTX-M 14, CTX-M 15, ermB, mefA, SHV, and TEM
CTX-M 14, CTX-M 15, ermB, mefA, SHV, and vanA
CTX-M 14, CTX-M 15, ermB, mefA, SHV, and vanB
CTX-M 14, CTX-M 15, ermB, mefA, SHV, and Sp
CTX-M 14, CTX-M 15, ermB, mefA, TEM, and vanA
CTX-M 14, CTX-M 15, ermB, mefA, TEM, and vanB
CTX-M 14, CTX-M 15, ermB, mefA, TEM, and Sp
CTX-M 14, CTX-M 15, ermB, mefA, vanA, and vanB
CTX-M 14, CTX-M 15, ermB, mefA, vanA, and Sp
CTX-M 14, CTX-M 15, ermB, mefA, vanB, and Sp
CTX-M 14, CTX-M 15, ermB, SHV, TEM, and vanA
CTX-M 14, CTX-M 15, ermB, SHV, TEM, and vanB
CTX-M 14, CTX-M 15, ermB, SHV, TEM, and Sp
CTX-M 14, CTX-M 15, ermB, SHV, vanA, and vanB
CTX-M 14, CTX-M 15, ermB, SHV, vanA, and Sp
CTX-M 14, CTX-M 15, ermB, SHV, vanB, and Sp
CTX-M 14, CTX-M 15, ermB, TEM, vanA, and vanB
CTX-M 14, CTX-M 15, ermB, TEM, vanA, and Sp
CTX-M 14, CTX-M 15, ermB, TEM, vanB, and Sp
CTX-M 14, CTX-M 15, ermB, vanA, vanB, and Sp,
CTX-M 14, CTX-M 15, mecA, mefA, SHV, and TEM
CTX-M 14, CTX-M 15, mecA, mefA, SHV, and vanA
CTX-M 14, CTX-M 15, mecA, mefA, SHV, and vanB
CTX-M 14, CTX-M 15, mecA, mefA, SHV, and Sp
CTX-M 14, CTX-M 15, mecA, mefA, TEM, and vanA
CTX-M 14, CTX-M 15, mecA, mefA, TEM, and vanB
CTX-M 14, CTX-M 15, mecA, mefA, TEM, and Sp
CTX-M 14, CTX-M 15, mecA, mefA, vanA, and vanB
CTX-M 14, CTX-M 15, mecA, mefA, vanA, and Sp
CTX-M 14, CTX-M 15, mecA, mefA, vanB, and Sp
CTX-M 14, CTX-M 15, mecA, SHV, TEM, and vanA
CTX-M 14, CTX-M 15, mecA, SHV, TEM, and vanB
CTX-M 14, CTX-M 15, mecA, SHV, TEM, and Sp
CTX-M 14, CTX-M 15, mecA, SHV, vanA, and vanB
CTX-M 14, CTX-M 15, mecA, SHV, vanA, and Sp
CTX-M 14, CTX-M 15, mecA, SHV, vanB, and Sp
CTX-M 14, CTX-M 15, mecA, TEM, vanA, and vanB
CTX-M 14, CTX-M 15, mecA, TEM, vanA, and Sp
CTX-M 14, CTX-M 15, mecA, TEM, vanB, and Sp
CTX-M 14, CTX-M 15, mecA, vanA, vanB, and Sp
CTX-M 14, CTX-M 15, mefA, SHV, TEM, and vanA
CTX-M 14, CTX-M 15, mefA, SHV, TEM, and vanB
CTX-M 14, CTX-M 15, mefA, SHV, TEM, and Sp
CTX-M 14, CTX-M 15, mefA, SHV, vanA, and vanB
CTX-M 14, CTX-M 15, mefA, SHV, vanA, and Sp
CTX-M 14, CTX-M 15, mefA, SHV, vanB, and Sp
CTX-M 14, CTX-M 15, mefA, TEM, vanA, and vanB
CTX-M 14, CTX-M 15, mefA, TEM, vanA, and Sp
CTX-M 14, CTX-M 15, mefA, TEM, vanB, and Sp
CTX-M 14, CTX-M 15, mefA, vanA, vanB, and Sp
CTX-M 14, CTX-M 15, SHV, TEM, vanA, and vanB
CTX-M 14, CTX-M 15, SHV, TEM, vanA, and Sp
CTX-M 14, CTX-M 15, SHV, TEM, vanB, and Sp
CTX-M 14, CTX-M 15, SHV, vanA, vanB, and Sp
CTX-M 14, CTX-M 15, TEM, vanA, vanB, and Sp
CTX-M 14, ermA, ermB, mecA, mefA, and SHV
CTX-M 14, ermA, ermB, mecA, mefA, and TEM
CTX-M 14, ermA, ermB, mecA, mefA, and vanA
CTX-M 14, ermA, ermB, mecA, mefA, and vanB
CTX-M 14, ermA, ermB, mecA, mefA, and Sp
CTX-M 14, ermA, ermB, mecA, SHV, and TEM
CTX-M 14, ermA, ermB, mecA, SHV, and vanA
CTX-M 14, ermA, ermB, mecA, SHV, and vanB
CTX-M 14, ermA, ermB, mecA, SHV, and Sp
CTX-M 14, ermA, ermB, mecA, TEM, and vanA
CTX-M 14, ermA, ermB, mecA, TEM, and vanB
CTX-M 14, ermA, ermB, mecA, TEM, and Sp
CTX-M 14, ermA, ermB, mecA, vanA, and vanB
CTX-M 14, ermA, ermB, mecA, vanA, and Sp
CTX-M 14, ermA, ermB, mecA, vanB, and Sp
CTX-M 14, ermA, ermB, mefA, SHV, and TEM
CTX-M 14, ermA, ermB, mefA, SHV, and vanA
CTX-M 14, ermA, ermB, mefA, SHV, and vanB
CTX-M 14, ermA, ermB, mefA, SHV, and Sp
CTX-M 14, ermA, ermB, mefA, TEM, and vanA
CTX-M 14, ermA, ermB, mefA, TEM, and vanB
CTX-M 14, ermA, ermB, mefA, TEM, and Sp
CTX-M 14, ermA, ermB, mefA, vanA, and vanB
CTX-M 14, ermA, ermB, mefA, vanA, and Sp
CTX-M 14, ermA, ermB, mefA, vanB, and Sp
CTX-M 14, ermA, ermB, SHV, TEM, and vanA
CTX-M 14, ermA, ermB, SHV, TEM, and vanB
CTX-M 14, ermA, ermB, SHV, TEM, and Sp
CTX-M 14, ermA, ermB, SHV, vanA, and vanB
CTX-M 14, ermA, ermB, SHV, vanA, and Sp
CTX-M 14, ermA, ermB, SHV, vanB, and Sp
CTX-M 14, ermA, ermB, TEM, vanA, and vanB
CTX-M 14, ermA, ermB, TEM, vanA, and Sp
CTX-M 14, ermA, ermB, TEM, vanB, and Sp
CTX-M 14, ermA, ermB, vanA, vanB, and Sp
CTX-M 14, ermA, mecA, mefA, SHV, and TEM
CTX-M 14, ermA, mecA, mefA, SHV, and vanA
CTX-M 14, ermA, mecA, mefA, SHV, and vanB
CTX-M 14, ermA, mecA, mefA, SHV, and Sp
CTX-M 14, ermA, mecA, mefA, TEM, and vanA
CTX-M 14, ermA, mecA, mefA, TEM, and vanB
CTX-M 14, ermA, mecA, mefA, TEM, and Sp
CTX-M 14, ermA, mecA, mefA, vanA, and vanB
CTX-M 14, ermA, mecA, mefA, vanA, and Sp
CTX-M 14, ermA, mecA, mefA, vanB, and Sp
CTX-M 14, ermA, mecA, SHV, TEM, and vanA
CTX-M 14, ermA, mecA, SHV, TEM, and vanB
CTX-M 14, ermA, mecA, SHV, TEM, and Sp
CTX-M 14, ermA, mecA, SHV, vanA, and vanB
CTX-M 14, ermA, mecA, SHV, vanA, and Sp
CTX-M 14, ermA, mecA, SHV, vanB, and Sp
CTX-M 14, ermA, mecA, TEM, vanA, and vanB
CTX-M 14, ermA, mecA, TEM, vanA, and Sp
CTX-M 14, ermA, mecA, TEM, vanB, and Sp
CTX-M 14, ermA, mecA, vanA, vanB, and Sp
CTX-M 14, ermA, mefA, SHV, TEM, and vanA
CTX-M 14, ermA, mefA, SHV, TEM, and vanB
CTX-M 14, ermA, mefA, SHV, TEM, Sp
CTX-M 14, ermA, mefA, SHV, vanA, and vanB
CTX-M 14, ermA, mefA, SHV, vanA, and Sp
CTX-M 14, ermA, mefA, SHV, vanB, and Sp
CTX-M 14, ermA, mefA, TEM, vanA, and vanB
CTX-M 14, ermA, mefA, TEM, vanA, and Sp
CTX-M 14, ermA, mefA, TEM, vanB, and Sp
CTX-M 14, ermA, mefA, vanA, vanB, and Sp
CTX-M 14, ermA, SHV, TEM, vanA, and vanB
CTX-M 14, ermA, SHV, TEM, vanA, and Sp
CTX-M 14, ermA, SHV, TEM, vanB, and Sp
CTX-M 14, ermA, SHV, vanA, vanB, and Sp
CTX-M 14, ermA, TEM, vanA, vanB, and Sp
CTX-M 14, ermB, mecA, mefA, SHV, and TEM
CTX-M 14, ermB, mecA, mefA, SHV, and vanA
CTX-M 14, ermB, mecA, mefA, SHV, and vanB
CTX-M 14, ermB, mecA, mefA, SHV, and Sp
CTX-M 14, ermB, mecA, mefA, TEM, and vanA
CTX-M 14, ermB, mecA, mefA, TEM, and vanB
CTX-M 14, ermB, mecA, mefA, TEM, and Sp
CTX-M 14, ermB, mecA, mefA, vanA, and vanB
CTX-M 14, ermB, mecA, mefA, vanA, and Sp
CTX-M 14, ermB, mecA, mefA, vanB, and Sp
CTX-M 14, ermB, mecA, SHV, TEM, and vanA
CTX-M 14, ermB, mecA, SHV, TEM, and vanB
CTX-M 14, ermB, mecA, SHV, TEM, and Sp
CTX-M 14, ermB, mecA, SHV, vanA, and vanB
CTX-M 14, ermB, mecA, SHV, vanA, and Sp
CTX-M 14, ermB, mecA, SHV, vanB, and Sp
CTX-M 14, ermB, mecA, TEM, vanA, and vanB
CTX-M 14, ermB, mecA, TEM, vanA, and Sp
CTX-M 14, ermB, mecA, TEM, vanB, and Sp
CTX-M 14, ermB, mecA, vanA, vanB, and Sp
CTX-M 14, ermB, mefA, SHV, TEM, and vanA
CTX-M 14, ermB, mefA, SHV, TEM, and vanB
CTX-M 14, ermB, mefA, SHV, TEM, and Sp
CTX-M 14, ermB, mefA, SHV, vanA, and vanB
CTX-M 14, ermB, mefA, SHV, vanA, and Sp
CTX-M 14, ermB, mefA, SHV, vanB, and Sp
CTX-M 14, ermB, mefA, TEM, vanA, and vanB
CTX-M 14, ermB, mefA, TEM, vanA, and Sp
CTX-M 14, ermB, mefA, TEM, vanB, and Sp
CTX-M 14, ermB, mefA, vanA, vanB, and Sp
CTX-M 14, ermB, SHV, TEM, vanA, and vanB
CTX-M 14, ermB, SHV, TEM, vanA, and Sp
CTX-M 14, ermB, SHV, TEM, vanB, and Sp
CTX-M 14, ermB, SHV, vanA, vanB, and Sp
CTX-M 14, ermB, TEM, vanA, vanB, and Sp
CTX-M 14, mecA, mefA, SHV, TEM, and vanA
CTX-M 14, mecA, mefA, SHV, TEM, and vanB
CTX-M 14, mecA, mefA, SHV, TEM, and Sp
CTX-M 14, mecA, mefA, SHV, vanA, and vanB
CTX-M 14, mecA, mefA, SHV, vanA, and Sp
CTX-M 14, mecA, mefA, SHV, vanB, and Sp
CTX-M 14, mecA, mefA, TEM, vanA, and vanB
CTX-M 14, mecA, mefA, TEM, vanA, and Sp
CTX-M 14, mecA, mefA, TEM, vanB, and Sp
CTX-M 14, mecA, mefA, vanA, vanB, and Sp
CTX-M 14, mecA, SHV, TEM, vanA, and vanB
CTX-M 14, mecA, SHV, TEM, vanA, and Sp
CTX-M 14, mecA, SHV, TEM, vanB, and Sp
CTX-M 14, mecA, SHV, vanA, vanB, and Sp
CTX-M 14, mecA, TEM, vanA, vanB, and Sp
CTX-M 14, mefA, SHV, TEM, vanA, and vanB
CTX-M 14, mefA, SHV, TEM, vanA, and Sp
CTX-M 14, mefA, SHV, TEM, vanB, and Sp
CTX-M 14, mefA, SHV, vanA, vanB, and Sp
CTX-M 14, mefA, TEM, vanA, vanB, and Sp
CTX-M 14, SHV, TEM, vanA, vanB, and Sp
CTX-M 15, ermA, ermB, mecA, mefA, and SHV
CTX-M 15, ermA, ermB, mecA, mefA, and TEM
CTX-M 15, ermA, ermB, mecA, mefA, and vanA
CTX-M 15, ermA, ermB, mecA, mefA, and vanB
CTX-M 15, ermA, ermB, mecA, mefA, and Sp
CTX-M 15, ermA, ermB, mecA, SHV, and TEM
CTX-M 15, ermA, ermB, mecA, SHV, and vanA
CTX-M 15, ermA, ermB, mecA, SHV, and vanB
CTX-M 15, ermA, ermB, mecA, SHV, and Sp
CTX-M 15, ermA, ermB, mecA, TEM, and vanA
CTX-M 15, ermA, ermB, mecA, TEM, and vanB
CTX-M 15, ermA, ermB, mecA, TEM, and Sp
CTX-M 15, ermA, ermB, mecA, vanA, and vanB
CTX-M 15, ermA, ermB, mecA, vanA, and Sp
CTX-M 15, ermA, ermB, mecA, vanB, and Sp
CTX-M 15, ermA, ermB, mefA, SHV, and TEM
CTX-M 15, ermA, ermB, mefA, SHV, and vanA
CTX-M 15, ermA, ermB, mefA, SHV, and vanB
CTX-M 15, ermA, ermB, mefA, SHV, and Sp
CTX-M 15, ermA, ermB, mefA, TEM, and vanA
CTX-M 15, ermA, ermB, mefA, TEM, and vanB
CTX-M 15, ermA, ermB, mefA, TEM, and Sp
CTX-M 15, ermA, ermB, mefA, vanA, and vanB
CTX-M 15, ermA, ermB, mefA, vanA, and Sp
CTX-M 15, ermA, ermB, mefA, vanB, and Sp
CTX-M 15, ermA, ermB, SHV, TEM, and vanA
CTX-M 15, ermA, ermB, SHV, TEM, and vanB
CTX-M 15, ermA, ermB, SHV, TEM, and Sp
CTX-M 15, ermA, ermB, SHV, vanA, and vanB
CTX-M 15, ermA, ermB, SHV, vanA, and Sp
CTX-M 15, ermA, ermB, SHV, vanB, and Sp
CTX-M 15, ermA, ermB, TEM, vanA, and vanB
CTX-M 15, ermA, ermB, TEM, vanA, and Sp
CTX-M 15, ermA, ermB, TEM, vanB, and Sp
CTX-M 15, ermA, ermB, vanA, vanB, and Sp
CTX-M 15, ermA, mecA, mefA, SHV, and TEM
CTX-M 15, ermA, mecA, mefA, SHV, and vanA
CTX-M 15, ermA, mecA, mefA, SHV, and vanB
CTX-M 15, ermA, mecA, mefA, SHV, and Sp
CTX-M 15, ermA, mecA, mefA, TEM, and vanA
CTX-M 15, ermA, mecA, mefA, TEM, and vanB
CTX-M 15, ermA, mecA, mefA, TEM, and Sp
CTX-M 15, ermA, mecA, mefA, vanA, and vanB
CTX-M 15, ermA, mecA, mefA, vanA, and Sp
CTX-M 15, ermA, mecA, mefA, vanB, and Sp
CTX-M 15, ermA, mecA, SHV, TEM, and vanA
CTX-M 15, ermA, mecA, SHV, TEM, and vanB
CTX-M 15, ermA, mecA, SHV, TEM, and Sp
CTX-M 15, ermA, mecA, SHV, vanA, and vanB
CTX-M 15, ermA, mecA, SHV, vanA, and Sp
CTX-M 15, ermA, mecA, SHV, vanB, and Sp
CTX-M 15, ermA, mecA, TEM, vanA, and vanB
CTX-M 15, ermA, mecA, TEM, vanA, and Sp
CTX-M 15, ermA, mecA, TEM, vanB, and Sp
CTX-M 15, ermA, mecA, vanA, vanB, and Sp
CTX-M 15, ermA, mefA, SHV, TEM, and vanA
CTX-M 15, ermA, mefA, SHV, TEM, and vanB
CTX-M 15, ermA, mefA, SHV, TEM, and Sp
CTX-M 15, ermA, mefA, SHV, vanA, and vanB
CTX-M 15, ermA, mefA, SHV, vanA, and Sp
CTX-M 15, ermA, mefA, SHV, vanB, and Sp
CTX-M 15, ermA, mefA, TEM, vanA, and vanB
CTX-M 15, ermA, mefA, TEM, vanA, and Sp
CTX-M 15, ermA, mefA, TEM, vanB, and Sp
CTX-M 15, ermA, mefA, vanA, vanB, and Sp
CTX-M 15, ermA, SHV, TEM, vanA, and vanB
CTX-M 15, ermA, SHV, TEM, vanA, and Sp
CTX-M 15, ermA, SHV, TEM, vanB, and Sp
CTX-M 15, ermA, SHV, vanA, vanB, and Sp
CTX-M 15, ermA, TEM, vanA, vanB, and Sp
CTX-M 15, ermB, mecA, mefA, SHV, and TEM
CTX-M 15, ermB, mecA, mefA, SHV, and vanA
CTX-M 15, ermB, mecA, mefA, SHV, and vanB
CTX-M 15, ermB, mecA, mefA, SHV, and Sp
CTX-M 15, ermB, mecA, mefA, TEM, and vanA
CTX-M 15, ermB, mecA, mefA, TEM, and vanB
CTX-M 15, ermB, mecA, mefA, TEM, and Sp
CTX-M 15, ermB, mecA, mefA, vanA, and vanB
CTX-M 15, ermB, mecA, mefA, vanA, and Sp
CTX-M 15, ermB, mecA, mefA, vanB, and Sp
CTX-M 15, ermB, mecA, SHV, TEM, and vanA
CTX-M 15, ermB, mecA, SHV, TEM, and vanB
CTX-M 15, ermB, mecA, SHV, TEM, and Sp
CTX-M 15, ermB, mecA, SHV, vanA, and vanB
CTX-M 15, ermB, mecA, SHV, vanA, and Sp
CTX-M 15, ermB, mecA, SHV, vanB, and Sp
CTX-M 15, ermB, mecA, TEM, vanA, and vanB
CTX-M 15, ermB, mecA, TEM, vanA, and Sp
CTX-M 15, ermB, mecA, TEM, vanB, and Sp
CTX-M 15, ermB, mecA, vanA, vanB, and Sp
CTX-M 15, ermB, mefA, SHV, TEM, and vanA
CTX-M 15, ermB, mefA, SHV, TEM, and vanB
CTX-M 15, ermB, mefA, SHV, TEM, and Sp
CTX-M 15, ermB, mefA, SHV, vanA, and vanB
CTX-M 15, ermB, mefA, SHV, vanA, and Sp
CTX-M 15, ermB, mefA, SHV, vanB, and Sp
CTX-M 15, ermB, mefA, TEM, vanA, and vanB
CTX-M 15, ermB, mefA, TEM, vanA, and Sp
CTX-M 15, ermB, mefA, TEM, vanB, and Sp
CTX-M 15, ermB, mefA, vanA, vanB, and Sp
CTX-M 15, ermB, SHV, TEM, vanA, and vanB
CTX-M 15, ermB, SHV, TEM, vanA, and Sp
CTX-M 15, ermB, SHV, TEM, vanB, and Sp
CTX-M 15, ermB, SHV, vanA, vanB, and Sp
CTX-M 15, ermB, TEM, vanA, vanB, and Sp
CTX-M 15, mecA, mefA, SHV, TEM, and vanA
CTX-M 15, mecA, mefA, SHV, TEM, and vanB
CTX-M 15, mecA, mefA, SHV, TEM, and Sp
CTX-M 15, mecA, mefA, SHV, vanA, and vanB
CTX-M 15, mecA, mefA, SHV, vanA, and Sp
CTX-M 15, mecA, mefA, SHV, vanB, and Sp
CTX-M 15, mecA, mefA, TEM, vanA, and vanB
CTX-M 15, mecA, mefA, TEM, vanA, and Sp
CTX-M 15, mecA, mefA, TEM, vanB, and Sp
CTX-M 15, mecA, mefA, vanA, vanB, and Sp
CTX-M 15, mecA, SHV, TEM, vanA, and vanB
CTX-M 15, mecA, SHV, TEM, vanA, and Sp
CTX-M 15, mecA, SHV, TEM, vanB, and Sp
CTX-M 15, mecA, SHV, vanA, vanB, and Sp
CTX-M 15, mecA, TEM, vanA, vanB, and Sp
CTX-M 15, mefA, SHV, TEM, vanA, and vanB
CTX-M 15, mefA, SHV, TEM, vanA, and Sp
CTX-M 15, mefA, SHV, TEM, vanB, and Sp
CTX-M 15, mefA, SHV, vanA, vanB, and Sp
CTX-M 15, mefA, TEM, vanA, vanB, and Sp
CTX-M 15, SHV, TEM, vanA, vanB, and Sp
ermA, ermB, mecA, mefA, SHV, and TEM
ermA, ermB, mecA, mefA, SHV, and vanA
ermA, ermB, mecA, mefA, SHV, and vanB
ermA, ermB, mecA, mefA, SHV, and Sp
ermA, ermB, mecA, mefA, TEM, and vanA
ermA, ermB, mecA, mefA, TEM, and vanB
ermA, ermB, mecA, mefA, TEM, and Sp
ermA, ermB, mecA, mefA, vanA, and vanB
ermA, ermB, mecA, mefA, vanA, and Sp
ermA, ermB, mecA, mefA, vanB, and Sp
ermA, ermB, mecA, SHV, TEM, and vanA
ermA, ermB, mecA, SHV, TEM, and vanB
ermA, ermB, mecA, SHV, TEM, and Sp
ermA, ermB, mecA, SHV, vanA, and vanB
ermA, ermB, mecA, SHV, vanA, and Sp
ermA, ermB, mecA, SHV, vanB, and Sp
ermA, ermB, mecA, TEM, vanA, and vanB
ermA, ermB, mecA, TEM, vanA, and Sp
ermA, ermB, mecA, TEM, vanB, and Sp
ermA, ermB, mecA, vanA, vanB, and Sp
ermA, ermB, mefA, SHV, TEM, and vanA
ermA, ermB, mefA, SHV, TEM, and vanB
ermA, ermB, mefA, SHV, TEM, and Sp
ermA, ermB, mefA, SHV, vanA, and vanB
ermA, ermB, mefA, SHV, vanA, and Sp
ermA, ermB, mefA, SHV, vanB, and Sp
ermA, ermB, mefA, TEM, vanA, and vanB
ermA, ermB, mefA, TEM, vanA, and Sp
ermA, ermB, mefA, TEM, vanB, and Sp
ermA, ermB, mefA, vanA, vanB, and Sp
ermA, ermB, SHV, TEM, vanA, and vanB
ermA, ermB, SHV, TEM, vanA, and Sp
ermA, ermB, SHV, TEM, vanB, and Sp
ermA, ermB, SHV, vanA, vanB, and Sp
ermA, ermB, TEM, vanA, vanB, and Sp
ermA, mecA, mefA, SHV, TEM, and vanA
ermA, mecA, mefA, SHV, TEM, and vanB
ermA, mecA, mefA, SHV, TEM, and Sp
ermA, mecA, mefA, SHV, vanA, and vanB
ermA, mecA, mefA, SHV, vanA, and Sp
ermA, mecA, mefA, SHV, vanB, and Sp
ermA, mecA, mefA, TEM, vanA, and vanB
ermA, mecA, mefA, TEM, vanA, and Sp
ermA, mecA, mefA, TEM, vanB, and Sp
ermA, mecA, mefA, vanA, vanB, and Sp
ermA, mecA, SHV, TEM, vanA, and vanB
ermA, mecA, SHV, TEM, vanA, and Sp
ermA, mecA, SHV, TEM, vanB, and Sp
ermA, mecA, SHV, vanA, vanB, and Sp
ermA, mecA, TEM, vanA, vanB, and Sp
ermA, mefA, SHV, TEM, vanA, and vanB
ermA, mefA, SHV, TEM, vanA, and Sp
ermA, mefA, SHV, TEM, vanB, and Sp
ermA, mefA, SHV, vanA, vanB, and Sp
ermA, mefA, TEM, vanA, vanB, and Sp
ermA, SHV, TEM, vanA, vanB, and Sp
ermB, mecA, mefA, SHV, TEM, and vanA
ermB, mecA, mefA, SHV, TEM, and vanB
ermB, mecA, mefA, SHV, TEM, and Sp
ermB, mecA, mefA, SHV, vanA, and vanB
ermB, mecA, mefA, SHV, vanA, and Sp
ermB, mecA, mefA, SHV, vanB, and Sp
ermB, mecA, mefA, TEM, vanA, and vanB
ermB, mecA, mefA, TEM, vanA, and Sp
ermB, mecA, mefA, TEM, vanB, and Sp
ermB, mecA, mefA, vanA, vanB, and Sp
ermB, mecA, SHV, TEM, vanA, and vanB
ermB, mecA, SHV, TEM, vanA, and Sp
ermB, mecA, SHV, TEM, vanB, and Sp
ermB, mecA, SHV, vanA, vanB, and Sp
ermB, mecA, TEM, vanA, vanB, and Sp
ermB, mefA, SHV, TEM, vanA, and vanB
ermB, mefA, SHV, TEM, vanA, and Sp
ermB, mefA, SHV, TEM, vanB, and Sp
ermB, mefA, SHV, vanA, vanB, and Sp
ermB, mefA, TEM, vanA, vanB, and Sp
ermB, SHV, TEM, vanA, vanB, and Sp
mecA, mefA, SHV, TEM, vanA, and vanB
mecA, mefA, SHV, TEM, vanA, and Sp
mecA, mefA, SHV, TEM, vanB, and Sp
mecA, mefA, SHV, vanA, vanB, and Sp
mecA, mefA, TEM, vanA, vanB, and Sp
mecA, SHV, TEM, vanA, vanB, and Sp
mefA, SHV, TEM, vanA, vanB, and Sp

TABLE 12
Seven-target combinations of Sp
(Streptococcus pneumoniae), CTX-M 14, CTX-M 15, ermA,
ermB, mecA, mefA, SHV, TEM, vanA, and vanB

CTX-M 14, CTX-M 15, ermA, ermB, mecA, mefA, and SHV
CTX-M 14, CTX-M 15, ermA, ermB, mecA, mefA, and TEM
CTX-M 14, CTX-M 15, ermA, ermB, mecA, mefA, and vanA
CTX-M 14, CTX-M 15, ermA, ermB, mecA, mefA, and vanB
CTX-M 14, CTX-M 15, ermA, ermB, mecA, mefA, and Sp
CTX-M 14, CTX-M 15, ermA, ermB, mecA, SHV, and TEM
CTX-M 14, CTX-M 15, ermA, ermB, mecA, SHV, and vanA
CTX-M 14, CTX-M 15, ermA, ermB, mecA, SHV, and vanB
CTX-M 14, CTX-M 15, ermA, ermB, mecA, SHV, and Sp
CTX-M 14, CTX-M 15, ermA, ermB, mecA, TEM, and vanA
CTX-M 14, CTX-M 15, ermA, ermB, mecA, TEM, and vanB
CTX-M 14, CTX-M 15, ermA, ermB, mecA, TEM, and Sp
CTX-M 14, CTX-M 15, ermA, ermB, mecA, vanA, and vanB
CTX-M 14, CTX-M 15, ermA, ermB, mecA, vanA, and Sp
CTX-M 14, CTX-M 15, ermA, ermB, mecA, vanB, and Sp
CTX-M 14, CTX-M 15, ermA, ermB, mefA, SHV, and TEM
CTX-M 14, CTX-M 15, ermA, ermB, mefA, SHV, and vanA
CTX-M 14, CTX-M 15, ermA, ermB, mefA, SHV, and vanB
CTX-M 14, CTX-M 15, ermA, ermB, mefA, SHV, and Sp
CTX-M 14, CTX-M 15, ermA, ermB, mefA, TEM, and vanA
CTX-M 14, CTX-M 15, ermA, ermB, mefA, TEM, and vanB
CTX-M 14, CTX-M 15, ermA, ermB, mefA, TEM, and Sp
CTX-M 14, CTX-M 15, ermA, ermB, mefA, vanA, vanB
CTX-M 14, CTX-M 15, ermA, ermB, mefA, vanA, and Sp
CTX-M 14, CTX-M 15, ermA, ermB, mefA, vanB and, Sp
CTX-M 14, CTX-M 15, ermA, ermB, SHV, TEM, and vanA
CTX-M 14, CTX-M 15, ermA, ermB, SHV, TEM, and vanB
CTX-M 14, CTX-M 15, ermA, ermB, SHV, TEM, and Sp
CTX-M 14, CTX-M 15, ermA, ermB, SHV, vanA, and vanB
CTX-M 14, CTX-M 15, ermA, ermB, SHV, vanA, and Sp
CTX-M 14, CTX-M 15, ermA, ermB, SHV, vanB, and Sp
CTX-M 14, CTX-M 15, ermA, ermB, TEM, vanA, and vanB
CTX-M 14, CTX-M 15, ermA, ermB, TEM, vanA, and Sp
CTX-M 14, CTX-M 15, ermA, ermB, TEM, vanB, and Sp
CTX-M 14, CTX-M 15, ermA, ermB, vanA, vanB, and Sp
CTX-M 14, CTX-M 15, ermA, mecA, mefA, SHV, and TEM
CTX-M 14, CTX-M 15, ermA, mecA, mefA, SHV, and vanA
CTX-M 14, CTX-M 15, ermA, mecA, mefA, SHV, and vanB
CTX-M 14, CTX-M 15, ermA, mecA, mefA, SHV, and Sp
CTX-M 14, CTX-M 15, ermA, mecA, mefA, TEM, and vanA
CTX-M 14, CTX-M 15, ermA, mecA, mefA, TEM, and vanB
CTX-M 14, CTX-M 15, ermA, mecA, mefA, TEM, and Sp
CTX-M 14, CTX-M 15, ermA, mecA, mefA, vanA, and vanB
CTX-M 14, CTX-M 15, ermA, mecA, mefA, vanA, and Sp
CTX-M 14, CTX-M 15, ermA, mecA, mefA, vanB, and Sp
CTX-M 14, CTX-M 15, ermA, mecA, SHV, TEM, and vanA
CTX-M 14, CTX-M 15, ermA, mecA, SHV, TEM, and vanB
CTX-M 14, CTX-M 15, ermA, mecA, SHV, TEM, and Sp
CTX-M 14, CTX-M 15, ermA, mecA, SHV, vanA, and vanB
CTX-M 14, CTX-M 15, ermA, mecA, SHV, vanA, and Sp
CTX-M 14, CTX-M 15, ermA, mecA, SHV, vanB, and Sp
CTX-M 14, CTX-M 15, ermA, mecA, TEM, vanA, and vanB
CTX-M 14, CTX-M 15, ermA, mecA, TEM, vanA, and Sp
CTX-M 14, CTX-M 15, ermA, mecA, TEM, vanB, and Sp
CTX-M 14, CTX-M 15, ermA, mecA, vanA, vanB, and Sp
CTX-M 14, CTX-M 15, ermA, mefA, SHV, TEM, and vanA
CTX-M 14, CTX-M 15, ermA, mefA, SHV, TEM, and vanB
CTX-M 14, CTX-M 15, ermA, mefA, SHV, TEM, and Sp
CTX-M 14, CTX-M 15, ermA, mefA, SHV, vanA, and vanB
CTX-M 14, CTX-M 15, ermA, mefA, SHV, vanA, and Sp
CTX-M 14, CTX-M 15, ermA, mefA, SHV, vanB, and Sp
CTX-M 14, CTX-M 15, ermA, mefA, TEM, vanA, and vanB
CTX-M 14, CTX-M 15, ermA, mefA, TEM, vanA, and Sp
CTX-M 14, CTX-M 15, ermA, mefA, TEM, vanB, and Sp
CTX-M 14, CTX-M 15, ermA, mefA, vanA, vanB, and Sp
CTX-M 14, CTX-M 15, ermA, SHV, TEM, vanA, and vanB
CTX-M 14, CTX-M 15, ermA, SHV, TEM, vanA, and Sp
CTX-M 14, CTX-M 15, ermA, SHV, TEM, vanB, and Sp
CTX-M 14, CTX-M 15, ermA, SHV, vanA, vanB, and Sp
CTX-M 14, CTX-M 15, ermA, TEM, vanA, vanB, and Sp
CTX-M 14, CTX-M 15, ermB, mecA, mefA, SHV, and TEM
CTX-M 14, CTX-M 15, ermB, mecA, mefA, SHV, and vanA
CTX-M 14, CTX-M 15, ermB, mecA, mefA, SHV, and vanB
CTX-M 14, CTX-M 15, ermB, mecA, mefA, SHV, and Sp
CTX-M 14, CTX-M 15, ermB, mecA, mefA, TEM, and vanA
CTX-M 14, CTX-M 15, ermB, mecA, mefA, TEM, and vanB
CTX-M 14, CTX-M 15, ermB, mecA, mefA, TEM, and Sp
CTX-M 14, CTX-M 15, ermB, mecA, mefA, vanA, and vanB
CTX-M 14, CTX-M 15, ermB, mecA, mefA, vanA, and Sp
CTX-M 14, CTX-M 15, ermB, mecA, mefA, vanB, and Sp
CTX-M 14, CTX-M 15, ermB, mecA, SHV, TEM, and vanA
CTX-M 14, CTX-M 15, ermB, mecA, SHV, TEM, and vanB
CTX-M 14, CTX-M 15, ermB, mecA, SHV, TEM, and Sp
CTX-M 14, CTX-M 15, ermB, mecA, SHV, vanA, and vanB
CTX-M 14, CTX-M 15, ermB, mecA, SHV, vanA, and Sp
CTX-M 14, CTX-M 15, ermB, mecA, SHV, vanB, and Sp
CTX-M 14, CTX-M 15, ermB, mecA, TEM, vanA, and vanB
CTX-M 14, CTX-M 15, ermB, mecA, TEM, vanA, and Sp
CTX-M 14, CTX-M 15, ermB, mecA, TEM, vanB, and Sp
CTX-M 14, CTX-M 15, ermB, mecA, vanA, vanB, and Sp
CTX-M 14, CTX-M 15, ermB, mefA, SHV, TEM, and vanA
CTX-M 14, CTX-M 15, ermB, mefA, SHV, TEM, and vanB
CTX-M 14, CTX-M 15, ermB, mefA, SHV, TEM, and Sp
CTX-M 14, CTX-M 15, ermB, mefA, SHV, vanA, and vanB
CTX-M 14, CTX-M 15, ermB, mefA, SHV, vanA, and Sp
CTX-M 14, CTX-M 15, ermB, mefA, SHV, vanB, Sp
CTX-M 14, CTX-M 15, ermB, mefA, TEM, vanA, and vanB
CTX-M 14, CTX-M 15, ermB, mefA, TEM, vanA, and Sp
CTX-M 14, CTX-M 15, ermB, mefA, TEM, vanB, and Sp
CTX-M 14, CTX-M 15, ermB, mefA, vanA, vanB, and Sp
CTX-M 14, CTX-M 15, ermB, SHV, TEM, vanA, and vanB
CTX-M 14, CTX-M 15, ermB, SHV, TEM, vanA, and Sp
CTX-M 14, CTX-M 15, ermB, SHV, TEM, vanB, and Sp
CTX-M 14, CTX-M 15, ermB, SHV, vanA, vanB, and Sp
CTX-M 14, CTX-M 15, ermB, TEM, vanA, vanB, and Sp
CTX-M 14, CTX-M 15, mecA, mefA, SHV, TEM, and vanA
CTX-M 14, CTX-M 15, mecA, mefA, SHV, TEM, and vanB
CTX-M 14, CTX-M 15, mecA, mefA, SHV, TEM, and Sp
CTX-M 14, CTX-M 15, mecA, mefA, SHV, vanA, and vanB
CTX-M 14, CTX-M 15, mecA, mefA, SHV, vanA, and Sp
CTX-M 14, CTX-M 15, mecA, mefA, SHV, vanB, and Sp
CTX-M 14, CTX-M 15, mecA, mefA, TEM, vanA, and vanB
CTX-M 14, CTX-M 15, mecA, mefA, TEM, vanA, and Sp
CTX-M 14, CTX-M 15, mecA, mefA, TEM, vanB, and Sp
CTX-M 14, CTX-M 15, mecA, mefA, vanA, vanB, and Sp
CTX-M 14, CTX-M 15, mecA, SHV, TEM, vanA, and vanB
CTX-M 14, CTX-M 15, mecA, SHV, TEM, vanA, and Sp
CTX-M 14, CTX-M 15, mecA, SHV, TEM, vanB, and Sp
CTX-M 14, CTX-M 15, mecA, SHV, vanA, vanB, and Sp
CTX-M 14, CTX-M 15, mecA, TEM, vanA, vanB, and Sp
CTX-M 14, CTX-M 15, mefA, SHV, TEM, vanA, and vanB
CTX-M 14, CTX-M 15, mefA, SHV, TEM, vanA, and Sp
CTX-M 14, CTX-M 15, mefA, SHV, TEM, vanB, and Sp
CTX-M 14, CTX-M 15, mefA, SHV, vanA, vanB, and Sp
CTX-M 14, CTX-M 15, mefA, TEM, vanA, vanB, and Sp
CTX-M 14, CTX-M 15, SHV, TEM, vanA, vanB, and Sp
CTX-M 14, ermA, ermB, mecA, mefA, SHV, and TEM
CTX-M 14, ermA, ermB, mecA, mefA, SHV, and vanA
CTX-M 14, ermA, ermB, mecA, mefA, SHV, and vanB
CTX-M 14, ermA, ermB, mecA, mefA, SHV, and Sp
CTX-M 14, ermA, ermB, mecA, mefA, TEM, and vanA
CTX-M 14, ermA, ermB, mecA, mefA, TEM, and vanB
CTX-M 14, ermA, ermB, mecA, mefA, TEM, and Sp
CTX-M 14, ermA, ermB, mecA, mefA, vanA, and vanB
CTX-M 14, ermA, ermB, mecA, mefA, vanA, and Sp
CTX-M 14, ermA, ermB, mecA, mefA, vanB, and Sp
CTX-M 14, ermA, ermB, mecA, SHV, TEM, and vanA
CTX-M 14, ermA, ermB, mecA, SHV, TEM, and vanB
CTX-M 14, ermA, ermB, mecA, SHV, TEM, and Sp
CTX-M 14, ermA, ermB, mecA, SHV, vanA, and vanB
CTX-M 14, ermA, ermB, mecA, SHV, vanA, and Sp
CTX-M 14, ermA, ermB, mecA, SHV, vanB, and Sp
CTX-M 14, ermA, ermB, mecA, TEM, vanA, and vanB
CTX-M 14, ermA, ermB, mecA, TEM, vanA, and Sp
CTX-M 14, ermA, ermB, mecA, TEM, vanB, and Sp
CTX-M 14, ermA, ermB, mecA, vanA, vanB, and Sp
CTX-M 14, ermA, ermB, mefA, SHV, TEM, and vanA
CTX-M 14, ermA, ermB, mefA, SHV, TEM, and vanB
CTX-M 14, ermA, ermB, mefA, SHV, TEM, and Sp
CTX-M 14, ermA, ermB, mefA, SHV, vanA, and vanB
CTX-M 14, ermA, ermB, mefA, SHV, vanA, and Sp
CTX-M 14, ermA, ermB, mefA, SHV, vanB, and Sp
CTX-M 14, ermA, ermB, mefA, TEM, vanA, and vanB
CTX-M 14, ermA, ermB, mefA, TEM, vanA, and Sp
CTX-M 14, ermA, ermB, mefA, TEM, vanB, and Sp
CTX-M 14, ermA, ermB, mefA, vanA, vanB, and Sp
CTX-M 14, ermA, ermB, SHV, TEM, vanA, and vanB
CTX-M 14, ermA, ermB, SHV, TEM, vanA, and Sp
CTX-M 14, ermA, ermB, SHV, TEM, vanB, and Sp
CTX-M 14, ermA, ermB, SHV, vanA, vanB, and Sp
CTX-M 14, ermA, ermB, TEM, vanA, vanB, and Sp
CTX-M 14, ermA, mecA, mefA, SHV, TEM, and vanA
CTX-M 14, ermA, mecA, mefA, SHV, TEM, and vanB
CTX-M 14, ermA, mecA, mefA, SHV, TEM, and Sp
CTX-M 14, ermA, mecA, mefA, SHV, vanA, and vanB
CTX-M 14, ermA, mecA, mefA, SHV, vanA, and Sp
CTX-M 14, ermA, mecA, mefA, SHV, vanB, and Sp
CTX-M 14, ermA, mecA, mefA, TEM, vanA, and vanB
CTX-M 14, ermA, mecA, mefA, TEM, vanA, and Sp
CTX-M 14, ermA, mecA, mefA, TEM, vanB, and Sp
CTX-M 14, ermA, mecA, mefA, vanA, vanB, and Sp
CTX-M 14, ermA, mecA, SHV, TEM, vanA, and vanB
CTX-M 14, ermA, mecA, SHV, TEM, vanA, and Sp
CTX-M 14, ermA, mecA, SHV, TEM, vanB, and Sp
CTX-M 14, ermA, mecA, SHV, vanA, vanB, and Sp
CTX-M 14, ermA, mecA, TEM, vanA, vanB, and Sp
CTX-M 14, ermA, mefA, SHV, TEM, vanA, and vanB
CTX-M 14, ermA, mefA, SHV, TEM, vanA, and Sp
CTX-M 14, ermA, mefA, SHV, TEM, vanB, and Sp
CTX-M 14, ermA, mefA, SHV, vanA, vanB, and Sp
CTX-M 14, ermA, mefA, TEM, vanA, vanB, and Sp
CTX-M 14, ermA, SHV, TEM, vanA, vanB, and Sp
CTX-M 14, ermB, mecA, mefA, SHV, TEM, and vanA
CTX-M 14, ermB, mecA, mefA, SHV, TEM, and vanB
CTX-M 14, ermB, mecA, mefA, SHV, TEM, and Sp
CTX-M 14, ermB, mecA, mefA, SHV, vanA, and vanB
CTX-M 14, ermB, mecA, mefA, SHV, vanA, and Sp
CTX-M 14, ermB, mecA, mefA, SHV, vanB, and Sp
CTX-M 14, ermB, mecA, mefA, TEM, vanA, and vanB
CTX-M 14, ermB, mecA, mefA, TEM, vanA, and Sp
CTX-M 14, ermB, mecA, mefA, TEM, vanB, and Sp
CTX-M 14, ermB, mecA, mefA, vanA, vanB, and Sp
CTX-M 14, ermB, mecA, SHV, TEM, vanA, and vanB
CTX-M 14, ermB, mecA, SHV, TEM, vanA, and Sp
CTX-M 14, ermB, mecA, SHV, TEM, vanB, and Sp
CTX-M 14, ermB, mecA, SHV, vanA, vanB, and Sp
CTX-M 14, ermB, mecA, TEM, vanA, vanB, and Sp
CTX-M 14, ermB, mefA, SHV, TEM, vanA, and vanB
CTX-M 14, ermB, mefA, SHV, TEM, vanA, and Sp
CTX-M 14, ermB, mefA, SHV, TEM, vanB, and Sp
CTX-M 14, ermB, mefA, SHV, vanA, vanB, and Sp
CTX-M 14, ermB, mefA, TEM, vanA, vanB, and Sp
CTX-M 14, ermB, SHV, TEM, vanA, vanB, and Sp
CTX-M 14, mecA, mefA, SHV, TEM, vanA, and vanB
CTX-M 14, mecA, mefA, SHV, TEM, vanA, and Sp
CTX-M 14, mecA, mefA, SHV, TEM, vanB, and Sp
CTX-M 14, mecA, mefA, SHV, vanA, vanB, and Sp
CTX-M 14, mecA, mefA, TEM, vanA, vanB, and Sp
CTX-M 14, mecA, SHV, TEM, vanA, vanB, and Sp
CTX-M 14, mefA, SHV, TEM, vanA, vanB, and Sp
CTX-M 15, ermA, ermB, mecA, mefA, SHV, and TEM
CTX-M 15, ermA, ermB, mecA, mefA, SHV, and vanA
CTX-M 15, ermA, ermB, mecA, mefA, SHV, and vanB
CTX-M 15, ermA, ermB, mecA, mefA, SHV, and Sp
CTX-M 15, ermA, ermB, mecA, mefA, TEM, and vanA
CTX-M 15, ermA, ermB, mecA, mefA, TEM, and vanB
CTX-M 15, ermA, ermB, mecA, mefA, TEM, and Sp
CTX-M 15, ermA, ermB, mecA, mefA, vanA, and vanB
CTX-M 15, ermA, ermB, mecA, mefA, vanA, and Sp
CTX-M 15, ermA, ermB, mecA, mefA, vanB, and Sp
CTX-M 15, ermA, ermB, mecA, SHV, TEM, and vanA
CTX-M 15, ermA, ermB, mecA, SHV, TEM, and vanB
CTX-M 15, ermA, ermB, mecA, SHV, TEM, and Sp
CTX-M 15, ermA, ermB, mecA, SHV, vanA, and vanB
CTX-M 15, ermA, ermB, mecA, SHV, vanA, and Sp
CTX-M 15, ermA, ermB, mecA, SHV, vanB, and Sp
CTX-M 15, ermA, ermB, mecA, TEM, vanA, and vanB
CTX-M 15, ermA, ermB, mecA, TEM, vanA, and Sp
CTX-M 15, ermA, ermB, mecA, TEM, vanB, and Sp
CTX-M 15, ermA, ermB, mecA, vanA, vanB, and Sp
CTX-M 15, ermA, ermB, mefA, SHV, TEM, and vanA
CTX-M 15, ermA, ermB, mefA, SHV, TEM, and vanB
CTX-M 15, ermA, ermB, mefA, SHV, TEM, and Sp
CTX-M 15, ermA, ermB, mefA, SHV, vanA, and vanB
CTX-M 15, ermA, ermB, mefA, SHV, vanA, and Sp
CTX-M 15, ermA, ermB, mefA, SHV, vanB, and Sp
CTX-M 15, ermA, ermB, mefA, TEM, vanA, and vanB
CTX-M 15, ermA, ermB, mefA, TEM, vanA, and Sp
CTX-M 15, ermA, ermB, mefA, TEM, vanB, and Sp
CTX-M 15, ermA, ermB, mefA, vanA, vanB, and Sp
CTX-M 15, ermA, ermB, SHV, TEM, vanA, and vanB
CTX-M 15, ermA, ermB, SHV, TEM, vanA, and Sp
CTX-M 15, ermA, ermB, SHV, TEM, vanB, and Sp
CTX-M 15, ermA, ermB, SHV, vanA, vanB, and Sp
CTX-M 15, ermA, ermB, TEM, vanA, vanB, and Sp
CTX-M 15, ermA, mecA, mefA, SHV, TEM, and vanA
CTX-M 15, ermA, mecA, mefA, SHV, TEM, and vanB
CTX-M 15, ermA, mecA, mefA, SHV, TEM, and Sp
CTX-M 15, ermA, mecA, mefA, SHV, vanA, and vanB
CTX-M 15, ermA, mecA, mefA, SHV, vanA, and Sp
CTX-M 15, ermA, mecA, mefA, SHV, vanB, and Sp
CTX-M 15, ermA, mecA, mefA, TEM, vanA, and vanB
CTX-M 15, ermA, mecA, mefA, TEM, vanA, and Sp
CTX-M 15, ermA, mecA, mefA, TEM, vanB, and Sp
CTX-M 15, ermA, mecA, mefA, vanA, vanB, and Sp
CTX-M 15, ermA, mecA, SHV, TEM, vanA, and vanB
CTX-M 15, ermA, mecA, SHV, TEM, vanA, and Sp
CTX-M 15, ermA, mecA, SHV, TEM, vanB, and Sp
CTX-M 15, ermA, mecA, SHV, vanA, vanB, and Sp
CTX-M 15, ermA, mecA, TEM, vanA, vanB, and Sp
CTX-M 15, ermA, mefA, SHV, TEM, vanA, and vanB
CTX-M 15, ermA, mefA, SHV, TEM, vanA, and Sp
CTX-M 15, ermA, mefA, SHV, TEM, vanB, and Sp
CTX-M 15, ermA, mefA, SHV, vanA, vanB, and Sp
CTX-M 15, ermA, mefA, TEM, vanA, vanB, and Sp
CTX-M 15, ermA, SHV, TEM, vanA, vanB, and Sp
CTX-M 15, ermB, mecA, mefA, SHV, TEM, and vanA
CTX-M 15, ermB, mecA, mefA, SHV, TEM, and vanB
CTX-M 15, ermB, mecA, mefA, SHV, TEM, and Sp
CTX-M 15, ermB, mecA, mefA, SHV, vanA, and vanB
CTX-M 15, ermB, mecA, mefA, SHV, vanA, and Sp
CTX-M 15, ermB, mecA, mefA, SHV, vanB, and Sp
CTX-M 15, ermB, mecA, mefA, TEM, vanA, and vanB
CTX-M 15, ermB, mecA, mefA, TEM, vanA, and Sp
CTX-M 15, ermB, mecA, mefA, TEM, vanB, and Sp
CTX-M 15, ermB, mecA, mefA, vanA, vanB, and Sp
CTX-M 15, ermB, mecA, SHV, TEM, vanA, and vanB
CTX-M 15, ermB, mecA, SHV, TEM, vanA, and Sp
CTX-M 15, ermB, mecA, SHV, TEM, vanB, and Sp
CTX-M 15, ermB, mecA, SHV, vanA, vanB, and Sp
CTX-M 15, ermB, mecA, TEM, vanA, vanB, and Sp
CTX-M 15, ermB, mefA, SHV, TEM, vanA, and vanB
CTX-M 15, ermB, mefA, SHV, TEM, vanA, and Sp
CTX-M 15, ermB, mefA, SHV, TEM, vanB, and Sp
CTX-M 15, ermB, mefA, SHV, vanA, vanB, and Sp
CTX-M 15, ermB, mefA, TEM, vanA, vanB, and Sp
CTX-M 15, ermB, SHV, TEM, vanA, vanB, and Sp
CTX-M 15, mecA, mefA, SHV, TEM, vanA, and vanB
CTX-M 15, mecA, mefA, SHV, TEM, vanA, and Sp
CTX-M 15, mecA, mefA, SHV, TEM, vanB, and Sp
CTX-M 15, mecA, mefA, SHV, vanA, vanB, and Sp
CTX-M 15, mecA, mefA, TEM, vanA, vanB, and Sp
CTX-M 15, mecA, SHV, TEM, vanA, vanB, and Sp
CTX-M 15, mefA, SHV, TEM, vanA, vanB, and Sp
ermA, ermB, mecA, mefA, SHV, TEM, and vanA
ermA, ermB, mecA, mefA, SHV, TEM, and vanB
ermA, ermB, mecA, mefA, SHV, TEM, and Sp
ermA, ermB, mecA, mefA, SHV, vanA, and vanB
ermA, ermB, mecA, mefA, SHV, vanA, and Sp
ermA, ermB, mecA, mefA, SHV, vanB, and Sp
ermA, ermB, mecA, mefA, TEM, vanA, and vanB
ermA, ermB, mecA, mefA, TEM, vanA, and Sp
ermA, ermB, mecA, mefA, TEM, vanB, and Sp
ermA, ermB, mecA, mefA, vanA, vanB, and Sp
ermA, ermB, mecA, SHV, TEM, vanA, and vanB
ermA, ermB, mecA, SHV, TEM, vanA, and Sp
ermA, ermB, mecA, SHV, TEM, vanB, and Sp
ermA, ermB, mecA, SHV, vanA, vanB, and Sp
ermA, ermB, mecA, TEM, vanA, vanB, and Sp
ermA, ermB, mefA, SHV, TEM, vanA, and vanB
ermA, ermB, mefA, SHV, TEM, vanA, and Sp
ermA, ermB, mefA, SHV, TEM, vanB, and Sp
ermA, ermB, mefA, SHV, vanA, vanB, and Sp
ermA, ermB, mefA, TEM, vanA, vanB, and Sp
ermA, ermB, SHV, TEM, vanA, vanB, and Sp
ermA, mecA, mefA, SHV, TEM, vanA, and vanB
ermA, mecA, mefA, SHV, TEM, vanA, and Sp
ermA, mecA, mefA, SHV, TEM, vanB, and Sp
ermA, mecA, mefA, SHV, vanA, vanB, and Sp
ermA, mecA, mefA, TEM, vanA, vanB, and Sp
ermA, mecA, SHV, TEM, vanA, vanB, and Sp
ermA, mefA, SHV, TEM, vanA, vanB, and Sp
ermB, mecA, mefA, SHV, TEM, vanA, and vanB
ermB, mecA, mefA, SHV, TEM, vanA, and Sp
ermB, mecA, mefA, SHV, TEM, vanB, and Sp
ermB, mecA, mefA, SHV, vanA, vanB, and Sp
ermB, mecA, mefA, TEM, vanA, vanB, and Sp
ermB, mecA, SHV, TEM, vanA, vanB, and Sp
ermB, mefA, SHV, TEM, vanA, vanB, and Sp
mecA, mefA, SHV, TEM, vanA, vanB, and Sp

TABLE 13
Eight-target combinations of Sp
(Streptococcus pneumoniae), CTX-M 14, CTX-M 15, ermA,
ermB, mecA, mefA, SHV, TEM, vanA, and vanB

CTX-M 14, CTX-M 15, ermA, ermB, mecA, mefA, SHV, and TEM
CTX-M 14, CTX-M 15, ermA, ermB, mecA, mefA, SHV, and vanA
CTX-M 14, CTX-M 15, ermA, ermB, mecA, mefA, SHV, and vanB
CTX-M 14, CTX-M 15, ermA, ermB, mecA, mefA, SHV, and Sp
CTX-M 14, CTX-M 15, ermA, ermB, mecA, mefA, TEM, and vanA
CTX-M 14, CTX-M 15, ermA, ermB, mecA, mefA, TEM, and vanB
CTX-M 14, CTX-M 15, ermA, ermB, mecA, mefA, TEM, and Sp
CTX-M 14, CTX-M 15, ermA, ermB, mecA, mefA, vanA, and vanB
CTX-M 14, CTX-M 15, ermA, ermB, mecA, mefA, vanA, and Sp
CTX-M 14, CTX-M 15, ermA, ermB, mecA, mefA, vanB, and Sp
CTX-M 14, CTX-M 15, ermA, ermB, mecA, SHV, TEM, and vanA
CTX-M 14, CTX-M 15, ermA, ermB, mecA, SHV, TEM, and vanB
CTX-M 14, CTX-M 15, ermA, ermB, mecA, SHV, TEM, and Sp
CTX-M 14, CTX-M 15, ermA, ermB, mecA, SHV, vanA, and vanB
CTX-M 14, CTX-M 15, ermA, ermB, mecA, SHV, vanA, and Sp
CTX-M 14, CTX-M 15, ermA, ermB, mecA, SHV, vanB, and Sp
CTX-M 14, CTX-M 15, ermA, ermB, mecA, TEM, vanA, and vanB
CTX-M 14, CTX-M 15, ermA, ermB, mecA, TEM, vanA, and Sp
CTX-M 14, CTX-M 15, ermA, ermB, mecA, TEM, vanB, and Sp
CTX-M 14, CTX-M 15, ermA, ermB, mecA, vanA, vanB, and Sp
CTX-M 14, CTX-M 15, ermA, ermB, mefA, SHV, TEM, and vanA
CTX-M 14, CTX-M 15, ermA, ermB, mefA, SHV, TEM, and vanB
CTX-M 14, CTX-M 15, ermA, ermB, mefA, SHV, TEM, and Sp
CTX-M 14, CTX-M 15, ermA, ermB, mefA, SHV, vanA, and vanB
CTX-M 14, CTX-M 15, ermA, ermB, mefA, SHV, vanA, and Sp
CTX-M 14, CTX-M 15, ermA, ermB, mefA, SHV, vanB, and Sp
CTX-M 14, CTX-M 15, ermA, ermB, mefA, TEM, vanA, and vanB
CTX-M 14, CTX-M 15, ermA, ermB, mefA, TEM, vanA, and Sp
CTX-M 14, CTX-M 15, ermA, ermB, mefA, TEM, vanB, and Sp
CTX-M 14, CTX-M 15, ermA, ermB, mefA, vanA, vanB, and Sp
CTX-M 14, CTX-M 15, ermA, ermB, SHV, TEM, vanA, and vanB
CTX-M 14, CTX-M 15, ermA, ermB, SHV, TEM, vanA, and Sp
CTX-M 14, CTX-M 15, ermA, ermB, SHV, TEM, vanB, and Sp
CTX-M 14, CTX-M 15, ermA, ermB, SHV, vanA, vanB, and Sp
CTX-M 14, CTX-M 15, ermA, ermB, TEM, vanA, vanB, and Sp
CTX-M 14, CTX-M 15, ermA, mecA, mefA, SHV, TEM, and vanA
CTX-M 14, CTX-M 15, ermA, mecA, mefA, SHV, TEM, and vanB
CTX-M 14, CTX-M 15, ermA, mecA, mefA, SHV, TEM, and Sp
CTX-M 14, CTX-M 15, ermA, mecA, mefA, SHV, vanA, and vanB
CTX-M 14, CTX-M 15, ermA, mecA, mefA, SHV, vanA, and Sp
CTX-M 14, CTX-M 15, ermA, mecA, mefA, SHV, vanB, and Sp
CTX-M 14, CTX-M 15, ermA, mecA, mefA, TEM, vanA, and vanB
CTX-M 14, CTX-M 15, ermA, mecA, mefA, TEM, vanA, and Sp
CTX-M 14, CTX-M 15, ermA, mecA, mefA, TEM, vanB, and Sp
CTX-M 14, CTX-M 15, ermA, mecA, mefA, vanA, vanB, and Sp
CTX-M 14, CTX-M 15, ermA, mecA, SHV, TEM, vanA, and vanB
CTX-M 14, CTX-M 15, ermA, mecA, SHV, TEM, vanA, and Sp
CTX-M 14, CTX-M 15, ermA, mecA, SHV, TEM, vanB, and Sp
CTX-M 14, CTX-M 15, ermA, mecA, SHV, vanA, vanB, and Sp
CTX-M 14, CTX-M 15, ermA, mecA, TEM, vanA, vanB, and Sp
CTX-M 14, CTX-M 15, ermA, mefA, SHV, TEM, vanA, and vanB
CTX-M 14, CTX-M 15, ermA, mefA, SHV, TEM, vanA, and Sp
CTX-M 14, CTX-M 15, ermA, mefA, SHV, TEM, vanB, and Sp
CTX-M 14, CTX-M 15, ermA, mefA, SHV, vanA, vanB, and Sp
CTX-M 14, CTX-M 15, ermA, mefA, TEM, vanA, vanB, and Sp
CTX-M 14, CTX-M 15, ermA, SHV, TEM, vanA, vanB, and Sp
CTX-M 14, CTX-M 15, ermB, mecA, mefA, SHV, TEM, and vanA
CTX-M 14, CTX-M 15, ermB, mecA, mefA, SHV, TEM, and vanB
CTX-M 14, CTX-M 15, ermB, mecA, mefA, SHV, TEM, and Sp
CTX-M 14, CTX-M 15, ermB, mecA, mefA, SHV, vanA, and vanB
CTX-M 14, CTX-M 15, ermB, mecA, mefA, SHV, vanA, and Sp
CTX-M 14, CTX-M 15, ermB, mecA, mefA, SHV, vanB, and Sp
CTX-M 14, CTX-M 15, ermB, mecA, mefA, TEM, vanA, and vanB
CTX-M 14, CTX-M 15, ermB, mecA, mefA, TEM, vanA, and Sp
CTX-M 14, CTX-M 15, ermB, mecA, mefA, TEM, vanB, and Sp
CTX-M 14, CTX-M 15, ermB, mecA, mefA, vanA, vanB, and Sp
CTX-M 14, CTX-M 15, ermB, mecA, SHV, TEM, vanA, and vanB
CTX-M 14, CTX-M 15, ermB, mecA, SHV, TEM, vanA, and Sp
CTX-M 14, CTX-M 15, ermB, mecA, SHV, TEM, vanB, and Sp
CTX-M 14, CTX-M 15, ermB, mecA, SHV, vanA, vanB, and Sp
CTX-M 14, CTX-M 15, ermB, mecA, TEM, vanA, vanB, and Sp
CTX-M 14, CTX-M 15, ermB, mefA, SHV, TEM, vanA, and vanB
CTX-M 14, CTX-M 15, ermB, mefA, SHV, TEM, vanA, and Sp
CTX-M 14, CTX-M 15, ermB, mefA, SHV, TEM, vanB, and Sp
CTX-M 14, CTX-M 15, ermB, mefA, SHV, vanA, vanB, and Sp
CTX-M 14, CTX-M 15, ermB, mefA, TEM, vanA, vanB, and Sp
CTX-M 14, CTX-M 15, ermB, SHV, TEM, vanA, vanB, and Sp
CTX-M 14, CTX-M 15, mecA, mefA, SHV, TEM, vanA, and vanB
CTX-M 14, CTX-M 15, mecA, mefA, SHV, TEM, vanA, and Sp
CTX-M 14, CTX-M 15, mecA, mefA, SHV, TEM, vanB, and Sp
CTX-M 14, CTX-M 15, mecA, mefA, SHV, vanA, vanB, and Sp
CTX-M 14, CTX-M 15, mecA, mefA, TEM, vanA, vanB, and Sp
CTX-M 14, CTX-M 15, mecA, SHV, TEM, vanA, vanB, and Sp
CTX-M 14, CTX-M 15, mefA, SHV, TEM, vanA, vanB, and Sp
CTX-M 14, ermA, ermB, mecA, mefA, SHV, TEM, and vanA
CTX-M 14, ermA, ermB, mecA, mefA, SHV, TEM, and vanB
CTX-M 14, ermA, ermB, mecA, mefA, SHV, TEM, and Sp
CTX-M 14, ermA, ermB, mecA, mefA, SHV, vanA, and vanB
CTX-M 14, ermA, ermB, mecA, mefA, SHV, vanA, and Sp
CTX-M 14, ermA, ermB, mecA, mefA, SHV, vanB, and Sp
CTX-M 14, ermA, ermB, mecA, mefA, TEM, vanA, and vanB
CTX-M 14, ermA, ermB, mecA, mefA, TEM, vanA, and Sp
CTX-M 14, ermA, ermB, mecA, mefA, TEM, vanB, and Sp
CTX-M 14, ermA, ermB, mecA, mefA, vanA, vanB, Sp
CTX-M 14, ermA, ermB, mecA, SHV, TEM, vanA, vanB
CTX-M 14, ermA, ermB, mecA, SHV, TEM, vanA, and Sp
CTX-M 14, ermA, ermB, mecA, SHV, TEM, vanB, and Sp
CTX-M 14, ermA, ermB, mecA, SHV, vanA, vanB, and Sp
CTX-M 14, ermA, ermB, mecA, TEM, vanA, vanB, and Sp
CTX-M 14, ermA, ermB, mefA, SHV, TEM, vanA, and vanB
CTX-M 14, ermA, ermB, mefA, SHV, TEM, vanA, and Sp
CTX-M 14, ermA, ermB, mefA, SHV, TEM, vanB, and Sp
CTX-M 14, ermA, ermB, mefA, SHV, vanA, vanB, and Sp
CTX-M 14, ermA, ermB, mefA, TEM, vanA, vanB, and Sp
CTX-M 14, ermA, ermB, SHV, TEM, vanA, vanB, and Sp
CTX-M 14, ermA, mecA, mefA, SHV, TEM, vanA, and vanB
CTX-M 14, ermA, mecA, mefA, SHV, TEM, vanA, and Sp
CTX-M 14, ermA, mecA, mefA, SHV, TEM, vanB, and Sp
CTX-M 14, ermA, mecA, mefA, SHV, vanA, vanB, and Sp
CTX-M 14, ermA, mecA, mefA, TEM, vanA, vanB, and Sp
CTX-M 14, ermA, mecA, SHV, TEM, vanA, vanB, and Sp
CTX-M 14, ermA, mefA, SHV, TEM, vanA, vanB, and Sp
CTX-M 14, ermB, mecA, mefA, SHV, TEM, vanA, and vanB
CTX-M 14, ermB, mecA, mefA, SHV, TEM, vanA, and Sp
CTX-M 14, ermB, mecA, mefA, SHV, TEM, vanB, and Sp
CTX-M 14, ermB, mecA, mefA, SHV, vanA, vanB, and Sp
CTX-M 14, ermB, mecA, mefA, TEM, vanA, vanB, and Sp
CTX-M 14, ermB, mecA, SHV, TEM, vanA, vanB, and Sp
CTX-M 14, ermB, mefA, SHV, TEM, vanA, vanB, and Sp
CTX-M 14, mecA, mefA, SHV, TEM, vanA, vanB, and Sp
CTX-M 15, ermA, ermB, mecA, mefA, SHV, TEM, and vanA
CTX-M 15, ermA, ermB, mecA, mefA, SHV, TEM, and vanB
CTX-M 15, ermA, ermB, mecA, mefA, SHV, TEM, Sp
CTX-M 15, ermA, ermB, mecA, mefA, SHV, vanA, and vanB
CTX-M 15, ermA, ermB, mecA, mefA, SHV, vanA, and Sp
CTX-M 15, ermA, ermB, mecA, mefA, SHV, vanB, and Sp
CTX-M 15, ermA, ermB, mecA, mefA, TEM, vanA, and vanB
CTX-M 15, ermA, ermB, mecA, mefA, TEM, vanA, and Sp
CTX-M 15, ermA, ermB, mecA, mefA, TEM, vanB, and Sp
CTX-M 15, ermA, ermB, mecA, mefA, vanA, vanB, and Sp
CTX-M 15, ermA, ermB, mecA, SHV, TEM, vanA, and vanB
CTX-M 15, ermA, ermB, mecA, SHV, TEM, vanA, and Sp
CTX-M 15, ermA, ermB, mecA, SHV, TEM, vanB, and Sp
CTX-M 15, ermA, ermB, mecA, SHV, vanA, vanB, and Sp
CTX-M 15, ermA, ermB, mecA, TEM, vanA, vanB, and Sp
CTX-M 15, ermA, ermB, mefA, SHV, TEM, vanA, and vanB
CTX-M 15, ermA, ermB, mefA, SHV, TEM, vanA, and Sp
CTX-M 15, ermA, ermB, mefA, SHV, TEM, vanB, and Sp
CTX-M 15, ermA, ermB, mefA, SHV, vanA, vanB, and Sp
CTX-M 15, ermA, ermB, mefA, TEM, vanA, vanB, and Sp
CTX-M 15, ermA, ermB, SHV, TEM, vanA, vanB, and Sp
CTX-M 15, ermA, mecA, mefA, SHV, TEM, vanA, and vanB
CTX-M 15, ermA, mecA, mefA, SHV, TEM, vanA, and Sp
CTX-M 15, ermA, mecA, mefA, SHV, TEM, vanB, and Sp
CTX-M 15, ermA, mecA, mefA, SHV, vanA, vanB, and Sp
CTX-M 15, ermA, mecA, mefA, TEM, vanA, vanB, and Sp
CTX-M 15, ermA, mecA, SHV, TEM, vanA, vanB, and Sp
CTX-M 15, ermA, mefA, SHV, TEM, vanA, vanB, and Sp
CTX-M 15, ermB, mecA, mefA, SHV, TEM, vanA, and vanB
CTX-M 15, ermB, mecA, mefA, SHV, TEM, vanA, and Sp
CTX-M 15, ermB, mecA, mefA, SHV, TEM, vanB, and Sp
CTX-M 15, ermB, mecA, mefA, SHV, vanA, vanB, and Sp
CTX-M 15, ermB, mecA, mefA, TEM, vanA, vanB, and Sp
CTX-M 15, ermB, mecA, SHV, TEM, vanA, vanB, and Sp
CTX-M 15, ermB, mefA, SHV, TEM, vanA, vanB, and Sp
CTX-M 15, mecA, mefA, SHV, TEM, vanA, vanB, and Sp
ermA, ermB, mecA, mefA, SHV, TEM, vanA, and vanB
ermA, ermB, mecA, mefA, SHV, TEM, vanA, and Sp
ermA, ermB, mecA, mefA, SHV, TEM, vanB, and Sp
ermA, ermB, mecA, mefA, SHV, vanA, vanB, and Sp
ermA, ermB, mecA, mefA, TEM, vanA, vanB, and Sp
ermA, ermB, mecA, SHV, TEM, vanA, vanB, and Sp
ermA, ermB, mefA, SHV, TEM, vanA, vanB, and Sp
ermA, mecA, mefA, SHV, TEM, vanA, vanB, and Sp
ermB, mecA, mefA, SHV, TEM, vanA, vanB, and Sp

TABLE 14
Nine-target combinations of Sp
(Streptococcus pneumoniae), CTX-M 14, CTX-M 15, ermA,
ermB, mecA, mefA, SHV, TEM, vanA, and vanB

CTX-M 14, CTX-M 15, ermA, ermB, mecA, mefA, SHV, TEM, and vanA
CTX-M 14, CTX-M 15, ermA, ermB, mecA, mefA, SHV, TEM, and vanB
CTX-M 14, CTX-M 15, ermA, ermB, mecA, mefA, SHV, TEM, and Sp
CTX-M 14, CTX-M 15, ermA, ermB, mecA, mefA, SHV, vanA, and vanB
CTX-M 14, CTX-M 15, ermA, ermB, mecA, mefA, SHV, vanA, and Sp
CTX-M 14, CTX-M 15, ermA, ermB, mecA, mefA, SHV, vanB, and Sp
CTX-M 14, CTX-M 15, ermA, ermB, mecA, mefA, TEM, vanA, and vanB
CTX-M 14, CTX-M 15, ermA, ermB, mecA, mefA, TEM, vanA, and Sp
CTX-M 14, CTX-M 15, ermA, ermB, mecA, mefA, TEM, vanB, and Sp
CTX-M 14, CTX-M 15, ermA, ermB, mecA, mefA, vanA, vanB, and Sp
CTX-M 14, CTX-M 15, ermA, ermB, mecA, SHV, TEM, vanA, and vanB
CTX-M 14, CTX-M 15, ermA, ermB, mecA, SHV, TEM, vanA, and Sp
CTX-M 14, CTX-M 15, ermA, ermB, mecA, SHV, TEM, vanB, and Sp
CTX-M 14, CTX-M 15, ermA, ermB, mecA, SHV, vanA, vanB, and Sp
CTX-M 14, CTX-M 15, ermA, ermB, mecA, TEM, vanA, vanB, and Sp
CTX-M 14, CTX-M 15, ermA, ermB, mefA, SHV, TEM, vanA, and vanB
CTX-M 14, CTX-M 15, ermA, ermB, mefA, SHV, TEM, vanA, and Sp
CTX-M 14, CTX-M 15, ermA, ermB, mefA, SHV, TEM, vanB, and Sp
CTX-M 14, CTX-M 15, ermA, ermB, mefA, SHV, vanA, vanB, and Sp
CTX-M 14, CTX-M 15, ermA, ermB, mefA, TEM, vanA, vanB, and Sp
CTX-M 14, CTX-M 15, ermA, ermB, SHV, TEM, vanA, vanB, and Sp
CTX-M 14, CTX-M 15, ermA, mecA, mefA, SHV, TEM, vanA, and vanB
CTX-M 14, CTX-M 15, ermA, mecA, mefA, SHV, TEM, vanA, and Sp
CTX-M 14, CTX-M 15, ermA, mecA, mefA, SHV, TEM, vanB, and Sp
CTX-M 14, CTX-M 15, ermA, mecA, mefA, SHV, vanA, vanB, and Sp
CTX-M 14, CTX-M 15, ermA, mecA, mefA, TEM, vanA, vanB, and Sp
CTX-M 14, CTX-M 15, ermA, mecA, SHV, TEM, vanA, vanB, and Sp
CTX-M 14, CTX-M 15, ermA, mefA, SHV, TEM, vanA, vanB, and Sp
CTX-M 14, CTX-M 15, ermB, mecA, mefA, SHV, TEM, vanA, and vanB
CTX-M 14, CTX-M 15, ermB, mecA, mefA, SHV, TEM, vanA, and Sp
CTX-M 14, CTX-M 15, ermB, mecA, mefA, SHV, TEM, vanB, and Sp
CTX-M 14, CTX-M 15, ermB, mecA, mefA, SHV, vanA, vanB, and Sp
CTX-M 14, CTX-M 15, ermB, mecA, mefA, TEM, vanA, vanB, and Sp
CTX-M 14, CTX-M 15, ermB, mecA, SHV, TEM, vanA, vanB, and Sp
CTX-M 14, CTX-M 15, ermB, mefA, SHV, TEM, vanA, vanB, and Sp
CTX-M 14, CTX-M 15, mecA, mefA, SHV, TEM, vanA, vanB, and Sp
CTX-M 14, ermA, ermB, mecA, mefA, SHV, TEM, vanA, and vanB
CTX-M 14, ermA, ermB, mecA, mefA, SHV, TEM, vanA, and Sp
CTX-M 14, ermA, ermB, mecA, mefA, SHV, TEM, vanB, and Sp
CTX-M 14, ermA, ermB, mecA, mefA, SHV, vanA, vanB, and Sp
CTX-M 14, ermA, ermB, mecA, mefA, TEM, vanA, vanB, and Sp
CTX-M 14, ermA, ermB, mecA, SHV, TEM, vanA, vanB, and Sp
CTX-M 14, ermA, ermB, mefA, SHV, TEM, vanA, vanB, and Sp
CTX-M 14, ermA, mecA, mefA, SHV, TEM, vanA, vanB, and Sp
CTX-M 14, ermB, mecA, mefA, SHV, TEM, vanA, vanB, and Sp
CTX-M 15, ermA, ermB, mecA, mefA, SHV, TEM, vanA, and vanB
CTX-M 15, ermA, ermB, mecA, mefA, SHV, TEM, vanA, and Sp
CTX-M 15, ermA, ermB, mecA, mefA, SHV, TEM, vanB, and Sp
CTX-M 15, ermA, ermB, mecA, mefA, SHV, vanA, vanB, and Sp
CTX-M 15, ermA, ermB, mecA, mefA, TEM, vanA, vanB, and Sp
CTX-M 15, ermA, ermB, mecA, SHV, TEM, vanA, vanB, and Sp
CTX-M 15, ermA, ermB, mefA, SHV, TEM, vanA, vanB, and Sp
CTX-M 15, ermA, mecA, mefA, SHV, TEM, vanA, vanB, and Sp
CTX-M 15, ermB, mecA, mefA, SHV, TEM, vanA, vanB, and Sp
ermA, ermB, mecA, mefA, SHV, TEM, vanA, vanB, and Sp

TABLE 15
Ten-target combinations of Sp
(Streptococcus pneumoniae), CTX-M 14, CTX-M 15, ermA,
ermB, mecA, mefA, SHV, TEM, vanA, and vanB

CTX-M 14, CTX-M 15, ermA, ermB, mecA, mefA, SHV, TEM, vanA, and vanB
CTX-M 14, CTX-M 15, ermA, ermB, mecA, mefA, SHV, TEM, vanA, and Sp
CTX-M 14, CTX-M 15, ermA, ermB, mecA, mefA, SHV, TEM, vanB, and Sp
CTX-M 14, CTX-M 15, ermA, ermB, mecA, mefA, SHV, vanA, vanB, and Sp
CTX-M 14, CTX-M 15, ermA, ermB, mecA, mefA, TEM, vanA, vanB, and Sp
CTX-M 14, CTX-M 15, ermA, ermB, mecA, SHV, TEM, vanA, vanB, and Sp
CTX-M 14, CTX-M 15, ermA, ermB, mefA, SHV, TEM, vanA, vanB, and Sp
CTX-M 14, CTX-M 15, ermA, mecA, mefA, SHV, TEM, vanA, vanB, and Sp
CTX-M 14, CTX-M 15, ermB, mecA, mefA, SHV, TEM, vanA, vanB, and Sp
CTX-M 14, ermA, ermB, mecA, mefA, SHV, TEM, vanA, vanB, and Sp
CTX-M 15, ermA, ermB, mecA, mefA, SHV, TEM, vanA, vanB, and Sp

Any of the panels described herein may be combined with any other panel described herein. For example, in some embodiments, any one of the panels described in Tables 1-6 may be combined with any one of the panels described in Tables 7-15.

Any of the preceding panels may be further configured to individually detect between 1 and 18 (e.g., 1, 2, 3, 4, 5, 6, 7, 8, 9, 10, 11, 12, 13, 14, 15, 16, 17, or 18) pathogens selected from the following: 10 Acinetobacter spp. (e.g., Acinetobacter baumannii, Acinetobacter pittii, and Acinetobacter nosocomialis), Enterobacteriaceae spp., Enterococcus spp. (e.g., Enterococcus faecium (including E. faecium with resistance marker vanA/B) and Enterococcus faecalis), Klebsiella spp. (e.g., Klebsiella pneumoniae (including, e.g., K. pneumoniae with resistance marker KPC) and Klebsiella oxytoca), Pseudomonas spp. (e.g., Pseudomonas aeruginosa), Staphylococcus spp. (including, e.g., Staphylococcus aureus (e.g., S. aureus with resistance marker mecA), Staphylococcus haemolyticus, Staphylococcus lugdunensis, Staphylococcus maltophilia, Staphylococcus saprophyticus, coagulase-positive Staphylococcus species, and coagulase-negative (CoNS) Staphylococcus species), Streptococcus spp. (e.g., Streptococcus mitis, Streptococcus pneumoniae, Streptococcus agalactae, Streptococcus anginosa, Streptococcus bovis, Streptococcus dysgalactiae, Streptococcus mutans, Streptococcus sanguinis, and Streptococcus 20 pyogenes), Escherichia spp. (e.g., Escherichia coli), Stenotrophomonas spp. (e.g., Stenotrophomonas maltophilia), Proteus spp. (e.g., Proteus mirabilis and Proteus vulgars), Serratia spp. (e.g., Serrata marcescens), Citrobacter spp. (e.g., Citrobacter freundii and Citrobacter koseri), Haemophilus spp. (e.g., Haemophilus influenzae), Listeria spp. (e.g., Listeria monocytogenes), Neisseria spp. (e.g., Neisseria meningitidis), Bacteroides spp. (e.g., Bacteroides fragilis), Burkholderia spp. (e.g., Burkholderia cepacia), Campylobacter (e.g., Campylobacter jejuni and Campylobacter coli), Clostridium spp. (e.g., Clostridium perfringens), Kingella spp. (e.g., Kingella kingae), Morganella spp. (e.g., Morganella morgana), Prevotella spp. (e.g., Prevotella buccae, Prevotella intermedia, and Prevotella melaninogenica), Propionibacterium spp. (e.g., Propionibacterium acnes), Salmonella spp. (e.g., Salmonella enterica), Shigella spp. (e.g., Shigella dysenteriae and Shigella flexneri), and Enterobacter spp. (e.g., Enterobacter aerogenes and Enterobacter cloacae), Borrelia spp., (e.g., Borrelia burgdorferi sensu lato (Borrelia burgdorferi, Borrelia afzelii, and Borrelia garinii) species), Rickettsia spp. (including Rickettsia rickettsii and Rickettsia parkeri), Ehrlichia spp. (including Ehrlichia chaffeensis, Ehrlichia ewingii, and Ehrlichia muris-like), Coxiella spp. (including Coxiella burnetii), Anaplasma spp. (including Anaplasma phagocytophilum), Francisella spp., (including Francisella tularensis (including Francisella tularensis subspp. holarctica, mediasiatica, and novicida)), Streptococcus spp. (including Streptococcus pneumonia), and Neisseria spp. (including Neisseria meningitidis). In some embodiments, the bacterial pathogen panel is further configured to detect a fungal pathogen, for example, Candida spp. (e.g., Candida albicans, Candida guilliermondii, Candida glabrata, Candida krusei, Candida lusitaniae, Candida parapsilosis, Candida dublinensis, and Candida tropicalis) and Aspergillus spp. (e.g., Aspergillus fumigatus). In some embodiments, the pathogen panel is further configured to detect a Candida spp. (including Candida albicans, Candida guilliermondii, Candida glabrata, Candida krusei, Candida lusitaniae, Candida parapsilosis, Candida dublinensis, and Candida tropicalis). In cases where multiple species of a genus are detected, the species may be detected using individual target nucleic acids or using target nucleic acids that are universal to all of the species, for example, target nucleic acids amplified using universal primers. Any of the preceding panels may be further configured to configured to individually detect one or more (e.g., 1, 2, 3, 4, 5, 6, or 7) of Acinetobacter baumannii, Enterococcus faecium, Enterococcus faecalis, Klebsiella pneumoniae, Pseudomonas aeruginosa, Escherichia coli, and Staphylococcus aureus. For example, in some embodiments, the panel may be configured to individually detect one or more (e.g., 1, 2, 3, 4, or 5) of Enterococcus faecium, Klebsiella pneumoniae, Pseudomonas aeruginosa, Escherichia coli, and Staphylococcus aureus, as in the FDA-cleared T2BACTERIA® panel (T2 Biosystems, Inc.).

Any of the preceding panels may be further configured to individually detect one or more (e.g., 1, 2, 3, 4, 5, 6, or 8) Candida spp. (e.g., Candida albicans, Candida guilliermondii, Candida glabrata, Candida krusei, Candida lusitaniae, Candida parapsilosis, Candida dublinensis, and Candida tropicalis). For example, in some embodiments, the panel may be configured to individually detect one or more (e.g., 1, 2, 3, 4, or 5) of Candida albicans, Candida tropicalis, Candida krusei, Candida glabrata, and Candida parapsilosis, as in the FDA-cleared T2CANDIDA® panel (T2 Biosystems, Inc.).

In some embodiments, the panel can be further configured to individually detect one, two, or three Borrelia burgdorferi sensu lato (Borrelia burgdorferi, Borrelia afzelii, and Borrelia garinii) species. These species may be detected using individual target nucleic acids or using target nucleic acids that are universal to all three species, for example, target nucleic acids amplified using universal primers. In some embodiments, the panel is configured to detect Borrelia burgdorferi. In some embodiments, the panel is configured to detect Borrelia afzelii. In some embodiments, the panel is configured to detect Borrelia garinii. In some embodiments, the panel is configured to detect Borrelia burgdorferi and Borrelia afzelii. In some embodiments, the panel is configured to detect Borrelia burgdorferi and Borrelia garinii. In some embodiments, the panel is configured to detect Borrelia afzelii and Borrelia garinii. In some embodiments, the panel is configured to detect Borrelia burgdoferi, Borrelia afzelii and Borrelia garinii. In some embodiments, the panel may be configured to individually detect one or more (e.g., 1, 2, 3, 4, 5, or 6) of Rickettsia rickettsii, Coxiella burnettii, Ehrlichia chaffeensis, Babesia microti, Francisella tularensis, and Anaplasma phagocytophilum.

In any of the above embodiments, the panel may be configured to detect a marker that is characteristic of a genus, for example, a pan-bacterial marker, a pan-Candida marker, or a pan-Borrelia marker. In any of the above panels, the analyte may be a nucleic acid (e.g., an amplified target nucleic acid, as described above), or a polypeptide (e.g., a polypeptide derived from the pathogen or a pathogen-specific antibody produced by a host subject, for example, an IgM or IgG antibody). In some embodiments, multiple analytes (e.g., multiple amplicons) are used to detect a target, e.g., a drug resistance (e.g., antibiotic resistance) marker (e.g., an antibiotic resistance gene selected from the group consisting of NDM, KPC, IMP, VIM, OXA-48-like, DHA, CMY, FOX, mecA, mecC, vanA, vanB, CTX-M 14, CTX-M 15, mefA, mefE, ermA, ermB, SHV, and TEM) or a pathogen. In any of the above panels, the biological sample may be a biological sample containing cells and/or cell debris including but not limited to blood (e.g., whole blood, a crude whole blood lysate, serum, or plasma), bloody fluids (e.g., wound exudate, phlegm, bile, and the like), tissue samples (e.g., tissue biopsies, including homogenized tissue samples), BAL, urine, or sputum. In some embodiments, the biological sample is blood (e.g., whole blood, a crude whole blood lysate, serum, or plasma). Such panels may be used, for example, to diagnose bloodstream infections and/or to select an optimized therapy, e.g., by selecting an antimicrobial agent for which the pathogen is predicted to be sensitive rather than resistant. In some embodiments, the biological sample may be a tissue sample, for example, a homogenized tissue sample. Such panels may be used, for example, to detect drug-resistant infections present in tissue, e.g., tissue biopsies of skin at the site of a tick bite to identify Borrelia spp. for diagnosis of Lyme disease.

In some embodiments, the panel can be further configured to detect one or more toxin genes. For example, in some embodiments, the toxin gene panel can include one or more of Bacillus anthracis toxin genes protective antigen (pagA), edema factor (cya), and lethal factor (lef); enteropathogenic E. coli translocated intimin receptor (Tir); Clostridium difficile toxins TcdA and TcdB; and Clostridium botulinum toxins BoNT/A, BoNT/B, BoNT/C, BoNT/D, BoNT/E, BoNT/F, and BoNT/G.

### Medical Conditions

The methods of the invention can also be used diagnose and monitor diseases and other medical conditions. In some embodiments, the methods of the invention may be used to diagnose and monitor diseases in a multiplexed, automated, no sample preparation system.

The methods and systems of the invention can be used to identify and monitor the pathogenesis of disease in a subject, to select therapeutic interventions, and to monitor the effectiveness of the selected treatment. For example, for a patient having or at risk of bacteremia and/or sepsis, the methods and systems of the invention can be used to identify the infectious drug-resistant pathogen, pathogen load, and to monitor white blood cell count and/or biomarkers indicative of the status of the infection. The presence or expression level of one or more drug resistance markers (e.g., antibiotic resistance genes) can be used to select an appropriate therapy. The identity of the pathogen (e.g., at a group-level and/or a species-level) can be used to select an appropriate therapy. In some embodiments, the methods may further include administering a therapeutic agent following monitoring or diagnosing an infectious disease. The therapeutic intervention (e.g., a particular antibiotic agent) can be monitored as well to correlate the treatment regimen to the circulating concentration of antibiotic agent and pathogen load to ensure that the patient is responding to treatment.

Exemplary diseases that can be diagnosed and/or monitored by the methods and systems of the invention include diseases caused by or associated with microbial pathogens (e.g., bacterial infection or fungal infection), Lyme disease, bloodstream infection (e.g., bacteremia or fungemia), pneumonia, peritonitis, osteomyeletis, meningitis, empyema, urinary tract infection, sepsis, septic shock, and septic arthritis) and diseases that may manifest with similar symptoms to diseases caused by or associated with microbial pathogens (e.g., SIRS). In any of the methods, the causative pathogen may be a drug-resistant pathogen (e.g., an antibiotic-resistant bacterium (e.g., an antibiotic-resistant gram negative bacterium)). For example, the methods and systems of the invention may be used to diagnose and/or monitor a disease caused by the following non-limiting examples of pathogens: bacterial pathogens, including Acinetobacter spp. (e.g., Acinetobacter baumannii, Acinetobacter pittii, and Acinetobacter nosocomialis), Enterobacteriaceae spp., Enterococcus spp. (e.g., Enterococcus faecium (including E. faecium with resistance marker vanA/B) and Enterococcus faecalis), Klebsiella spp. (e.g., Klebsiella pneumoniae (e.g., K. pneumoniae with resistance marker KPC) and Klebsiella oxytoca), Pseudomonas spp. (e.g., Pseudomonas aeruginosa), Staphylococcus spp. (e.g., Staphylococcus aureus (e.g., S. aureus with resistance marker mecA), Staphylococcus haemolyticus, Staphylococcus lugdunensis, Staphylococcus maltophilia, Staphylococcus saprophyticus, coagulase-positive Staphylococcus species, and coagulase-negative (CoNS) Staphylococcus species), Streptococcus spp. (e.g., Streptococcus mitis, Streptococcus pneumoniae, Streptococcus agalactiae, Streptococcus anginosa, Streptococcus bovis, Streptococcus dysgalactiae, Streptococcus mutans, Streptococcus sanguinis, and Streptococcus pyogenes), Escherichia spp. (e.g., Escherichia coli), Stenotrophomonas spp. (e.g., Stenotrophomonas maltophilia), Proteus spp. (e.g., Proteus mirabilis and Proteus vulgaris), Serratia spp. (e.g., Serratia marcescens), Citrobacter spp. (e.g., Citrobacter freundii and Citrobacter koseri), Haemophilus spp. (e.g., Haemophilus influenzae), Listeria spp. (e.g., Listeria monocytogenes), Neisseria spp. (e.g., Neisseria meningitidis), Bacteroides spp. (e.g., Bacteroides fragilis), Burkholderia spp. (e.g., Burkholderia cepacia), Campylobacter (e.g., Campylobacter jejuni and Campylobacter coli), Clostridium spp. (e.g., Clostridium perfringens), Kingella spp. (e.g., Kingella kingae), Morganella spp. (e.g., Morganella morgana), Prevotella spp. (e.g., Prevotella buccae, Prevotella intermedia, and Prevotella melaninogenica), Propionibacterium spp. (e.g., Propionibacterium acnes), Salmonella spp. (e.g., Salmonella enterica), Shigella spp. (e.g., Shigella dysenteriae and Shigella flexneri), and Enterobacter spp. (e.g., Enterobacter aerogenes and Enterobacter cloacae); and fungal pathogens including but not limited to Candida spp. (e.g., Candida albicans, Candida glabrata, Candida krusei, C. parapsilosis, Candida auris, Candida lusitaniae, Candida haemulonii, Candida duobushaemulonii, Candida pseudohaemulonii, Candida guilliermondii, and C. tropicalis) and Aspergillus spp. (e.g., Aspergillus fumigatus). In some embodiments, the pathogen may be a Borrelia spp., including Borrelia burgdorferi sensu lato (Borrelia burgdoferi, Borrelia afzelii, and Borrelia garinii) species, Borrelia americana, Borrelia andersonii, Borrelia bavarensis, Borrelia bissettii, Borrelia carolinensis, Borrelia califomiensis, Borrelia chilensis, Borrelia genomosp. 1 and 2, Borrelia japonica, Borrelia kurtenbachii, Borrelia lustaniae, Borrelia myomatoii, Borrelia sinica, Borrelia spielmanii, Borrelia tanukii, Borrelia turdi, Borrelia valaisiana and unclassified Borrelia spp. In other embodiments, the pathogen may be selected from the following: Rickettsia spp. (including Rickettsia rickettsii and Rickettsia parker), Ehrlichia spp. (including Ehrlichia chaffeensis, Ehrlichia ewingii, and Ehrlichia muris-like), Coxiella spp. (including Coxiella burnetii), Babesia spp. (including Babesia microti and Babesia divergens), Anaplasma spp. (including Anaplasma phagocytophilum), Francisella spp., (including Francisella tularensis (including Francisella tularensis subspp. holarctica, mediasiatica, and novicida)), Streptococcus spp. (including Streptococcus pneumonia), and Neisseria spp. (including Neisseria meningitidis). In some embodiments, the pathogen is a viral pathogen (e.g., a retrovirus (e.g., HIV), an adeno-associated virus (AAV), an adenovirus, Ebolavirus, hepatitis (e.g., hepatitis A, B, C, or E), herpesvirus, human papillomavirus (HPV), rhinovirus, influenza, parainfluenza, measles, rotavirus, West Nile virus, zika virus, and the like). In some embodiments, the pathogen is a biothreat species, e.g., Bacillus anthracis, Francisella tularensis, Burkholderia spp. (e.g., B. mallei or B. pseudomallei), Yersinia pests, or Rickettsia prowazekii.

### Treatment

In some embodiments, the methods further include administering a therapeutic agent (e.g., an antimicrobial agent (e.g., an antibiotic agent)) or a composition thereof (e.g., a pharmaceutical composition) to a subject following a diagnosis or identification of a drug-resistant pathogen. Typically, the identification of a particular drug resistance marker (e.g., an antibiotic resistance gene) and/or a pathogen in a biological sample obtained from the subject (e.g., a complex sample containing host cells and/or cell debris, e.g., blood (e.g., whole blood, a crude whole blood lysate, serum, or plasma), bloody fluids (e.g., wound exudate, phlegm, bile, and the like), tissue samples (e.g., tissue biopsies (e.g., skin biopsies, muscle biopsies, or lymph node biopsies), including homogenized tissue samples), urine, BAL, CSF, SF, or sputum) will guide the selection of the appropriate therapeutic agent (e.g., antimicrobial agent, e.g., an antibiotic, an anti-fungal agent, and the like). In other embodiments, provided herein is a method of treating a patient that includes administering a therapeutic agent (e.g., an antimicrobial agent (e.g., an antibiotic agent)) or a composition thereof (e.g., a pharmaceutical composition) to a subject, wherein the patient has been identified as being infected by a pathogen and/or having a drug resistance marker (e.g., an antibiotic resistance marker) according to the methods described herein.

For example, for a bacterial infection (e.g., bacteremia), a therapy may include an antibiotic. In some instances, an antibiotic may be administered orally. In other instances, the antibiotic may be administered intravenously. Exemplary non-limiting antibiotics that may be used in the methods of the invention include but are not limited to, acrosoxacin, amifioxacin, amikacin, amoxycillin, ampicillin, aspoxicillin, azidocillin, azithromycin, aztreonam, balofloxacin, benzylpenicillin, biapenem, brodimoprim, cefaclor, cefadroxil, cefatrizine, cefcapene, cefdinir, cefetamet, ceftmetazole, cefoxitin, cefprozil, cefroxadine, ceftarolin, ceftazidime, ceftibuten, ceftobiprole, cefuroxime, cephalexin, cephalonium, cephaloridine, cephamandole, cephazolin, cephradine, chlorquinaldol, chlortetracycline, ciclacillin, cinoxacin, ciprofloxacin, clarithromycin, clavulanic acid, clindamycin, clofazimine, cloxacillin, colistin, danofloxacin, dapsone, daptomycin, demeclocycline, dicloxacillin, difloxacin, doripenem, doxycycline, enoxacin, enrofloxacin, erythromycin, fleroxacin, flomoxef, flucloxacillin, flumequine, fosfomycin, gentamycin, isoniazid, imipenem, kanamycin, levofloxacin, linezolid, mandelic acid, mecillinam, meropenem, metronidazole, minocycline, moxalactam, mupirocin, nadifloxacin, nafcillin, nalidixic acid, netilmycin, netromycin, nifuirtoinol, nitrofurantoin, nitroxoline, norfloxacin, ofloxacin, oxacillin, oxytetracycline, panipenem, pefloxacin, phenoxymethylpenicillin, pipemidic acid, piromidic acid, pivampicillin, pivmecillinam, polymixin-b, prulifloxacin, rufloxacin, sparfloxacin, sulbactam, sulfabenzamide, sulfacytine, sulfametopyrazine, sulphacetamide, sulphadiazine, sulphadimidine, sulphamethizole, sulphamethoxazole, sulphanilamide, sulphasomidine, sulphathiazole, teicoplanin, temafioxacin, tetracycline, tetroxoprim, tigecycline, tinidazole, tobramycin, tosufloxacin, trimethoprim, vancomycin, and pharmaceutically acceptable salts or esters thereof.

In some embodiments, a method of treatment may include administering a treatment to an asymptomatic patient, for example, based on the detection and/or identification of a pathogen present in a biological sample derived from the patient by the methods of the invention. In other embodiments, a method of treatment may include administering a treatment to a symptomatic patient based on the detection of identification of a pathogen present in a biological sample derived from the patient by the methods of the invention. In several embodiments, the biological sample may contain cells, cell debris, and/or nucleic acids (e.g., DNA or RNA (e.g., mRNA)) derived from both the host subject and a pathogen, including but not limited to blood (e.g., whole blood, a crude whole blood lysate, serum, or plasma), bloody fluids (e.g., wound exudate, phlegm, bile, and the like), tissue samples (e.g., tissue biopsies (e.g., skin biopsies, muscle biopsies, or lymph node biopsies), including homogenized tissue samples), CSF, SF, urine, BAL, or sputum (e.g., purulent sputum or bloody sputum). In some embodiments, the biological sample is blood (e.g., whole blood, a crude whole blood lysate, serum, or plasma) or a bloody fluid (e.g., wound exudate, phlegm, bile, and the like). In particular embodiments, the biological sample is whole blood. In other particular embodiments, the biological sample is a crude whole blood lysate.

In some embodiments, the treatment selected for a patient is based on the detection and/or identification of a pathogen by the methods of the invention. Appropriate treatments for different pathogen species are known in the art. In one example, if a Gram positive bacterium is detected in a biological sample derived from a patient, a method of treatment may involve administration of vancomycin. In another example, if a Gram negative bacterium is detected in a biological sample derived from a patient, a method of treatment may involve administration of pipercillin-tazobactam. In another example, in some embodiments, if an Acinetobacter spp. (e.g., Acinetobacter baumannii) is detected in a biological sample derived from a patient, a method of treatment may involve administration of colistin, meropenem, and/or gentamicin. In another example, in some embodiments, if a Klebsiella spp. (e.g., Klebsiella pneumoniae) is detected in a biological sample derived from a patient, a method of treatment may involve administration of meropenem. In yet another example, in some embodiments, if a Pseudomonas spp. (e.g., Pseudomonas aeruginosa) is detected in a biological sample derived from a patient, a method of treatment may involve administration of pipercillin-tazobactam. In a further example, in some embodiments, if an Escherichia spp. (e.g., Escherichia coli) is detected in a biological sample derived from a patient, a method of treatment may involve administration of meropenem. In another example, in some embodiments, if an Enterococcus spp. (e.g., Enterococcus faecium) is detected in a biological sample derived from a patient, a method of treatment may involve administration of daptomycin.

Exemplary therapies for the treatment of carbapenem-resistant infections may include treatment with a core therapy, either as the sole therapeutic agent (monotherapy) or in combination with one or more adjunct drugs (combination therapy). Treatments can be delivered using any suitable administration route, for example, per os, by intramuscular injection, or intravenously by traditional infusion, prolonged infusion (e.g., over 4 hours), or continuous infusion.

In general, monotherapies or core therapies for carbapenem-resistant infections that can be used include but are not limited to high-dose meropenem or doripenem, polymyxin B, colistin, tigecycline, ceftazidime-avibactam, meropenem-vaborbactam, aztreonam, and fosfomycin. Exemplary adjunct drugs include one or more of aminoglycosides, colistin, tigecycline, fosfomycin, gentamicin, tobramycin, amikacin, plazomicin, rimfampin, meropenem, doripenem, ertapenem, and imipenem.

Exemplary monotherapies or core therapies for carbapenem-resistant infections of the bloodstream include high-dose meropenem or doripenem and polymyxin B Suitable exemplary adjunct drugs for carbapenem-resistant infections of the bloodstream include one or more of an aminoglycoside, tigecycline, fosfomycin, and rimfampin.

Exemplary monotherapies or core therapies for carbapenem-resistant infections of the lung include high-dose meropenem or doripenem and polymyxin B. Suitable exemplary adjunct drugs for carbapenem-resistant infections of the lung include one or more of an aminoglycoside, tigecycline, fosfomycin, and rimfampin.

Exemplary monotherapies or core therapies for carbapenem-resistant infections of the gastrointestinal and/or biliary tract include high-dose meropenem or doripenem, polymyxin B, and high-dose tigecycline. Suitable exemplary adjunct drugs for carbapenem-resistant infections of the gastrointestinal and/or biliary tract include one or both of fosfomycin and rimfampin.

Exemplary monotherapies or core therapies for carbapenem-resistant infections of the urinary tract include high-dose meropenem or doripenem, an aminoglycoside, and fosfomycin. Suitable exemplary adjunct drugs for carbapenem-resistant infections of the urinary tract include one or both of colistin and an aminoglycoside.

High-dose meropenam can be defined as, e.g., 2000 mg q8h over 4 h by IV. High-dose doripenam can be defined as, e.g., 1000-2000 mg q8h over 4 h by IV. Ertapenem can be administered at, e.g., 1000 mg q24h by IV. Gentamicin can be administered at, e.g., 5-10 mg/kg daily dose by IV. Tobramycin can be administered at, e.g., 5-10 mg/kg daily dose by IV. Amikacin can be administered at, e.g., 10-15 mg/kg daily dose by IV. Tigecycline can be administered at, e.g., 100-200 mg loading dose, then 50 mg q12h mg/kg daily dose by IV. Fosfomycin can be administered at, e.g., 3 grams once or every 2-3 days per orum or 1-16 g daily by IV. Colistin can be administered at, e.g., 5 mg colistin base activity (CBA) per kg followed by maintenance doses of, e.g., 2.5-3 mg/kg per day. Polymyxin B can be administered at, e.g., 2-2.5 mg/kg followed by maintenance doses of, e.g., 2.5-3 mg/kg per day.

In one example, the invention provides a method for identifying a patient infected with an antibiotic resistant bacterial pathogen, the method including: (a) providing a biological sample obtained from the subject; and (b) detecting the presence of an antibiotic resistance gene in the biological sample according to any of the methods described herein, wherein the presence of an antibiotic resistance gene in the biological sample obtained from the subject identifies the subject as one who may be infected with an antibiotic resistant bacterial pathogen. In some embodiments, the method further includes selecting an optimized anti-bacterial therapy for the patient based on the presence of the antibiotic resistance gene. In some embodiments, the method further includes administering the optimized anti-bacterial therapy to the patient. In some embodiments, the optimized anti-bacterial therapy includes one or more antibiotic agents. In some embodiments, the one or more antibiotic agents is selected from the group consisting of polymyxin B, colistin, tigecycline, ceftazidime-avibactam, meropenem-vaborbactam, aztreonam, and fosfomycin. In some embodiments, the antibiotic agent is administered as a monotherapy. In some embodiments, the antibiotic agent is administered as a combination therapy. In some embodiments, the combination therapy includes one or more additional antibiotic agents selected from the group consisting of an aminoglycoside, colistin, tigecycline, fosfomycin, gentamicin, tobramycin, amikacin, plazomicin, rimfampin, meropenem, doripenem, ertapenem, and imipenem. In some embodiments, the optimized antibacterial therapy is administered to the patient orally, intravenously, intramuscularly, intra-arterially, subcutaneously, or intraperitoneally.

### Systems and Cartridges

The invention provides systems for carrying out the methods of the invention. The invention features methods and systems that may involve one or more cartridge units to provide a convenient method for placing all of the assay reagents and consumables onto the system. For example, the system may be customized to perform a specific function, or adapted to perform more than one function, e.g., via changeable cartridge units containing arrays of micro wells with customized magnetic particles contained therein. The system can include a replaceable and/or interchangeable cartridge containing an array of wells pre-loaded with magnetic particles, and designed for detection and/or concentration measurement of a particular analyte. Alternatively, the system may be usable with different cartridges, each designed for detection and/or concentration measurements of different analytes, or configured with separate cartridge modules for reagent and detection for a given assay. The cartridge may be sized to facilitate insertion into and ejection from a housing for the preparation of a liquid sample which is transferred to other units in the system (e.g., a magnetic assisted agglomeration unit, or an NMR unit). The cartridge unit itself can interface directly with manipulation stations as well as with the MR reader(s). The cartridge unit can be a modular cartridge having an inlet module that can be sterilized independent of the reagent module. The systems may include one or more NMR units, MAA units, cartridge units, and agitation units, as described in WO 2012/054639. Any of the systems described in WO 2012/054639 may be used for embodiments that involve T2MR detection, e.g., for providing group-level information to focus or narrow subsequent sequencing. For example, FIG. 42 of WO 2012/054639 depicts a system that can be used for embodiments involving T2MR detection. In some embodiments, the system stores a sample containing one or more amplified target nucleic acids for downstream sequencing.

For example, in some embodiments, the systems include one or more sequencing units. In other embodiments, the system results in production of a sample that can be sequenced separately, for example, a sample that requires one or more further steps for sequencing (e.g., adaptor ligation and tagging). Such systems may further include other components for carrying out an automated assay of the invention, such as a thermocycling unit for the amplification of oligonucleotides; a centrifuge, a robotic arm for delivery an liquid sample from unit to unit within the system; one or more incubation units; a fluid transfer unit (i.e., pipetting device) for combining assay reagents and a biological sample (e.g., a biological sample containing cells and/or cell debris including but not limited to blood (e.g., whole blood, a crude whole blood lysate, serum, or plasma), bloody fluids (e.g., wound exudate, phlegm, bile, and the like), tissue samples (e.g., tissue biopsies, including homogenized tissue samples), urine, BAL, CSF, SF, or sputum) to form the liquid sample; a computer with a programmable processor for storing data, processing data, and for controlling the activation and deactivation of the various units according to a one or more preset protocols; and a cartridge insertion system for delivering pre-filled cartridges to the system, optionally with instructions to the computer identifying the reagents and protocol to be used in conjunction with the cartridge.

The sequencing unit may include any system or device that is known in the art for sequencing, e.g., massively parallel sequencing, long-read sequencing, or Sanger sequencing. Exemplary sequencing devices include but are not limited to ILLUMINA® systems (e.g., the ILLUMINA® iSeq 100 system, MiniSeq® system, MiSeq® systems, NextSeq® series platforms, HiSeq® series platforms, HiSeq X® series platforms, and NovaSeq® 6000 system); the BGISEQ-500 system; the 10× Genomics Chromium™ system; Ion Torrent sequencing systems (e.g., Ion PGM™, Ion Proton™, Ion S5™, and Ion S5 XL); Oxford Nanopore systems (e.g., MinION and PromethION); Pacific Biosystems systems (e.g., PacBio RS II or PacBio Sequel); and the Roche 454 system. Other sequencing systems are known in the art.

The systems of the invention can provide an effective means for high throughput detection and/or sequencing of analytes present in sample, e.g., an environmental sample or a biological sample from a subject. The detection methods may be used in a wide variety of circumstances including, without limitation, sequencing of nucleic acids, identification and/or quantification of analytes that are associated with specific biological processes, physiological conditions, disorders or stages of disorders. As such, the systems have a broad spectrum of utility in, for example, disease diagnosis, identification of drug resistance (e.g., antibiotic resistance), disease onset and recurrence, individual response to treatment versus population bases, and monitoring of therapy. The devices and systems can provide a flexible system for personalized medicine. The system of the invention can be changed or interchanged along with a protocol or instructions to a programmable processor of the system to perform a wide variety of assays as described herein. The systems of the invention offer many advantages of a laboratory setting contained in a desk-top or smaller size automated instrument.

The invention provides methods and systems that may involve one or more cartridge units to provide a convenient method for placing all of the assay reagents (e.g., sequencing reagents) and consumables onto the system. For example, the cartridge units can include reagents for sequencing. Such reagents include, e.g., library preparation reagents (e.g., tagmentation reagents such as NEXTERA® XT library preparation reagents), buffers, adaptors, primers, enzymes (e.g., thermostable polymerases), and the like. The system can include a replaceable and/or interchangeable cartridge containing an array of wells pre-loaded, e.g., with sequencing reagents or magnetic particles, and designed for detection and/or sequencing of a particular analyte, e.g., a particular target nucleic acid. Alternatively, the system may be usable with different cartridges, each designed for detection and/or concentration measurements of different analytes, or configured with separate cartridge modules for reagent and detection for a given assay. The cartridge may be sized to facilitate insertion into and ejection from a housing for the preparation of a liquid sample which is transferred to other units in the system (e.g., a sequencing unit or an NMR unit). Any of the cartridges described in WO 2012/054639 can be used in the methods and systems described herein.

For example, provided herein is a system for the detection of one or more antibiotic resistance genes, the system including: (a) a first unit including (i) a permanent magnet defining a magnetic field; (ii) a support defining a well holding a liquid sample including magnetic particles having a mean particle diameter between 600 and 1200 nm, preferably between 650 and 950 nm, and the one or more antibiotic resistance genes and having an RF coil disposed about the well, the RF coil configured to detect a signal produced by exposing the liquid sample to a bias magnetic field created using the permanent magnet and an RF pulse sequence; and (iii) one or more electrical elements in communication with the RF coil, the electrical elements configured to amplify, rectify, transmit, and/or digitize the signal; and (b) a second unit including a removable cartridge sized to facilitate insertion into and removal from the system, wherein the removable cartridge is a modular cartridge including (i) a reagent module for holding one or more assay reagents, (ii) a detection module including a detection chamber for holding a liquid sample including the magnetic particles and the one or more analytes, and, optionally, (iii) a sterilizable inlet module, wherein the reagent module, the detection module, and, optionally, the sterilizable inlet module, can be assembled into the modular cartridge prior to use, and wherein the detection chamber is removable from the modular cartridge, preferably, wherein the system further includes a system computer with processor for implementing an assay protocol and storing assay data, and wherein the removable cartridge further includes (i) a readable label indicating the analyte to be detected, (ii) a readable label indicating the assay protocol to be implemented, (iii) a readable label indicating a patient identification number, (iv) a readable label indicating the position of assay reagents contained in the cartridge, or (v) a readable label including instructions for the programmable processor.

A modular cartridge can provide a simple means for cross contamination control during certain assays, including but not limited to distribution of amplification (e.g., PCR) products into multiple detection or sequencing aliquots. In addition, a modular cartridge can be compatible with automated fluid dispensing, and provides a way to hold reagents at very small volumes for long periods of time (in excess of a year). Finally, pre-dispensing these reagents allows concentration and volumetric accuracy to be set by the manufacturing process and provides for a point of care use instrument that is more convenient as it can require much less precise pipetting.

The modular cartridge can be designed for a multiplexed assay. The challenge in multiplexing assays is combining multiple assays which have incompatible assay requirements (i.e., different incubation times and/or temperatures) on one cartridge. The cartridge format depicted in FIGS. 14A-14C of WO 2012/054639 allows for the combination of different assays with dramatically different assay requirements. The cartridge features two main components: (i) a reagent module (i.e., the reagent strip portion) that contains all of the individual reagents required for the full assay panel (for example, a panel as described below), and (ii) the detection module. In some embodiments, a cartridge may be configured to detect and/or sequence from 2 to 24 or more target nucleic acids (e.g., 2, 3, 4, 5, 6, 7, 8, 9, 10, 11, 12, 13, 14, 15, 16, 17, 18, 19, 20, 21, 22, 23, 24, or more target nucleic acids), such as drug (e.g., antibiotic) resistance genes. In some embodiments, a cartridge may be configured to detect and/or sequence target nucleic acids from 2 to 24 or more pathogens (e.g., 2, 3, 4, 5, 6, 7, 8, 9, 10, 11, 12, 13, 14, 15, 16, 17, 18, 19, 20, 21, 22, 23, 24, or more pathogens). The detection modules contain only the parts of the cartridge that carry through the incubation, and can carry single assays or several assays, as needed. The cartridge units can further include one or more populations of magnetic particles, either as a liquid suspension or dried magnetic particles which are reconstituted prior to use. For example, the cartridge units of the invention can include a compartment including from 1×10⁶ to 1×10¹³ magnetic particles (e.g., from 1×10⁶ to 1×10⁸, 1×10⁷ to 1×10⁹, 1×10⁸ to 1×10¹⁰, 1×10⁹ to 1×10¹¹, 1×10¹⁰ to 1×10¹², 1×10¹¹ to 1×10¹³, or from 1×10⁷ to 5×10⁸ magnetic particles) for assaying a single liquid sample.

The number of cartridges utilized can be scaled based on the number of analytes being detected. For example, the targets may be detected across 1, 2, 3, 4, 5, 6, 7, 8, 9, 10, or more cartridges.

For example, provided herein is a removable cartridge comprising a well comprising any of the magnetic particles described herein. In some embodiments, the well includes a magnetic particle conjugated to one or more nucleic acid probes comprising a nucleic acid sequence selected from SEQ ID NOs: 17-32, 57-78, 83, 84, 86, 87, 89, 90, and 93-96 or a nucleic acid sequence having at least 95% sequence identity to any one of SEQ ID NOs: 17-32, 57-78, 83, 84, 86, 87, 89, 90, and 93-96. In some embodiments, the removable cartridge further includes one or more chambers for holding a plurality of reagent modules for holding one or more assay reagents. In some embodiments, the removable cartridge further includes a chamber comprising beads for lysing cells. In some embodiments, the removable cartridge further includes a chamber comprising a polymerase. In some embodiments, the removable cartridge further includes a chamber comprising one or more primers. In some embodiments, the one or more primers comprising a nucleic acid sequence selected from SEQ ID NOs: 1-16, 33-54, 81, 82, 85, 88, 91, and 92, or a nucleic acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to anyone of SEQ ID NOs: 1-16, 33-54, 81, 82, 85, 88, 91, and 92.

In another example, provided herein is a system for the detection of two or more antibiotic resistance genes selected from the group consisting of NDM, KPC, IMP, VIM, OXA-48-like, DHA, CMY, FOX, mecA, mecC, vanA, vanB, CTX-M 14, CTX-M 15, mefA, mefE, ermA, ermB, SHV, and TEM, the system including: (a) a first unit comprising (i) a permanent magnet defining a magnetic field; (ii) a support defining a well holding a liquid sample comprising magnetic particles having a mean particle diameter between 600 and 1200 nm, preferably between 650 and 950 nm, and the one or more antibiotic resistance genes and having an RF coil disposed about the well, the RF coil configured to detect a signal produced by exposing the liquid sample to a bias magnetic field created using the permanent magnet and an RF pulse sequence; and (iii) one or more electrical elements in communication with the RF coil, the electrical elements configured to amplify, rectify, transmit, and/or digitize the signal; and (b) a second unit comprising a removable cartridge sized to facilitate insertion into and removal from the system, wherein the removable cartridge is a modular cartridge comprising (i) a reagent module for holding one or more assay reagents, (ii) a detection module comprising a detection chamber for holding a liquid sample comprising the magnetic particles and the one or more analytes, and, optionally, (iii) a sterilizable inlet module, wherein the reagent module, the detection module, and, optionally, the sterilizable inlet module, can be assembled into the modular cartridge prior to use, and wherein the detection chamber is removable from the modular cartridge, preferably, wherein the system further comprises a system computer with processor for implementing an assay protocol and storing assay data, and wherein the removable cartridge further comprises (i) a readable label indicating the analyte to be detected, (ii) a readable label indicating the assay protocol to be implemented, (iii) a readable label indicating a patient identification number, (iv) a readable label indicating the position of assay reagents contained in the cartridge, or (v) a readable label comprising instructions for the programmable processor. In some embodiments, the two or more antibiotic resistance genes comprises at least one of IMP, OXA-48-like, DHA, CMY, FOX, CTX-M 14, CTX-M 15, mecC, mefA, mefE, ermA, ermB, or TEM. In some embodiments, the two or more antibiotic resistance genes comprises at least one of IMP, OXA-48-like, DHA, CMY, CTX-M 14, CTX-M 15, mecC, mefA, mefE, ermA, ermB, or TEM.

### Magnetic Particles and T2MR

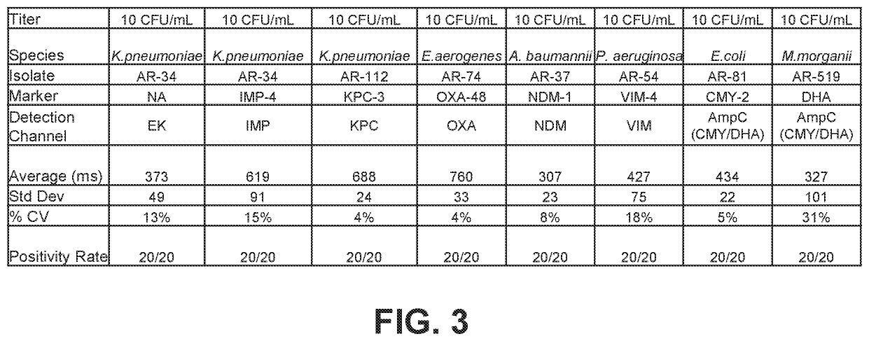

In some embodiments, the methods and systems of the invention may involve use of magnetic particles and NMR (e.g., T2MR). For example, T2MR can be used, for example, to rapidly and sensitively detect a target nucleic acid (e.g., an antibiotic resistance gene) and/or to obtain group-level information regarding a target nucleic acid, which can be used to narrow or focus sequencing analysis. The magnetic particles can be coated with a binding moiety (e.g., oligonucleotide, antibody, and the like) such that in the presence of analyte, or multivalent binding agent, aggregates are formed. Aggregation depletes portions of the sample from the microscopic magnetic non-uniformities that disrupt the solvent's T₂ signal, leading to an increase in T₂ relaxation (see, e.g., FIG. 3 of International Patent Application Publication No. WO 2012/054639, which is incorporated herein by reference in its entirety). Any NMR-based detection approach described in WO 2012/054639 may be used in the methods and systems described herein.

The T₂ measurement is a single measure of all spins in the ensemble, measurements lasting typically 1-10 seconds, which allows the solvent to travel hundreds of microns, a long distance relative to the microscopic non-uniformities in the liquid sample. Each solvent molecule samples a volume in the liquid sample and the T₂ signal is an average (net total signal) of all (nuclear spins) on solvent molecules in the sample; in other words, the T₂ measurement is a net measurement of the entire environment experienced by a solvent molecule, and is an average measurement of all microscopic non-uniformities in the sample.

The observed T₂ relaxation rate for the solvent molecules in the liquid sample is dominated by the magnetic particles, which in the presence of a magnetic field form high magnetic dipole moments. In the absence of magnetic particles, the observed T₂ relaxation rates for a liquid sample are typically long (i.e., T₂ (water)=approximately 2000 ms, T₂ (blood)=approximately 1500 ms). As particle concentration increases, the microscopic non-uniformities in the sample increase and the diffusion of solvent through these microscopic non-uniformities leads to an increase in spin decoherence and a decrease in the T₂ value. The observed T₂ value depends upon the particle concentration in a non-linear fashion, and on the relaxivity per particle parameter.

In embodiments that involve NMR detection, e.g., to provide rapid and sensitive detection and/or to obtain initial group-level information, the number of magnetic particles, and if present the number of agglomerant particles, remain constant during the assay. The spatial distribution of the particles changes when the particles cluster. Aggregation changes the average “experience” of a solvent molecule because particle localization into clusters is promoted rather than more even particle distributions. At a high degree of aggregation, many solvent molecules do not experience microscopic non-uniformities created by magnetic particles and the T₂ approaches that of solvent. As the fraction of aggregated magnetic particles increases in a liquid sample, the observed T₂ is the average of the non-uniform suspension of aggregated and single (unaggregated) magnetic particles. The assays of the invention are designed to maximize the change in T₂ with aggregation to increase the sensitivity of the assay to the presence of analytes, and to differences in analyte concentration.

For example, provided herein is a magnetic particle conjugated to a nucleic acid probe, wherein the nucleic acid probe is specific for an antibiotic resistance gene selected from the group consisting of NDM, KPC, IMP, VIM, OXA-48-like, DHA, CMY, FOX, mecA, mecC, vanA, vanB, CTX-M 14, CTX-M 15, mefA, mefE, ermA, ermB, SHV, and TEM. In some embodiments, the magnetic particle further includes an additional nucleic acid probe, wherein the second nucleic acid probe is specific for a second antibiotic resistance gene selected from the group consisting of NDM, KPC, IMP, VIM, OXA-48-like, DHA, CMY, FOX, mecA, mecC, vanA, vanB, CTX-M 14, CTX-M 15, mefA, mefE, ermA, ermB, SHV, and TEM. In some embodiments, the second antibiotic resistance gene is selected from IMP, OXA-48-like, DHA, CMY, FOX, CTX-M 14, CTX-M 15, mecC, mefA, mefE, ermA, ermB, or TEM. In some embodiments, the magnetic particle includes a first nucleic acid probe specific for DHA, and a second nucleic acid probe specific for CMY.

Also provided herein is a magnetic particle conjugated to one or more nucleic acid probes comprising a nucleic acid sequence selected from SEQ ID NOs: 17-32, 57-78, 83, 84, 86, 87, 89, 90, and 93-96, or a nucleic acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) to any one of SEQ ID NOs: 17-32, 57-78, 83, 84, 86, 87, 89, 90, and 93-96.

In some embodiments, the methods of the invention involve contacting a solution (e.g., a sample, e.g., a liquid sample, that includes whole blood or a crude whole blood lysate) with between from 1×10⁶ to 1×10¹³ magnetic particles per milliliter of the liquid sample (e.g., from 1×10⁶ to 1×10⁸, 1×10⁷ to 1×10⁸, 1×10⁷ to 1×10⁹, 1×10⁸ to 1×10¹⁰, 1×10⁹ to 1×10¹¹, or 1×10¹⁰ to 1×10¹³ magnetic particles per milliliter).

In some embodiments, the magnetic particles used in the methods and systems of the invention have a mean diameter of from 150 nm to 1200 nm (e.g., from 150 to 250, 200 to 350, 250 to 450, 300 to 500, 450 to 650, 500 to 700 nm, 700 to 850, 800 to 950, 900 to 1050, or from 1000 to 1200 nm). For example, in some embodiments, the magnetic particles used in the methods of the invention may have a mean diameter of from 150 nm to 699 nm (e.g., from 150 to 250, 200 to 350, 250 to 450, 300 to 500, 450 to 650, or from 500 to 699 nm). In other embodiments, the magnetic particles used in the methods of the invention may have a mean diameter of from 700 nm to 1200 nm (e.g., from 650 to 850, 650 to 950, 650 to 1050, 650 to 1200, 700 to 850, 800 to 950, 900 to 1050, or from 1000 to 1200 nm). In particular embodiments, the magnetic particles may have a mean diameter of from 700 nm to 950 nm (e.g., from 700 to 750, 700 to 800, 700 to 850, or from 700 to 900 nm).

In some embodiments, the magnetic particles used in the methods of the invention may have a T₂ relaxivity per particle of from 1×10⁸ to 1×10¹² mM⁻¹ s⁻¹ (e.g., from 1×10⁸ to 1×10⁹, 1×10⁸ to 1×¹⁰, 1×10⁹ to 1×10¹⁰, 1×10⁹ to 1×10¹¹, or from 1×10¹⁰ to 1×10¹² mM⁻¹ s⁻¹). In some embodiments, the magnetic particles have a T₂ relaxivity per particle of from 1×10⁹ to 1×10¹² mM⁻¹ s⁻¹ (e.g., from 1×10⁹ to 1×10¹⁰, 1×10⁹ to 1×10¹¹, or from 1×10¹⁰ to 1×10¹² mM⁻¹ s⁻¹).

In some embodiments, the magnetic particles may be substantially monodisperse. In some embodiments, the magnetic particles in a liquid sample (e.g., a biological sample containing cells and/or cell debris, including but not limited to blood (e.g., whole blood, a crude whole blood lysate, serum, or plasma), bloody fluids (e.g., wound exudate, phlegm, bile, and the like), tissue samples (e.g., tissue biopsies (e.g., skin biopsies, muscle biopsies, or lymph node biopsies), including homogenized tissue samples), urine, BAL, or sputum) may exhibit nonspecific reversibility in the absence of the one or more analytes and/or multivalent binding agent. In some embodiments, the magnetic particles may further include a surface decorated with a blocking agent selected from albumin, fish skin gelatin, gamma globulin, lysozyme, casein, peptidase, and an amine-bearing moiety (e.g., amino polyethyleneglycol, glycine, ethylenediamine, or amino dextran.

The above methods can be used with any of the following categories of detection of aggregation or disaggregation described herein, including those described in WO 2012/054639, e.g., at pages 110-111.

### Assay Reagents

The methods and compositions (e.g., systems, devices, kits, or cartridges) described herein may include any suitable reagents, for example, magnetic particles, surfactants, buffer components, additives, chelating agents, and the like. The surfactant may be selected from a wide variety of soluble non-ionic surface active agents including surfactants that are generally commercially available under the IGEPAL® trade name from GAF Company. The IGEPAL® liquid non-ionic surfactants are polyethylene glycol p-isooctylphenyl ether compounds and are available in various molecular weight designations, for example, IGEPAL® CA720, IGEPAL® CA630, and IGEPAL® CA890. Other suitable non-ionic surfactants include those available under the trade name TETRONIC® 909 from BASF Corporation. This material is a tetra-functional block copolymer surfactant terminating in primary hydroxyl groups. Suitable non-ionic surfactants are also available under the ALPHONIC® trade name from Vista Chemical Company and such materials are ethoxylates that are non-ionic biodegradables derived from linear primary alcohol blends of various molecular weights. The surfactant may also be selected from poloxamers, such as polyoxyethylene-polyoxypropylene block copolymers, such as those available under the trade names SYNPERONIC® PE series (ICI), PLURONIC® series (BASF), Supronic, MONOLAN®, PLURACARE®, and PLURODAC®, polysorbate surfactants, such as TWEEN®20 (PEG-20 sorbitan monolaurate), and glycols such as ethylene glycol and propylene glycol.

Such non-ionic surfactants may be selected to provide an appropriate amount of detergency for an assay without having a deleterious effect on assay reactions. In particular, surfactants may be included in a reaction mixture for the purpose of suppressing non-specific interactions among various ingredients of the aggregation assays of the invention. The non-ionic surfactants are typically added to the liquid sample prior in an amount from 0.01% (w/w) to 5% (w/w).

The non-ionic surfactants may be used in combination with one or more proteins (e.g., albumin, fish skin gelatin, lysozyme, or transferrin) also added to the liquid sample prior in an amount from 0.01% (w/w) to 5% (w/w).

Furthermore, the assays, methods, and cartridge units of the invention can include additional suitable buffer components (e.g., Tris base, selected to provide a pH of about 7.8 to 8.2 in the reaction milieu); and chelating agents to scavenge cations (e.g., ethylene diamine tetraacetic acid (EDTA), EDTA disodium, citric acid, tartaric acid, glucuronic acid, saccharic acid or suitable salts thereof).

### Primers and Probes

The invention provides primers and probes for use in the methods, systems, cartridges, and kits provided herein. For example, provided herein are primers and probes useful for detecting and/or sequencing drug resistance markers (e.g., antibiotic resistance genes) from pathogens (e.g., drug-resistant pathogens, e.g., carbapenem-resistant Gram-negative bacteria) in complex samples (e.g., blood).

For example, provided herein is a nucleic acid probe comprising a nucleic acid sequence selected from SEQ ID NOs: 17-32, 57-78, 83, 84, 86, 87, 89, 90, and 93-96, or a nucleic acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to any one of SEQ ID NOs: 17-32, 57-78, 83, 84, 86, 87, 89, 90, and 93-96.

In another example, provided herein is a nucleic acid primer comprising a nucleic acid sequence selected from SEQ ID NOs: 1-16, 33-54, 81, 82, 85, 88, 91, and 92, or a nucleic acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to any one of SEQ ID NOs: 1-16, 33-54, 81, 82, 85, 88, 91, and 92.

Any of the primer and/or probe sequences described in International Patent Publication Nos. WO2012/054639, WO 2016/118766, WO 2017/127731, or WO 2018/213641, each of which is incorporated herein by reference in its entirety, may be used in the methods, systems, cartridges, and kits provided herein.

In some embodiments, any of the preceding primers or probes may include one or more modified bases, for example, 2,6-Diaminopurine (abbreviated herein as “/i6diPr/”), deoxyinosine (abbreviated herein as “/ideoxyl/”), nitroindole (abbreviated herein as /35NiTInd/ or NitInd) or other modified bases known in the art.

### Contamination Control

One potential problem in the use of amplification methods such as PCR as an analytical tool is the risk of having new reactions contaminated with old, amplified products. Such contamination could potentially affect downstream sequencing results as well. Potential sources of contamination include a) large numbers of target organisms in clinical specimens that may result in cross-contamination, b) plasmid clones derived from organisms that have been previously analyzed and that may be present in larger numbers in the laboratory environment, and c) repeated amplification of the same target sequence leading to accumulation of amplification products in the laboratory environment. A common source of the accumulation of the PCR amplicon is aerosolization of the product. Typically, if uncontrolled aerosolization occurs, the amplicon will contaminate laboratory reagents, equipment, and ventilation systems. When this happens, all reactions will be positive, and it is not possible to distinguish between amplified products from the contamination or a true, positive sample. In addition to taking precautions to avoid or control this carry-over of old products, preferred embodiments include a blank reference reaction in every PCR experiment to check for carry-over. For example, carry-over contamination will be visible on the agarose gel as faint bands or fluorescent signal when TaqMan® probes, MolBeacons®, or intercalating dyes, among others, are employed as detection mechanisms. Furthermore, it is preferred to include a positive sample. As an example, in some embodiments, contamination control is performed using any of the approaches and methods described in WO 2012/054639. In some embodiments, a bleach solution is used to neutralize potential amplicons, for example, in a reaction tube of a T2Dx® device being used to perform a method of the invention. In some embodiments, contamination control includes the use of ethylene oxide (EtO) treatment, for example, of cartridge components.

Typically, the instrumentation and processing areas for samples that undergo amplification are split into pre- and post-amplification zones. This minimizes the chances of contamination of samples with amplicon prior to amplification. For example, the T2DX® instrument design is such that the pre- and post-amplification instrumentation and processing areas are integrated into a single instrument. This is made possible as described in the sections below.

Amplifying Multiple Amplicons Characteristic of a Target for Improved Sensitivity and/or Specificity

In some embodiments, the methods of the invention may involve amplification and detection of more than one amplicon characteristic of a target in a biological sample containing cells and/or cell debris including but not limited to blood (e.g., whole blood, a crude whole blood lysate, serum, or plasma), bloody fluids (e.g., wound exudate, phlegm, bile, and the like), tissue samples (e.g., tissue biopsies, including homogenized tissue samples), urine, CSF, SF, or sputum. In some embodiments, amplification of more than one target nucleic acid characteristic of a target increases the total amount of amplicons characteristic of the target in an assay (in other words, the amount of analyte is increased in the assay). This increase may allow, for example, an increase in sensitivity and/or specificity of detection of the target compared to a method that involves amplification and detection of a single amplicon characteristic of a target, e.g., for T2MR detection. In some embodiments, the methods of the invention may involve amplifying 2, 3, 4, 5, 6, 7, 8, 9, or 10 amplicons characteristic of a species.

In some embodiments, multiple (e.g., 2, 3, 4, 5, 6, 7, 8, 9, or 10) single-copy loci from a target are amplified and detected. In some embodiments, 2 single-copy loci from a target are amplified and detected. In some embodiments, amplification and detection of multiple single-copy loci from a species may allow for a sensitivity of detection comparable with methods that involve detecting an amplicon that is derived from a multi-copy locus. In some embodiments, methods involving detection of multiple single-copy loci amplified from a microbial species can detect from about 1-10 cells/mL (e.g., 1, 2, 3, 4, 5, 6, 7, 8, 9 or 10 cells/mL) of the microbial species in a liquid sample. In some embodiments, methods involving detection of multiple single-copy loci amplified from a target have at least 95% correct detection when the microbial species is present in the liquid sample at a frequency of less than or equal to 5 cells/mL (e.g., 1, 2, 3, 4, or 5 cells/mL) of liquid sample.

The invention also provides embodiments in which at least three amplicons are produced by amplification of two target nucleic acids, each of which is characteristic of a target. For example, in some embodiments, a first target nucleic acid and a second target nucleic acid to be amplified may be separated (for example, on a chromosome or on a plasmid) by a distance ranging from about 50 base pairs to about 1000 1500 base pairs (bp), e.g., about 50, 100, 150, 200, 250, 300, 400, 500, 600, 700, 800, 900, or 1000, 1100, 1200, 1300, 1400, or 1500 bp base pairs. In some embodiments, a first target nucleic acid and a second target nucleic acid to be amplified may be separated (for example, on a chromosome or on a plasmid) by a distance ranging from about 50 bp to about 1000 bp (e.g., about 50, 100, 150, 200, 250, 300, 400, 500, 600, 700, 800, 900, or 1000 bp). In some embodiments the first target nucleic acid and the second target nucleic acid to be amplified may be separated by a distance ranging from about 50 bp to about 1500 bp, from about 50 bp to about 1400 bp, from about 50 bp to about 1300 bp, from about 50 bp to about 1200 bp, from about 50 bp to about 1100 bp, from about 50 bp to about 1000 bp, from about 50 bp to about 950 bp, from about 50 bp to about 900 bp, from about 50 bp to about 850 bp, from about 50 bp to about 800 bp, from about 50 bp to about 800 bp, from about 50 bp to about 750 bp, from about 50 bp to about 700 bp, from about 50 bp to about 650 bp, from about 50 bp to about 600 bp, from about 50 bp to about 550 bp, from about 50 bp to about 500 bp, from about 50 bp to about 500 bp, from about 50 bp to about 450 bp, from about 50 bp to about 400 bp, from about 50 bp to about 350 bp, from about 50 bp to about 300 bp, from about 50 bp to about 250 bp, from about 50 bp to about 200 bp, from about 50 bp to about 150 bp, or from about 50 bp to about 100 bp. In some embodiments, amplification of the first and second target nucleic acids using individual primer pairs (each having a forward and a reverse primer) may lead to amplification of an amplicon that includes the first target nucleic acid, an amplicon that includes the second target nucleic acid, and an amplicon that contains both the first and the second target nucleic acid. This may result in an increase in sensitivity of detection of the target compared to samples in which the third amplicon is not present. In any of the preceding embodiments, amplification may be by asymmetric PCR.

The invention provides magnetic particles decorated with nucleic acid probes to detect two or more amplicons characteristic of a target. For example, in some embodiments, the magnetic particles include two populations, wherein each population is conjugated to probes such that the magnetic particle that can operably bind each of the two or more amplicons. For instance, in embodiments where two target nucleic acids have been amplified to form a first amplicon and a second amplicon, a pair of particles each of which have a mix of capture probes on their surface may be used. In some embodiments, the first population of magnetic particles may be conjugated to a nucleic acid probe that operably binds a first segment of the first amplicon and a nucleic acid probe that operably binds a first segment of the second amplicon, and the second population of magnetic particles may be conjugated to a nucleic acid probe that operably binds a second segment of the first amplicon and a nucleic acid probe that operably binds a second segment of the second amplicon. For instance, one particle population may be conjugated with a 5′ capture probe specific to the first amplicon and a 5′ capture probe specific to second amplicon, and the other particle population may be conjugated with a 3′ capture probe specific to the first amplicon and a 3′ capture probe specific to the second amplicon. In such embodiments, the magnetic particles may aggregate in the presence of the first amplicon and aggregate in the presence of the second amplicon. Aggregation may occur to a greater extent when both amplicons are present.

In some embodiments, a magnetic particle may be conjugated to two, three, four, five, six, seven, eight, nine, or ten nucleic acid probes, each of which operably binds a segment of a distinct target nucleic acid. In some embodiments, a magnetic particle may be conjugated to a first nucleic acid probe and a second nucleic acid probe, wherein the first nucleic acid probe operably binds to a first target nucleic acid, and the second nucleic acid probe operably binds to a second target nucleic acid. In other embodiments, a magnetic particle may be conjugated to a first nucleic acid probe that operably binds a first target nucleic acid, a second nucleic acid probe that operably binds a second target nucleic acid, and a third nucleic acid that operably binds a third target nucleic acid. In yet other embodiments, a magnetic particle may be conjugated to a first nucleic acid probe that operably binds a first target nucleic acid, a second nucleic acid probe that operably binds a second target nucleic acid, a third nucleic acid that operably binds a third target nucleic acid, and a fourth nucleic acid probe that operably binds a fourth target nucleic acid. In still other embodiments, a magnetic particle may be conjugated to a first nucleic acid probe that operably binds a first target nucleic acid, a second nucleic acid probe that operably binds a second target nucleic acid, a third nucleic acid that operably binds a third target nucleic acid, a fourth nucleic acid probe that operably binds a fourth target nucleic acid, and a fifth nucleic acid probe that operably binds a fifth target nucleic acid. In some embodiments, one population of magnetic particles includes the 5′ capture probe for each amplicon to be detected, and the other population of magnetic particles includes the 3′ capture probe for each amplicon to be detected.

### Kits

The invention provides kits and articles of manufacture that can be used for carrying out the methods described herein. The kit may include one or more containers for holding the components of the kit (e.g., tubes (e.g., microcentrifuge tubes), plates (e.g., microtiter plates), trays, packaging materials (e.g., boxes), and the like. The kit may also include instructions (e.g., printed instructions for using the kit).

For example, a kit may include one or more, or all, of the following: one or more containers (e.g., tubes) that contain erythrocyte lysis buffers, one or more containers containing buffers or buffered solutions (e.g., TE buffer); one or more containers that contain primers (e.g., any of the primers described herein), one or more containers that contain control nucleic acids or total process controls, one or more containers containing lysis reagents (e.g., beads for beadbeating), and/or one or more containers containing amplification reagents (e.g., buffers, thermostable DNA polymerases, nucleotides, magnesium (e.g., MgCl₂), and the like). The kit may further include reagents for sequencing (e.g., buffers, library preparation reagents, enzymes, adaptors, and the like). The kit may further include reagents for T2MR detection (e.g., magnetic particles, probes, conjugated magnetic particles, and the like).

### EXAMPLES

The following examples are put forth so as to provide those of ordinary skill in the art with a complete disclosure and description of how the devices, systems, and methods described herein are performed, made, and evaluated, and are intended to be purely exemplary of the invention and are not intended to limit the scope of what the inventors regard as their invention.

### Example 1: Assay for Detection of Antibiotic Resistance Genes

A method was developed for the detection of gram-negative carbapenem resistance genes and bacterial species (e.g., Enterobacter and Klebsiella species) in complex samples such as whole blood patient test samples Such a method can be used, for example, in determining whether one or more species of bacteria or one or more antibiotic resistance genes is present in a sample.

A. Selection of targets for assay

FIG. 1 shows an exemplary panel of targets chosen for detection in the assay. These targets include the resistance genes New Delhi metallo-β-lactamase (NDM; also referred to as bla_{NDM}), Klebsiella pneumonia carbapenemase (KPC; also referred to as bla_{KPC}), imipenemase (IMP; also referred to as bla_{IMP}), Verona integron-encoded metallo-β-lactamase (VIM; also referred to as bla_{VIM}), the oxacillinase-48 like (OXA-48 like) subgroup (also referred to as bla_{OXA}), Dharhan β-lactamase (DHA; also referred to as bla_{DHA}), cephamycinase (CMY; also referred to as bla_{CMY}), a target nucleic acid characteristic of Enterobacter and Klebsiella spp. (also referred to as EK), and an internal control (e.g., an orange internal control (OIC)).

A multiplexed amplification (e.g., PCR) reaction can be used to amplify the targets of the panel. Targets may be detected using T2 magnetic resonance using magnetic particles that are conjugated to probes that bind to the amplified target nucleic acids, e.g., using the T2DX® instrument. See, e.g., International Patent Application Publication No. WO 2012/054639. In one example, NDM, KPC, VIM, IMP, OXA-48, and EK can be individually identified (e.g., with magnetic particles having a first subpopulation conjugated to a nucleic acid probe that binds the 5′ end of the amplicon, and a second subpopulation conjugated to a nucleic acid probe that binds the 3′ end of the amplicon), and a double particle configuration can be used to detect the AmpC genes CMY and DHA as a group, resulting in 8 reported channels. In the double particle configuration, each of the two subpopulations of magnetic particles includes a probe that binds CMY and a probe that binds DHA. The targets may also be detected using sequencing, e.g., as described in U.S. Provisional Patent Application No. 62/729,375, which is incorporated by reference herein in its entirety.

B. Evaluation of Inclusivity for Primer and Probe Designs

Primers were developed for the amplification of the targets listed in Example 1 A and FIG. 1. Each resistance gene has many variants, which can differ by only a few sporadic nucleotides or have as low as 80% similarity. The primers were tested for inclusivity of (i.e., the ability to amplify) multiple variants of the targets: these variants are listed in FIG. 2. Inclusion or exclusion of the variants by the primers was assayed either by lab testing or by in silico testing. Lab testing was performed by spiking variant DNA in blood lysate or cellular isolates containing different variants into whole blood. Variants were deemed inclusive if sensitive detection was achieved (<50 copies DNA/reaction or <20 CFU/mL). In silico testing was performed using a nested series of BLAST searches to calculate identity and coverage percentages of variants, primers, and probes. Inclusive variants in FIG. 2 are those with identity and coverage >90%. The IMP gene is known to have poor homology between variants, and therefore the in silico inclusivity list was divided into >90 and >95% categories. Wet testing was performed on variants in each category, confirming that the primers and probes had broad inclusivity for this group.

The primer and probe sequences for this panel are shown below in Table 16.

TABLE 16
Primer and Probe Sequences

| SEQ | | |
|---|---|---|
| ID NO | Name | Sequence |
| 1. | NDM F Primer | CCAGCTCGCACCGAATGTCT |
| 2. | NDM R Primer | CATCTTGTCCTGATGCGCGTGAGTCA |
| 3. | KPC F Primer | TTTCTGCCACCGCGCTGAC |
| 4. | KPC R Primer | GCAGCAAGAAAGCCCTTGAATG |
| 5. | IMP F Primer | ACATTTCCATAGCGACAGCACGGGCGGAAT |
| 6. | IMP R Primer | GGACTTTGGCCAAGCTTCTAAATTTGCGTC |
| 7. | VIM F Primer | CGTGCAGTCTCCACGCACT |
| 8. | VIM R Primer | TCGAATGCGCAGCACCGGGATAG |
| 9. | OXA-48-like | GGCTGTGTTTTTGGTGGCATCGATTATC |
| | F Primer | |
| 10. | OXA-48-like | TCCCACTTAAAGACTTGGTGTTCATCC |
| | R Primer | |
| 11. | DHA F Primer | ACGGGCCGGTAATGCGGATCTGGA |
| 12. | DHA R Primer | TATTCGCCAGAATCACAATCGCCACCTGT |
| 13. | CMY F Primer | CCGCGGCGAAATTAAGCTCAGCGA |
| 14. | CMY R Primer | CCAAACAGACCAATGCTGGAGTTAG |
| 15. | EK F Primer | ATTCGTTGCACTATCGTTAACTGAATACA |
| 16. | EK R Primer | CTGTACCGTCGGACTTTCCAGAC |
| 17. | NDM 5′ Probe | GCGACCGGCAGGTTGATCT |
| 18. | NDM 3′ Probe | CATGTCGAGATAGGAAGTGTGCTGC |
| 19. | KPC 5′ Probe | CGGAACCATTCGCTAAACTC |
| 20. | KPC 3′ Probe | AGGCGCAACTGTAAGTTACCG |
| 21. | IMP 5′ Probe | GCTTAATTCTCAATCTATCCCCACGTAT |
| 22. | IMP 3′ Probe | CTCCAGATAACGTAGTGGTTTGGCTG |
| 23. | VIM 5′ Probe | CTTTCATGACGACCGCGTCGG |
| 24. | VIM 3′ Probe | CTCTAGAAGGACTCTCATCGAGC |
| 25. | OXA-48-like | ATTTTAAAGGTAGATGCGGG |
| | 5′ Probe | |
| 26. | OXA-48-like | CGCCCTGTGATTTATGTTCA |
| | 3′ Probe | |
| 27. | DHA 5′ Probe | GTTTTATGCACCCAGGAAGC |
| 28. | DHA 3′ Probe | TCTGCTGCGGCCAGTCATA |
| 29. | CMY 5′ Probe | GCGGCTGCCAGTTTTGATAA |
| 30. | CMY 3′ Probe | GTGGCTAAGTGCAGCAGGC |
| 31. | EK 5′ Probe | CGTTCCACTAACACACAAGCTGATTCAG |
| 32. | EK 3′ Probe | ATCTCGGTTGATTTCTTTTCCTCGGG |

C. Analytical Sensitivity for Targets (10 CFU/mL)

The limit of detection (LoD) was defined as the lowest concentration at which a 95% positivity rate (i.e., the rate at which the test result is positive for the target) can be achieved for N≥20 samples. All targets listed in Example 1A were tested at a titer of 10 colony-forming units (CFU)/mL. Bacterial isolates as shown in FIG. 3 were spiked into whole blood, amplified, and detected using T2MR essentially as described in WO 2012/054639. Antibiotic resistance marker genes were detected in the appropriate detection channel, as follows: not applicable (NA) in channel EK; IMP-4 in channel IMP; KPC-3 in channel KPC; OXA-48 in channel OXA; NDM-1 in channel NDM; VIM-4 in channel VIM; CMY2 and DHA in channel AmpC (CMY/DHA). The average T2 signal (“Average (ms)”) was measured for 20 replicates using a T2MR reader and standard deviation (“Std Dev”), percent coefficient of variation (“% CV”), and positivity rate were measured. Results of this analytical sensitivity experiment are shown in FIG. 3. These data show that each of the tested targets were identified at a titer of 10 CFU/mL, and that the LoD is s 10 CFU/mL.

D. Analytical Sensitivity for Targets (2 CFU/mL)

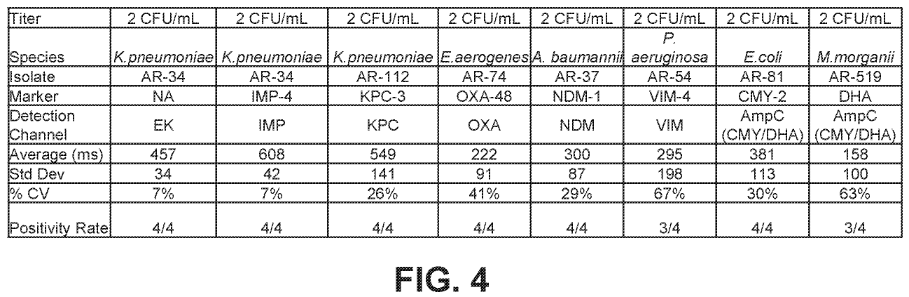

An analytical sensitivity experiment was performed to determine whether the targets named in Example 1A could be identified at a titer of 2 CFU/mL. Bacterial isolates as shown in FIG. 3 were spiked into whole blood, amplified, and detected using T2MR essentially as described in WO 2012/054639. Markers were detected in the appropriate channel, as described in Example 1D. Average T2 signal (ms) was measured for 4 replicates using a T2MR reader, and standard deviation, % CV, and positivity rate were measured. Results of this LoD assay are shown in FIG. 4. These data show that each of the tested targets were identified at a titer of 2 CFU/mL.

E. Cross-Reactivity Assay for Targets

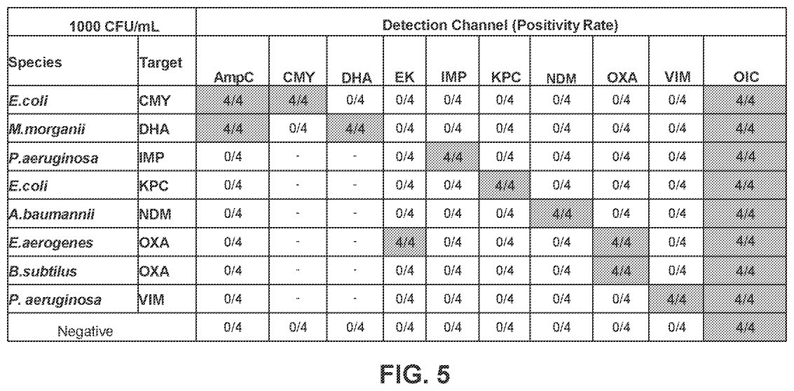

Cross-reactivity assays were performed to assess detection of the targets listed in Example 1A in non-target channels. Assays were performed at a titer of 1000 CFU/mL essentially as described in Example 1C. Each sample was assayed for the target in its intended channel (e.g., VIM in the channel VIM) and in all other channels (e.g., VIM in the channels AmpC, EK, IMP, KPC, NDM, OXA, VIM, and OIC). A negative control (i.e., not containing bacteria) was also assayed. CMY, DHA, and the negative control were additionally tested in the CMY and DHA channels. This experiment shows that there is no cross-reactivity between the individual CMY and DHA channels. Positivity rate was measured and reported for 4 replicates in each channel. Results of this cross-reactivity assay are shown in FIG. 5.

F. Assay for Interfering Substances

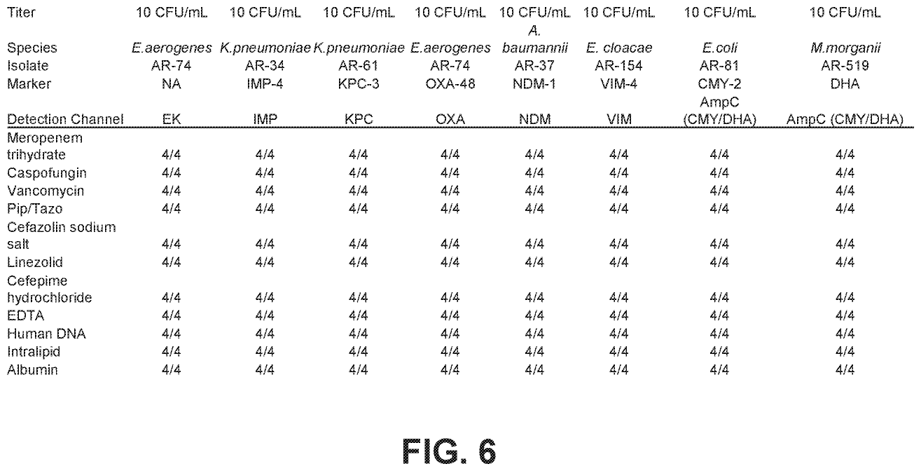

An assay for substances potentially interfering with the detection of the targets was performed. Assays were performed at a titer of 10 colony-forming units (CFU)/mL. Each sample was assayed for the target in its intended channel (indicated in FIG. 3) in the presence of one of meropenem trihydrate, caspofungin, vancomycin, piperacillin/tazobactam (“pip/tazo”), cefazolin sodium salt, linezolid, cefepime hydrochloride, EDTA, human DNA, intralipid, or albumin. Positivity rate was measured and reported for 4 replicates of each potentially interfering substance in each channel. Results of this assay are shown in FIG. 6. These data show that the assay achieved high-sensitivity detection of the targets despite the presence of potentially interfering substances.

G. Assay for Competitive Inhibition

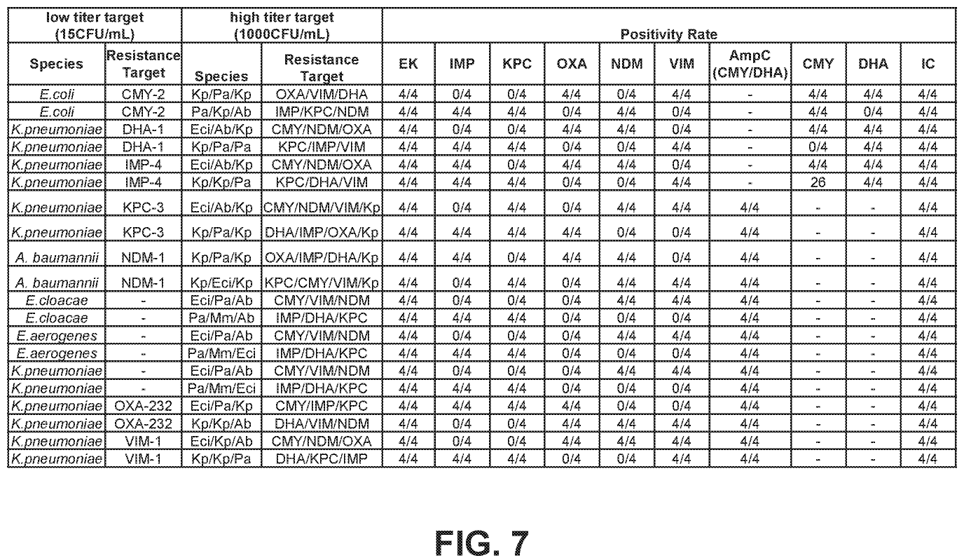

An assay for competitive inhibition was performed. In this experiment, a low titer target (15 CFU/mL) and several high titer targets (1000 CFU/mL) were spiked into the same samples. The assay was performed as described in Example 1C the positivity rate in each channel was assessed. The results of this experiment are shown in FIG. 7. These data show that the presence of the high titer targets does not inhibit the signal from the individual low titer targets.

H. Multi-Spike Experiment

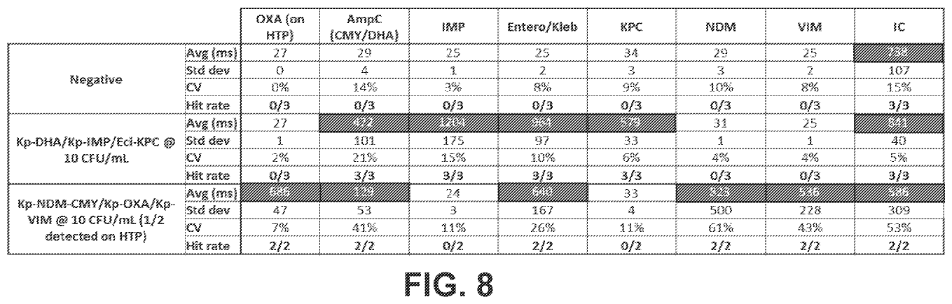

Two multi-spike experiments, at a titer of 10 CFU/mL per species, were performed. The spikes were Klebsiella pneumoniae (Kp)-DHA/Kp-IMP/Escherichia coli (Eci)-KPC and Kp-NDM/Kp-OXA/Kp-VIM. The samples were processed using a T2DX® instrument essentially as described in Example 1C. Three negative samples were run, and there were no false positives. 100% detection of all resistance targets and EK species was achieved (FIG. 8). This experiment demonstrates that multi-spikes (e.g., triple-spikes) can be detected using the automated T2DX® instrument

### Example 2: T2MR- and Sequencing-Based Detection of Antibiotic Resistance Genes in Complex Samples

A method was developed for the detection of antibiotic resistance genes by T2MR and sequencing (e.g., next-generation sequencing (NGS)) in complex samples such as blood. The method provides a therapeutically meaningful rapid result via T2MR-based detection, which can be combined with sequencing to enable typing and higher-resolution analyses (e.g., identification of antibiotic resistance variants).

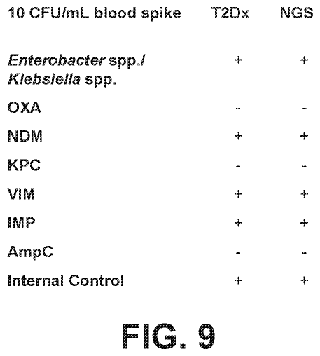

10 CFU/mL blood samples spiked with multiple resistant organisms were run on a T2DX® device with the T2CARBA Resistance+Panel and sequenced using ILLUMINA® NGS essentially as described in U.S. Provisional Patent Application No. 62/729,375. Concordance occurred when a target was detected both by T2DX® and by sequencing, and in this experiment, 100% concordance was achieved. Results are shown in FIG. 9. In this experiment, samples were run with and without amplicon purification prior to sequencing; both had 100% concordance, and amplicon purification only impacted background of human sequences.

The rapid, lower-cost T2MR result can be used to screen for samples from which more information can be obtained by sequencing. Pre-screening with T2MR allows for negative samples not to be tested; pooling of samples on sequencing runs can reduce the cost-per-sample sequenced, and narrows the window of data analysis. Sequencing can provide higher resolution information to expand on the T2MR menu and to detect SNPs, and resistance variants that cannot be distinguished by molecular diagnostics. The multi-staged results from T2MR and sequencing provide information on an accelerated, clinically meaningful timeline.

### Example 3: T2Resistance Panel

The T2Resistance Panel is a qualitative molecular diagnostic assay that employs a whole blood compatible PCR amplification followed by T2 magnetic resonance (T2MR) detection or sequencing-based detection. The T2Resistance Panel may be performed on the T2DX® Instrument, which executes all steps after specimen loading, with up to seven blood specimens loaded at the same time. Individually, a K₂EDTA whole blood specimen containing a minimum of 3 mL is loaded directly onto the T2Resistance Sample Inlet, which is then placed on the T2Resistance cartridge along with the T2Resistance reagent tray. The cartridge and reagent tray contain a lysis reagent, an internal control, primers, enzyme, buffer and probe-coupled superparamagnetic particles for each detected target. In other examples, the T2Resistance Panel may be detected by sequencing.

After loading into the T2DX®, the blood specimen is mixed with the red blood cell lysing reagent and the bacterial cells and human cellular debris are concentrated by centrifugation. The internal control is added to the concentrated pellet; a bead-beating process then lyses the bacterial cells. The supernatant containing the DNA from the lysed bacterial cells and the internal control is amplified using the target and internal control-specific primers. The generated amplified product is aliquoted into individual tubes containing target-specific detection particles for each detected target and the internal control. The amplified DNA is hybridized to target-specific probes attached to superparamagnetic particles causing clustering of the particles. The hybridization occurring in individual tubes is analyzed in the T2MR reader and a signal for each target is generated and is detected by T2MR indicating the presence of the target organism.

When running the first specimen in a series or a single specimen, the result is reported in 3.5 hours from the time the specimen is loaded onto the instrument, days earlier than what is currently available using conventional microbiology methods or using rapid culture-dependent tests. The results can be interpreted by the device software as valid or invalid (based on the result of the internal control or target detections), and if valid, results are reported as “Positive” or “Target not Detected” for each specific target. The T2Resistance Panel will provide to clinicians information about the presence or absence of resistance genes days earlier than currently possible, enabling clinicians to make better informed therapy decisions for patients suspected of bloodstream infections far earlier in the progression of infection.

### Indications for Use

The T2Resistance Panel can be used in a qualitative T2 magnetic resonance (T2MR) assay for the direct detection of bacterial species and resistance genes in K₂EDTA human whole blood specimens from patients with suspected bacteremia. The T2Resistance Panel detects the following thirteen bacterial resistance gene sequences and categorizes them into the following seven groups:

KPC (bla_{KPC})

CTX-M 14 and CTX-M 15

Metallo-beta-lactamases (bla_{NDM}, bla_{VIM} & bla_{IMP})

bla_{OXA-48}

vanA and vanB

mecA and mecC

AmpC (CMY and DHA)

The T2Resistance Panel does not distinguish between CTX-M 14 and CTX-M 15.
The T21Resistance Panel does not distinguish between bla_{NDM}, bla_{VIM} and bla_{IMP}.
The T21Resistance Panel does not distinguish between vanA and vanB.
The T2Resistance Panel does not distinguish between mecA and mecC.
The T2Resistance Panel does not distinguish between CMY and DHA.

Exemplary primer and probe nucleic acid sequences for the T21Resistance panel are shown below in Table 17.

TABLE 17
Primer and Probe Sequences for T2Resistance Panel

| SEQ ID NO: | Name | Sequence |
|---|---|---|
| 3 | KPC F Primer | TTTCTGCCACCGCGCTGAC |
| 4 | KPC R Primer | GCAGCAAGAAAGCCCTTGAATG |
| 19 | KPC 5′ Probe | CGGAACCATTCGCTAAACTC |
| 20 | KPC 3′ Probe | AGGCGCAACTGTAAGTTACCG |
| 33 | CTX-M 14 F Primer | ACGCTTTCCAATGTGCAGTACCAGTA |
| 34 | CTX-M 14 R Primer | TGCGATCCAGACGAAACGTCTCATCG |
| 57 | CTX-M 14 5′ Probe | CTGTCGAGATCAAGCCTGCCGA |
| 58 | CTX-M 14 3′ Probe | ACAAATTGATTGCCCAGCTCGGT |
| 81 | CTX-M 15 F Primer | CCTCGGGCAATGGCGCAAAC |
| 82 | CTX-M 15 R Primer | ATCGCGACGGCTTTCTGCCTTA |
| 83 | CTX-M 15 5′ Probe | GGGCGCAGCTGGTGACAT |
| 84 | CTX-M 15 3′ Probe | AAGATCGTGCGCCGCTGATT |
| 85 | vanA F Primer (F2) | TATTCATCAGGAAGTCGAGCCGGA |
| 50 | vanA R Primer | CAGTTCGGGAAGTGCAATACCTGCA |
| 86 | vanA 5′ Probe (#2) | GCAGTTATAACCGTTCCCGCAG |
| 87 | vanA 3′ Probe (#2) | TAACGGCCGCATTGTACTGAACG |
| 51 | vanB F Primer | AATTGAGCAAGCGATTTCGGGCTGT |
| 88 | vanB R Primer (R2) | AAGATCAACACGGGCAAGCCCTCT |
| 75 | vanB 5′ Probe | GCACCCGATATACTTTCTTTGCC |
| 76 | vanB 3′ Probe | CGCCGACAATCAAATCATCCT |
| 9 | OXA-48-like F Primer | GGCTGTGTTTTTGGTGGCATCGATTATC |
| 10 | OXA-48-like R Primer | TCCCACTTAAAGACTTGGTGTTCATCC |
| 25 | OXA-48-like 5′ Probe | ATTTTAAAGGTAGATGCGGG |
| 26 | OXA-48-like 3′ Probe | CGCCCTGTGATTTATGTTCA |
| 41 | mecA F Primer | ATGTTGGTCCCATTAACTCTGAAGAA |
| 42 | mecA R Primer | CACCTGTTTGAGGGTGGATAGCAGTA |
| 89 | mecA 5′ Probe (#2) | AAAGGCTATAAAGATGATGCAG |
| 90 | mecA 3′ Probe (#2) | GAGTATTTATAACAACATGAAAAATGATT |
| 91 | mecC F Primer | ATGTGGGTCCAATTAATTCTGACGAG |
| 92 | mecC R Primer | CTCCAGTTTTTGGTTGTAATGCTGTA |
| 93 | mecC 5′ Probe | GGCTTAGAACGCCTCTATGAT |
| 94 | mecC 3′ Probe | AGAGTACAAGAAAGTATTTATAAACATATGA |
| 13 | CMY F Primer | CCGCGGCGAAATTAAGCTCAGCGA |
| 14 | CMY R Primer | CCAAACAGACCAATGCTGGAGTTAG |
| 29 | CMY 5′ Probe | GCGGCTGCCAGTTTTGATAA |
| 30 | CMY 3′ Probe | GTGGCTAAGTGCAGCAGGC |
| 11 | DHA F Primer | ACGGGCCGGTAATGCGGATCTGGA |
| 12 | DHA R Primer | TATTCGCCAGAATCACAATCGCCACCTGT |
| 27 | DHA 5′ Probe | GTTTTATGCACCCAGGAAGC |
| 28 | DHA 3′ Probe | TCTGCTGCGGCCAGTCATA |
| 1 | NDM F Primer | CCAGCTCGCACCGAATGTCT |
| 2 | NDM R Primer | CATCTTGTCCTGATGCGCGTGAGTCA |
| 17 | NDM 5′ Probe | GCGACCGGCAGGTTGATCT |
| 18 | NDM 3′ Probe | CATGTCGAGATAGGAAGTGTGCTGC |
| 5 | IMP F Primer | ACATTTCCATAGCGACAGCACGGGCGGAAT |
| 6 | IMP R Primer | GGACTTTGGCCAAGCTTCTAAATTTGCGTC |
| 21 | IMP 5′ Probe | GCTTAATTCTCAATCTATCCCCACGTAT |
| 22 | IMP 3′ Probe | CTCCAGATAACGTAGTGGTTTGGCTG |
| 7 | VIM F Primer | CGTGCAGTCTCCACGCACT |
| 8 | VIM R Primer | TCGAATGCGCAGCACCGGGATAG |
| 23 | VIM 5′ Probe | CTTTCATGACGACCGCGTCGG |
| 24 | VIM 3′ Probe | CTCTAGAAGGACTCTCATCGAGC |

The T2Resistance Panel may be used as an aid in the diagnosis of bacteremia and results may be used in conjunction with other clinical and laboratory data. Concomitant blood cultures can be used to recover organisms for further identification and for organisms not detected by the T2Resistance Panel.

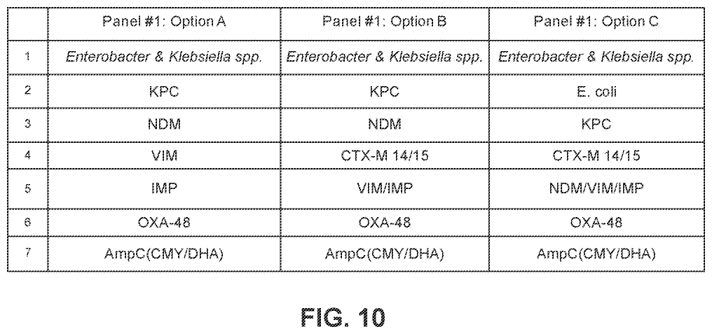

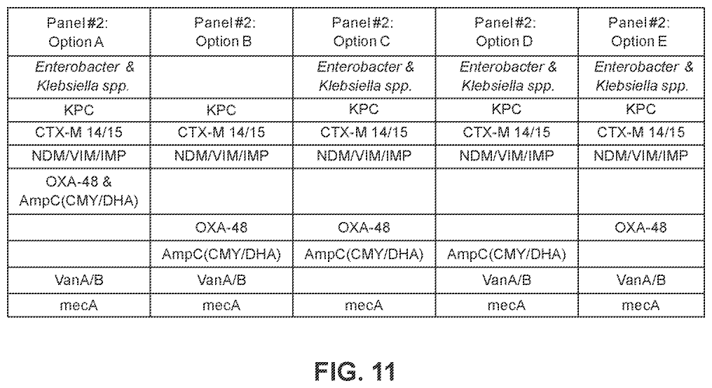

The members of this panel can be detected using a variety of assay configurations (see FIGS. 10 and 11). For example, in some examples, CTX-M 14 and 15 can be omitted (see FIG. 10, panel #1, Option A). In this example, NDM, VIM, and IMP may be individually detected in separate channels. In another example, the panel includes a CTX-M 14/15 channel and a VIM/IMP channel (see FIG. 10, panel #1, Option B). In another example, the panel includes a CTX-M 14/15 channel, a VIM/IMP/NDM channel, and an E. coli channel (see FIG. 10, Panel #1, Option C). Additional exemplary panel configurations are shown in FIG. 11.

### Species Selection/Clinical Benefit of T2Resistance

The T2Resistance Panel includes detection and identification of several classes of resistance genes in both Gram positive and Gram negative pathogens. These include detection of the major carbapenem resistance genes (bla_{KPC}, bla_{OXA}, bla_{NDM}, bla_{VIM}, bla_{IMP}), which are listed on the urgent CDC Resistance Threat list; detection of extended spectrum beta lactamases (ESBL) known as CTX-M 14 and CTX-M 15; detection of the gram positive vanA/B resistance genes, which are responsible for vancomycin resistant enterococcus; detection of the MRSA (methicillin resistant Staphylococcus aureus) associated resistance genes mecA and mecC, and detection of the AmpC beta-lactamases bla_{CMY} and bla_{DHA}.

Bloodstream infections by organisms containing the resistance genes detected by the T2Resistance Panel are life threatening due to their association with high mortality rates. Patients with CRE bloodstream infections have been shown to have mortality rates >38% when they receive therapy within 5 days and delay of therapy beyond 5 days has been shown to be associated with an increase in mortality rate of 60.7%. Similarly, blood stream infections caused by ESBL Enterobacteriasceae were shown to have a 21-day mortality rate of 18% when patients were placed on correct therapy within a few hours of infection onset but this rate increase to 59% for individuals that were not treated correctly within 72 hours. ESBL infections world-wide are increasingly being associated with the presence of CTX-M genotype. Vancomycin resistant Enterococcus (VRE) infections have been increasing and the CDC reported in 2013 that 30% of Enterococcus infections were caused by vancomycin resistant organisms. A meta-analysis of mortality associated with VRE demonstrated that compared to infections caused by vancomycin sensitive Enterococcus there was significant increase in mortality with an odds ratio of 2.52 (95% CI 1.9-3.4).

### Results

Table 18 shows detection data from whole blood samples spiked with bacterial strains that contained detected targets. These studies demonstrated 100% detection with levels of bacteria of 5-10 CFU/mL.

TABLE 18
Detection data from whole blood samples
spiked with bacterial targets

| Target | CFU/mL | Number Tested | Percent Positive (%) |
|---|---|---|---|
| KPC (AR-0112) | 5 | 36 | 100 |
| NDM (AR-0037) | 5 | 36 | 94 |
| OXA (AR-0074) | 5 | 36 | 100 |
| VIM (AR-0154) | 10 | 36 | 100 |
| IMP (AR-0103 | 5 | 36 | 100 |
| CTX-M 14 (AR-0079) | 5 | 36 | 100 |
| CTX-M 15 (AR-0044) | 5 | 36 | 100 |
| vanA (BEI NR-31928) | 10 | 36 | 100 |
| vanB (JMI 1031982) | 10 | 36 | 100 |
| mecA (AR-0215) | 2 | 36 | 100 |
| mecC (BAA-2312) | 5 | 36 | 100 |
| CMY (AR-0081) | 5 | 36 | 100 |
| DHA (AR-0079) | 5 | 36 | 100 |

These data demonstrate that the T2Resistance panel can be used for rapid (less than about 4 h) and sensitive detection (detection of a concentration of 1-10 CFU/mL) of the members of the panel in a direct-from-blood assay.

### Example 4: Evaluation of the T2Carba Resistance+ and T2ARx Panels

A study was performed to evaluate detection of bacterial species and resistance genes using panels of the invention, including the T2Carba Resistance+panel and the T2ARx panel. Table 19 shows the members of these panels as well as a T2Bacteria panel. A. baumanni can be omitted from the T2Bacteria panel, as in the FDA-cleared T2BACTERIA panel. These panels were developed to provide rapid and sensitive direct-from-blood diagnostic assays to detect infectious pathogens and antibiotic resistance genes, including those on the Centers for Disease Control and Prevention (CDC) Urgent, Serious, and Concerning Threats list. The panels are designed to detect their targets within 4 hours of patient blood draw and with a limit of detection of s 10 CFU/mL (e.g., 1-10 CFU/mL).

The panels can be used, for example, with the FDA-cleared T2DX® device (T2 Biosystems, Inc., Lexington, Mass.). The T2DX® is a fully automated sample-to-results system that performs all steps in the Panel after specimen loading. After the operator loads a K₂EDTA blood sample onto the cartridge, the T2DX® pipettes 2 mL of blood and treats it with a detergent to selectively lyse the red blood cells. Pathogen cells are then concentrated via centrifugation followed by supernatant removal. After concentration, the pathogen cells and other cellular debris are subjected to bead beating to release target DNA. This lysate is then amplified with a whole blood multiplexed asymmetric PCR method. After amplification, target amplicon is aliquoted into separate tubes for hybridization with unique capture probe functionalized superparamagnetic particles, which causes particle aggregation and a change in T2. The Panel includes an Internal Control (IC) that monitors the amplification and detection process. T2MR can detect in complex matrices and therefore enables a true direct-from-blood measurement that can be tailored for other sample matrices such as urine, wound swabs, dialysis, and sputum. Consequently, sensitivity and speed are maximized since neither sample purification or blood culture is necessary. However, other approaches can also be used for detection, including sequencing-based detection approaches.

TABLE 19
T2 panel assays for detection of bacterial
species and resistance genes

| T2 Panel | | |
|---|---|---|
| Assay | N | Species or resistance genes identified |
| T2Bacteria | 6 | A. baumannii, E. coli, E. faecium, |
| | | K. pneumoniae, P. aeruginosa, S. aureus |
| T2Carba | 13 | Enterobacter spp. (E. aerogenes, E. cloacae, |
| Resistance+ | | E. hormachei, K. pneumoniae, K. varicola and |
| | | K. oxytoca), AmpC (CMY, DHA), IMP, KPC, |
| | | NDM, VIM, OXA-48 |
| T2ARx | 11 | S. pneumoniae, CTX-M 14, CTX-M 15, ermA, |
| | | ermB, mecA, mef, blaSHV, blaTEM, vanA, vanB |

The primers and probes used in the T2Carba Resistance+panel are shown in Table 16 (see Example 1). The detection of Enterobacter spp. in the T2Carba Resistance+panel is designed to be inclusive of most Enterobacter and Klebsiella strains (also referred to as the “EK channel”), and the following were tested as part of the this study: E. aerogenes, E cloacae, E. hormachei, K. pneumoniae, K. varicola and K. oxytoca.

The primers and probes used in the T2ARx panel are shown in Table 20.

TABLE 20
Primers and Probes for T2Arx panel

| SEQ ID | | |
|---|---|---|
| NO | Name | Sequence |
| 33. | CTX-M 14 F Primer | ACGCTTTCCAATGTGCAGTACCAGTA |
| 34. | CTX-M 14 R Primer | TGCGATCCAGACGAAACGTCTCATCG |
| 35. | CTX-M 15 F Primer | GTGATACCACTTCACCTCGGGCAA |
| 36. | CTX-M 15 R Primer | AATACATCGCGACGGCTTTCTGCC |
| 37. | ermA F Primer | AGAATTACCTTTGAAAGTCAGGC |
| 38. | ermA R Primer | GCTTCAAAGCCTGTCGGAATTGGTTT |
| 39. | ermB F Primer | GGGCATTTAACGACGAAACTGGCTA |
| 40. | ermB R Primer | GTGTTCGGTGAATATCCAAGGTACGC |
| 41. | mecA F Primer | ATGTTGGTCCCATTAACTCTGAAGAA |
| 42. | mecA R Primer | CACCTGTTTGAGGGTGGATAGCAGTA |
| 43. | mefA F Primer | GCAGGGCAAGCAGTATCATTAATCAC |
| 44. | mefA R Primer | AATTAAATCAGCACCAATCATTATCTTCTTCC |
| 45. | SHV F Primer | AAGCTGCTGACCAGCCAGCGTCTGA |
| 46. | SHV R Primer | CGGCGATTTGCTGATTTCGCTCG |
| 47. | TEM F Primer | TGCAGTGCTGCCATAACCATGAGTGA |
| 48. | TEM R Primer | AGCGCAGAAGTGGTCCTGCAACTTT |
| 49. | vanA F Primer | CAGTACGGRATOTTTCGTATTCATCAGGA |
| 50. | vanA R Primer | CAGTTCGGGAAGTGCAATACCTGCA |
| 51. | vanB F Primer | AATTGAGCAAGCGATTTCGGGCTGT |
| 52. | vanB R Primer | CGTTTAGAACGATGCCGCCATCCT |
| 53. | spr0075 F Primer | CCTTGGACGGAAATGTAGCTGGCA |
| | (Streptococcus | |
| | pneumoniae) | |
| 54. | spr0075 R Primer | AATCACATGGTTGACACCTGCTGTG |
| | (Streptococcus | |
| | pneumoniae) | |
| 57. | CTX-M 14 5′ Probe | CTGTCGAGATCAAGCCTGCCGA |
| 58. | CTX-M 14 3′ Probe | ACAAATTGATTGCCCAGCTCGGT |
| 59. | CTX-M 15 5′ Probe | AATCAGCGGCGCACGATCTTT |
| 60. | CTX-M 15 3′ Probe | AATGCTCGCTGCACCGGTGGTAT |
| 61. | ermA 5′ Probe | AAATCTGCAACGAGCTTTGGG |
| 62. | ermA 3′ Probe | GTTTATAAGTGGGTAAACCGTGAATATC |
| 63. | ermB 5′ Probe | TTCGTGTCACTTTAATTCACCAAGAT |
| 64. | ermB 3′ Probe | AAAGCCATGCGTCTGACATCT |
| 65. | mecA 5′ Probe | AAGCTCCAACATGAAGATGGCT |
| 66. | mecA 3′ Probe | AGATGGCAAAGATATTCAACTAAC |
| 67. | mefA 5′ Probe | AGTGCCATCTTGCAAATGGCGAT |
| 68. | mefA 3′ Probe | TGCAATTGGTGTGTTAGTGGATCGTCATGATA |
| 69. | SHV 5′ Probe | CAGTGGATGGTGGACGATCGGGT |
| 70. | SHV 3′ Probe | TTGTGGTGATTTATCTGCGGGATACT |
| 71. | TEM 5′ Probe | CGCCAGTTAATAGTTTGCGCAACG |
| 72. | TEM 3′ Probe | AAAGCGGTTAGCTCCTTCGGTCCT |
| 73. | vanA 5′ Probe | CGTTCAGTACAATGCGGCCGTTA |
| 74. | vanA 3′ Probe | CTGCGGGAACGGTTATAACTGC |
| 75. | vanB 5′ Probe | GCACCCGATATACTTTCTTTGCC |
| 76. | vanB 3′ Probe | CGCCGACAATCAAATCATCCT |
| 77. | spr0075 5′ Probe | TTGACCAGTTCCGAGCAAATGGTA |
| | (Streptococcus | |
| | pneumoniae) | |
| | Spn2rc_3′ | |
| 78. | spr0075 3′ Probe | GACAGTATCGATGTTCCAGCAGCT |
| | (Streptococcus | |
| | pneumoniae) | |
| | Spnrc_5′ | |

In these assays, the OIC internal control primer and probe sequences can be used. These sequences are shown below in Table 21.

TABLE 21
OIC Primer and Probe Sequences

| SEQ ID | | |
|---|---|---|
| NO | Name | Sequence |
| 55 | OIC F Primer | GGAAATCTAACGAGAGAGCATGCT |
| 56 | OIC R Primer | CGATGCGTGACACCCAGGC |
| 79 | OIC 3′ Probe | GAGACGTTTTGGATACATGTGAAAGA |
| | | AGGC |
| 80 | OIC 5′ Probe | CGATGGTTCACGGGATTCTGCAATTC |

With respect to the T2ARx panel, CTX-M resistance variants are grouped into five families. Based on limited similarity and homology between these families the assay was designed to be inclusive of the most common variants, CTX-M 14 and CTX-M 15. This design detects all CTX-M variants within the CTX-M group 1 and group 9 families. ERM detection was designed to detect both ermA and ermB resistance genes. VAN detection was designed to detect bath vanA and vanB resistance genes.

These panels can be used individually or in combination. In some examples, the T2Carba Resistance+panel may be used with the T2Bacteria panel. In other examples, the T2Carba Resistance+panel may be used with the T2ARx panel. In yet other examples, the T2ARx panel may be used with the T2Bacteria panel. In further examples, the T2Carba Resistance+panel, the T2ARx panel, and the T2Bacteria panel are used. For combination panels, in one example, each of the T2Bacteria, T2Carba Resistance+, and T2ARx panels can be contained in its own cartridge, and patient samples (e.g., whole blood) can be split into separate aliquots for testing with each cartridge (see Table 22).

TABLE 22
Exemplary layout of panel members in T2DX ® cartridges

| | | Cartridge #2: | |
|---|---|---|---|
| Gene | Cartridge #1: | T2Carba | Cartridge #3: |
| type | T2Bacteria | Resistance+ | T2ARx |
| Bacteria | 1. Acinetobacter | 1. Enterobacter spp. | 1. Streptococcus |
| species | baumannii (may | (e.g., E. aerogenes, | pneumoniae |
| | be omitted) | E. cloacae, | |
| | 2. Enterococcus | E. hormachei, | |
| | faecium | K. pneumoniae, | |
| | 3. Escherichia coli | K. varicola and | |
| | 4. Klebsiella | K. oxytoca), | |
| | pneumoniae | | |
| | 5. Pseudomonas | | |
| | aeruginosa | | |
| | 6. Staphylococcus | | |
| | aureus | | |
| Resistance | — | 2. AmpC | 2. CTX |
| markers | | (CMY and DHA) | 3. ERM |
| | | 3. IMP | 4. mecA |
| | | 4. KPC | 5. mefA |
| | | 5. NDM | 6. SHV/TEM |
| | | 6. OXA-48 | 7. VAN |
| | | 7. VIM | |

### Materials and Methods

### A. High Titer Study

Table 23 shows the bacterial species and strains used in this study. Resistance markers in bald text are those detected by the assays described herein. ATCC indicates American Type Culture Collection, JMI indicates JMI Labs, BEI indicates BEI Resources, and CDC indicates Centers for Disease Control and Prevention.

TABLE 23
Bacterial species and strains used in high titer testing

| | Strain | | | Resistance |
|---|---|---|---|---|
| T2 Panel | ID | Source | Species | Markers |
| T2Bacteria | 17978 | ATCC | Acinetobacter | Unknown |
| | | | baumannii | |
| | 17904 | ATCC | Acinetobacter | Unknown |
| | | | baumannii | |
| | 27270 | ATCC | Enterococcus | Unknown |
| | | | faecium | |
| | 49224 | ATCC | Enterococcus | Unknown |
| | | | faecium | |
| | 10798 | ATCC | Escherichia | Unknown |
| | | | coli | |
| | 35150 | ATCC | Escherichia | Unknown |
| | | | coli | |
| | 27736 | ATCC | Klebsiella | Unknown |
| | | | pneumoniae | |
| | BAA-1898 | ATCC | Klebsiella | Unknown |
| | | | pneumoniae | |
| | 27853 | ATCC | Pseudomonas | Unknown |
| | | | aeruginosa | |
| | 14203 | ATCC | Pseudomonas | Unknown |
| | | | aeruginosa | |
| | 29213 | ATCC | Staphylococcus | Unknown |
| | | | aureus | |
| | 11371 | ATCC | Staphylococcus | Unknown |
| | | | aureus | |
| T2Carba | AR-0034 | CDC | Klebsiella | aac(3)-IId, catB3, |
| Resistance+ | | | pneumoniae | IMP-4, |
| | | | | mph(A), oqxA, |
| | | | | oqxA, QnrB2, |
| | | | | SHV-11, sul1, |
| | | | | TEM-1B |
| | BAA-830 | ATCC | Klebsiella | Unknown |
| | | | variicola | |
| | Z115 | ATCC | Klebsiella | Unknown |
| | | | oxytoca | |
| | 23355 | ATCC | Enterobacter | Unknown |
| | | | cloacae | |
| | 49162 | ATCC | Enterobacter | Unknown |
| | | | hormaechei | |
| | AR-0112 | CDC | Klebsiella | aac(6′), aph(3′), |
| | | | pneumoniae | aph(4), catA1, |
| | | | | cmlA1, dfrA12, |
| | | | | KPC-3, |
| | | | | mph(A), oqxA, |
| | | | | oqxA, oqxB, |
| | | | | sul1, sul3 |
| | AR-0074 | CDC | Enterobacter | ARR-3, OXA-48 |
| | | | aerogenes | |
| | AR-0037 | CDC | Acinetobacter | NDM-1, |
| | | | baumannii | OXA-94, sul2 |
| | AR-0054 | CDC | Pseudomonas | strB, OXA-50, |
| | | | aeruginosa | aadB, PAO, |
| | | | | tet(A), VIM-4, |
| | | | | strA, catB7 |
| | AR-0090 | CDC | Pseudomonas | KPC-5, |
| | | | aeruginosa | OXA-50, PAO |
| | AR-0081 | CDC | Escherichia | TEM-1B, CMY-2 |
| | | | coli | |
| | AR-0061 | CDC | Escherichia | KPC-3, |
| | | | coli | OXA-9, TEM-1A |
| | AR-0519 | CDC | Morganella | DHA-type |
| | | | morganii | |
| T2ARx | BAA-1407 | ATCC | Streptococcus | mefE, ermB |
| | | | pneumoniae | |
| | NR-31928 | BEI | Enterococcus | vanA, ermB |
| | | | faecium | |
| | AR-0051 | CDC | Klebsiella | aadA2, armA, |
| | | | ozaenae | aac(3)-IId, |
| | | | | aac(6′)Ib-cr, |
| | | | | CTX-M-15, |
| | | | | OXA-181, |
| | | | | SHV-26, |
| | | | | catA1, ARR-3, |
| | | | | sul1, sul2, |
| | | | | tet(A), dfrA12, |
| | | | | dfrA14, OmpK35 |
| | AR-0215 | CDC | Staphylococcus | aadD, spc, blaZ, |
| | | | aureus | mecA, erm(A) |
| | AR-0506 | CDC | Klebsiella | CTX-M-14, |
| | | | pneumoniae | CTX-M-15, |
| | | | | NDM-1, |
| | | | | SHV-ESBL(u), |
| | | | | TEM-OSBL(b) |
| | 1031982 | JMI | Enterococcus | vanB, ermB |
| | | | faecium | |

### T2Bacteria Panel Testing

The T2Bacteria Panel has been optimized for processing on the T2DX® instrument and thus all testing was done on the instrument. Whole blood samples were spiked with target bacterial species (Table 23) at specific concentrations. Two strains for each bacterial species were tested. The amount of bacteria spiked into samples was confirmed by growing an aliquot of the bacterial suspension used for spiking on agar plates to determine CFU/mL. Spiked blood samples were processed on the T2DX® device using the T2Bacteria cartridge and reagent tray according to manufacturer's instructions. Limit of detection was determined to be the lowest concentration of spiked bacteria that resulted in ≥95% sensitivity (minimum of 19/20 positive detections) (Table 24). Testing was done using two different strains for each species and two reagent lots with an n a 20 per test and a total n a 80 per species (Table 25).

TABLE 24
T2Bacteria percent positive detection

| | | Strain 1 | | Strain 2 | |
|---|---|---|---|---|---|
| | | Reagent | Reagent | Reagent | Reagent |
| Species | CFU/mL | Lot 1 | Lot 2 | Lot 1 | Lot 2 |
| A. baumannii | 3 | 100% | 100% | 100% | 100% |
| E. faecium | 5 | 100% | 100% | 95% | 100% |
| E. coli | 11 | 95% | 100% | 100% | 100% |
| K. pneumoniae | 1 | 90% | NT | 100% | 100% |
| | 2 | 95% | 100% | NT | NT |
| P. aeruginosa | 3 | 100% | 100% | 45% | NT |
| | 5 | NT | NT | 100% | 100% |
| S. aureus | 2 | 100% | 100% | 100% | 100% |

TABLE 25
T2Bacteria Limit of Detection by species and strain

| | | | | | Final Confirmed | |
|---|---|---|---|---|---|---|
| Species | Strain 1 LoD | | Strain 2 LoD | | LoD | |
| A. baumannii | 3 | CFU/mL | 3 | CFU/mL | 3 | CFU/mL |
| E. faecium | 5 | CFU/mL | 5 | CFU/mL | 5 | CFU/mL |
| E. coli | 11 | CFU/mL | 11 | CFU/mL | 11 | CFU/mL |
| K. pneumoniae | 2 | CFU/mL | 1 | CFU/mL | 2 | CFU/mL |
| P. aeruginosa | 3 | CFU/mL | 5 | CFU/mL | 5 | CFU/mL |
| S. aureus | 2 | CFU/mL | 2 | CFU/mL | 2 | CFU/mL |

### T2CarbaResistance+Panel Testing

Whole blood samples were spiked with targeted bacterial species or with bacterial strains that contained targeted resistance genes at 10 or 15 CFU/mL. An aliquot of the bacterial suspension used for spiking samples was grown on agar plates to confirm spike concentrations (CFU/mL). Whole blood samples were prepared to contain either multiple or single bacterial species as shown in Table 26. The total number of replicates for each target was 20 samples. As a negative control, whole blood containing no bacteria was tested.

TABLE 26
Spiked samples produced for T2Carba Resistance+ testing

| Spiked | Bacterial | Strain | Detected | Concentration | Number |
|---|---|---|---|---|---|
| Sample | Species | ID | Targets | CFU/mL | Tested |
| M1 | M. morganii | AR-0519 | DHA (AmpC) | 15 | 12 |
| | K. pneumoniae | AR-0034 | K. pneumoniae, | 10 | |
| | | | IMP-4 | | |
| | A. baumannii | AR-0037 | NDM-1 | 10 | |
| | P. aeruginosa | AR-0054 | VIM-4 | 10 | |
| M2 | E. coli | AR-0081 | CMY-2 | 10 | 8 |
| | | | (AmpC) | | |
| | E. coli | AR-0061 | KPC-3 | 10 | |
| | E. aerogenes | AR-0074 | E. aerogenes, | 10 | |
| | | | OXA-48 | | |
| S1 | M. morganii | AR-0519 | DHA (AmpC) | 15 | 8 |
| S2 | E. coli | AR-0081 | CMY-2 | 10 | 16 |
| | | | (AmpC) | | |
| S3 | K. pneumoniae | AR-0034 | K. pneumoniae, | 10 | 16 |
| | | | IMP-4 | | |
| S4 | E. coli | AR-0061 | KPC-3 | 10 | 8 |
| S5 | P. aeruginosa | AR-0090 | KPC-5 | 10 | 8 |
| S6 | A. baumannii | AR-0037 | NDM-1 | 10 | 16 |
| S7 | E. aerogenes | AR-0074 | E. aerogenes, | 10 | 16 |
| | | | OXA-48 | | |
| S8 | P. aeruginosa | AR-0054 | VIM-4 | 10 | 8 |
| S9 | K. variicola | BAA-830 | K. variicola | 10 | 20 |
| S10 | K. oxytoca | Z115 | K. oxytoca | 10 | 20 |
| S11 | E. cloacae | 23355 | E. cloacae | 10 | 20 |
| S12 | E. hormaechei | 49162 | E. hormaechei | 10 | 20 |
| | None | NA | Negative | NA | 12 |
| | | | Control | | |

Samples were processed using a manual assay that mimics testing on the T2DX® device. 2 mL of the blood sample containing the bacterial spike was added to a lysis tube that contained beads and lysis reagent, which lysed red blood cells in the sample. The sample was centrifuged to concentrate the pathogens. The supernatant was removed and buffer containing the internal control was added. Pathogens were lysed using mechanical disruption. Bacterial lysate was added to a multiplexed amplification reaction (single amplification reaction that will amplify all target DNA). Post amplification, DNA was added to different tubes each containing different probe-coated superparamagnetic particles specific for each target detected. In these experiments, the target DNA binds to probes on the particles and results in particle clustering. Samples were detected using T2 magnetic resonance to give a T2 value (ins). Particle clustering results in a higher value. For these studies, any T2 value above 65 ms was considered a positive result. Testing of the T2Carba Resistance+panel with either multi-spiked or single spiked samples resulted in 100% detection of all targets. Testing of negative controls resulted in no positive detection in any channel. Table 27 shows a summary of the results of these experiments.

TABLE 2627
Summary of T2Carba Resistance+ panel testing

| Target | DHA | CMY | IMP | KPC | NDM | OXA | VIM |
|---|---|---|---|---|---|---|---|
| CFU/mL | 15 | 10 | 10 | 10 | 10 | 10 | 10 |
| Number | 20 | 24 | 28 | 24 | 28 | 24 | 20 |
| Tested | | | | | | | |
| Percent | 100% | 100% | 100% | 100% | 100% | 100% | 100% |
| Positive | | | | | | | |

| | K. | K. | K. | E. | E. | E. |
|---|---|---|---|---|---|---|
| Target | pneumoniae | variicola | oxytoca | aerogeneses | cloacae | homaechei |
| CFU/mL | 10 | 10 | 10 | 10 | 10 | 10 |
| Number | 20 | 20 | 20 | 20 | 20 | 20 |
| Tested | | | | | | |
| Percent | 100% | 100% | 100% | 100% | 100% | 100% |
| Positive | | | | | | |

### T2ARx Panel Testing

Whole blood samples were spiked with targeted bacterial species or with bacterial strains that contained targeted resistance genes. Bacteria were spiked at 50 CFU/mL and an aliquot of the bacterial suspension used for spiking samples was grown on agar plates to confirm concentration (CFU/mL). Whole blood samples containing multiple bacterial species were used for testing (Table 28). As a control whole blood containing no bacteria were tested. Samples were processed using a manual assay as described above with respect to the T2Carba Resistance+panel. Results with T2 values above 65 ms were considered a positive detection.

TABLE 28
Spiked samples produced for T2ARx testing

| Spiked | Bacterial | Strain | Targets | | Number |
|---|---|---|---|---|---|
| Sample | Species | ID | expressed | CFU/mL | tested |
| M1 | E. faecium | NR-31928 | vanA | 50 | 20 |
| | S. pneumoniae | BAA-1407 | S. pneumoniae, | 50 | |
| | | | mef, ermB | | |
| | K. ozaenae | AR-0051 | CTX-M 15, | 50 | |
| | | | SHV-26 | | |
| M2 | E. faecium | 1031982 | vanB | 50 | 20 |
| | S. aureus | AR-0215 | ermA, mecA | 50 | |
| | K. pneumoniae | AR-0506 | CTX-M 15, | 50 | |
| | | | CTX-M 14, | | |
| | | | TEM-1, | | |
| | | | SHV-ESVL | | |
| | Negative | NA | None | NA | 8 |

Testing of two multi-spiked blood samples resulted in 100% (20/20) positive detection when bacteria were at 50 CFU/mL. Although not annotated to be erythromycin resistant, subsequent screening of E. faecium 1031982 and NR-31928 determined that these strains were resistant to erythromycin and were confirmed to contain the ermB marker based on sequencing. Processing of whole blood samples that contained no spiked bacteria resulted in no positive detections. Testing resulted in three false positives; two for blaTEM and one for mefA. A summary of these results is shown in Table 29.

TABLE 29
Summary of T2ARx high titer detection

| | CTX | CTX | | | | | | | | | S. |
|---|---|---|---|---|---|---|---|---|---|---|---|
| Target | M-14 | M-15 | ermA | ermB | mecA | mefA | SHV | TEM | vanA | vanB | pneumoniae |
| CFU/mL | 50 | 50 | 50 | 50 | 50 | 50 | 50 | 50 | 50 | 50 | 50 |
| Number | 20 | 20 | 20 | 20 | 20 | 20 | 20 | 20 | 20 | 20 | 20 |
| Tested | | | | | | | | | | | |
| Percent | 100% | 100% | 100% | 100% | 100% | 100% | 100% | 100% | 100% | 100% | 100% |
| Positive | | | | | | | | | | | |
| CFU/mL | 50 | 50 | 50 | 50 | 50 | 50 | 50 | 50 | 50 | 50 | 50 |

### B. Cross Reactivity Study

Table 30 shows the bacterial species and strains used in the cross reactivity studies (resistance markers in bold text are detected by panels).

TABLE 30
Bacterial species and strains used in high titer testing

| | Strain | | | Resistance |
|---|---|---|---|---|
| T2 Panel | ID | Source | Species | Markers |
| T2Bacteria | 17978 | ATCC | Acinetobacter | Unknown |
| | | | baumannii | |
| | 27270 | ATCC | Enterococcus | Unknown |
| | | | faecium | |
| | 10798 | ATCC | Escherichia | Unknown |
| | | | coli | |
| | 27736 | ATCC | Klebsiella | Unknown |
| | | | pneumoniae | |
| | 27853 | ATCC | Pseudomonas | Unknown |
| | | | aeruginosa | |
| | 29213 | ATCC | Staphylococcus | Unknown |
| | | | aureus | |
| T2Carba | AR-0054 | CDC | Pseudomonas | strB, OXA-50, |
| Resistance+ | | | aeruginosa | aadB, PAO, |
| | | | | tet(A), |
| | | | | VIM-4, |
| | | | | strA, catB7 |
| | AR-0061 | CDC | Escherichia | KPC-3, |
| | | | coli | OXA-9, TEM-1A |
| | AR-0074 | CDC | Enterobacter | ARR-3, |
| | | | aerogenes | OXA-48 |
| | AR-0037 | CDC | Acinetobacter | NDM-1, |
| | | | baumannii | OXA-94, sul2 |
| | PY79 | BGST | Bacillus | None |
| | | | subtilis | |
| | AR-0103 | CDC | Pseudomonas | IMP-1, |
| | | | aeruginosa | OXA-50, PAO, |
| | | | | aadB, strA, |
| | | | | strB, catB7, |
| | | | | tet(A), tet(G) |
| | AR-0081 | CDC | Escherichia | TEM-1B, |
| | | | coli | CMY-2 |
| | AR-0519 | CDC | Morganella | DHA-type |
| | | | morganii | |
| | 13048 | ATCC | E. aerogenes | Unknown |
| | 23355 | ATCC | Enterobacter | Unknown |
| | | | cloacae | |
| | 49162 | ATCC | Enterobacter | Unknown |
| | | | hormaechei | |
| | 27736 | ATCC | K. pneumoniae | Unknown |
| | BAA-830 | ATCC | Klebsiella | Unknown |
| | | | variicola | |
| T2ARx | BAA-1407 | ATCC | Streptococcus | mefE, ermB |
| | | | pneumoniae | |
| | 1029038 | JMI | E. faecium | ermB, vanA |
| | AR-0051 | CDC | Klebsiella | aadA2, armA, |
| | | | ozaenae | aac(3)-IId, |
| | | | | aac(6′)Ib-cr, |
| | | | | CTX-M-15, |
| | | | | OXA-181, |
| | | | | SHV-26, |
| | | | | catA1, ARR-3, |
| | | | | sul1, sul2, |
| | | | | tet(A), dfrA12, |
| | | | | dfrA14, OmpK35 |
| | AR-0215 | CDC | Staphylococcus | aadD, spc, |
| | | | aureus | blaZ, mecA, |
| | | | | erm(A) |
| | 1031982 | JMI | Enterococcus | vanB, ermB |
| | | | faecium | |
| | AR-0079 | CDC | Klebsiella | aac3-IId, |
| | | | pneumoniae | aaadA2, |
| | | | | rmtB, strA, |
| | | | | strB, |
| | | | | CTX-M-14, |
| | | | | DHA-1, |
| | | | | SHV-11, |
| | | | | TEM-1 B, |
| | | | | erm(42), oqxA, |
| | | | | oqxB, QnrB4, |
| | | | | sul2, tetG, |
| | | | | dfrA 12 |
| | AR-0044 | CDC | Klebsiella | aac6-Ib, aadA 1, |
| | | | pneumoniae | CTX-M-15, |
| | | | | OXA-1, OXA-9, |
| | | | | SHV-12, |
| | | | | TEM-1A, |
| | | | | fosA, catA1, |
| | | | | oqxA/B, sul2, |
| | | | | dfrA14 |
| | AR-0524 | CDC | Klebsiella | KPC-3, |
| | | | pneumoniae | SHV-12 |
| | AR-0552 | CDC | Klebsiella | Aac6-Ib, aadA5, |
| | | | pneumoniae | strB, KPC-2, |
| | | | | SHV-12, |
| | | | | TEM-1 B, |
| | | | | mphA, oqxA/B, |
| | | | | sul1/2, tetA, |
| | | | | dfrA17 |
| | 569013 | JMI | Streptococcus | mefA |
| | | | pyoqenes | |
| | 1030283 | JMI | Enterococcus | ermB, vanA |
| | | | faecium | |
| | 1004470 | JMI | Enterococcus | ermB, vanB |
| | | | faecium | |

### T2Bacteria Panel Testing

Whole blood samples were spiked with a single bacterial species at 1,000 CFU/ml (Table 31) and tested with three replicates of each species. Spiked blood samples were processed on the T2DX® device using the T2Bacteria® cartridge and T2Bacteria® reagent tray according to manufacturer's instructions. Table 31 shows detection results from testing. Initial testing with K. pneumoniae resulted in one false positive detection for E. coli. Repeat testing with new K. pneumoniae spiked samples was performed and no false positives were observed in any detection channel. No false positive results were obtained for any other species tested.

TABLE 31
Detection Results from T2Bacteria testing of samples
spiked with 1000 CFU/mL of given bacterial species

| | |
|---|---|
| 1,000 CFU/mL | Number Positive/Number Tested |

| Species | Ab | Kp | Eci | Efm | Pa | Sa |
|---|---|---|---|---|---|---|
| A. baumannii | 3/3 | 0/3 | 0/3 | 0/3 | 0/3 | 0/3 |
| (Ab) | | | | | | |
| K. pneumoniae | 0/3 | 3/3 | 1/3 | 0/3 | 0/3 | 0/3 |
| (Kp) | | | | | | |
| K. pneumoniae | 0/6 | 6/6 | 0/6 | 0/6 | 0/6 | 0/6 |
| repeat testing | | | | | | |
| E. coli (Eci) | 0/3 | 0/3 | 3/3 | 0/3 | 0/3 | 0/3 |
| E. faecium | 0/3 | 0/3 | 0/3 | 3/3 | 0/3 | 0/3 |
| (Efm) | | | | | | |
| P. aeruginosa | 0/3 | 0/3 | 0/3 | 0/3 | 3/3 | 0/3 |
| (Pa) | | | | | | |
| S. aureus (Sa) | 0/3 | 0/3 | 0/3 | 0/3 | 0/3 | 3/3 |

### T2Carba Resistance+ Panel Testing

Whole blood samples were spiked with targeted bacterial species or with bacterial strains that contained targeted resistance genes at 1,000 CFU/ml. As a negative control, whole blood containing no bacteria was also tested. There was no bacterial strain available that expressed the OXA-48 resistance gene that would not also be positive for a second channel within the panel (i.e., E. cloacae strain AR-0074 would be positive for bath EK and OXA detection). To produce a bacterial strain that contained the OXA-48 gene but would not contain genes for other targets, OXA-48 was cloned into B. subtilis as a single copy, chromosomal based target. B. subtilis with OXA-48 was tested 1,000 CFU/ml. Amp detection is specific for both CMY and DHA. EK detection is specific for most Enterobacter and Klebsiella species. The following species were tested in these studies: E. aerogenes, E. cloacae, E. hormaechei, E. asbudiae, K. pneumoniae, and K. variicola. Samples were processed using a manual assay that mimics testing on the T2DX® device (see above). Four replicates of each sample were tested. Results with T2 values above 65 ms were considered a positive detection.

All samples were valid based on a positive OIC (internal control) detection for each sample. Table 32 shows detection results from testing. There were no false positive samples detected.

TABLE 32
Detection results from T2Carba Resistance+ testing
of samples spiked with 1,000 CFU/ml of given bacterial species

| 1000 CFU/mL | | | Number Positive/Number Tested | | | | | | | |
|---|---|---|---|---|---|---|---|---|---|---|
| Species | Strain ID | Target | AmpC | EK | IMP | KPC | NDM | OXA | VIM | OIC |
| E. coli | AR-0081 | CMY | 4/4 | 0/4 | 0/4 | 0/4 | 0/4 | 0/4 | 0/4 | 4/4 |
| M. morganii | AR-0519 | DHA | 4/4 | 0/4 | 0/4 | 0/4 | 0/4 | 0/4 | 0/4 | 4/4 |
| P. aeruginosa | AR-0103 | IMP | 0/4 | 0/4 | 4/4 | 0/4 | 0/4 | 0/4 | 0/4 | 4/4 |
| E. coli | AR-0061 | KPC | 0/4 | 0/4 | 0/4 | 4/4 | 0/4 | 0/4 | 0/4 | 4/4 |
| A. baumannii | AR 00037 | NDM | 0/4 | 0/4 | 0/4 | 0/4 | 4/4 | 0/4 | 0/4 | 4/4 |
| E. aerogenes | AR-0074 | OXA, | 0/4 | 4/4 | 0/4 | 0/4 | 0/4 | 4/4 | 0/4 | 4/4 |
| | | EK | | | | | | | | |
| B. subtilis | NA | OXA | 0/4 | 0/4 | 0/4 | 0/4 | 0/4 | 4/4 | 0/4 | 4/4 |
| P. aeruginosa | AR-0054 | VIM | 0/4 | 0/4 | 0/4 | 0/4 | 0/4 | 0/4 | 4/4 | 4/4 |
| E. aerogenes | ATCC | EK | 0/4 | 4/4 | 0/4 | 0/4 | 0/4 | 0/4 | 0/4 | 4/4 |
| | 13048 | | | | | | | | | |
| E. cloacae | ATCC | EK | 0/4 | 4/4 | 0/4 | 0/4 | 0/4 | 0/4 | 0/4 | 4/4 |
| | 23355 | | | | | | | | | |
| E. hormaechei | ATCC | EK | 0/4 | 4/4 | 0/4 | 0/4 | 0/4 | 0/4 | 0/4 | 4/4 |
| | 49162 | | | | | | | | | |
| K. pneumoniae | ATCC | EK | 0/4 | 4/4 | 0/4 | 0/4 | 0/4 | 0/4 | 0/4 | 4/4 |
| | 27736 | | | | | | | | | |
| K. variicola | BAA-830 | EK | 0/4 | 4/4 | 0/4 | 0/4 | 0/4 | 0/4 | 0/4 | 4/4 |

| | | | | | | | | | |
|---|---|---|---|---|---|---|---|---|---|
| Negative | None | 0/4 | 0/4 | 0/4 | 0/4 | 0/4 | 0/4 | 0/4 | 4/4 |

### T2ARx Panel Testing

Because bacterial strains that contained a single target gene were difficult to find for T2ARx panel targets, each of the resistance genes were cloned into a pUC19 vector, which was used for testing. Testing was done by adding 1,000 copies of target DNA into the amplification reaction, which also contained a blood lysate. As a negative control, blood lysate was added to the amplification reaction with no addition of DNA. Generated amplicon was tested using probe conjugated particles to determine T2 values (ins) for each target. For bacterial spikes, whole blood samples were spiked with targeted bacterial species or with bacterial strains that contained targeted resistance genes. Bacteria were spiked at 1,000 CFU/mL. As a control whole blood containing no bacteria were tested. Samples were processed using a manual assay that mimics testing on the T2DX® device (see above). The pUC 19 plasmid contains an ampicillin resistance gene. It was determined that this gene is 100/homologous with TEM. All TEM detections would be positive and therefore were not evaluated in this test. Testing resulted in one mefA false positive with ermB DNA and one mefA false positive in the negative controls (Table 33).

TABLE 33
Detection results from T2ARx testing of blood lysate samples
spiked with 1,000 copies of plasmid DNA for each target.

| 1,000 | | | | | | | | | | | |
|---|---|---|---|---|---|---|---|---|---|---|---|
| copies/ | | | | | | | | | | | |
| reaction | Number Positive/Number Tested | | | | | | | | | | |
| Spiked | CTX- | CTX- | | | | | | | | | |
| Target | M 14 | M 15 | ermA | ermB | mecA | mef | SHV | TEM | vanA | vanB | QIC |
| CTX-M 14 | 4/4 | 0/4 | 0/4 | 0/4 | 0/4 | 0/4 | 0/4 | 0/4 | 0/4 | 0/4 | 4/4 |
| CTX-M 15 | 0/4 | 4/4 | 0/4 | 0/4 | 0/4 | 0/4 | 0/4 | 0/4 | 0/4 | 0/4 | 4/4 |
| ermA | 0/4 | 0/4 | 4/4 | 0/4 | 0/4 | 0/4 | 0/4 | 0/4 | 0/4 | 0/4 | 4/4 |
| ermB | 0/4 | 0/4 | 0/4 | 4/4 | 0/4 | 1/4 | 0/4 | 0/4 | 0/4 | 0/4 | 4/4 |
| mecA | 0/4 | 0/4 | 0/4 | 0/4 | 4/4 | 0/4 | 0/4 | 0/4 | 0/4 | 0/4 | 4/4 |
| mefA | 0/4 | 0/4 | 0/4 | 0/4 | 0/4 | 4/4 | 0/4 | 0/4 | 0/4 | 0/4 | 4/4 |
| SHV | 0/4 | 0/4 | 0/4 | 0/4 | 0/4 | 0/4 | 4/4 | 0/4 | 0/4 | 0/4 | 4/4 |
| TEM | 0/4 | 0/4 | 0/4 | 0/4 | 0/4 | 0/4 | 0/4 | 4/4 | 0/4 | 0/4 | 4/4 |
| vanA | 0/4 | 0/4 | 0/4 | 0/4 | 0/4 | 0/4 | 0/4 | 0/4 | 4/4 | 0/4 | 4/4 |
| vanB | 0/4 | 0/4 | 0/4 | 0/4 | 0/4 | 0/4 | 0/4 | 0/4 | 0/4 | 4/4 | 4/4 |
| Negative | 0/4 | 0/4 | 0/4 | 0/4 | 0/4 | 1/4 | 0/4 | 0/4 | 0/4 | 0/4 | 4/4 |

Table 34 shows detection results from testing of whole blood samples spiked with 1,000 CFU/ml of given bacterial species. Initial testing with S. aureus AR-0215 resulted in one false positive for TEM. New S. aureus AR-0215 spiked samples were made and test with an n=8, no false positives were observed in any detection with these samples. Initial testing with E. faecium 1031982 resulted in one false positive for TEM. New samples spiked with E. faecium 1031982 were produced and tested with an n=8, no false positives were observed in any detection with these samples. No false positives were observed with any other samples.

TABLE 34
Positive detection results

| 1,000 CFU | | Number Positive/Number Tested | | | | | | | | | | |
|---|---|---|---|---|---|---|---|---|---|---|---|---|
| Species/ | | CTX- | CTX- | | | | | | | | | |
| Strain ID | Targets | M 14 | M 15 | ermA | ermB | mecA | mef | SHV | Spn | TEM | vanA | vanB |
| K. pneumoniae | CTX-M 14, | 4/4 | 0/4 | 0/4 | 0/4 | 0/4 | 0/4 | 4/4 | 0/4 | 4/4 | 0/4 | 0/4 |
| AR-0079 | SHV-11, | | | | | | | | | | | |
| | TEM-1B | | | | | | | | | | | |
| K. ozaenae | CTX-M 15, | 0/4 | 4/4 | 0/4 | 0/4 | 0/4 | 0/4 | 4/4 | 0/4 | 0/4 | 0/4 | 0/4 |
| AR-0051 | SHV-26 | | | | | | | | | | | |
| K. pneumoniae | CTX-M 15, | 0/4 | 4/4 | 0/4 | 0/4 | 0/4 | 0/4 | 4/4 | 0/4 | 4/4 | 0/4 | 0/4 |
| AR-0044 | SHV-12, | | | | | | | | | | | |
| | TEM-1A | | | | | | | | | | | |
| S. aureus | ermA, | 0/4 | 0/4 | 4/4 | 0/4 | 4/4 | 0/4 | 0/4 | 0/4 | 1/4 | 0/4 | 0/4 |
| AR-0215 | mecA | | | | | | | | | | | |
| S. aureus | ermA, | 0/8 | 0/8 | 8/8 | 0/8 | 8/8 | 0/8 | 0/8 | 0/8 | 0/8 | 0/8 | 0/8 |
| AR-0215 | mecA | | | | | | | | | | | |
| repeat test | | | | | | | | | | | | |
| E. faecium | ermB, | 0/4 | 0/4 | 0/4 | 4/4 | 0/4 | 0/4 | 0/4 | 0/4 | 1/4 | 0/4 | 4/4 |
| 1031982 | vanB | | | | | | | | | | | |
| E. faecium | ermB, | 0/8 | 0/8 | 0/8 | 8/8 | 0/8 | 0/8 | 0/8 | 0/8 | 0/8 | 0/8 | 8/8 |
| 1031982 | vanB | | | | | | | | | | | |
| repeat test | | | | | | | | | | | | |
| E. faecium | ermB, | 0/4 | 0/4 | 0/4 | 4/4 | 0/4 | 0/4 | 0/4 | 0/4 | 0/4 | 0/4 | 4/4 |
| 1004470 | vanB | | | | | | | | | | | |
| E. faecium | ermB, | 0/4 | 0/4 | 0/4 | 4/4 | 0/4 | 0/4 | 0/4 | 0/4 | 0/4 | 4/4 | 0/4 |
| 1029038 | vanA | | | | | | | | | | | |
| E. faecium | ermB, | 0/4 | 0/4 | 0/4 | 4/4 | 0/4 | 0/4 | 0/4 | 0/4 | 0/4 | 4/4 | 0/4 |
| 1030283 | vanA | | | | | | | | | | | |
| S. pyogenes | mef | 0/8 | 0/8 | 0/8 | 0/8 | 0/8 | 8/8 | 0/8 | 0/8 | 0/8 | 0/8 | 0/8 |
| 569013 | | | | | | | | | | | | |
| K. pneumoniae | SHV-12 | 0/4 | 0/4 | 0/4 | 0/4 | 0/4 | 0/4 | 4/4 | 0/4 | 0/4 | 0/4 | 0/4 |
| AR-0524 | | | | | | | | | | | | |
| K. pneumoniae | SHV-12, | 0/4 | 0/4 | 0/4 | 0/4 | 0/4 | 0/4 | 4/4 | 0/4 | 4/4 | 0/4 | 0/4 |
| AR-0552 | TEM1B | | | | | | | | | | | |
| S. pneumoniae | NA | 0/4 | 0/4 | 0/4 | 0/4 | 0/4 | 0/4 | 0/4 | 4/4 | 0/4 | 0/4 | 0/4 |
| BAA-255 | | | | | | | | | | | | |
| S. pneumoniae | mefE, | 0/4 | 0/4 | 0/4 | 4/4 | 0/4 | 4/4 | 0/4 | 4/4 | 0/4 | 0/4 | 0/4 |
| BAA-1407 | ermB | | | | | | | | | | | |
| E. faecium | NA | 0/4 | 0/4 | 0/4 | 0/4 | 0/4 | 0/4 | 0/4 | 0/4 | 0/4 | 0/4 | 0/4 |
| 27270 | | | | | | | | | | | | |
| Negatives | NA | 0/8 | 0/8 | 0/8 | 0/8 | 0/8 | 0/8 | 0/8 | 0/8 | 0/8 | 0/8 | 0/8 |

### Summary of Results

### A. High Titer Detection

Table 35 shows a summary of the high titer detection experiments described above. Detection of ≤100 CFU/mL was demonstrated for thirteen bacterial targets and seventeen resistance genes for a total of thirty targets with a ≥99% sensitivity when at least 20 replicates were tested. These results demonstrate that the panels described herein can detect the indicated species and resistance gene targets spiked into whole blood at concentrations of 1-50 CFU/mL, including 1-10 CFU/mL.

TABLE 35
Summary of High Titer Detection Experiments

| | Species or | Detected | |
|---|---|---|---|
| T2 Panel | Resistance Gene | Concentration | |
| T2 Bacteria | A. baumannii | 3 | CFU/mL |
| | E. coli | 11 | CFU/mL |
| | E. faecium | 5 | CFU/mL |
| | K. pneumoniae | 2 | CFU/mL |
| | P. aeruginosa | 5 | CFU/mL |
| | S. aureus | 2 | CFU/mL |
| T2Carba | E. aerogenes | 10 | CFU/mL |
| Resistance+ | E. cloacae | 10 | CFU/mL |
| | E. hormachei | 10 | CFU/mL |
| | K. pneumoniae | 10 | CFU/mL |
| | K. variicola | 10 | CFU/mL |
| | K. oxytoca | 10 | CFU/mL |
| | IMP | 10 | CFU/mL |
| | KPC | 10 | CFU/mL |
| | OXA | 10 | CFU/mL |
| | NDM | 10 | CFU/mL |
| | VIM | 10 | CFU/mL |
| | AmpC-CMY-2 | 10 | CFU/mL |
| | AmpC-DHA | 15 | CFU/mL |
| T2ARx | S. pneumoniae | 50 | CFU/mL |
| | CTX-M 15 | 50 | CFU/mL |
| | CTX-M 14 | 50 | CFU/mL |
| | vanA | 50 | CFU/mL |
| | vanB | 50 | CFU/mL |
| | ermA | 50 | CFU/mL |
| | ermB | 50 | CFU/mL |
| | mefA | 50 | CFU/mL |
| | mecA | 50 | CFU/mL |
| | blaSHV | 50 | CFU/mL |
| | blaTEM | 50 | CFU/mL |

### B. Cross Reactivity Study

Testing with T2Bacteria, T2Carba Resistance+ and T2ARx panels of bacterial samples spiked at 1,000 CFU/ml did not result in repeatable off target detection, demonstrating a lack of cross reactivity. T2Bacteria and T2ARx had 1 and 2 false positives, respectively, however repeat testing with newly prepared samples did not result in any off target detection, confirming a lack of cross reactivity.

### Example 5: Multiplexed Detection of 9 or More Resistance Genes

The menu size for detection of resistance markers and pathogen targets can be increased using parallel processing of multiplexed panels having 9, 10, 11, 12, or more targets. In this example, a nine-plex reaction (9-primer sets) was used to detect the targets of the T2Carba Resistance+panel as shown in Table 36. Samples spiked with 15 CFU/mL were processed using a manual assay as described above (see Example 4 and U.S. Provisional Patent Application No. 62/729,375). The primers and probes shown in Table 16 were used. As shown in Table 36, 100% detection was achieved (n=16 samples per evaluated spiked species) at 15 CFU/mL.

These data demonstrate that the targets described herein can be detected in multiplexed reactions, including multiplexed reactions that include nine, ten, eleven, twelve, thirteen, fourteen, fifteen, or more targets at low concentration (e.g., 1-15 CFU/mL, e.g., 1-10 CFU/mL).

TABLE 36
Demonstration of 9-plex (9 primer set) detection at 15 CFU/mL using T2MR manual assay, resistance
genes, and genus level species (Enterobacter spp. and Klebsiella spp.).

| | | | | | | | | Enterobacter | | Klebsiella | |
|---|---|---|---|---|---|---|---|---|---|---|---|
| Target | DHA | CMY-2 | IMP-4 | NDM-1 | VIM-1 | OXA-48 | KPC-3 | spp. | | spp. | IC |
| Spiked | M. | E. | K. | A. | E. | E. | E. | E. | E. | K. | IC |
| species | morganii | coli | pneumoniae | baumanii | cloacae | aerogenes | coli | cloacae | aerogenes | pneumoniae | |
| containing | | | | | | | | | | | |
| target | | | | | | | | | | | |
| Isolate | AR-519 | AR-81 | AR-34 | AR-37 | AR-154 | AR-74 | AR-61 | AR-154 | AR-74 | AR-34 | N/A |
| Primers | DHA | CMY | IMP | NDM | VIM | OXA | KPC | Enterobacter/Klebsiella | | | IC |
| | | | | | | | | spp. (EK) | | | |
| Spiked | 15 | 15 | 15 | 15 | 15 | 15 | 15 | 15 | 15 | 15 | N/A |
| titer level | | | | | | | | | | | |
| (CFU/mL) | | | | | | | | | | | |
| Positivity | 100 | 100 | 100 | 100 | 100 | 100 | 100 | 100 | 100 | 100 | 100 |
| (%) | | | | | | | | | | | |
| Total | 16 | 16 | 16 | 16 | 16 | 16 | 16 | 16 | 16 | 16 | 16 |
| samples (N) | | | | | | | | | | | |

### Example 6: Alternate Probes for Detection of NDM

An alternate probe pair for detection of NDM was developed. The nucleotide sequence of the alternate 5′ NDM probe may include GCGACCGGCAGGTTGATCTCCTGC (SEQ ID NO: 95) and the nucleotide sequence of the alternate 3′ NDM probe may include CGGCATGTCGAGATAGGAAGTGTGCTGC (SEQ ID NO: 96). This probe pair can be used for detection of NDM amplicons produced by a forward primer including the nucleotide sequence of CCAGCTCGCACCGAATGTCT (SEQ ID NO: 1) and a reverse primer including the nucleotide sequence of CATCTTGTCCTGATGCGCGTGAGTCA (SEQ ID NO: 2). For example, this alternate probe pair can be used in place of, or in addition to, the probe pair of GCGACCGGCAGGTTGATCT (SEQ ID NO: 17) and CATGTCGAGATAGGAAGTGTGCTGC (SEQ ID NO: 18), for example, in any of the panels described herein (e.g., the panel shown in Table 17 or the panel shown in Table 18).

Sequence Listing

| SEQ ID | |
|---|---|
| NO | Sequence |
| 1. | CCAGCTCGCACCGAATGTCT |
| 2. | CATCTTGTCCTGATGCGCGTGAGTCA |
| 3. | TTTCTGCCACCGCGCTGAC |
| 4. | GCAGCAAGAAAGCCCTTGAATG |
| 5. | ACATTTCCATAGCGACAGCACGGGCGGAAT |
| 6. | GGACTTTGGCCAAGCTTCTAAATTTGCGTC |
| 7. | CGTGCAGTCTCCACGCACT |
| 8. | TCGAATGCGCAGCACCGGGATAG |
| 9. | GGCTGTGTTTTTGGTGGCATCGATTATC |
| 10. | TCCCACTTAAAGACTTGGTGTTCATCC |
| 11. | ACGGGCCGGTAATGCGGATCTGGA |
| 12. | TATTCGCCAGAATCACAATCGCCACCTGT |
| 13. | CCGCGGCGAAATTAAGCTCAGCGA |
| 14. | CCAAACAGACCAATGCTGGAGTTAG |
| 15. | ATTCGTTGCACTATCGTTAACTGAATACA |
| 16. | CTGTACCGTCGGACTTTCCAGAC |
| 17. | GCGACCGGCAGGTTGATCT |
| 18. | CATGTCGAGATAGGAAGTGTGCTGC |
| 19. | CGGAACCATTCGCTAAACTC |
| 20. | AGGCGCAACTGTAAGTTACCG |
| 21. | GCTTAATTCTCAATCTATCCCCACGTAT |
| 22. | CTCCAGATAACGTAGTGGTTTGGCTG |
| 23. | CTTTCATGACGACCGCGTCGG |
| 24. | CTCTAGAAGGACTCTCATCGAGC |
| 25. | ATTTTAAAGGTAGATGCGGG |
| 26. | CGCCCTGTGATTTATGTTCA |
| 27. | GTTTTATGCACCCAGGAAGC |
| 28. | TCTGCTGCGGCCAGTCATA |
| 29. | GCGGCTGCCAGTTTTGATAA |
| 30. | GTGGCTAAGTGCAGCAGGC |
| 31. | CGTTCCACTAACACACAAGCTGATTCAG |
| 32. | ATCTCGGTTGATTTCTTTTCCTCGGG |
| 33. | ACGCTTTCCAATGTGCAGTACCAGTA |
| 34. | TGCGATCCAGACGAAACGTCTCATCG |
| 35. | GTGATACCACTTCACCTCGGGCAA |
| 36. | AATACATCGCGACGGCTTTCTGCC |
| 37. | AGAATTACCTTTGAAAGTCAGGC |
| 38. | GCTTCAAAGCCTGTCGGAATTGGTTT |
| 39. | GGGCATTTAACGACGAAACTGGCTA |
| 40. | GTGTTCGGTGAATATCCAAGGTACGC |
| 41. | ATGTTGGTCCCATTAACTCTGAAGAA |
| 42. | CACCTGTTTGAGGGTGGATAGCAGTA |
| 43. | GCAGGGCAAGCAGTATCATTAATCAC |
| 44. | AATTAAATCAGCACCAATCATTATCTTCTTCC |
| 45. | AAGCTGCTGACCAGCCAGCGTCTGA |
| 46. | CGGCGATTTGCTGATTTCGCTCG |
| 47. | TGCAGTGCTGCCATAACCATGAGTGA |
| 48. | AGCGCAGAAGTGGTCCTGCAACTTT |
| 49. | CAGTACGGAAICITICGTATTCATCAGGA |
| 50. | CAGTTCGGGAAGTGCAATACCTGCA |
| 51. | AATTGAGCAAGCGATTTCGGGCTGT |
| 52. | CGTTTAGAACGATGCCGCCATCCT |
| 53. | CCTTGGACGGAAATGTAGCTGGCA |
| 54. | AATCACATGGTTGACACCTGCTGTG |
| 55. | GGAAATCTAACGAGAGAGCATGCT |
| 56. | CGATGCGTGACACCCAGGC |
| 57. | CTGTCGAGATCAAGCCTGCCGA |
| 58. | ACAAATTGATTGCCCAGCTCGGT |
| 59. | AATCAGCGGCGCACGATCTTT |
| 60. | AATGCTCGCTGCACCGGTGGTAT |
| 61. | AAATCTGCAACGAGCTTTGGG |
| 62. | GTTTATAAGTGGGTAAACCGTGAATATC |
| 63. | TTCGTGTCACTTTAATTCACCAAGAT |
| 64. | AAAGCCATGCGTCTGACATCT |
| 65. | AAGCTCCAACATGAAGATGGCT |
| 66. | AGATGGCAAAGATATTCAACTAAC |
| 67. | AGTGCCATCTTGCAAATGGCGAT |
| 68. | TGCAATTGGTGTGTTAGTGGATCGTCATGATA |
| 69. | CAGTGGATGGTGGACGATCGGGT |
| 70. | TTGTGGTGATTTATCTGCGGGATACT |
| 71. | CGCCAGTTAATAGTTTGCGCAACG |
| 72. | AAAGCGGTTAGCTCCTTCGGTCCT |
| 73. | CGTTCAGTACAATGCGGCCGTTA |
| 74. | CTGCGGGAACGGTTATAACTGC |
| 75. | GCACCCGATATACTTTCTTTGCC |
| 76. | CGCCGACAATCAAATCATCCT |
| 77. | TTGACCAGTTCCGAGCAAATGGTA |
| 78. | GACAGTATCGATGTTCCAGCAGCT |
| 79. | GAGACGTTTTGGATACATGTGAAAGAAGGC |
| 80. | CGATGGTTCACGGGATTCTGCAATTC |
| 81. | CCTCGGGCAATGGCGCAAAC |
| 82. | ATCGCGACGGCTTTCTGCCTTA |
| 83. | GGGCGCAGCTGGTGACAT |
| 84. | AAGATCGTGCGCCGCTGATT |
| 85. | TATTCATCAGGAAGTCGAGCCGGA |
| 86. | GCAGTTATAACCGTTCCCGCAG |
| 87. | TAACGGCCGCATTGTACTGAACG |
| 88. | AAGATCAACACGGGCAAGCCCTCT |
| 89. | AAAGGCTATAAAGATGATGCAG |
| 90. | GAGTATTTATAACAACATGAAAAATGATT |
| 91. | ATGTGGGTCCAATTAATTCTGACGAG |
| 92. | CTCCAGTTTTTGGTTGTAATGCTGTA |
| 93. | GGCTTAGAACGCCTCTATGAT |
| 94. | AGAGTACAAGAAAGTATTTATAAACATATGA |
| 95. | GCGACCGGCAGGTTGATCTCCTGC |
| 96. | CGGCATGTCGAGATAGGAAGTGTGCTGC |

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims.

What is claimed is:

1. A method for detecting the presence of an antibiotic resistance gene in a biological sample, the method comprising:
(a) amplifying in a biological sample or a fraction thereof one or more antibiotic resistance target nucleic acids in a multiplexed amplification reaction, wherein the multiplexed amplification reaction is configured to amplify target nucleic acids characteristic of two or more antibiotic resistance genes selected from the group consisting of NDM, KPC, IMP, VIM, OXA-48-like, DHA, CMY, FOX, mecA, mecC, vanA, vanB, CTX-M 14, CTX-M 15, mefA, mefE, ermA, ermB, SHV, and TEM, wherein the two or more antibiotic resistance genes comprises at least one of IMP, OXA-48-like, DHA, CMY, FOX, CTX-M 14, CTX-M 15, mecC, mefA, mefE, ermA, ermB, or TEM; and
(b) detecting the one or more amplified antibiotic resistance target nucleic acids to determine whether one or more of the antibiotic resistance genes is present in the biological sample,
wherein the method individually detects an antibiotic resistance gene of a pathogen present at a concentration of 10 cells/mL of biological sample or less.

2. The method of claim 1, wherein:
(i) the method comprises amplifying and/or detecting at least three, at least four, at least five, at least six, or all seven antibiotic resistance genes selected from the group consisting of NDM, KPC, IMP, VIM, OXA-48-like, DHA, and CMY; and/or
(ii) the multiplexed amplification reaction is configured to amplify at least three, at least four, at least five, at least six, or all seven antibiotic resistance genes selected from the group consisting of NDM, KPC, IMP, VIM, OXA-48-like, DHA, and CMY.

3. The method of claim 1 or 2, wherein:
(i) the method comprises amplifying and/or detecting NDM, KPC, IMP, VIM, OXA-48-like, DHA, and CMY; and/or
(ii) the multiplexed amplification reaction is configured to amplify NDM, KPC, IMP, VIM, OXA-48-like, DHA, and CMY.

4. The method of claim 2 or 3, wherein:
(i) NDM is amplified in the presence of a forward primer comprising the nucleotide sequence CCAGCTCGCACCGAATGTCT (SEQ ID NO: 1) and a reverse primer comprising the nucleotide sequence CATCTTGTCCTGATGCGCGTGAGTCA (SEQ ID NO: 2);
(ii) KPC is amplified in the presence of a forward primer comprising the nucleotide sequence TTTCTGCCACCGCGCTGAC (SEQ ID NO: 3) and a reverse primer comprising the nucleotide sequence GCAGCAAGAAAGCCCTTGAATG (SEQ ID NO: 4);
(iii) IMP is amplified in the presence of a forward primer comprising the nucleotide sequence ACATTTCCATAGCGACAGCACGGGCGGAAT (SEQ ID NO: 5) and a reverse primer comprising the nucleotide sequence GGACTTTGGCCAAGCTTCTAAATTTGCGTC (SEQ ID NO: 6);
(iv) VIM is amplified in the presence of a forward primer comprising the nucleotide sequence CGTGCAGTCTCCACGCACT (SEQ ID NO: 7) and a reverse primer comprising the nucleotide sequence TCGAATGCGCAGCACCGGGATAG (SEQ ID NO: 8);
(v) OXA-48-like is amplified in the presence of a forward primer comprising the nucleotide sequence GGCTGTGTTTTGGTGGCATCGATTATC (SEQ ID NO: 9) and a reverse primer comprising the nucleotide sequence TCCCACTTAAAGACTTGGTGTTCATCC (SEQ ID NO: 10);
(vi) DHA is amplified in the presence of a forward primer comprising the nucleotide sequence ACGGGCCGGTAATGCGGATCTGGA (SEQ ID NO: 11) and a reverse primer comprising the nucleotide sequence TATTCGCCAGAATCACAATCGCCACCTGT (SEQ ID NO: 12); and/or
(vii) CMY is amplified in the presence of a forward primer comprising the nucleotide sequence CCGCGGCGAAATTAAGCTCAGCGA (SEQ ID NO: 13) and a reverse primer comprising the nucleotide sequence CCAAACAGACCAATGCTGGAGTTAG (SEQ ID NO: 14).

5. The method of claim 1, wherein:
(i) the method comprises amplifying and/or detecting at least three, at least four, at least five, at least six, at least seven, at least eight, at least nine, at least ten, at least eleven, at least twelve, or all thirteen antibiotic resistance genes selected from the group consisting of KPC, CTX-M 14, CTX-M 15, NDM, VIM, IMP, vanA, vanB, mecA, mecC, OXA-48-like, CMY, and DHA; and/or
(ii) the multiplexed amplification reaction is configured to amplify at least three, at least four, at least five, at least six, at least seven, at least eight, at least nine, at least ten, at least eleven, at least twelve, or all thirteen antibiotic resistance genes selected from the group consisting of KPC, CTX-M 14, CTX-M 15, NDM, VIM, IMP, vanA, vanB, mecA, mecC, OXA-48-like, CMY, and DHA.

6. The method of claim 1 or 5, wherein:
(i) the method comprises amplifying and/or detecting KPC, CTX-M 14, CTX-M 15, NDM, VIM, IMP, vanA, vanB, mecA, mecC, OXA-48-like, CMY, and DHA; and/or
(ii) the multiplexed amplification reaction is configured to amplify KPC, CTX-M 14, CTX-M 15, NDM, VIM, IMP, vanA, vanB, mecA, mecC, OXA-48-like, CMY, and DHA.

7. The method of claim 5 or 6, wherein:
(i) KPC is amplified in the presence of a forward primer comprising the nucleotide sequence TTTCTGCCACCGCGCTGAC (SEQ ID NO: 3) and a reverse primer comprising the nucleotide sequence GCAGCAAGAAAGCCCTTGAATG (SEQ ID NO: 4);
(ii) CTX-M 14 is amplified in the presence of a forward primer comprising the nucleotide sequence ACGCTTTCCAATGTGCAGTACCAGTA (SEQ ID NO: 33) and a reverse primer comprising the nucleotide sequence TGCGATCCAGACGAAACGTCTCATCG (SEQ ID NO: 34);
(iii) CTX-M 15 is amplified in the presence of a forward primer comprising the nucleotide sequence CCTCGGGCAATGGCGCAAAC (SEQ ID NO: 81) and a reverse primer comprising the nucleotide sequence ATCGCGACGGCTTTCTGCCTTA (SEQ ID NO: 82);
(iv) NDM is amplified in the presence of a forward primer comprising the nucleotide sequence CCAGCTCGCACCGAATGTCT (SEQ ID NO: 1) and a reverse primer comprising the nucleotide sequence CATCTTGTCCTGATGCGCGTGAGTCA (SEQ ID NO: 2);
(v) VIM is amplified in the presence of a forward primer comprising the nucleotide sequence CGTGCAGTCTCCACGCACT (SEQ ID NO: 7) and a reverse primer comprising the nucleotide sequence TCGAATGCGCAGCACCGGGATAG (SEQ ID NO: 8);
(vi) IMP is amplified in the presence of a forward primer comprising the nucleotide sequence ACATTTCCATAGCGACAGCACGGGCGGAAT (SEQ ID NO: 5) and a reverse primer comprising the nucleotide sequence GGACTTTGGCCAAGCTTCTAAATTTGCGTC (SEQ ID NO: 6);
(vii) vanA is amplified in the presence of a forward primer comprising the nucleotide sequence TATTCATCAGGAAGTCGAGCCGGA (SEQ ID NO: 85) and a reverse primer comprising the nucleotide sequence CAGTTCGGGAAGTGCAATACCTGCA (SEQ ID NO: 50);
(viii) vanB is amplified in the presence of a forward primer comprising the nucleotide sequence AATTGAGCAAGCGATTTCGGGCTGT (SEQ ID NO: 51) and a reverse primer comprising the nucleotide sequence AAGATCAACACGGGCAAGCCCTCT (SEQ ID NO: 88);
(ix) mecA is amplified in the presence of a forward primer comprising the nucleotide sequence ATGTTGGTCCCATTAACTCTGAAGAA (SEQ ID NO: 41) and a reverse primer comprising the nucleotide sequence CACCTGTTTGAGGGTGGATAGCAGTA (SEQ ID NO: 42);
(x) mecC is amplified in the presence of a forward primer comprising the nucleotide sequence ATGTGGGTCCAATTAATTCTGACGAG (SEQ ID NO: 91) and a reverse primer comprising the nucleotide sequence CTCCAGTTTTGGTTGTAATGCTGTA (SEQ ID NO: 92);
(xi) OXA-48-like is amplified in the presence of a forward primer comprising the nucleotide sequence GGCTGTGTTTTGGTGGCATCGATTATC (SEQ ID NO: 9) and a reverse primer comprising the nucleotide sequence TCCCACTTAAAGACTTGGTGTTCATCC (SEQ ID NO: 10);
(xii) CMY is amplified in the presence of a forward primer comprising the nucleotide sequence CCGCGGCGAAATTAAGCTCAGCGA (SEQ ID NO: 13) and a reverse primer comprising the nucleotide sequence CCAAACAGACCAATGCTGGAGTTAG (SEQ ID NO: 14); and/or
(xiii) DHA is amplified in the presence of a forward primer comprising the nucleotide sequence ACGGGCCGGTAATGCGGATCTGGA (SEQ ID NO: 11) and a reverse primer comprising the nucleotide sequence TATTCGCCAGAATCACAATCGCCACCTGT (SEQ ID NO: 12).

8. The method of claim 1, wherein:
(i) the method comprises amplifying and/or detecting at least three, at least four, at least five, at least six, at least seven, at least eight, at least nine, or all ten antibiotic resistance genes selected from the group consisting of CTX-M 14, CTX-M 15, ermA, ermB, mecA, mefA, SHV, TEM, vanA, and vanB; and/or
(ii) the multiplexed amplification reaction is configured to amplify at least three, at least four, at least five, at least six, at least seven, at least eight, at least nine, or all ten antibiotic resistance genes selected from the group consisting of CTX-M 14, CTX-M 15, ermA, ermB, mecA, mefA, SHV, TEM, vanA, and vanB.

9. The method of claim 1 or 8, wherein:
(i) the method comprises amplifying and/or detecting CTX-M 14, CTX-M 15, ermA, ermB, mecA, mefA, SHV, TEM, vanA, and vanB; and/or
(ii) the multiplexed amplification reaction is configured to amplify CTX-M 14, CTX-M 15, ermA, ermB, mecA, mefA, SHV, TEM, vanA, and vanB.

10. The method of claim 8 or 9, wherein:
(i) CTX-M 14 is amplified in the presence of a forward primer comprising the nucleotide sequence ACGCTTTCCAATGTGCAGTACCAGTA (SEQ ID NO: 33) and a reverse primer comprising the nucleotide sequence TGCGATCCAGACGAAACGTCTCATCG (SEQ ID NO: 34);
(ii) CTX-M 15 is amplified in the presence of a forward primer comprising the nucleotide sequence GTGATACCACTTCACCTCGGGCAA (SEQ ID NO: 35) and a reverse primer comprising the nucleotide sequence AATACATCGCGACGGCTTTCTGCC (SEQ ID NO: 36);
(iii) ermA is amplified in the presence of a forward primer comprising the nucleotide sequence AGAATTACCTTTGAAAGTCAGGC (SEQ ID NO: 37) and a reverse primer comprising the nucleotide sequence GCTTCAAAGCCTGTCGGAATTGGTTT (SEQ ID NO: 38);
(iv) ermB is amplified in the presence of a forward primer comprising the nucleotide sequence GGGCATTTAACGACGAAACTGGCTA (SEQ ID NO: 39) and a reverse primer comprising the nucleotide sequence GTGTTCGGTGAATATCCAAGGTACGC (SEQ ID NO: 40);
(v) mecA is amplified in the presence of a forward primer comprising the nucleotide sequence ATGTTGGTCCCATTAACTCTGAAGAA (SEQ ID NO: 41) and a reverse primer comprising the nucleotide sequence CACCTGTTTGAGGGTGGATAGCAGTA (SEQ ID NO: 42);
(vi) mefA is amplified in the presence of a forward primer comprising the nucleotide sequence GCAGGGCAAGCAGTATCATTAATCAC (SEQ ID NO: 43) and a reverse primer comprising the nucleotide sequence AATTAAATCAGCACCAATCATTATCTTCTTCC (SEQ ID NO: 44);
(vii) SHV is amplified in the presence of a forward primer comprising the nucleotide sequence AAGCTGCTGACCAGCCAGCGTCTGA (SEQ ID NO: 45) and a reverse primer comprising the nucleotide sequence CGGCGATTTGCTGATTTCGCTCG (SEQ ID NO: 46);
(viii) TEM is amplified in the presence of a forward primer comprising the nucleotide sequence TGCAGTGCTGCCATAACCATGAGTGA (SEQ ID NO: 47) and a reverse primer comprising the nucleotide sequence AGCGCAGAAGTGGTCCTGCAACTTT (SEQ ID NO: 48);
(ix) vanA is amplified in the presence of a forward primer comprising the nucleotide sequence CAGTACGGAATCTTTCGTATTCATCAGGA (SEQ ID NO: 49) and a reverse primer comprising the nucleotide sequence CAGTTCGGGAAGTGCAATACCTGCA (SEQ ID NO: 50); and/or
(x) vanB is amplified in the presence of a forward primer comprising the nucleotide sequence AATTGAGCAAGCGATTTCGGGCTGT (SEQ ID NO: 51) and a reverse primer comprising the nucleotide sequence CGTTTAGAACGATGCCGCCATCCT (SEQ ID NO: 52).

11. The method of any one of claims 1-10, wherein:
(i) amplifying step (a) further comprises amplifying a target nucleic acid characteristic of a bacterial pathogen, and detecting step (b) further comprises detecting the amplified target nucleic acid characteristic of a bacterial pathogen to determine whether the bacterial pathogen is present; and/or
(ii) the multiplexed amplification reaction is configured to amplify a target nucleic acid characteristic of a bacterial pathogen.

12. The method of claim 11, wherein the target nucleic acid characteristic of a bacterial pathogen is characteristic of an Enterobacter spp., a Klebsiella spp., or Streptococcus pneumoniae.

13. The method of claim 11 or 12, wherein the target nucleic acid characteristic of a bacterial pathogen is characteristic of Enterobacter spp. and Klebsiella spp.

14. The method of claim 13, wherein the target nucleic acid characteristic of Enterobacter spp. and Klebsiella spp. is amplified in the presence of a forward primer comprising the nucleotide sequence ATTCGTTGCACTATCGTTAACTGAATACA (SEQ ID NO: 15) and a reverse primer comprising the nucleotide sequence CTGTACCGTCGGACTTTCCAGAC (SEQ ID NO: 16).

15. The method of claim 11 or 12, wherein the target nucleic acid characteristic of a bacterial pathogen is characteristic of Streptococcus pneumoniae.

16. The method of claim 15, wherein the target nucleic acid characteristic of Streptococcus pneumoniae is amplified in the presence of a forward primer comprising the nucleotide sequence CCTTGGACGGAAATGTAGCTGGCA (SEQ ID NO: 53) and a reverse primer comprising the nucleotide sequence AATCACATGGTTGACACCTGCTGTG (SEQ ID NO: 54).

17. The method of any one of claims 1-16, wherein:
(i) amplifying step (a) further comprises amplifying an internal amplification control (IC) target nucleic acid and detecting step (b) further comprises detecting the amplified IC target nucleic acid; and/or
(ii) the multiplexed amplification reaction is configured to amplify an amplified IC target nucleic acid.

18. The method of any one of claims 1-17, wherein the method detects an antibiotic resistance gene of a pathogen present at a concentration of 2 cells/mL of biological sample or less.

19. The method of claim 18, wherein the method detects an antibiotic resistance gene of a pathogen present at a concentration of 1 cells/mL of biological sample.

20. The method of any one of claims 1-19, wherein the detecting of step (b) comprises magnetic, sequencing, optical, fluorescent, mass, density, chromatographic, and/or electrochemical detection.

21. The method of claim 20, wherein the detecting of step (b) comprises T2 magnetic resonance (T2MR).

22. The method of claim 20, wherein the detecting of step (b) comprises sequencing.

23. A method for detecting the presence of an antibiotic resistance gene in a biological sample, the method comprising:
(a) providing a biological sample;
(b) lysing pathogen cells in the biological sample;
(c) amplifying in the product of step (b) one or more antibiotic resistance target nucleic acids in a multiplexed amplification reaction to form an amplified biological sample, wherein the multiplexed amplification reaction is configured to amplify target nucleic acids characteristic of two or more antibiotic resistance genes selected from the group consisting of NDM, KPC, IMP, VIM, OXA-48-like, DHA, CMY, FOX, mecA, vanA, vanB, CTX-M 14, CTX-M 15, mefA, mecC, mefE, ermA, ermB, SHV, and TEM, wherein the two or more antibiotic resistance genes comprises at least one of IMP, OXA-48-like, DHA, CMY, FOX, CTX-M 14, CTX-M 15, mecC, mefA, mefE, ermA, ermB, or TEM;
(d) preparing a first assay sample by contacting a portion of the amplified biological sample with a first population of magnetic particles, wherein the magnetic particles of the first population have binding moieties characteristic of a first antibiotic resistance gene on their surface, the binding moieties operative to alter aggregation of the magnetic particles in the presence of a first amplified antibiotic resistance target nucleic acid;
(e) preparing a second assay sample by contacting a portion of the amplified biological sample with a second population of magnetic particles, wherein the magnetic particles of the second population have binding moieties characteristic of a second antibiotic resistance gene on their surface, the binding moieties operative to alter aggregation of the magnetic particles in the presence of a second amplified antibiotic resistance target nucleic acid;
(f) providing each assay sample in a detection tube within a device, the device comprising a support defining a well for holding the detection tube comprising the assay sample, and having an RF coil configured to detect a signal produced by exposing the mixture to a bias magnetic field created using one or more magnets and an RF pulse sequence;
(g) exposing each assay sample to a bias magnetic field and an RF pulse sequence;
(h) following step (g), measuring the signal produced by each assay sample; and
(i) on the basis of the result of step (h), detecting whether one or more of the antibiotic resistance genes is present in the biological sample.

24. The method of claim 23, wherein:
the method comprises amplifying and/or detecting at least three, at least four, at least five, at least six, or all seven antibiotic resistance genes selected from the group consisting of NDM, KPC, IMP, VIM, OXA-48-like, DHA, and CMY; and/or
the multiplexed amplification reaction is configured to amplify at least three, at least four, at least five, at least six, or all seven antibiotic resistance genes selected from the group consisting of NDM, KPC, IMP, VIM, OXA-48-like, DHA, and CMY.

25. The method of claim 23 or 24, wherein:
the method comprises amplifying and/or detecting NDM, KPC, IMP, VIM, OXA-48-like, DHA, and CMY; and/or
the multiplexed amplification reaction is configured to amplify NDM, KPC, IMP, VIM, OXA-48-like, DHA, and CMY.

26. The method of claim 24 or 25, wherein:
(i) NDM is amplified in the presence of a forward primer comprising the nucleotide sequence CCAGCTCGCACCGAATGTCT (SEQ ID NO: 1) and a reverse primer comprising the nucleotide sequence CATCTTGTCCTGATGCGCGTGAGTCA (SEQ ID NO: 2);
(ii) KPC is amplified in the presence of a forward primer comprising the nucleotide sequence TTTCTGCCACCGCGCTGAC (SEQ ID NO: 3) and a reverse primer comprising the nucleotide sequence GCAGCAAGAAAGCCCTTGAATG (SEQ ID NO: 4);
(iii) IMP is amplified in the presence of a forward primer comprising the nucleotide sequence ACATTTCCATAGCGACAGCACGGGCGGAAT (SEQ ID NO: 5) and a reverse primer comprising the nucleotide sequence GGACTTTGGCCAAGCTTCTAAATTTGCGTC (SEQ ID NO: 6);
(iv) VIM is amplified in the presence of a forward primer comprising the nucleotide sequence CGTGCAGTCTCCACGCACT (SEQ ID NO: 7) and a reverse primer comprising the nucleotide sequence TCGAATGCGCAGCACCGGGATAG (SEQ ID NO: 8);
(v) OXA-48-like is amplified in the presence of a forward primer comprising the nucleotide sequence GGCTGTGTTTTGGTGGCATCGATTATC (SEQ ID NO: 9) and a reverse primer comprising the nucleotide sequence TCCCACTTAAAGACTTGGTGTTCATCC (SEQ ID NO: 10);
(vi) DHA is amplified in the presence of a forward primer comprising the nucleotide sequence ACGGGCCGGTAATGCGGATCTGGA (SEQ ID NO: 11) and a reverse primer comprising the nucleotide sequence TATTCGCCAGAATCACAATCGCCACCTGT (SEQ ID NO: 12); and/or
(vii) CMY is amplified in the presence of a forward primer comprising the nucleotide sequence CCGCGGCGAAATTAAGCTCAGCGA (SEQ ID NO: 13) and a reverse primer comprising the nucleotide sequence CCAAACAGACCAATGCTGGAGTTAG (SEQ ID NO: 14).

27. The method of claim 23, wherein:
(i) the method comprises amplifying and/or detecting at least three, at least four, at least five, at least six, at least seven, at least eight, at least nine, at least ten, at least eleven, at least twelve, or all thirteen antibiotic resistance genes selected from the group consisting of KPC, CTX-M 14, CTX-M 15, NDM, VIM, IMP, vanA, vanB, mecA, mecC, OXA-48-like, CMY, and DHA; and/or
(ii) the multiplexed amplification reaction is configured to amplify at least three, at least four, at least five, at least six, at least seven, at least eight, at least nine, at least ten, at least eleven, at least twelve, or all thirteen antibiotic resistance genes selected from the group consisting of KPC, CTX-M 14, CTX-M 15, NDM, VIM, IMP, vanA, vanB, mecA, mecC, OXA-48-like, CMY, and DHA.

28. The method of claim 23 or 27, wherein:
(i) the method comprises amplifying and/or detecting KPC, CTX-M 14, CTX-M 15, NDM, VIM, IMP, vanA, vanB, mecA, mecC, OXA-48-like, CMY, and DHA; and/or
(ii) the multiplexed amplification reaction is configured to amplify KPC, CTX-M 14, CTX-M 15, NDM, VIM, IMP, vanA, vanB, mecA, mecC, OXA-48-like, CMY, and DHA.

29. The method of claim 27 or 28, wherein:
(i) KPC is amplified in the presence of a forward primer comprising the nucleotide sequence TTTCTGCCACCGCGCTGAC (SEQ ID NO: 3) and a reverse primer comprising the nucleotide sequence GCAGCAAGAAAGCCCTTGAATG (SEQ ID NO: 4);
(ii) CTX-M 14 is amplified in the presence of a forward primer comprising the nucleotide sequence ACGCTTTCCAATGTGCAGTACCAGTA (SEQ ID NO: 33) and a reverse primer comprising the nucleotide sequence TGCGATCCAGACGAAACGTCTCATCG (SEQ ID NO: 34);
(iii) CTX-M 15 is amplified in the presence of a forward primer comprising the nucleotide sequence CCTCGGGCAATGGCGCAAAC (SEQ ID NO: 81) and a reverse primer comprising the nucleotide sequence ATCGCGACGGCTTTCTGCCTTA (SEQ ID NO: 82);
(iv) NDM is amplified in the presence of a forward primer comprising the nucleotide sequence CCAGCTCGCACCGAATGTCT (SEQ ID NO: 1) and a reverse primer comprising the nucleotide sequence CATCTTGTCCTGATGCGCGTGAGTCA (SEQ ID NO: 2);
(v) VIM is amplified in the presence of a forward primer comprising the nucleotide sequence CGTGCAGTCTCCACGCACT (SEQ ID NO: 7) and a reverse primer comprising the nucleotide sequence TCGAATGCGCAGCACCGGGATAG (SEQ ID NO: 8);
(vi) IMP is amplified in the presence of a forward primer comprising the nucleotide sequence ACATTTCCATAGCGACAGCACGGGCGGAAT (SEQ ID NO: 5) and a reverse primer comprising the nucleotide sequence GGACTTTGGCCAAGCTTCTAAATTTGCGTC (SEQ ID NO: 6);
(vii) vanA is amplified in the presence of a forward primer comprising the nucleotide sequence TATTCATCAGGAAGTCGAGCCGGA (SEQ ID NO: 85) and a reverse primer comprising the nucleotide sequence CAGTTCGGGAAGTGCAATACCTGCA (SEQ ID NO: 50);
(viii) vanB is amplified in the presence of a forward primer comprising the nucleotide sequence AATTGAGCAAGCGATTTCGGGCTGT (SEQ ID NO: 51) and a reverse primer comprising the nucleotide sequence AAGATCAACACGGGCAAGCCCTCT (SEQ ID NO: 88);
(ix) mecA is amplified in the presence of a forward primer comprising the nucleotide sequence ATGTTGGTCCCATTAACTCTGAAGAA (SEQ ID NO: 41) and a reverse primer comprising the nucleotide sequence CACCTGTTTGAGGGTGGATAGCAGTA (SEQ ID NO: 42);
(x) mecC is amplified in the presence of a forward primer comprising the nucleotide sequence ATGTGGGTCCAATTAATTCTGACGAG (SEQ ID NO: 91) and a reverse primer comprising the nucleotide sequence CTCCAGTTTTGGTTGTAATGCTGTA (SEQ ID NO: 92);
(xi) OXA-48-like is amplified in the presence of a forward primer comprising the nucleotide sequence GGCTGTGTTTTGGTGGCATCGATTATC (SEQ ID NO: 9) and a reverse primer comprising the nucleotide sequence TCCCACTTAAAGACTTGGTGTTCATCC (SEQ ID NO: 10);
(xii) CMY is amplified in the presence of a forward primer comprising the nucleotide sequence CCGCGGCGAAATTAAGCTCAGCGA (SEQ ID NO: 13) and a reverse primer comprising the nucleotide sequence CCAAACAGACCAATGCTGGAGTTAG (SEQ ID NO: 14); and/or
(xiii) DHA is amplified in the presence of a forward primer comprising the nucleotide sequence ACGGGCCGGTAATGCGGATCTGGA (SEQ ID NO: 11) and a reverse primer comprising the nucleotide sequence TATTCGCCAGAATCACAATCGCCACCTGT (SEQ ID NO: 12).

30. The method of claim 23, wherein:
(i) the method comprises amplifying and/or detecting at least three, at least four, at least five, at least six, at least seven, at least eight, at least nine, or all ten antibiotic resistance genes selected from the group consisting of CTX-M 14, CTX-M 15, ermA, ermB, mecA, mefA, SHV, TEM, vanA, and vanB; and/or
(ii) the multiplexed amplification reaction is configured to amplify at least three, at least four, at least five, at least six, at least seven, at least eight, at least nine, or all ten antibiotic resistance genes selected from the group consisting of CTX-M 14, CTX-M 15, ermA, ermB, mecA, mefA, SHV, TEM, vanA, and vanB.

31. The method of claim 23 or 30, wherein:
(i) the method comprises amplifying and/or detecting CTX-M 14, CTX-M 15, ermA, ermB, mecA, mefA, SHV, TEM, vanA, and vanB; and/or
(ii) the multiplexed amplification reaction is configured to amplify CTX-M 14, CTX-M 15, ermA, ermB, mecA, mefA, SHV, TEM, vanA, and vanB.

32. The method of claim 30 or 31, wherein:
(i) CTX-M 14 is amplified in the presence of a forward primer comprising the nucleotide sequence ACGCTTTCCAATGTGCAGTACCAGTA (SEQ ID NO: 33) and a reverse primer comprising the nucleotide sequence TGCGATCCAGACGAAACGTCTCATCG (SEQ ID NO: 34);
(ii) CTX-M 15 is amplified in the presence of a forward primer comprising the nucleotide sequence GTGATACCACTTCACCTCGGGCAA (SEQ ID NO: 35) and a reverse primer comprising the nucleotide sequence AATACATCGCGACGGCTTTCTGCC (SEQ ID NO: 36);
(iii) ermA is amplified in the presence of a forward primer comprising the nucleotide sequence AGAATTACCTTTGAAAGTCAGGC (SEQ ID NO: 37) and a reverse primer comprising the nucleotide sequence GCTTCAAAGCCTGTCGGAATTGGTTT (SEQ ID NO: 38);
(iv) ermB is amplified in the presence of a forward primer comprising the nucleotide sequence GGGCATTTAACGACGAAACTGGCTA (SEQ ID NO: 39) and a reverse primer comprising the nucleotide sequence GTGTTCGGTGAATATCCAAGGTACGC (SEQ ID NO: 40);
(v) mecA is amplified in the presence of a forward primer comprising the nucleotide sequence ATGTTGGTCCCATTAACTCTGAAGAA (SEQ ID NO: 41) and a reverse primer comprising the nucleotide sequence CACCTGTTTGAGGGTGGATAGCAGTA (SEQ ID NO: 42);
(vi) mefA is amplified in the presence of a forward primer comprising the nucleotide sequence GCAGGGCAAGCAGTATCATTAATCAC (SEQ ID NO: 43) and a reverse primer comprising the nucleotide sequence AATTAAATCAGCACCAATCATTATCTTCTTCC (SEQ ID NO: 44);
(vii) SHV is amplified in the presence of a forward primer comprising the nucleotide sequence AAGCTGCTGACCAGCCAGCGTCTGA (SEQ ID NO: 45) and a reverse primer comprising the nucleotide sequence CGGCGATTTGCTGATTTCGCTCG (SEQ ID NO: 46);
(viii) TEM is amplified in the presence of a forward primer comprising the nucleotide sequence TGCAGTGCTGCCATAACCATGAGTGA (SEQ ID NO: 47) and a reverse primer comprising the nucleotide sequence AGCGCAGAAGTGGTCCTGCAACTTT (SEQ ID NO: 48);
(ix) vanA is amplified in the presence of a forward primer comprising the nucleotide sequence CAGTACGGAATCTTTCGTATTCATCAGGA (SEQ ID NO: 49) and a reverse primer comprising the nucleotide sequence CAGTTCGGGAAGTGCAATACCTGCA (SEQ ID NO: 50); and/or
(x) vanB is amplified in the presence of a forward primer comprising the nucleotide sequence AATTGAGCAAGCGATTTCGGGCTGT (SEQ ID NO: 51) and a reverse primer comprising the nucleotide sequence CGTTTAGAACGATGCCGCCATCCT (SEQ ID NO: 52).

33. The method of any one of claims 23-32, wherein:
amplifying step (c) further comprises amplifying a target nucleic acid characteristic of a bacterial pathogen, and step (i) further comprises detecting the amplified target nucleic acid characteristic of a bacterial pathogen to determine whether the bacterial pathogen is present; and/or
the multiplexed amplification reaction is configured to amplify a target nucleic acid characteristic of a bacterial pathogen.

34. The method of claim 33, wherein the target nucleic acid characteristic of a bacterial pathogen is characteristic of an Enterobacter spp., a Klebsiella spp., or Streptococcus pneumoniae.

35. The method of claim 33 or 34, wherein the target nucleic acid characteristic of a bacterial pathogen is characteristic of Enterobacter spp. and Klebsiella spp.

36. The method of claim 35, wherein the target nucleic acid characteristic of Enterobacter spp. and Klebsiella spp. is amplified in the presence of a forward primer comprising the nucleotide sequence ATTCGTTGCACTATCGTTAACTGAATACA (SEQ ID NO: 15) and a reverse primer comprising the nucleotide sequence CTGTACCGTCGGACTTTCCAGAC (SEQ ID NO: 16).

37. The method of claim 33 or 34, wherein the target nucleic acid characteristic of a bacterial pathogen is characteristic of Streptococcus pneumoniae.

38. The method of claim 37, wherein the target nucleic acid characteristic of Streptococcus pneumoniae is amplified in the presence of a forward primer comprising the nucleotide sequence CCTTGGACGGAAATGTAGCTGGCA (SEQ ID NO: 53) and a reverse primer comprising the nucleotide sequence AATCACATGGTTGACACCTGCTGTG (SEQ ID NO: 54).

39. The method of any one of claims 23-38, wherein:
amplifying step (c) further comprises amplifying an IC target nucleic acid and step (i) further comprises detecting the amplified IC target nucleic acid; and/or
the multiplexed amplification reaction is configured to amplify an amplified IC target nucleic acid.

40. The method of any one of claims 23-39, wherein the magnetic particles of each population comprise two subpopulations, a first subpopulation bearing a first probe on its surface, and a second subpopulation bearing a second probe on its surface.

41. The method of any one of claims 23-40, wherein the magnetic particles of each population comprise two subpopulations, a first subpopulation bearing a first probe and a second probe on its surface, and a second subpopulation bearing a third probe and a fourth probe on its surface.

42. The method of claim 40 or 41, wherein the method comprises amplifying two or more antibiotic resistance genes selected from the group consisting of NDM, KPC, IMP, VIM, OXA-48-like, DHA, and/or CMY, and wherein:
(i) a probe pair comprising a 5′ probe comprising the nucleotide sequence GCGACCGGCAGGTTGATCT (SEQ ID NO: 17) or GCGACCGGCAGGTTGATCTCCTGC (SEQ ID NO: 95) and a 3′ probe comprising the nucleotide sequence CATGTCGAGATAGGAAGTGTGCTGC (SEQ ID NO: 18) or CGGCATGTCGAGATAGGAAGTGTGCTGC (SEQ ID NO: 96) is used for detection of NDM;
(ii) a probe pair comprising a 5′ probe comprising the nucleotide sequence CGGAACCATTCGCTAAACTC (SEQ ID NO: 19) and a 3′ probe comprising the nucleotide sequence AGGCGCAACTGTAAGTTACCG (SEQ ID NO: 20) is used for detection of KPC;
(iii) a probe pair comprising a 5′ probe comprising the nucleotide sequence GCTTAATTCTCAATCTATCCCCACGTAT (SEQ ID NO: 21) and a 3′ probe comprising the nucleotide sequence CTCCAGATAACGTAGTGGTTTGGCTG (SEQ ID NO: 22) is used for detection of IMP;
(iv) a probe pair comprising a 5′ probe comprising the nucleotide sequence CTTTCATGACGACCGCGTCGG (SEQ ID NO: 23) and a 3′ probe comprising the nucleotide sequence CTCTAGAAGGACTCTCATCGAGC (SEQ ID NO: 24) is used for detection of VIM;
(v) a probe pair comprising a 5′ probe comprising the nucleotide sequence ATTTTAAAGGTAGATGCGGG (SEQ ID NO: 25) and a 3′ probe comprising the nucleotide sequence CGCCCTGTGATTTATGTTCA (SEQ ID NO: 26) is used for detection of OXA-48-like;
(vi) a probe pair comprising a 5′ probe comprising the nucleotide sequence GTTTTATGCACCCAGGAAGC (SEQ ID NO: 27) and a 3′ probe comprising the nucleotide sequence TCTGCTGCGGCCAGTCATA (SEQ ID NO: 28) is used for detection of DHA; and/or
(vii) a probe pair comprising a 5′ probe comprising the nucleotide sequence GCGGCTGCCAGTTTTGATAA (SEQ ID NO: 29) and a 3′ probe comprising the nucleotide sequence GTGGCTAAGTGCAGCAGGC (SEQ ID NO: 30) is used for detection of CMY.

43. The method of any one of claims 40-42, wherein the method comprises amplifying a target nucleic acid characteristic of Enterobacter spp. and Klebsiella spp., and a probe pair comprising a 5′ probe comprising the nucleotide sequence CGTTCCACTAACACACAAGCTGATTCAG (SEQ ID NO: 31) and a 3′ probe comprising the nucleotide sequence ATCTCGGTTGATTTCTTTTCCTCGGG (SEQ ID NO: 32) is used for detection of the target nucleic acid characteristic of Enterobacter spp. and Klebsiella spp.

44. The method of claim 40 or 41, wherein the method comprises amplifying two or more antibiotic resistance genes selected from the group consisting of KPC, CTX-M 14, CTX-M 15, NDM, VIM, IMP, vanA, vanB, mecA, mecC, OXA-48-like, CMY, and DHA, and wherein:
(i) a probe pair comprising a 5′ probe comprising the nucleotide sequence CGGAACCATTCGCTAAACTC (SEQ ID NO: 19) and a 3′ probe comprising the nucleotide sequence AGGCGCAACTGTAAGTTACCG (SEQ ID NO: 20) is used for detection of KPC;
(ii) a probe pair comprising a 5′ probe comprising the nucleotide sequence CTGTCGAGATCAAGCCTGCCGA (SEQ ID NO: 57) and a 3′ probe comprising the nucleotide sequence ACAAATTGATTGCCCAGCTCGGT (SEQ ID NO: 58) is used for detection of CTX-M 14;
(iii) a probe pair comprising a 5′ probe comprising the nucleotide sequence GGGCGCAGCTGGTGACAT (SEQ ID NO: 83) and a 3′ probe comprising the nucleotide sequence AAGATCGTGCGCCGCTGATT (SEQ ID NO: 84) is used for detection of CTX-M 15;
(iv) a probe pair comprising a 5′ probe comprising the nucleotide sequence GCGACCGGCAGGTTGATCT (SEQ ID NO: 17) or GCGACCGGCAGGTTGATCTCCTGC (SEQ ID NO: 95) and a 3′ probe comprising the nucleotide sequence CATGTCGAGATAGGAAGTGTGCTGC (SEQ ID NO: 18) or CGGCATGTCGAGATAGGAAGTGTGCTGC (SEQ ID NO: 96) is used for detection of NDM;
(v) a probe pair comprising a 5′ probe comprising the nucleotide sequence CTTTCATGACGACCGCGTCGG (SEQ ID NO: 23) and a 3′ probe comprising the nucleotide sequence CTCTAGAAGGACTCTCATCGAGC (SEQ ID NO: 24) is used for detection of VIM;
(vi) a probe pair comprising a 5′ probe comprising the nucleotide sequence GCTTAATTCTCAATCTATCCCCACGTAT (SEQ ID NO: 21) and a 3′ probe comprising the nucleotide sequence CTCCAGATAACGTAGTGGTTTGGCTG (SEQ ID NO: 22) is used for detection of IMP;
(vii) a probe pair comprising a 5′ probe comprising the nucleotide sequence GCAGTTATAACCGTTCCCGCAG (SEQ ID NO: 86) and a 3′ probe comprising the nucleotide sequence TAACGGCCGCATTGTACTGAACG (SEQ ID NO: 87) is used for detection of vanA;
(viii) a probe pair comprising a 5′ probe comprising the nucleotide sequence GCACCCGATATACTTTCTTTGCC (SEQ ID NO: 75) and a 3′ probe comprising the nucleotide sequence CGCCGACAATCAAATCATCCT (SEQ ID NO: 76) is used for detection of vanB;
(ix) a probe pair comprising a 5′ probe comprising the nucleotide sequence AAAGGCTATAAAGATGATGCAG (SEQ ID NO: 89) and a 3′ probe comprising the nucleotide sequence GAGTATTTATAACAACATGAAAAATGATT (SEQ ID NO: 90) is used for detection of mecA;
(x) a probe pair comprising a 5′ probe comprising the nucleotide sequence GGCTTAGAACGCCTCTATGAT (SEQ ID NO: 93) and a 3′ probe comprising the nucleotide sequence AGAGTACAAGAAAGTATTTATAAACATATGA (SEQ ID NO: 94) is used for detection of mecC;
(xi) a probe pair comprising a 5′ probe comprising the nucleotide sequence ATTTTAAAGGTAGATGCGGG (SEQ ID NO: 25) and a 3′ probe comprising the nucleotide sequence CGCCCTGTGATTTATGTTCA (SEQ ID NO: 26) is used for detection of OXA-48-like;
(xii) a probe pair comprising a 5′ probe comprising the nucleotide sequence GCGGCTGCCAGTTTTGATAA (SEQ ID NO: 29) and a 3′ probe comprising the nucleotide sequence GTGGCTAAGTGCAGCAGGC (SEQ ID NO: 30) is used for detection of CMY; and/or
(xiii) a probe pair comprising a 5′ probe comprising the nucleotide sequence GTTTTATGCACCCAGGAAGC (SEQ ID NO: 27) and a 3′ probe comprising the nucleotide sequence TCTGCTGCGGCCAGTCATA (SEQ ID NO: 28) is used for detection of DHA.

45. The method of claim 41 or 42, wherein the method comprises amplifying two or more antibiotic resistance genes selected from the group consisting of CTX-M 14, CTX-M 15, ermA, ermB, mecA, mefA, SHV, TEM, vanA, and vanB, and wherein:
(i) a probe pair comprising a 5′ probe comprising the nucleotide sequence CTGTCGAGATCAAGCCTGCCGA (SEQ ID NO: 57) and a 3′ probe comprising the nucleotide sequence ACAAATTGATTGCCCAGCTCGGT (SEQ ID NO: 58) is used for detection of CTX-M 14;
(ii) a probe pair comprising a 5′ probe comprising the nucleotide sequence AATCAGCGGCGCACGATCTTT (SEQ ID NO: 59) and a 3′ probe comprising the nucleotide sequence AATGCTCGCTGCACCGGTGGTAT (SEQ ID NO: 60) is used for detection of CTX-M 15;
(iii) a probe pair comprising a 5′ probe comprising the nucleotide sequence AAATCTGCAACGAGCTTTGGG (SEQ ID NO: 61) and a 3′ probe comprising the nucleotide sequence GTTTATAAGTGGGTAAACCGTGAATATC (SEQ ID NO: 62) is used for detection of ermA;
(iv) a probe pair comprising a 5′ probe comprising the nucleotide sequence TTCGTGTCACTTTAATTCACCAAGAT (SEQ ID NO: 63) and a 3′ probe comprising the nucleotide sequence AAAGCCATGCGTCTGACATCT (SEQ ID NO: 64) is used for detection of ermB;
(v) a probe pair comprising a 5′ probe comprising the nucleotide sequence AAGCTCCAACATGAAGATGGCT (SEQ ID NO: 65) and a 3′ probe comprising the nucleotide sequence AGATGGCAAAGATATTCAACTAAC (SEQ ID NO: 66) is used for detection of mecA;
(vi) a probe pair comprising a 5′ probe comprising the nucleotide sequence AGTGCCATCTTGCAAATGGCGAT (SEQ ID NO: 67) and a 3′ probe comprising the nucleotide sequence TGCAATTGGTGTGTTAGTGGATCGTCATGATA (SEQ ID NO: 68) is used for detection of mefA;
(vii) a probe pair comprising a 5′ probe comprising the nucleotide sequence CAGTGGATGGTGGACGATCGGGT (SEQ ID NO: 69) and a 3′ probe comprising the nucleotide sequence TTGTGGTGATTTATCTGCGGGATACT (SEQ ID NO: 70) is used for detection of SHV;
(viii) a probe pair comprising a 5′ probe comprising the nucleotide sequence CGCCAGTTAATAGTTTGCGCAACG (SEQ ID NO: 71) and a 3′ probe comprising the nucleotide sequence AAAGCGGTTAGCTCCTTCGGTCCT (SEQ ID NO: 72) is used for detection of TEM;
(ix) a probe pair comprising a 5′ probe comprising the nucleotide sequence CGTTCAGTACAATGCGGCCGTTA (SEQ ID NO: 73) and a 3′ probe comprising the nucleotide sequence CTGCGGGAACGGTTATAACTGC (SEQ ID NO: 74) is used for detection of vanA; and/or
(x) a probe pair comprising a 5′ probe comprising the nucleotide sequence GCACCCGATATACTTTCTTTGCC (SEQ ID NO: 75) and a 3′ probe comprising the nucleotide sequence CGCCGACAATCAAATCATCCT (SEQ ID NO: 76) is used for detection of vanB.

46. The method of claim 45, wherein the method comprises amplifying a target nucleic acid characteristic of Streptococcus pneumoniae, and a probe pair comprising a 5′ probe comprising the nucleotide sequence TTGACCAGTTCCGAGCAAATGGTA (SEQ ID NO: 77) and a 3′ probe comprising the nucleotide sequence GACAGTATCGATGTTCCAGCAGCT (SEQ ID NO: 78) is used for detection of the target nucleic acid characteristic of Streptococcus pneumoniae.

47. The method of any one of claims 23-46, wherein an assay sample is contacted with 1×10⁶ to 1×10¹³ magnetic particles per milliliter of the biological sample.

48. The method of any one of claims 23-47, wherein step (h) comprises measuring the T₂ relaxation response of the assay sample, and wherein increasing agglomeration in the assay sample produces an increase in the observed T₂ relaxation time of the assay sample.

49. The method of any one of claims 23-48, wherein the magnetic particles have a mean diameter of from 600 nm to 1200 nm.

50. The method of claim 49, wherein the magnetic particles have a mean diameter of from 650 nm to 950 nm.

51. The method of claim 50, wherein the magnetic particles have a mean diameter of from 670 nm to 890 nm.

52. The method of any one of claims 23-51, wherein the magnetic particles have a T₂ relaxivity per particle of from 1×10⁹ to 1×10¹² mM⁻¹ s⁻¹.

53. The method of any one of claims 23-52, wherein the magnetic particles are substantially monodisperse.

54. The method of any one of claims 23-53, further comprising sequencing the first and/or second amplified antibiotic resistance target nucleic acid.

55. A method for detecting the presence of an antibiotic resistance gene in a biological sample obtained from a subject, wherein the biological sample comprises subject-derived cells or cell debris, the method comprising:
(a) amplifying in the biological sample or a fraction thereof one or more antibiotic resistance target nucleic acids in a multiplexed amplification reaction, wherein the multiplexed amplification reaction is configured to amplify target nucleic acids characteristic of two or more antibiotic resistance genes selected from the group consisting of NDM, KPC, IMP, VIM, OXA-48-like, DHA, CMY, FOX, mecA, mecC, vanA, vanB, CTX-M 14, CTX-M 15, mefA, mefE, ermA, ermB, SHV, and TEM, wherein the two or more antibiotic resistance genes comprises at least one of IMP, OXA-48-like, DHA, CMY, FOX, CTX-M 14, CTX-M 15, mecC, mefA, mefE, ermA, ermB, or TEM; and
(b) sequencing the one or more amplified antibiotic resistance target nucleic acids to detect whether one or more of the antibiotic resistance genes is present in the biological sample,
wherein the method is capable of detecting an antibiotic resistance gene of a pathogen present at a concentration of 10 cells/mL of biological sample or less.

56. The method of claim 55, wherein:
(i) the method comprises amplifying and/or sequencing at least three, at least four, at least five, at least six, or all seven antibiotic resistance genes selected from the group consisting of NDM, KPC, IMP, VIM, OXA-48-like, DHA, and CMY; and/or
(ii) the multiplexed amplification reaction is configured to amplify at least three, at least four, at least five, at least six, or all seven antibiotic resistance genes selected from the group consisting of NDM, KPC, IMP, VIM, OXA-48-like, DHA, and CMY.

57. The method of claim 55 or 56, wherein:
(i) the method comprises amplifying and/or sequencing NDM, KPC, IMP, VIM, OXA-48-like, DHA, and CMY; and/or
(ii) the multiplexed amplification reaction is configured to amplify NDM, KPC, IMP, VIM, OXA-48-like, DHA, and CMY.

58. The method of claim 56 or 57, wherein:
(i) NDM is amplified in the presence of a forward primer comprising the nucleotide sequence CCAGCTCGCACCGAATGTCT (SEQ ID NO: 1) and a reverse primer comprising the nucleotide sequence CATCTTGTCCTGATGCGCGTGAGTCA (SEQ ID NO: 2);
(ii) KPC is amplified in the presence of a forward primer comprising the nucleotide sequence TTTCTGCCACCGCGCTGAC (SEQ ID NO: 3) and a reverse primer comprising the nucleotide sequence GCAGCAAGAAAGCCCTTGAATG (SEQ ID NO: 4);
(iii) IMP is amplified in the presence of a forward primer comprising the nucleotide sequence ACATTTCCATAGCGACAGCACGGGCGGAAT (SEQ ID NO: 5) and a reverse primer comprising the nucleotide sequence GGACTTTGGCCAAGCTTCTAAATTTGCGTC (SEQ ID NO: 6);
(iv) VIM is amplified in the presence of a forward primer comprising the nucleotide sequence CGTGCAGTCTCCACGCACT (SEQ ID NO: 7) and a reverse primer comprising the nucleotide sequence TCGAATGCGCAGCACCGGGATAG (SEQ ID NO: 8);
(v) OXA-48-like is amplified in the presence of a forward primer comprising the nucleotide sequence GGCTGTGTTTTGGTGGCATCGATTATC (SEQ ID NO: 9) and a reverse primer comprising the nucleotide sequence TCCCACTTAAAGACTTGGTGTTCATCC (SEQ ID NO: 10);
(vi) DHA is amplified in the presence of a forward primer comprising the nucleotide sequence ACGGGCCGGTAATGCGGATCTGGA (SEQ ID NO: 11) and a reverse primer comprising the nucleotide sequence TATTCGCCAGAATCACAATCGCCACCTGT (SEQ ID NO: 12); and/or
(vii) CMY is amplified in the presence of a forward primer comprising the nucleotide sequence CCGCGGCGAAATTAAGCTCAGCGA (SEQ ID NO: 13) and a reverse primer comprising the nucleotide sequence CCAAACAGACCAATGCTGGAGTTAG (SEQ ID NO: 14).

59. The method of claim 55, wherein:
(i) the method comprises amplifying and/or sequencing at least three, at least four, at least five, at least six, at least seven, at least eight, at least nine, at least ten, at least eleven, at least twelve, or all thirteen antibiotic resistance genes selected from the group consisting of KPC, CTX-M 14, CTX-M 15, NDM, VIM, IMP, vanA, vanB, mecA, mecC, OXA-48-like, CMY, and DHA; and/or
(ii) the multiplexed amplification reaction is configured to amplify at least three, at least four, at least five, at least six, at least seven, at least eight, at least nine, at least ten, at least eleven, at least twelve, or all thirteen antibiotic resistance genes selected from the group consisting of KPC, CTX-M 14, CTX-M 15, NDM, VIM, IMP, vanA, vanB, mecA, mecC, OXA-48-like, CMY, and DHA.

60. The method of claim 55 or 59, wherein:
(i) the method comprises amplifying and/or sequencing KPC, CTX-M 14, CTX-M 15, NDM, VIM, IMP, vanA, vanB, mecA, mecC, OXA-48-like, CMY, and DHA; and/or
(ii) the multiplexed amplification reaction is configured to amplify KPC, CTX-M 14, CTX-M 15, NDM, VIM, IMP, vanA, vanB, mecA, mecC, OXA-48-like, CMY, and DHA.

61. The method of claim 59 or 60, wherein:
(i) KPC is amplified in the presence of a forward primer comprising the nucleotide sequence TTTCTGCCACCGCGCTGAC (SEQ ID NO: 3) and a reverse primer comprising the nucleotide sequence GCAGCAAGAAAGCCCTTGAATG (SEQ ID NO: 4);
(ii) CTX-M 14 is amplified in the presence of a forward primer comprising the nucleotide sequence ACGCTTTCCAATGTGCAGTACCAGTA (SEQ ID NO: 33) and a reverse primer comprising the nucleotide sequence TGCGATCCAGACGAAACGTCTCATCG (SEQ ID NO: 34);
(iii) CTX-M 15 is amplified in the presence of a forward primer comprising the nucleotide sequence CCTCGGGCAATGGCGCAAAC (SEQ ID NO: 81) and a reverse primer comprising the nucleotide sequence ATCGCGACGGCTTTCTGCCTTA (SEQ ID NO: 82);
(iv) NDM is amplified in the presence of a forward primer comprising the nucleotide sequence CCAGCTCGCACCGAATGTCT (SEQ ID NO: 1) and a reverse primer comprising the nucleotide sequence CATCTTGTCCTGATGCGCGTGAGTCA (SEQ ID NO: 2);
(v) VIM is amplified in the presence of a forward primer comprising the nucleotide sequence CGTGCAGTCTCCACGCACT (SEQ ID NO: 7) and a reverse primer comprising the nucleotide sequence TCGAATGCGCAGCACCGGGATAG (SEQ ID NO: 8);
(vi) IMP is amplified in the presence of a forward primer comprising the nucleotide sequence ACATTTCCATAGCGACAGCACGGGCGGAAT (SEQ ID NO: 5) and a reverse primer comprising the nucleotide sequence GGACTTTGGCCAAGCTTCTAAATTTGCGTC (SEQ ID NO: 6);
(vii) vanA is amplified in the presence of a forward primer comprising the nucleotide sequence TATTCATCAGGAAGTCGAGCCGGA (SEQ ID NO: 85) and a reverse primer comprising the nucleotide sequence CAGTTCGGGAAGTGCAATACCTGCA (SEQ ID NO: 50);
(viii) vanB is amplified in the presence of a forward primer comprising the nucleotide sequence AATTGAGCAAGCGATTTCGGGCTGT (SEQ ID NO: 51) and a reverse primer comprising the nucleotide sequence AAGATCAACACGGGCAAGCCCTCT (SEQ ID NO: 88);
(ix) mecA is amplified in the presence of a forward primer comprising the nucleotide sequence ATGTTGGTCCCATTAACTCTGAAGAA (SEQ ID NO: 41) and a reverse primer comprising the nucleotide sequence CACCTGTTTGAGGGTGGATAGCAGTA (SEQ ID NO: 42);
(x) mecC is amplified in the presence of a forward primer comprising the nucleotide sequence ATGTGGGTCCAATTAATTCTGACGAG (SEQ ID NO: 91) and a reverse primer comprising the nucleotide sequence CTCCAGTTTTGGTTGTAATGCTGTA (SEQ ID NO: 92);
(xi) OXA-48-like is amplified in the presence of a forward primer comprising the nucleotide sequence GGCTGTGTTTTGGTGGCATCGATTATC (SEQ ID NO: 9) and a reverse primer comprising the nucleotide sequence TCCCACTTAAAGACTTGGTGTTCATCC (SEQ ID NO: 10);
(xii) CMY is amplified in the presence of a forward primer comprising the nucleotide sequence CCGCGGCGAAATTAAGCTCAGCGA (SEQ ID NO: 13) and a reverse primer comprising the nucleotide sequence CCAAACAGACCAATGCTGGAGTTAG (SEQ ID NO: 14); and/or
(xiii) DHA is amplified in the presence of a forward primer comprising the nucleotide sequence ACGGGCCGGTAATGCGGATCTGGA (SEQ ID NO: 11) and a reverse primer comprising the nucleotide sequence TATTCGCCAGAATCACAATCGCCACCTGT (SEQ ID NO: 12).

62. The method of claim 55, wherein:
(i) the method comprises amplifying and/or sequencing at least three, at least four, at least five, at least six, at least seven, at least eight, at least nine, or all ten antibiotic resistance genes selected from the group consisting of CTX-M 14, CTX-M 15, ermA, ermB, mecA, mefA, SHV, TEM, vanA, and vanB; and/or
(ii) the multiplexed amplification reaction is configured to amplify at least three, at least four, at least five, at least six, at least seven, at least eight, at least nine, or all ten antibiotic resistance genes selected from the group consisting of CTX-M 14, CTX-M 15, ermA, ermB, mecA, mefA, SHV, TEM, vanA, and vanB.

63. The method of claim 55 or 62, wherein:
(i) the method comprises amplifying and/or sequencing CTX-M 14, CTX-M 15, ermA, ermB, mecA, mefA, SHV, TEM, vanA, and vanB; and/or
(ii) the multiplexed amplification reaction is configured to amplify CTX-M 14, CTX-M 15, ermA, ermB, mecA, mefA, SHV, TEM, vanA, and vanB.

64. The method of claim 62 or 63, wherein:
(i) CTX-M 14 is amplified in the presence of a forward primer comprising the nucleotide sequence ACGCTTTCCAATGTGCAGTACCAGTA (SEQ ID NO: 33) and a reverse primer comprising the nucleotide sequence TGCGATCCAGACGAAACGTCTCATCG (SEQ ID NO: 34);
(ii) CTX-M 15 is amplified in the presence of a forward primer comprising the nucleotide sequence GTGATACCACTTCACCTCGGGCAA (SEQ ID NO: 35) and a reverse primer comprising the nucleotide sequence AATACATCGCGACGGCTTTCTGCC (SEQ ID NO: 36);
(iii) ermA is amplified in the presence of a forward primer comprising the nucleotide sequence AGAATTACCTTTGAAAGTCAGGC (SEQ ID NO: 37) and a reverse primer comprising the nucleotide sequence GCTTCAAAGCCTGTCGGAATTGGTTT (SEQ ID NO: 38);
(iv) ermB is amplified in the presence of a forward primer comprising the nucleotide sequence GGGCATTTAACGACGAAACTGGCTA (SEQ ID NO: 39) and a reverse primer comprising the nucleotide sequence GTGTTCGGTGAATATCCAAGGTACGC (SEQ ID NO: 40);
(v) mecA is amplified in the presence of a forward primer comprising the nucleotide sequence ATGTTGGTCCCATTAACTCTGAAGAA (SEQ ID NO: 41) and a reverse primer comprising the nucleotide sequence CACCTGTTTGAGGGTGGATAGCAGTA (SEQ ID NO: 42);
(vi) mefA is amplified in the presence of a forward primer comprising the nucleotide sequence GCAGGGCAAGCAGTATCATTAATCAC (SEQ ID NO: 43) and a reverse primer comprising the nucleotide sequence AATTAAATCAGCACCAATCATTATCTTCTTCC (SEQ ID NO: 44);
(vii) SHV is amplified in the presence of a forward primer comprising the nucleotide sequence AAGCTGCTGACCAGCCAGCGTCTGA (SEQ ID NO: 45) and a reverse primer comprising the nucleotide sequence CGGCGATTTGCTGATTTCGCTCG (SEQ ID NO: 46);
(viii) TEM is amplified in the presence of a forward primer comprising the nucleotide sequence TGCAGTGCTGCCATAACCATGAGTGA (SEQ ID NO: 47) and a reverse primer comprising the nucleotide sequence AGCGCAGAAGTGGTCCTGCAACTTT (SEQ ID NO: 48);
(ix) vanA is amplified in the presence of a forward primer comprising the nucleotide sequence CAGTACGGAATCTTTCGTATTCATCAGGA (SEQ ID NO: 49) and a reverse primer comprising the nucleotide sequence CAGTTCGGGAAGTGCAATACCTGCA (SEQ ID NO: 50); and/or
(x) vanB is amplified in the presence of a forward primer comprising the nucleotide sequence AATTGAGCAAGCGATTTCGGGCTGT (SEQ ID NO: 51) and a reverse primer comprising the nucleotide sequence CGTTTAGAACGATGCCGCCATCCT (SEQ ID NO: 52).

65. The method of any one of claims 55-64, wherein:
(i) amplifying step (a) further comprises amplifying a target nucleic acid characteristic of a bacterial pathogen, and step (b) further comprises sequencing the amplified target nucleic acid characteristic of a bacterial pathogen to determine whether the bacterial pathogen is present; and/or
(ii) the multiplexed amplification reaction is configured to amplify a target nucleic acid characteristic of a bacterial pathogen.

66. The method of claim 65, wherein the target nucleic acid characteristic of a bacterial pathogen is characteristic of an Enterobacter spp., a Klebsiella spp., or Streptococcus pneumoniae.

67. The method of claim 65 or 66, wherein the target nucleic acid characteristic of a bacterial pathogen is characteristic of Enterobacter spp. and Klebsiella spp.

68. The method of claim 67, wherein the target nucleic acid characteristic of Enterobacter spp. and Klebsiella spp. is amplified in the presence of a forward primer comprising the nucleotide sequence ATTCGTTGCACTATCGTTAACTGAATACA (SEQ ID NO: 15) and a reverse primer comprising the nucleotide sequence CTGTACCGTCGGACTTTCCAGAC (SEQ ID NO: 16).

69. The method of claim 65 or 66, wherein the target nucleic acid characteristic of a bacterial pathogen is characteristic of Streptococcus pneumoniae.

70. The method of claim 69, wherein the target nucleic acid characteristic of Streptococcus pneumoniae is amplified in the presence of a forward primer comprising the nucleotide sequence CCTTGGACGGAAATGTAGCTGGCA (SEQ ID NO: 53) and a reverse primer comprising the nucleotide sequence AATCACATGGTTGACACCTGCTGTG (SEQ ID NO: 54).

71. The method of any one of claims 55-70, wherein step (a) comprises amplifying the one or more antibiotic resistance target nucleic acids in a lysate produced by lysing cells in the biological sample.

72. The method of claim 71, wherein the lysate has at least about a 2:1, a 5:1, a 10:1, a 20:1, a 40:1, or a 60:1 higher concentration of cell debris relative to the biological sample.

73. The method of claim 72, wherein the cell debris is solid material.

74. The method of any one of claims 1-73, wherein the biological sample has a volume of about 0.1 mL to about 5 mL.

75. The method of claim 74, wherein the biological sample has a volume of about 2 mL.

76. The method of any one of claims 1-75, wherein the biological sample is selected from the group consisting of blood, a bloody fluid, a tissue sample, bronchiolar lavage (BAL), urine, cerebrospinal fluid (CSF), synovial fluid (SF), and sputum.

77. The method of claim 76, wherein the blood is whole blood, a crude blood lysate, serum, or plasma.

78. The method of claim 77, wherein the whole blood is ethylenediaminetetraacetic acid (EDTA) whole blood, sodium citrate whole blood, sodium heparin whole blood, lithium heparin whole blood, or potassium oxylate/sodium fluoride whole blood.

79. The method of claim 76, wherein the bloody fluid is wound exudate, wound aspirate, phlegm, or bile.

80. The method of claim 76, wherein the tissue sample is a tissue sample from a transplant, a tissue biopsy (e.g., a skin biopsy, muscle biopsy, or lymph node biopsy), a homogenized tissue sample, or bone.

81. The method of claim 76, wherein the biological sample is urine or BAL.

82. The method of any one of claims 1-81, wherein the biological sample is a swab.

83. The method of any one of claims 55-82, further comprising detecting the amplified target pathogen nucleic acid(s) using T2 magnetic resonance (T2MR).

84. A method for detecting the presence of an antibiotic resistance gene in a whole blood sample, the method comprising:
(a) contacting a whole blood sample suspected of containing one or more pathogen cells with an erythrocyte lysis agent, thereby lysing red blood cells;
(b) centrifuging the product of step (a) to form a supernatant and a pellet;
(c) discarding some or all of the supernatant of step (b) and resuspending the pellet to form an extract, optionally washing the pellet one or more times prior to resuspending the pellet;
(d) lysing the remaining cells in the extract of step (c) to form a lysate, the lysate containing both subject cell nucleic acid and pathogen nucleic acid;
(e) amplifying in the lysate of step (d) one or more antibiotic resistance target nucleic acids in a multiplexed amplification reaction, wherein the multiplexed amplification reaction is configured to amplify target nucleic acids characteristic of two or more antibiotic resistance genes selected from the group consisting of NDM, KPC, IMP, VIM, OXA-48-like, DHA, CMY, FOX, mecA, mecC, vanA, vanB, CTX-M 14, CTX-M 15, mefA, mefE, ermA, ermB, SHV, and TEM, wherein the two or more antibiotic resistance genes comprises at least one of IMP, OXA-48-like, DHA, CMY, FOX, CTX-M 14, CTX-M 15, mecC, mefA, mefE, ermA, ermB, or TEM; and
(f) detecting the one or more amplified target antibiotic resistance target nucleic acids, thereby detecting the presence of the one or more of the antibiotic resistance genes in the sample.

85. The method of any one of claims 1-84, wherein:
(i) the method comprises amplifying and/or detecting at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, or all nineteen antibiotic resistance genes selected from the group consisting of NDM, KPC, IMP, VIM, OXA-48-like, DHA, CMY, mecA, mecC, vanA, vanB, CTX-M 14, CTX-M 15, mefA, mefE, ermA, ermB, SHV, and TEM; and/or
(ii) the multiplexed amplification reaction is configured to amplify at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, or all nineteen antibiotic resistance genes selected from the group consisting of NDM, KPC, IMP, VIM, OXA-48-like, DHA, CMY, mecA, mecC, vanA, vanB, CTX-M 14, CTX-M 15, mefA, mefE, ermA, ermB, SHV, and TEM.

86. The method of claim 84 or 85, wherein:
(i) the method comprises amplifying and/or detecting at least three, at least four, at least five, at least six, or all seven antibiotic resistance genes selected from the group consisting of NDM, KPC, IMP, VIM, OXA-48-like, DHA, and CMY; and/or
(ii) the multiplexed amplification reaction is configured to amplify at least three, at least four, at least five, at least six, or all seven antibiotic resistance genes selected from the group consisting of NDM, KPC, IMP, VIM, OXA-48-like, DHA, and CMY.

87. The method of any one of claims 84-86, wherein:
(i) the method comprises amplifying and/or detecting NDM, KPC, IMP, VIM, OXA-48-like, DHA, and CMY; and/or
(ii) the multiplexed amplification reaction is configured to amplify NDM, KPC, IMP, VIM, OXA-48-like, DHA, and CMY.

88. The method of claim 86 or 87, wherein:
(i) NDM is amplified in the presence of a forward primer comprising the nucleotide sequence CCAGCTCGCACCGAATGTCT (SEQ ID NO: 1) and a reverse primer comprising the nucleotide sequence CATCTTGTCCTGATGCGCGTGAGTCA (SEQ ID NO: 2);
(ii) KPC is amplified in the presence of a forward primer comprising the nucleotide sequence TTTCTGCCACCGCGCTGAC (SEQ ID NO: 3) and a reverse primer comprising the nucleotide sequence GCAGCAAGAAAGCCCTTGAATG (SEQ ID NO: 4);
(iii) IMP is amplified in the presence of a forward primer comprising the nucleotide sequence ACATTTCCATAGCGACAGCACGGGCGGAAT (SEQ ID NO: 5) and a reverse primer comprising the nucleotide sequence GGACTTTGGCCAAGCTTCTAAATTTGCGTC (SEQ ID NO: 6);
(iv) VIM is amplified in the presence of a forward primer comprising the nucleotide sequence CGTGCAGTCTCCACGCACT (SEQ ID NO: 7) and a reverse primer comprising the nucleotide sequence TCGAATGCGCAGCACCGGGATAG (SEQ ID NO: 8);
(v) OXA-48-like is amplified in the presence of a forward primer comprising the nucleotide sequence GGCTGTGTTTTGGTGGCATCGATTATC (SEQ ID NO: 9) and a reverse primer comprising the nucleotide sequence TCCCACTTAAAGACTTGGTGTTCATCC (SEQ ID NO: 10);
(vi) DHA is amplified in the presence of a forward primer comprising the nucleotide sequence ACGGGCCGGTAATGCGGATCTGGA (SEQ ID NO: 11) and a reverse primer comprising the nucleotide sequence TATTCGCCAGAATCACAATCGCCACCTGT (SEQ ID NO: 12); and/or
(vii) CMY is amplified in the presence of a forward primer comprising the nucleotide sequence CCGCGGCGAAATTAAGCTCAGCGA (SEQ ID NO: 13) and a reverse primer comprising the nucleotide sequence CCAAACAGACCAATGCTGGAGTTAG (SEQ ID NO: 14).

89. The method of claim 84 or 85, wherein:
(i) the method comprises amplifying and/or detecting at least three, at least four, at least five, at least six, at least seven, at least eight, at least nine, at least ten, at least eleven, at least twelve, or all thirteen antibiotic resistance genes selected from the group consisting of KPC, CTX-M 14, CTX-M 15, NDM, VIM, IMP, vanA, vanB, mecA, mecC, OXA-48-like, CMY, and DHA; and/or
(ii) the multiplexed amplification reaction is configured to amplify at least three, at least four, at least five, at least six, at least seven, at least eight, at least nine, at least ten, at least eleven, at least twelve, or all thirteen antibiotic resistance genes selected from the group consisting of KPC, CTX-M 14, CTX-M 15, NDM, VIM, IMP, vanA, vanB, mecA, mecC, OXA-48-like, CMY, and DHA.

90. The method of claim 84, 85, or 89, wherein:
(i) the method comprises amplifying and/or detecting KPC, CTX-M 14, CTX-M 15, NDM, VIM, IMP, vanA, vanB, mecA, mecC, OXA-48-like, CMY, and DHA; and/or
(ii) the multiplexed amplification reaction is configured to amplify KPC, CTX-M 14, CTX-M 15, NDM, VIM, IMP, vanA, vanB, mecA, mecC, OXA-48-like, CMY, and DHA.

91. The method of claim 89 or 90, wherein:
(i) KPC is amplified in the presence of a forward primer comprising the nucleotide sequence TTTCTGCCACCGCGCTGAC (SEQ ID NO: 3) and a reverse primer comprising the nucleotide sequence GCAGCAAGAAAGCCCTTGAATG (SEQ ID NO: 4);
(ii) CTX-M 14 is amplified in the presence of a forward primer comprising the nucleotide sequence ACGCTTTCCAATGTGCAGTACCAGTA (SEQ ID NO: 33) and a reverse primer comprising the nucleotide sequence TGCGATCCAGACGAAACGTCTCATCG (SEQ ID NO: 34);
(iii) CTX-M 15 is amplified in the presence of a forward primer comprising the nucleotide sequence CCTCGGGCAATGGCGCAAAC (SEQ ID NO: 81) and a reverse primer comprising the nucleotide sequence ATCGCGACGGCTTTCTGCCTTA (SEQ ID NO: 82);
(iv) NDM is amplified in the presence of a forward primer comprising the nucleotide sequence CCAGCTCGCACCGAATGTCT (SEQ ID NO: 1) and a reverse primer comprising the nucleotide sequence CATCTTGTCCTGATGCGCGTGAGTCA (SEQ ID NO: 2);
(v) VIM is amplified in the presence of a forward primer comprising the nucleotide sequence CGTGCAGTCTCCACGCACT (SEQ ID NO: 7) and a reverse primer comprising the nucleotide sequence TCGAATGCGCAGCACCGGGATAG (SEQ ID NO: 8);
(vi) IMP is amplified in the presence of a forward primer comprising the nucleotide sequence ACATTTCCATAGCGACAGCACGGGCGGAAT (SEQ ID NO: 5) and a reverse primer comprising the nucleotide sequence GGACTTTGGCCAAGCTTCTAAATTTGCGTC (SEQ ID NO: 6);
(vii) vanA is amplified in the presence of a forward primer comprising the nucleotide sequence TATTCATCAGGAAGTCGAGCCGGA (SEQ ID NO: 85) and a reverse primer comprising the nucleotide sequence CAGTTCGGGAAGTGCAATACCTGCA (SEQ ID NO: 50);
(viii) vanB is amplified in the presence of a forward primer comprising the nucleotide sequence AATTGAGCAAGCGATTTCGGGCTGT (SEQ ID NO: 51) and a reverse primer comprising the nucleotide sequence AAGATCAACACGGGCAAGCCCTCT (SEQ ID NO: 88);
(ix) mecA is amplified in the presence of a forward primer comprising the nucleotide sequence ATGTTGGTCCCATTAACTCTGAAGAA (SEQ ID NO: 41) and a reverse primer comprising the nucleotide sequence CACCTGTTTGAGGGTGGATAGCAGTA (SEQ ID NO: 42);
(x) mecC is amplified in the presence of a forward primer comprising the nucleotide sequence ATGTGGGTCCAATTAATTCTGACGAG (SEQ ID NO: 91) and a reverse primer comprising the nucleotide sequence CTCCAGTTTTGGTTGTAATGCTGTA (SEQ ID NO: 92);
(xi) OXA-48-like is amplified in the presence of a forward primer comprising the nucleotide sequence GGCTGTGTTTTGGTGGCATCGATTATC (SEQ ID NO: 9) and a reverse primer comprising the nucleotide sequence TCCCACTTAAAGACTTGGTGTTCATCC (SEQ ID NO: 10);
(xii) CMY is amplified in the presence of a forward primer comprising the nucleotide sequence CCGCGGCGAAATTAAGCTCAGCGA (SEQ ID NO: 13) and a reverse primer comprising the nucleotide sequence CCAAACAGACCAATGCTGGAGTTAG (SEQ ID NO: 14); and/or
(xiii) DHA is amplified in the presence of a forward primer comprising the nucleotide sequence ACGGGCCGGTAATGCGGATCTGGA (SEQ ID NO: 11) and a reverse primer comprising the nucleotide sequence TATTCGCCAGAATCACAATCGCCACCTGT (SEQ ID NO: 12).

92. The method of claim 84 or 85, wherein:
(i) the method comprises amplifying and/or detecting at least three, at least four, at least five, at least six, at least seven, at least eight, at least nine, or all ten antibiotic resistance genes selected from the group consisting of CTX-M 14, CTX-M 15, ermA, ermB, mecA, mefA, SHV, TEM, vanA, and vanB; and/or
(ii) the multiplexed amplification reaction is configured to amplify at least three, at least four, at least five, at least six, at least seven, at least eight, at least nine, or all ten antibiotic resistance genes selected from the group consisting of CTX-M 14, CTX-M 15, ermA, ermB, mecA, mefA, SHV, TEM, vanA, and vanB.

93. The method of claim 84, 85, or 92, wherein:
(i) the method comprises amplifying and/or detecting CTX-M 14, CTX-M 15, ermA, ermB, mecA, mefA, SHV, TEM, vanA, and vanB; and/or
(ii) the multiplexed amplification reaction is configured to amplify CTX-M 14, CTX-M 15, ermA, ermB, mecA, mefA, SHV, TEM, vanA, and vanB.

94. The method of claim 92 or 93, wherein:
(i) CTX-M 14 is amplified in the presence of a forward primer comprising the nucleotide sequence ACGCTTTCCAATGTGCAGTACCAGTA (SEQ ID NO: 33) and a reverse primer comprising the nucleotide sequence TGCGATCCAGACGAAACGTCTCATCG (SEQ ID NO: 34);
(ii) CTX-M 15 is amplified in the presence of a forward primer comprising the nucleotide sequence GTGATACCACTTCACCTCGGGCAA (SEQ ID NO: 35) and a reverse primer comprising the nucleotide sequence AATACATCGCGACGGCTTTCTGCC (SEQ ID NO: 36);
(iii) ermA is amplified in the presence of a forward primer comprising the nucleotide sequence AGAATTACCTTTGAAAGTCAGGC (SEQ ID NO: 37) and a reverse primer comprising the nucleotide sequence GCTTCAAAGCCTGTCGGAATTGGTTT (SEQ ID NO: 38);
(iv) ermB is amplified in the presence of a forward primer comprising the nucleotide sequence GGGCATTTAACGACGAAACTGGCTA (SEQ ID NO: 39) and a reverse primer comprising the nucleotide sequence GTGTTCGGTGAATATCCAAGGTACGC (SEQ ID NO: 40);
(v) mecA is amplified in the presence of a forward primer comprising the nucleotide sequence ATGTTGGTCCCATTAACTCTGAAGAA (SEQ ID NO: 41) and a reverse primer comprising the nucleotide sequence CACCTGTTTGAGGGTGGATAGCAGTA (SEQ ID NO: 42);
(vi) mefA is amplified in the presence of a forward primer comprising the nucleotide sequence GCAGGGCAAGCAGTATCATTAATCAC (SEQ ID NO: 43) and a reverse primer comprising the nucleotide sequence AATTAAATCAGCACCAATCATTATCTTCTTCC (SEQ ID NO: 44);
(vii) SHV is amplified in the presence of a forward primer comprising the nucleotide sequence AAGCTGCTGACCAGCCAGCGTCTGA (SEQ ID NO: 45) and a reverse primer comprising the nucleotide sequence CGGCGATTTGCTGATTTCGCTCG (SEQ ID NO: 46);
(viii) TEM is amplified in the presence of a forward primer comprising the nucleotide sequence TGCAGTGCTGCCATAACCATGAGTGA (SEQ ID NO: 47) and a reverse primer comprising the nucleotide sequence AGCGCAGAAGTGGTCCTGCAACTTT (SEQ ID NO: 48);
(ix) vanA is amplified in the presence of a forward primer comprising the nucleotide sequence CAGTACGGAATCTTTCGTATTCATCAGGA (SEQ ID NO: 49) and a reverse primer comprising the nucleotide sequence CAGTTCGGGAAGTGCAATACCTGCA (SEQ ID NO: 50); and/or
(x) vanB is amplified in the presence of a forward primer comprising the nucleotide sequence AATTGAGCAAGCGATTTCGGGCTGT (SEQ ID NO: 51) and a reverse primer comprising the nucleotide sequence CGTTTAGAACGATGCCGCCATCCT (SEQ ID NO: 52).

95. The method of any one of claims 84-94, wherein:
(i) amplifying step (e) further comprises amplifying a target nucleic acid characteristic of a bacterial pathogen, and step (f) further comprises detecting the amplified target nucleic acid characteristic of a bacterial pathogen to determine whether the bacterial pathogen is present; and/or
(ii) the multiplexed amplification reaction is configured to amplify a target nucleic acid characteristic of a bacterial pathogen.

96. The method of claim 95, wherein the target nucleic acid characteristic of a bacterial pathogen is characteristic of an Enterobacter spp., a Klebsiella spp., or Streptococcus pneumoniae.

97. The method of claim 95 or 96, wherein the target nucleic acid characteristic of a bacterial pathogen is characteristic of Enterobacter spp. and Klebsiella spp.

98. The method of claim 97, wherein the target nucleic acid characteristic of Enterobacter spp. and Klebsiella spp. is amplified in the presence of a forward primer comprising the nucleotide sequence ATTCGTTGCACTATCGTTAACTGAATACA (SEQ ID NO: 15) and a reverse primer comprising the nucleotide sequence CTGTACCGTCGGACTTTCCAGAC (SEQ ID NO: 16).

99. The method of claim 95 or 96, wherein the target nucleic acid characteristic of a bacterial pathogen is characteristic of Streptococcus pneumoniae.

100. The method of claim 99, wherein the target nucleic acid characteristic of Streptococcus pneumoniae is amplified in the presence of a forward primer comprising the nucleotide sequence CCTTGGACGGAAATGTAGCTGGCA (SEQ ID NO: 53) and a reverse primer comprising the nucleotide sequence AATCACATGGTTGACACCTGCTGTG (SEQ ID NO: 54).

101. The method of any one of claims 84-100, wherein step (c) comprises washing the pellet one time prior to resuspending the pellet.

102. The method of any one of claims 84-101, wherein the washing or resuspending is performed with a wash buffer solution.

103. The method of claim 102, wherein the wash buffer solution is Tris-EDTA (TE) buffer.

104. The method of claim 102 or 103, wherein the washing is performed with a wash buffer solution having a volume of about 100 μL to about 500 μL.

105. The method of claim 104, wherein the volume is about 150 μL.

106. The method of any one of claims 84-105, wherein resuspending of step (c) is performed with a wash buffer solution having a volume of about 50 μL to about 150 μL.

107. The method of claim 106, wherein the volume is about 100 μL.

108. The method of any one of claims 55-107, wherein:
(i) the amplifying further comprises amplifying an IC target nucleic acid and the method further comprises the amplified IC target nucleic acid; and/or
(ii) the multiplexed amplification reaction is configured to amplify an amplified IC target nucleic acid.

109. The method of any one of claims 17, 39, or 108, wherein the IC target nucleic acid is amplified in the presence of a forward primer comprising the nucleotide sequence GGAAATCTAACGAGAGAGCATGCT (SEQ ID NO: 55) and a reverse primer comprising the nucleotide sequence CGATGCGTGACACCCAGGC (SEQ ID NO: 56)

110. The method of any one of claims 84-109, wherein the wash buffer solution further comprises an IC nucleic acid.

111. The method of any one of claims 84-110, wherein step (a) further comprises adding a total process control (TPC) to the whole blood sample.

112. The method of claim 111, wherein the TPC is an engineered cell comprising a control target nucleic acid.

113. The method of any one of claims 55-112, wherein amplifying is in the presence of whole blood proteins and non-target nucleic acids.

114. The method of any one of claims 23-54 or 71-113, wherein lysing comprises mechanical lysis or heat lysis.

115. The method of claim 114, wherein the mechanical lysis is beadbeating or sonicating.

116. The method of any one of claims 1-115, wherein the steps of the method are completed within 5 hours.

117. The method of claim 116, wherein the steps of the method are completed within 4 hours.

118. The method of claim 117, wherein the steps of the method are completed within 3 hours.

119. The method of any one of claims 84-118, wherein the detecting comprises T2MR.

120. The method of any one of claims 84-119, wherein the detecting comprises sequencing.

121. The method of any one of claims 1-120, wherein the amplifying comprises polymerase chain reaction (PCR), ligase chain reaction (LCR), multiple displacement amplification (MDA), strand displacement amplification (SDA), rolling circle amplification (RCA), loop mediated isothermal amplification (LAMP), nucleic acid sequence based amplification (NASBA), helicase dependent amplification, recombinase polymerase amplification, nicking enzyme amplification reaction, or ramification amplification (RAM).

122. The method of claim 121, wherein the amplifying comprises PCR.

123. The method of claim 122, wherein the PCR is symmetric PCR or asymmetric PCR.

124. The method of any one of claims 22, 54-83, or 120-123, wherein the sequencing comprises massively parallel sequencing, Sanger sequencing, or single-molecule sequencing.

125. The method of claim 124, wherein the massively parallel sequencing comprises sequencing by synthesis or sequencing by ligation.

126. The method of claim 125, wherein the massively parallel sequencing comprises sequencing by synthesis.

127. The method of claim 125 or 126, wherein the sequencing by synthesis comprises ILLUMINA™ dye sequencing, ion semiconductor sequencing, or pyrosequencing.

128. The method of claim 127, wherein the sequencing by synthesis comprises ILLUMINA™ dye sequencing.

129. The method of claim 125, wherein the sequencing by ligation comprises sequencing by oligonucleotide ligation and detection (SOLiD™) sequencing or polony-based sequencing.

130. The method of claim 124, wherein the single-molecule sequencing is nanopore sequencing, single-molecule real-time (SMRT™) sequencing, or Helicos™ sequencing.

131. The method of any one of claims 1-130, wherein the pathogen is a gram negative bacterial pathogen or a gram positive bacterial pathogen.

132. The method of any one of claims 1-131, wherein the method comprises detecting any of the panels set forth in Tables 1-15, 19, or 22.

133. A method for identifying a patient infected with an antibiotic resistant pathogen, the method comprising:
(a) providing a biological sample obtained from the subject; and
(b) detecting the presence of an antibiotic resistance gene in the biological sample according to the method of any one of claims 1-132,
wherein the presence of an antibiotic resistance gene in the biological sample obtained from the subject identifies the subject as one who may be infected with an antibiotic resistant bacterial pathogen.

134. The method of claim 133, further comprising selecting an optimized anti-bacterial therapy for the patient based on the presence of the antibiotic resistance gene.

135. The method of any one of claims 1-134, further comprising administering the optimized anti-bacterial therapy to the patient.

136. The method of claim 135, wherein the optimized anti-bacterial therapy comprises one or more antibiotic agents.

137. The method of claim 136, wherein the one or more antibiotic agents is selected from the group consisting of polymyxin B, colistin, tigecycline, ceftazidime-avibactam, meropenem-vaborbactam, aztreonam, and fosfomycin.

138. The method of claim 136 or 137, wherein the antibiotic agent is administered as a monotherapy.

139. The method of claim 136 or 137, wherein the antibiotic agent is administered as a combination therapy.

140. The method of claim 139, wherein the combination therapy comprises one or more additional antibiotic agents selected from the group consisting of an aminoglycoside, colistin, tigecycline, fosfomycin, gentamicin, tobramycin, amikacin, plazomicin, rimfampin, meropenem, doripenem, ertapenem, and imipenem.

141. The method of any one of claims 135-140, wherein the optimized antibacterial therapy is administered to the patient orally, intravenously, intramuscularly, intra-arterially, subcutaneously, or intraperitoneally.

142. A magnetic particle conjugated to a nucleic acid probe, wherein the nucleic acid probe is specific for an antibiotic resistance gene selected from the group consisting of NDM, KPC, IMP, VIM, OXA-48-like, DHA, CMY, FOX, mecA, mecC, vanA, vanB, CTX-M 14, CTX-M 15, mefA, mefE, ermA, ermB, SHV, and TEM.

143. The magnetic particle of claim 137, further comprising an additional nucleic acid probe, wherein the second nucleic acid probe is specific for a second antibiotic resistance gene selected from the group consisting of NDM, KPC, IMP, VIM, OXA-48-like, DHA, CMY, FOX, mecA, mecC, vanA, vanB, CTX-M 14, CTX-M 15, mefA, mefE, ermA, ermB, SHV, and TEM, wherein the second antibiotic resistance gene is selected from IMP, OXA-48-like, DHA, CMY, FOX, CTX-M 14, CTX-M 15, mecC, mefA, mefE, ermA, ermB, or TEM.

144. The magnetic particle of claim 143, comprising a first nucleic acid probe specific for DHA, and a second nucleic acid probe specific for CMY.

145. A magnetic particle conjugated to one or more nucleic acid probes comprising a nucleic acid sequence selected from SEQ ID NOs: 17-32, 57-78, 83, 84, 86, 87, 89, 90, and 93-96, or a nucleic acid sequence having at least 95% sequence identity to any one of SEQ ID NOs: 17-32, 57-78, 83, 84, 86, 87, 89, 90, and 93-96.

146. A removable cartridge comprising a well comprising the magnetic particle of any one of claims 142-145.

147. The removable cartridge of claim 146, further comprising one or more chambers for holding a plurality of reagent modules for holding one or more assay reagents.

148. The removable cartridge of claim 146 or 147, further comprising a chamber comprising beads for lysing cells.

149. The removable cartridge of any one of claims 146-148, further comprising a chamber comprising a polymerase.

150. The removable cartridge of any one of claims 146-149, further comprising a chamber comprising one or more primers.

151. The removable cartridge of claim 150, wherein the one or more primers comprising a nucleic acid sequence selected from SEQ ID NOs:1-16, 33-54, 81, 82, 85, 88, 91, and 92, or a nucleic acid sequence having at least 95% sequence identity to any one of SEQ ID NOs:1-16, 33-54, 81, 82, 85, 88, 91, and 92.

152. A system for the detection of two or more antibiotic resistance genes selected from the group consisting of NDM, KPC, IMP, VIM, OXA-48-like, DHA, CMY, FOX, mecA, mecC, vanA, vanB, CTX-M 14, CTX-M 15, mefA, mefE, ermA, ermB, SHV, and TEM, wherein the two or more antibiotic resistance genes comprises at least one of IMP, OXA-48-like, DHA, CMY, FOX, CTX-M 14, CTX-M 15, mecC, mefA, mefE, ermA, ermB, or TEM, the system comprising:
(a) a first unit comprising (i) a permanent magnet defining a magnetic field; (ii) a support defining a well holding a liquid sample comprising magnetic particles having a mean particle diameter between 600 and 1200 nm, preferably between 650 and 950 nm, and the one or more antibiotic resistance genes and having an RF coil disposed about the well, the RF coil configured to detect a signal produced by exposing the liquid sample to a bias magnetic field created using the permanent magnet and an RF pulse sequence; and (iii) one or more electrical elements in communication with the RF coil, the electrical elements configured to amplify, rectify, transmit, and/or digitize the signal; and
(b) a second unit comprising a removable cartridge sized to facilitate insertion into and removal from the system, wherein the removable cartridge is a modular cartridge comprising (i) a reagent module for holding one or more assay reagents, (ii) a detection module comprising a detection chamber for holding a liquid sample comprising the magnetic particles and the one or more analytes, and, optionally, (iii) a sterilizable inlet module,
wherein the reagent module, the detection module, and, optionally, the sterilizable inlet module, can be assembled into the modular cartridge prior to use, and wherein the detection chamber is removable from the modular cartridge, preferably,
wherein the system further comprises a system computer with processor for implementing an assay protocol and storing assay data, and wherein the removable cartridge further comprises (i) a readable label indicating the analyte to be detected, (ii) a readable label indicating the assay protocol to be implemented, (iii) a readable label indicating a patient identification number, (iv) a readable label indicating the position of assay reagents contained in the cartridge, or (v) a readable label comprising instructions for the programmable processor.

153. A nucleic acid probe comprising a nucleic acid sequence selected from SEQ ID NOs: 17-32, 57-78, 83, 84, 86, 87, 89, 90, and 93-96, or a nucleic acid sequence having at least 95% sequence identity to any one of SEQ ID NOs: 17-32, 57-78, 83, 84, 86, 87, 89, 90, and 93-96.

154. A nucleic acid primer comprising a nucleic acid sequence selected from SEQ ID NOs: 1-16, 33-54, 81, 82, 85, 88, 91, and 92, or a nucleic acid sequence having at least 95% sequence identity to any one of SEQ ID NOs: 1-16, 33-54, 81, 82, 85, 88, 91, and 92.


SEQUENCE LISTING (96 sequences)

SEQ ID NO: 1 - DNA, 20 bp, Artificial Sequence
  Synthetic Construct
ccagctcgcaccgaatgtct

SEQ ID NO: 2 - DNA, 26 bp, Artificial Sequence
  Synthetic Construct
catcttgtcctgatgcgcgtgagtca

SEQ ID NO: 3 - DNA, 19 bp, Artificial Sequence
  Synthetic Construct
tttctgccaccgcgctgac

SEQ ID NO: 4 - DNA, 22 bp, Artificial Sequence
  Synthetic Construct
gcagcaagaaagcccttgaatg

SEQ ID NO: 5 - DNA, 30 bp, Artificial Sequence
  Synthetic Construct
acatttccatagcgacagcacgggcggaat

SEQ ID NO: 6 - DNA, 30 bp, Artificial Sequence
  Synthetic Construct
ggactttggccaagcttctaaatttgcgtc

SEQ ID NO: 7 - DNA, 19 bp, Artificial Sequence
  Synthetic Construct
cgtgcagtctccacgcact

SEQ ID NO: 8 - DNA, 23 bp, Artificial Sequence
  Synthetic Construct
tcgaatgcgcagcaccgggatag

SEQ ID NO: 9 - DNA, 28 bp, Artificial Sequence
  Synthetic Construct
ggctgtgtttttggtggcatcgattatc

SEQ ID NO: 10 - DNA, 27 bp, Artificial Sequence
  Synthetic Construct
tcccacttaaagacttggtgttcatcc

SEQ ID NO: 11 - DNA, 24 bp, Artificial Sequence
  Synthetic Construct
acgggccggtaatgcggatctgga

SEQ ID NO: 12 - DNA, 29 bp, Artificial Sequence
  Synthetic Construct
tattcgccagaatcacaatcgccacctgt

SEQ ID NO: 13 - DNA, 24 bp, Artificial Sequence
  Synthetic Construct
ccgcggcgaaattaagctcagcga

SEQ ID NO: 14 - DNA, 25 bp, Artificial Sequence
  Synthetic Construct
ccaaacagaccaatgctggagttag

SEQ ID NO: 15 - DNA, 29 bp, Artificial Sequence
  Synthetic Construct
attcgttgcactatcgttaactgaataca

SEQ ID NO: 16 - DNA, 23 bp, Artificial Sequence
  Synthetic Construct
ctgtaccgtcggactttccagac

SEQ ID NO: 17 - DNA, 19 bp, Artificial Sequence
  Synthetic Construct
gcgaccggcaggttgatct

SEQ ID NO: 18 - DNA, 25 bp, Artificial Sequence
  Synthetic Construct
catgtcgagataggaagtgtgctgc

SEQ ID NO: 19 - DNA, 20 bp, Artificial Sequence
  Synthetic Construct
cggaaccattcgctaaactc

SEQ ID NO: 20 - DNA, 21 bp, Artificial Sequence
  Synthetic Construct
aggcgcaactgtaagttaccg

SEQ ID NO: 21 - DNA, 28 bp, Artificial Sequence
  Synthetic Construct
gcttaattctcaatctatccccacgtat

SEQ ID NO: 22 - DNA, 26 bp, Artificial Sequence
  Synthetic Construct
ctccagataacgtagtggtttggctg

SEQ ID NO: 23 - DNA, 21 bp, Artificial Sequence
  Synthetic Construct
ctttcatgacgaccgcgtcgg

SEQ ID NO: 24 - DNA, 23 bp, Artificial Sequence
  Synthetic Construct
ctctagaaggactctcatcgagc

SEQ ID NO: 25 - DNA, 20 bp, Artificial Sequence
  Synthetic Construct
attttaaaggtagatgcggg

SEQ ID NO: 26 - DNA, 20 bp, Artificial Sequence
  Synthetic Construct
cgccctgtgatttatgttca

SEQ ID NO: 27 - DNA, 20 bp, Artificial Sequence
  Synthetic Construct
gttttatgcacccaggaagc

SEQ ID NO: 28 - DNA, 19 bp, Artificial Sequence
  Synthetic Construct
tctgctgcggccagtcata

SEQ ID NO: 29 - DNA, 20 bp, Artificial Sequence
  Synthetic Construct
gcggctgccagttttgataa

SEQ ID NO: 30 - DNA, 19 bp, Artificial Sequence
  Synthetic Construct
gtggctaagtgcagcaggc

SEQ ID NO: 31 - DNA, 28 bp, Artificial Sequence
  Synthetic Construct
cgttccactaacacacaagctgattcag

SEQ ID NO: 32 - DNA, 26 bp, Artificial Sequence
  Synthetic Construct
atctcggttgatttcttttcctcggg

SEQ ID NO: 33 - DNA, 26 bp, Artificial Sequence
  Synthetic Construct
acgctttccaatgtgcagtaccagta

SEQ ID NO: 34 - DNA, 26 bp, Artificial Sequence
  Synthetic Construct
tgcgatccagacgaaacgtctcatcg

SEQ ID NO: 35 - DNA, 24 bp, Artificial Sequence
  Synthetic Construct
gtgataccacttcacctcgggcaa

SEQ ID NO: 36 - DNA, 24 bp, Artificial Sequence
  Synthetic Construct
aatacatcgcgacggctttctgcc

SEQ ID NO: 37 - DNA, 23 bp, Artificial Sequence
  Synthetic Construct
agaattacctttgaaagtcaggc

SEQ ID NO: 38 - DNA, 26 bp, Artificial Sequence
  Synthetic Construct
gcttcaaagcctgtcggaattggttt

SEQ ID NO: 39 - DNA, 25 bp, Artificial Sequence
  Synthetic Construct
gggcatttaacgacgaaactggcta

SEQ ID NO: 40 - DNA, 26 bp, Artificial Sequence
  Synthetic Construct
gtgttcggtgaatatccaaggtacgc

SEQ ID NO: 41 - DNA, 26 bp, Artificial Sequence
  Synthetic Construct
atgttggtcccattaactctgaagaa

SEQ ID NO: 42 - DNA, 26 bp, Artificial Sequence
  Synthetic Construct
cacctgtttgagggtggatagcagta

SEQ ID NO: 43 - DNA, 26 bp, Artificial Sequence
  Synthetic Construct
gcagggcaagcagtatcattaatcac

SEQ ID NO: 44 - DNA, 32 bp, Artificial Sequence
  Synthetic Construct
aattaaatcagcaccaatcattatcttcttcc

SEQ ID NO: 45 - DNA, 25 bp, Artificial Sequence
  Synthetic Construct
aagctgctgaccagccagcgtctga

SEQ ID NO: 46 - DNA, 23 bp, Artificial Sequence
  Synthetic Construct
cggcgatttgctgatttcgctcg

SEQ ID NO: 47 - DNA, 26 bp, Artificial Sequence
  Synthetic Construct
tgcagtgctgccataaccatgagtga

SEQ ID NO: 48 - DNA, 25 bp, Artificial Sequence
  Synthetic Construct
agcgcagaagtggtcctgcaacttt

SEQ ID NO: 49 - DNA, 29 bp, Artificial Sequence
  Synthetic Construct
cagtacggaatctttcgtattcatcagga

SEQ ID NO: 50 - DNA, 25 bp, Artificial Sequence
  Synthetic Construct
cagttcgggaagtgcaatacctgca

SEQ ID NO: 51 - DNA, 25 bp, Artificial Sequence
  Synthetic Construct
aattgagcaagcgatttcgggctgt

SEQ ID NO: 52 - DNA, 24 bp, Artificial Sequence
  Synthetic Construct
cgtttagaacgatgccgccatcct

SEQ ID NO: 53 - DNA, 24 bp, Artificial Sequence
  Synthetic Construct
ccttggacggaaatgtagctggca

SEQ ID NO: 54 - DNA, 25 bp, Artificial Sequence
  Synthetic Construct
aatcacatggttgacacctgctgtg

SEQ ID NO: 55 - DNA, 24 bp, Artificial Sequence
  Synthetic Construct
ggaaatctaacgagagagcatgct

SEQ ID NO: 56 - DNA, 19 bp, Artificial Sequence
  Synthetic Construct
cgatgcgtgacacccaggc

SEQ ID NO: 57 - DNA, 22 bp, Artificial Sequence
  Synthetic Construct
ctgtcgagatcaagcctgccga

SEQ ID NO: 58 - DNA, 23 bp, Artificial Sequence
  Synthetic Construct
acaaattgattgcccagctcggt

SEQ ID NO: 59 - DNA, 21 bp, Artificial Sequence
  Synthetic Construct
aatcagcggcgcacgatcttt

SEQ ID NO: 60 - DNA, 23 bp, Artificial Sequence
  Synthetic Construct
aatgctcgctgcaccggtggtat

SEQ ID NO: 61 - DNA, 21 bp, Artificial Sequence
  Synthetic Construct
aaatctgcaacgagctttggg

SEQ ID NO: 62 - DNA, 28 bp, Artificial Sequence
  Synthetic Construct
gtttataagtgggtaaaccgtgaatatc

SEQ ID NO: 63 - DNA, 26 bp, Artificial Sequence
  Synthetic Construct
ttcgtgtcactttaattcaccaagat

SEQ ID NO: 64 - DNA, 21 bp, Artificial Sequence
  Synthetic Construct
aaagccatgcgtctgacatct

SEQ ID NO: 65 - DNA, 22 bp, Artificial Sequence
  Synthetic Construct
aagctccaacatgaagatggct

SEQ ID NO: 66 - DNA, 24 bp, Artificial Sequence
  Synthetic Construct
agatggcaaagatattcaactaac

SEQ ID NO: 67 - DNA, 23 bp, Artificial Sequence
  Synthetic Construct
agtgccatcttgcaaatggcgat

SEQ ID NO: 68 - DNA, 32 bp, Artificial Sequence
  Synthetic Construct
tgcaattggtgtgttagtggatcgtcatgata

SEQ ID NO: 69 - DNA, 23 bp, Artificial Sequence
  Synthetic Construct
cagtggatggtggacgatcgggt

SEQ ID NO: 70 - DNA, 26 bp, Artificial Sequence
  Synthetic Construct
ttgtggtgatttatctgcgggatact

SEQ ID NO: 71 - DNA, 24 bp, Artificial Sequence
  Synthetic Construct
cgccagttaatagtttgcgcaacg

SEQ ID NO: 72 - DNA, 24 bp, Artificial Sequence
  Synthetic Construct
aaagcggttagctccttcggtcct

SEQ ID NO: 73 - DNA, 23 bp, Artificial Sequence
  Synthetic Construct
cgttcagtacaatgcggccgtta

SEQ ID NO: 74 - DNA, 22 bp, Artificial Sequence
  Synthetic Construct
ctgcgggaacggttataactgc

SEQ ID NO: 75 - DNA, 23 bp, Artificial Sequence
  Synthetic Construct
gcacccgatatactttctttgcc

SEQ ID NO: 76 - DNA, 21 bp, Artificial Sequence
  Synthetic Construct
cgccgacaatcaaatcatcct

SEQ ID NO: 77 - DNA, 24 bp, Artificial Sequence
  Synthetic Construct
ttgaccagttccgagcaaatggta

SEQ ID NO: 78 - DNA, 24 bp, Artificial Sequence
  Synthetic Construct
gacagtatcgatgttccagcagct

SEQ ID NO: 79 - DNA, 30 bp, Artificial Sequence
  Synthetic Construct
gagacgttttggatacatgtgaaagaaggc

SEQ ID NO: 80 - DNA, 26 bp, Artificial Sequence
  Synthetic Construct
cgatggttcacgggattctgcaattc

SEQ ID NO: 81 - DNA, 20 bp, Artificial Sequence
  Synthetic Construct
cctcgggcaatggcgcaaac

SEQ ID NO: 82 - DNA, 22 bp, Artificial Sequence
  Synthetic Construct
atcgcgacggctttctgcctta

SEQ ID NO: 83 - DNA, 18 bp, Artificial Sequence
  Synthetic Construct
gggcgcagctggtgacat

SEQ ID NO: 84 - DNA, 20 bp, Artificial Sequence
  Synthetic Construct
aagatcgtgcgccgctgatt

SEQ ID NO: 85 - DNA, 24 bp, Artificial Sequence
  Synthetic Construct
tattcatcaggaagtcgagccgga

SEQ ID NO: 86 - DNA, 22 bp, Artificial Sequence
  Synthetic Construct
gcagttataaccgttcccgcag

SEQ ID NO: 87 - DNA, 23 bp, Artificial Sequence
  Synthetic Construct
taacggccgcattgtactgaacg

SEQ ID NO: 88 - DNA, 24 bp, Artificial Sequence
  Synthetic Construct
aagatcaacacgggcaagccctct

SEQ ID NO: 89 - DNA, 22 bp, Artificial Sequence
  Synthetic Construct
aaaggctataaagatgatgcag

SEQ ID NO: 90 - DNA, 29 bp, Artificial Sequence
  Synthetic Construct
gagtatttataacaacatgaaaaatgatt

SEQ ID NO: 91 - DNA, 26 bp, Artificial Sequence
  Synthetic Construct
atgtgggtccaattaattctgacgag

SEQ ID NO: 92 - DNA, 26 bp, Artificial Sequence
  Synthetic Construct
ctccagtttttggttgtaatgctgta

SEQ ID NO: 93 - DNA, 21 bp, Artificial Sequence
  Synthetic Construct
ggcttagaacgcctctatgat

SEQ ID NO: 94 - DNA, 31 bp, Artificial Sequence
  Synthetic Construct
agagtacaagaaagtatttataaacatatga

SEQ ID NO: 95 - DNA, 24 bp, Artificial Sequence
  Synthetic Construct
gcgaccggcaggttgatctcctgc

SEQ ID NO: 96 - DNA, 28 bp, Artificial Sequence
  Synthetic Construct
cggcatgtcgagataggaagtgtgctgc